(19) United States
(12) Patent Application Publication
(10) Pub. No.: US 2025/0263753 A1
(43) Pub. Date: Aug. 21, 2025

(54) BRIDGED CYCLE-BASED INHIBITORS OF DNA-DEPENDENT PROTEIN KINASE AND COMPOSITIONS AND APPLICATION IN GENE EDITING

(71) Applicant: Juno Therapeutics, Inc., Seattle, WA (US)

(72) Inventors: Yucheng Mu, Cambridge, MA (US); Jennifer X. Qiao, Cambridge, MA (US); Yilin Meng, Cambridge, MA (US); Laura Akullian D'Agostino, Sudbury, MA (US); Cédric Cleyrat, Seattle, WA (US); Ashley Thelen, Seattle, WA (US); Jenna Fernandez, Lynnwood, WA (US)

(21) Appl. No.: 19/169,991

(22) Filed: Apr. 3, 2025

Related U.S. Application Data
(63) Continuation of application No. PCT/US2024/043446, filed on Aug. 22, 2024.
(60) Provisional application No. 63/647,467, filed on May 14, 2024.
(60) Provisional application No. 63/578,851, filed on Aug. 25, 2023.

(51) Int. Cl. C12N 15/90; C07D 487/04; C07D 519/00; C12N 9/22; C12N 15/11

(52) U.S. Cl. CPC C12N 15/907; C07D 487/04; C07D 519/00; C12N 9/226; C12N 15/11; C12N 2310/20

(57) ABSTRACT

The present disclosure is directed to DNA-PK inhibitors having the Formula (I),

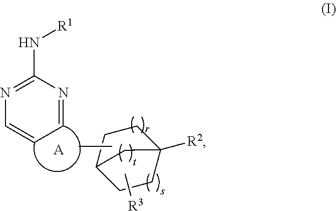

or a pharmaceutically acceptable salt, stereoisomer, solvate, prodrug, or tautomer thereof, methods of preparing the foregoing, and compositions thereof, as well as methods of use for the compounds of Formula (I), in combination with a DNA cutting agent.

### CROSS-REFERENCE

This application is a continuation of and claims priority to the International Patent Application No. PCT/US2024/043446, filed Aug. 22, 2024 which claims the benefit of and priority to U.S. Provisional Patent Application No. 63/578,851 filed on Aug. 25, 2023, and U.S. Provisional Patent Application No. 63/647,467 filed on May 14, 2024. The entire contents of each of the foregoing is incorporated herein by reference in their entireties.

### FIELD

The present disclosure relates generally to compounds, compositions, methods, and kits for increasing genome editing efficiency by administering an inhibitor of DNA protein-kinase (DNA-PK) of general formula (I) and a genome editing system to a eukaryotic cell(s). The present disclosure further relates to compositions including the DNA-PK inhibitors of general formula (I), methods of inserting a polynucleotide of interest into the genome of a eukaryotic cell, and kits for inserting a gene of interest into the genome of a eukaryotic cell. The methods and kits can improve the efficiency of CRISPR/Cas-mediated polynucleotide insertion in cells, in particular in CRISPR-engineered CAR-T cells.

### INCORPORATION BY REFERENCE OF SEQUENCE LISTING

This application contains a Sequence Listing which has been submitted in .XML format via EFS-WEB and is hereby incorporated by reference in its entirety. Said .XML copy, created on Aug. 24, 2023 is named 055920-611P01US_SeqList_ST26.xml and is 75 KB in size.

### BACKGROUND

The development of cost-efficient and reliable methods for precise targeted alterations to the genome of living cells has been a long-standing goal. Genome editing has the potential to eliminate genes responsible for a particular disorder (i.e., a gene “knock-out”), or alternatively, provide a means for gene manipulation or insertion to correct a genetic deficiency or enhance a biological process via a gene “knock-in.” Genome editing can be applied for treatment of a multitude of disorders, including treatment of inherited disorders, hematological disorders and cancer, and in methods of immunotherapy. Clustered Regularly Interspaced Short Palindromic Repeats (CRISPR) and CRISPR associated (Cas) systems are prokaryotic immune systems (Ishino et al., Journal of Bacteriology 169:5429-5433 (1987)), which provide immunity against viruses and plasmids by targeting the nucleic acids of the viruses and plasmids in a sequence-specific manner (Soret et al., Nature Reviews Microbiology 6:181-186 (2008)). Since its original discovery, multiple groups have performed extensive research around potential applications of the CRISPR system in genetic engineering, including gene editing (Jinek et al., Science 337(6096):816-821 (2012); Cong et al, Science 339(6121):819-823 (2013); and Mali et al., Science 339(6121):823-826 (2013)). The CRISPR-Cas9 gene editing system has been used successfully in a wide range of organisms and cell lines.

The Cas9 endonuclease generates a double-stranded DNA break at the target sequence, upstream of a protospacer adjacent motif (PAM). The target sequence can then be removed, or a sequence of interest can be inserted into the target sequence using an endogenous repair pathway of the cell. Endogenous DNA repair pathways include the Nonhomology Mediated End-Joining (NHEJ) pathway, Microhomology Mediated End-Joining (MMEJ) pathway, and the Homology Directed Repair (HDR) pathway.

NHEJ, MMEJ, and HDR pathways repair double-stranded DNA breaks, but repair of such double-stranded DNA breaks may result in insertions or deletions at the double stranded break site. In NHEJ, a homologous template is not required for repairing breaks in the DNA. NHEJ repair can be error-prone, although errors are decreased when the DNA break includes compatible overhangs. NHEJ and MMEJ are mechanistically distinct DNA repair pathways with different subsets of DNA repair enzymes involved in each of them. Unlike NHEJ, which can be precise in some cases, or error-prone in some cases, MMEJ is always error-prone and results in both deletion and insertions at the site under repair. MMEJ-associated deletions are due to the micro-homologies (2-10 base pairs) at both sides of a double-strand break. In contrast, HDR requires a homologous template to direct repair, but HDR repairs are typically high-fidelity and less error prone. HDR-driven repair of double-stranded DNA breaks is therefore preferable to NHEJ- or MMEJ-mediated repair; however, in many cell types HDR is limited by the activity of NHEJ at all cell cycle stages, and HDR is primarily utilized in the S/G2 phase of cell growth (Mao et al., Cell Cycle, 7:2902-2906 (2008)).

The ability to modify the genome of any cell at a precise location has improved with the recent discovery and implementation of CRISPR/Cas9 editing technology. However, the capacity to introduce specific directed changes at given loci is hindered by the fact that the major cellular repair pathway that occurs following Cas9-mediated DNA cleavage is the erroneous non-homologous end joining (NHEJ) pathway. Homology-directed recombination (HDR) is less efficient than NHEJ, reducing editing efficiencies in eukaryotic cells. While the achievement of insertion or deletions (indels) from NHEJ is up to 70% effective in some reports, the efficiency of HDR remains challenging, with rates at less than 1%. Accordingly, there is a need for increasing genome editing efficiency, in particular, HDR efficiency.

Studies have shown that reduced NHEJ activity in vivo results in increases in HDR activity, and this phenomenon can be exploited to increase the efficiency of HDR-mediated CRISPR/Cas9 precision genome engineering (Pierce et al. Genes Dev., 15, 3237-3242 (2001); Ma et al. RNA Biol., 13, 605-612 (2016); Maruyama, et al. Nat. Biotechnol., 33, 538-542 (2015); Robert et al. Genome Med., 7, 93 (2015)).

DNA-dependent protein kinase (DNA-PK) is a nuclear serine/threonine kinase that has been shown to be essential in DNA double stranded break repair machinery. In mammals, the predominant pathway for repair of double stranded DNA breaks is the non-homologous end joining (NHEJ) pathway which is functional regardless of the phase of the cell cycle and acts by removing non-ligatable ends and ligating ends of double strand breaks. There is a need for potent and selective DNA-PK inhibitors (DNA-PKi) that transiently block the NHEJ pathway to promote DNA repair via the desirable HDR pathway, therefore, improving the efficiency of CRISPR/Cas-mediated polynucleotide insertion in cells, such as CRISPR CAR-T cells.

### SUMMARY

A first aspect of the present disclosure relates to compounds of Formula (I)

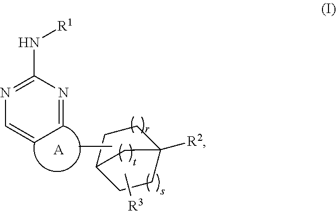

or a pharmaceutically acceptable salt, stereoisomer, solvate, prodrug, or tautomer thereof, wherein:
A is a 5- or 6-membered heteroaryl or heterocycloalkyl group containing at least one heteroatom selected from the group consisting of N, O, and S, wherein the heteroaryl or heterocycloalkyl is optionally substituted with one or more R⁴.
R¹ is an aryl or heteroaryl containing at least one heteroatom selected from the group consisting of N, O, S, and Se, wherein the aryl or heteroaryl is optionally substituted with one or more R⁵;
R² is H, halogen, —(CH₂)ₙ—CN, —OH, —(CH₂)ₙ—O—C₁-C₄ alkyl, C₁-C₄ alkyl, C₁-C₄ alkoxy, C₁-C₄ haloalkyl, C₂-C₄ alkenyl, C₂-C₄ alkynyl, or C(O)NH₂;
R³ is independently selected from the group consisting of H, F, C₂-C₄ alkenyl, C₂-C₄ alkynyl, CN, OH, CH₂OH, NH₂, CH₂NH₂, and C₁-C₄ alkyl;
each R⁴ is independently selected from the group consisting of halogen, oxo, thioxo, C₁-C₄ alkyl, C₁-C₄ alkoxy, CD₃, CD₂CD₃, and C₁-C₆ haloalkyl; or
two geminal R⁴ together with the intervening geminal carbon atom, form a C₃-C₆ cycloalkyl;
each R⁵ is independently selected from the group consisting of halogen, NH₂, C(O)NH₂, OH, —CN, C(O)NHR⁷, C₁-C₄ alkyl, C₂-C₄ alkenyl, C₂-C₄ alkynyl, CD₃, CD₂CD₃, C₁-C₆ alkoxy, C₁-C₆ haloalkyl, C₃-C₆ cycloalkyl, heterocycloalkyl, heteroaryl, and aryl, wherein the alkyl, alkoxy, haloalkyl, cycloalkyl, heterocycloalkyl, heteroaryl, or aryl is optionally substituted with one or more R⁶.
each R⁶ is independently selected from the group consisting of halogen, OH, oxo, NH₂, CHO, C₁-C₄ alkyl, and C₁-C₆ alkoxy;
each R⁷ is independently selected from H and C₁-C₄ alkyl;
each n is independently an integer from 0-4;
r is an integer from 0 to 2;
s is an integer from 0 to 2; and
t is an integer from 1 to 2,
provided that when A is a 5-membered heterocycle containing N—C(O)—N in the ring and R¹ is

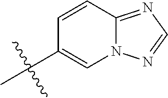 [SMILES: *c1ccc2ncnn2c1]

optionally substituted with one or more R⁵, then

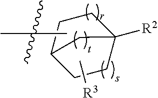

is not

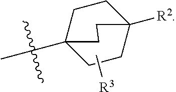

Another aspect of the present disclosure relates to compounds of Formula (I):

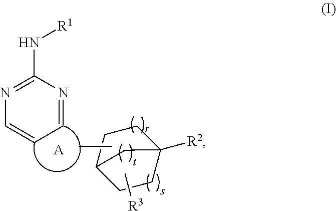

or a pharmaceutically acceptable salt, stereoisomer, solvate, prodrug, or tautomer thereof, wherein:
A is a 5- or 6-membered heteroaryl or heterocycloalkyl group containing at least one heteroatom selected from the group consisting of N, O, and S, wherein the heteroaryl or heterocycloalkyl is optionally substituted with one to three R⁴;
R¹ is an aryl or heteroaryl containing at least one heteroatom selected from the group consisting of N, O, S, and Se, wherein the aryl or heteroaryl is optionally substituted with one to four R⁵.
R² is H, halogen, —(CH₂)ₙ—CN, —OH, —(CH₂)ₙ—O—C₁-C₄ alkyl, C₁-C₄ alkyl, C₁-C₄ alkoxy, C₁-C₄ haloalkyl, C₂-C₄ alkenyl, C₂-C₄ alkynyl, or C(O)NH₂;
R³ is independently selected from the group consisting of H, F, C₂-C₄ alkenyl, C₂-C₄ alkynyl, CN, OH, CH₂OH, NH₂, CH₂NH₂, and C₁-C₄ alkyl;
each R⁴ is independently selected from the group consisting of halogen, oxo, thioxo, C₁-C₄ alkyl, C₁-C₄ alkoxy, and CD₃, CD₂CD₃, C₁-C₆ haloalkyl; or
two geminal R⁴ together with the intervening geminal carbon atom, form a C₃-C₆ cycloalkyl;
each R⁵ is independently selected from the group consisting of halogen, NH₂, OH, —CN, C(O)NH₂, C(O)NHR⁷, C₁-C₄ alkyl, C₂-C₄ alkenyl, C₂-C₄ alkynyl, CD₃, CD₂CD₃, C₁-C₆ alkoxy, C₁-C₆ haloalkyl, C₃-C₆ cycloalkyl, heterocycloalkyl, heteroaryl, and aryl, wherein the alkyl, alkoxy, haloalkyl, cycloalkyl, heterocycloalkyl, heteroaryl, or aryl is optionally substituted with one to three R⁶;
each R⁶ is independently selected from the group consisting of halogen, OH, oxo, NH₂, CHO, C₁-C₄ alkyl, and C₁-C₆ alkoxy;
each R⁷ is independently selected from H and C₁-C₄ alkyl;
each n is independently an integer from 0-4;
r is an integer from 0 to 2;
s is an integer from 0 to 2; and
t is an integer from 1 to 2,
provided that when A is a 5-membered heterocycle containing N—C(O)—N in the ring and R¹ is

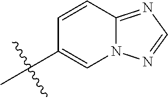 [SMILES: *c1ccc2ncnn2c1]

optionally substituted with one to four R⁵, then

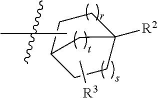

is not

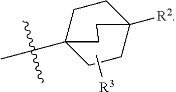

Another aspect of the present disclosure is directed to pharmaceutical compositions comprising a compound of Formula (I), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the present disclosure is directed to a composition comprising (a) a DNA protein kinase inhibitor (DNA-PKI) and (b) a DNA cutting agent, wherein the DNA-PKI is a compound of Formula (I), or a pharmaceutically acceptable salt, hydrate, solvate, prodrug, stereoisomer, or tautomer thereof.

Another aspect of the present disclosure is directed to a method for targeted genome editing in a cell, comprising contacting the cell with a DNA cutting agent and a DNA-PKI, wherein the DNA-PKI is a compound of Formula (I), or a pharmaceutically acceptable salt, hydrate, solvate, prodrug, stereoisomer, or tautomer thereof.

Another aspect of the present disclosure is directed to a method for repairing a double stranded DNA break in the genome of a cell, comprising contacting the cell with a DNA cutting agent and a DNA-PKI, wherein the DNA-PKI is a compound of Formula (I), or a pharmaceutically acceptable salt, hydrate, solvate, prodrug, stereoisomer, or tautomer thereof.

Another aspect of the present disclosure is directed to a method for inhibiting or suppressing repair of a DNA break in a cell via a nonhomologous end joining (NHEJ) pathway, comprising contacting the cell with a DNA cutting agent and a DNA-PKI, wherein the DNA-PKI is a compound of Formula (I), or a pharmaceutically acceptable salt, hydrate, solvate, prodrug, stereoisomer, or tautomer thereof.

Another aspect of the present disclosure is directed to a method for targeted insertion of a donor DNA into the genome of a cell, comprising contacting the cell with a DNA cutting agent, the donor DNA, and a DNA-PKI, wherein the DNA-PKI is a compound of Formula (I), or a pharmaceutically acceptable salt, hydrate, solvate, prodrug, stereoisomer, or tautomer thereof.

Another aspect of the present disclosure relates to compounds of Formula (I), and pharmaceutically acceptable salts, hydrates, solvates, prodrugs, stereoisomers, tautomers, or pharmaceutical compositions thereof, for use in the manufacture of a medicament for cell therapy.

Another aspect of the present disclosure relates to the use of a compound of Formula (I), or a pharmaceutically acceptable salt, hydrate, solvate, prodrug, stereoisomer, tautomer, or pharmaceutical composition thereof, in the treatment of a cell.

In some aspects, the present disclosure provides a method of preparing a compound of the present disclosure.

In some aspects, the present disclosure provides a method of preparing a compound, comprising one or more steps described herein.

Other features and advantages of the disclosure will be apparent from the following detailed description and claims.

### BRIEF DESCRIPTION OF DRAWINGS

FIG. 1 shows the effect of DNA-PK inhibitor compounds on cell viability 5 days after electroporation. Live cells are shown as a percentage of total cells.

FIG. 2 shows the effect of DNA-PK inhibitor compounds on T cell proliferation 5 days after electroporation. Total live cell counts (×10e6) are shown.

FIG. 3 shows the effect of DNA-PK inhibitor compounds on CAR insertion into the TRAC locus 5 days after electroporation. Frequency of CAR+ T cells is shown as a percentage of total live cells.

FIG. 4 shows the effect of DNA-PK inhibitor compounds on CAR insertion into the TRAC locus 5 days after electroporation. KI efficiency is shown as percentage change over the untreated control condition (calculated by dividing the % CAR+ of DNA-PKi treated by untreated, subtracting 1, and multiplying by 100).

FIG. 5 shows the effect of DNA-PK inhibitor compounds on total CAR+ cell yields 5 days after electroporation. Relative CAR+ yield is shown as percentage change over the untreated control condition (calculated by dividing the number of CAR+ cells in DNA-PKi treated condition by untreated, subtracting 1, and multiplying by 100).

FIG. 6 shows the effect of DNA-PK inhibitor compounds (Compound 1 and Compound 78) on total CAR+ cell yields 5 days after electroporation. Relative CAR+ yield is shown as percentage change over the untreated control condition (calculated by dividing the number of CAR+ cells in DNA-PKi treated condition by untreated, subtracting 1, and multiplying by 100).

### DETAILED DESCRIPTION

### Definitions

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed present disclosure. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —COOH, —CH₂CN, —O—(C₁-C₆) alkyl, (C₁-C₆) alkyl, (C₁-C₆) alkoxy, (C₁-C₆) haloalkyl, (C₁-C₆) haloalkoxy, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, —NH((C₁-C₆) alkyl), —N((C₁-C₆) alkyl)₂, —NHC(O)(C₁-C₆) alkyl, —C(O)NH(C₁-C₆) alkyl, —S(O)₂(C₁-C₆) alkyl, —S(O)NH(C₁-C₆) alkyl, and S(O)N((C₁-C₆) alkyl)₂. The substituents can themselves be optionally substituted. “Optionally substituted” as used herein also refers to substituted or unsubstituted whose meaning is described below.

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a RM, and formulation into an efficacious therapeutic agent. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

As used herein, “Alkyl” refers to optionally substituted, straight and branched chain aliphatic groups having from 1 to 30 carbon atoms. “C1, C2, C3, C4, C5 or C6 alkyl” or “C₁-C₆ alkyl” is intended to include C1, C2, C3, C4, C5 or C6 straight chain (linear) saturated aliphatic hydrocarbon groups and C3, C4, C5 or C6 branched saturated aliphatic hydrocarbon groups. For example, C₁-C₆ alkyl is intends to include C1, C2, C3, C4, C5 and C6 alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms. The term “heteroalkyl” as used herein contemplates an alkyl with one or more heteroatoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Alkoxy” refers to a straight or branched chain saturated hydrocarbon containing 1-30 carbon atoms containing a terminal “O” in the chain, i.e., —O(alkyl). Examples of alkoxy groups include without limitation, methoxy, ethoxy, propoxy, butoxy, t-butoxy, or pentoxy groups.

As used herein, the term “alkenyl” includes unsaturated or partially unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂₋₆ for straight chain, C₃₋₆ for branched chain). The term “C₂₋₆” includes alkenyl groups containing two to six carbon atoms. The term “C₃₋₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂₋₆ for straight chain, C₃₋₆ for branched chain). The term “C₂₋₆” includes alkynyl groups containing two to six carbon atoms. The term “C₃₋₆” includes alkynyl groups containing three to six carbon atoms.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro) system having 3 to 30 carbon atoms (e.g., C₃₋₂, C₃₋₁₀, C₃₋₈, or C₃₋₆). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic or bicyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofurran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocycloalkyl, only one of the rings in the heterocycloalkyl needs to be non-aromatic (e.g., 1,3-dihydrobenzo[c]isoxazol-3-yl).

As used herein, the term “optionally substituted heterocycloalkyl” refers to unsubstituted heterocycloalkyl having designated substituents replacing one or more hydrogen atoms on one or more carbon or heteroatom. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless otherwise specifically defined, the term “aryl” refers to cyclic, aromatic hydrocarbon groups that have 1 to 3 aromatic rings, including monocyclic or bicyclic groups such as phenyl, biphenyl, or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁₋₆) alkyl, (C₁₋₆) alkyl, —O—(C₂₋₆) alkenyl, —O—(C₂₋₆) alkynyl, (C₂₋₆) alkenyl, (C₂₋₆) alkynyl, —OH, —OP(O)(OH)2, —OC(O)(C₁₋₆) alkyl, —C(O)(C₁₋₆) alkyl, —OC(O)O(C₁₋₆) alkyl, —NH2, NH((C₁₋₆) alkyl), N((C₁₋₆)alkyl)2, —S(O)2-(C₁₋₆) alkyl, —S(O)NH(C₁₋₆) alkyl, and —S(O)N((C₁₋₆) alkyl)2. The substituents can themselves be optionally substituted. Furthermore, when containing two or more fused rings, the aryl groups herein defined may have a saturated or partially unsaturated ring fused with a fully unsaturated aromatic ring Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, 10,11-dihydro-5H-dibenzo[a,d][7]annulenyl, and the like. Furthermore, when containing two or more fused rings, the aryl groups herein defined may have a saturated or partially unsaturated heterocyclic ring fused with a fully unsaturated aromatic ring. Exemplary ring systems of these aryl groups include, but are not limited to, benzo[d][1,3]dioxol-5-yl, 2,3-dihydrobenzo[b][1,4]dioxin-6-yl, benzo[d]isoxazol-3(2H)-on-6-yl, benzo[d]oxazol-2(3H)-on-6-yl, and benzo[d]oxazol-2(3H)-on-5-yl.

Unless otherwise specifically defined, “heteroaryl” means a monovalent monocyclic or polycyclic aromatic radical of 5 to 24 ring atoms, containing one or more ring heteroatoms selected from N, O, S, P, Se, or B, the remaining ring atoms being C. Heteroaryl as herein defined also means a bicyclic heteroaromatic group wherein the heteroatom is selected from N, O, S, P, Se, or B. Heteroaryl as herein defined also means a tricyclic heteroaromatic group containing one or more ring heteroatoms selected from N, O, S, P, Se, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolinyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1λ2-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzoxazolyl, benzisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with a fully unsaturated aromatic ring, e.g., a 5-membered heteroaromatic ring containing 1 to 3 heteroatoms selected from N, O, S, P, Se, or B, or a 6-membered heteroaromatic ring containing 1 to 3 nitrogens, wherein the saturated or partially unsaturated ring includes 0 to 4 heteroatoms selected from N, O, S, P, Se, or B, and is optionally substituted with one or more oxo. In heteroaryl ring systems containing more than two fused rings, a saturated or partially unsaturated ring may further be fused with a saturated or partially unsaturated ring described herein. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-11H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, or benzo[c][1,2]oxaborol-1(3H)-olyl.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

When a bond to a substituent is shown to cross a bond connecting two atoms in a ring, then such substituent may be bonded to any atom in the ring. For example, in the structure

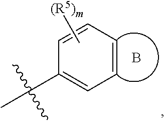

an R⁵ substituent may replace any hydrogen attached to an atom in the ring, including hydrogens attached to atoms of the ring indicated by B. When a substituent is listed without indicating the atom via which such substituent is bonded to the rest of the compound of a given formula, then such substituent may be bonded via any atom in such formula. For example, the structure

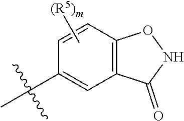

encompasses

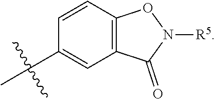

Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an OH or —O—.

As used herein, the term “halo” or “halogen” refers to fluoro, chloro, bromo and iodo.

The term “haloalkyl” or “haloalkoxyl” refers to an alkyl or alkoxyl substituted with one or more halogen atoms. Examples of haloalkyl groups include, but are not limited to, trifluoromethyl, difluoromethyl, pentafluoroethyl, trichloromethyl, etc. Examples of haloalkoxy groups include, but are not limited to, trifluoromethoxy, difluoromethoxy, pentafluoroethoxy, trichloromethoxy, etc.

As used herein, the term “cyano” refers to a nitrile radical (e.g., —CN).

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, and if the solvent is alcohol, the solvate formed is an alcoholate. Hydrates are formed by the combination of one or more molecules of water with one molecule of the substance in which the water retains its molecular state as H₂O.

As described herein, isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.” The compounds of Formula (I) may have one or more asymmetric carbon atom and may occur as racemates, racemic mixtures and as individual enantiomers or diastereomers.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism. In keto-enol tautomerism a simultaneous shift of electrons and a hydrogen atom occurs.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

The present disclosure also contemplates isotopically-labelled compounds of Formula I (e.g., those labeled with D, ²H, or ¹⁴C). Substitution with heavier isotopes such as deuterium may afford certain therapeutic advantages resulting from greater metabolic stability (e.g., increased in vivo half-life or reduced dosage requirements). Isotopically labelled compounds of Formula I can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising an effective amount of a disclosed compound and a pharmaceutically acceptable carrier.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton presents in the parent compound either is replaced by a metal ion, e.g., an alkali metal ion, an alkaline earth ion, or an aluminum ion, or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” when used in connection with a compound is an amount effective for use in a cell therapy.

The term “carrier” as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug” as used in this disclosure, means a compound which is convertible in vivo by metabolic means (e.g., by hydrolysis) to a disclosed compound.

The present disclosure relates to compounds and compositions that are capable of inhibiting DNA-dependent protein kinase (DNA-PK) in a subject or in a biological sample. In a first aspect of the present disclosure, the compounds of Formula (I) are described:

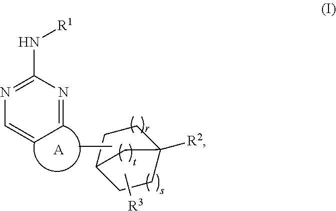

and pharmaceutically acceptable salts, hydrates, solvates, prodrugs, stereoisomers, and tautomers thereof, wherein A, R¹, R², R³, r, s, and t are described herein, provided that when A is a 5-membered heterocycle containing N—C(O)—N in the ring and R¹ is

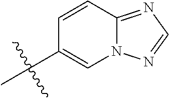 [SMILES: *c1ccc2ncnn2c1]

optionally substituted with one or more R⁵, then

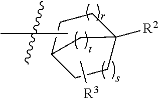

is not

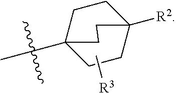

The details of the present disclosure are set forth in the accompanying description below. Although methods and materials similar or equivalent to those described herein can be used in the practice or testing of the present disclosure, illustrative methods and materials are now described. Other features, objects, and advantages of the present disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this present disclosure belongs. All patents and publications cited in this specification are incorporated herein by reference in their entireties.

In some embodiments, R¹ is an aryl or heteroaryl containing at least one heteroatom selected from the group consisting of N, O, S, and Se, wherein the aryl or heteroaryl is optionally substituted with one or more R⁵, wherein each R⁵ is independently selected from the group consisting of halogen, NH₂, OH, —CN, C(O)NH₂, C(O)NHR⁷, C₁-C₄ alkyl, C₂-C₄ alkenyl, C₂-C₄ alkynyl, CD₃, CD₂CD₃, C₁-C₆ alkoxy, C₁-C₆ haloalkyl, C₃-C₆ cycloalkyl, heterocycloalkyl, heteroaryl, and aryl, wherein the alkyl, alkoxy, haloalkyl, cycloalkyl, heterocycloalkyl, heteroaryl, or aryl is optionally substituted with one or more R⁶, and wherein each R⁷ is independently selected from H and C₁-C₄ alkyl. In some embodiments, R¹ is a 6- to 10-membered aryl or 5- to 12-membered heteroaryl containing at least one heteroatom selected from the group consisting of N, O, S, and Se, wherein the aryl or heteroaryl is optionally substituted with one or more R⁵. In some embodiments, R¹ is a 6- to 10-membered aryl or 5- to 12-membered heteroaryl containing one to four heteroatoms selected from the group consisting of N, O, S, and Se, wherein the aryl or heteroaryl is optionally substituted with one or more R⁵. In some embodiments, R¹ is a 6- to 10-membered aryl or 5- to 12-membered heteroaryl containing one to four heteroatoms selected from the group consisting of N, O, S, and Se, wherein the aryl or heteroaryl is optionally substituted with one to four R⁵. In some embodiments, R¹ is a 6- to 10-membered aryl or 5- to 12-membered heteroaryl containing one to three heteroatoms selected from the group consisting of N, O, S, and Se, wherein the aryl or heteroaryl is optionally substituted with one to four R⁵. In some embodiments, R¹ is a 6- to 10-membered aryl or 6- to 12-membered heteroaryl containing one to three heteroatoms selected from the group consisting of N, O, S, and Se, wherein the aryl or heteroaryl is optionally substituted with one to four R⁵.

In some embodiments, each R⁵ is independently selected from the group consisting of halogen, NH₂, OH, —CN, C(O)NH₂, C(O)NHR⁷, C₁-C₄ alkyl, C₂-C₄ alkenyl, C₂-C₄ alkynyl, CD₃, CD₂CD₃, C₁-C₆ alkoxy, C₁-C₆ haloalkyl, C₃-C₆ cycloalkyl, heterocycloalkyl, heteroaryl, and aryl, wherein the alkyl, alkoxy, haloalkyl, cycloalkyl, heterocycloalkyl, heteroaryl, or aryl is optionally substituted with one or more R⁶. In some embodiments, each R⁵ is independently selected from the group consisting of halogen, C₁-C₄ alkyl, CD₃, CD₂CD₃, C(O)NH₂, C(O)NHR⁷, and heteroaryl, wherein the heteroaryl is optionally substituted with one or more R⁶, and wherein each R⁷ is independently selected from H and C₁-C₄ alkyl.

In some embodiments, each R⁶ is independently selected from the group consisting of halogen, OH, oxo, NH₂, CHO, C₁-C₄ alkyl, CD₃, and C₁-C₆ alkoxy. In some embodiments, each R⁶ is C₁-C₄ alkyl.

In some embodiments, R¹ is:

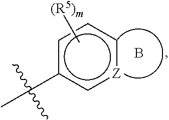

wherein Z is N or C; B is a 5- or 6-membered aryl, heterocyclyl, or heteroaryl, wherein the heterocyclyl or heteroaryl contains at least one heteroatom selected from the group consisting of N, O, S, and Se; and m is an integer from 1 to 4. In some embodiments, Z is N or C; B is a 5- or 6-membered aryl or heteroaryl containing at least one heteroatom selected from the group consisting of N, O, S, and Se; and m is an integer from 1 to 4. In some embodiments, Z is N or C; B is a 5- or 6-membered heterocyclyl or heteroaryl containing at least one heteroatom selected from the group consisting of N, O, S, and Se; and m is an integer from 1 to 4. In some embodiments, Z is N or C; B is a 5- or 6-membered aryl, heterocyclyl, or heteroaryl, wherein the heterocyclyl or heteroaryl contains at least one heteroatom selected from the group consisting of N, O, S, and Se; and m is an integer from 1 to 3. In some embodiments, Z is N or C; B is a 5- or 6-membered aryl or heteroaryl containing at least one heteroatom selected from the group consisting of N, O, S, and Se; and m is an integer from 1 to 3. In some embodiments, Z is N or C; B is a 5- or 6-membered heterocyclyl or heteroaryl containing at least one heteroatom selected from the group consisting of N, O, S, and Se; and m is an integer from 1 to 3. In some embodiments, at least one R⁵ is CD₃ or methyl.

In some embodiments, R¹ is:

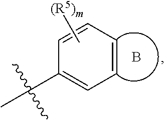

wherein B is a 5- or 6-membered aryl, heterocyclyl, or heteroaryl, wherein the heterocyclyl or heteroaryl contains at least one heteroatom selected from the group consisting of N, O, S, and Se; and m is an integer from 1 to 4. In some embodiments, B is a 5- or 6-membered aryl or heteroaryl containing at least one heteroatom selected from the group consisting of N, O, S, and Se; and m is an integer from 1 to 4. In some embodiments, B is a 5- or 6-membered heterocyclyl or heteroaryl containing at least one heteroatom selected from the group consisting of N, O, S, and Se; and m is an integer from 1 to 4. In some embodiments, B is a 5- or 6-membered aryl, heterocyclyl, or heteroaryl, wherein the heterocyclyl or heteroaryl contains at least one heteroatom selected from the group consisting of N, O, S, and Se; and m is an integer from 1 to 3. In some embodiments, B is a 5- or 6-membered aryl or heteroaryl containing at least one heteroatom selected from the group consisting of N, O, S, and Se; and m is an integer from 1 to 3. In some embodiments, B is a 5- or 6-membered heterocyclyl or heteroaryl containing at least one heteroatom selected from the group consisting of N, O, S, and Se; and m is an integer from 1 to 3. In some embodiments, at least one R⁵ is CD₃ or methyl.

In some embodiments, R¹ is selected from the group consisting of:

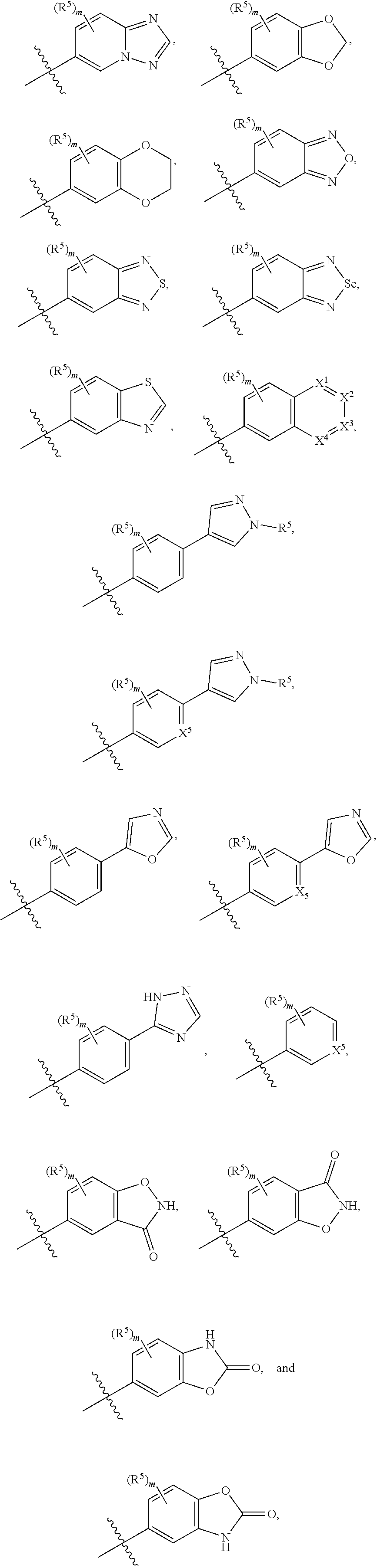

wherein X¹, X², X³, X⁴ and X⁵ are each independently N, CH, or CQ(R⁵); and m is an integer from 1 to 3, as valency allows.

In some embodiments, R¹ is:

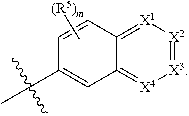

In some embodiments, R¹ is selected from the group consisting of:

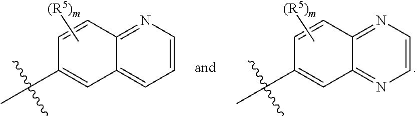

In some embodiments, R¹ is selected from the group consisting of:

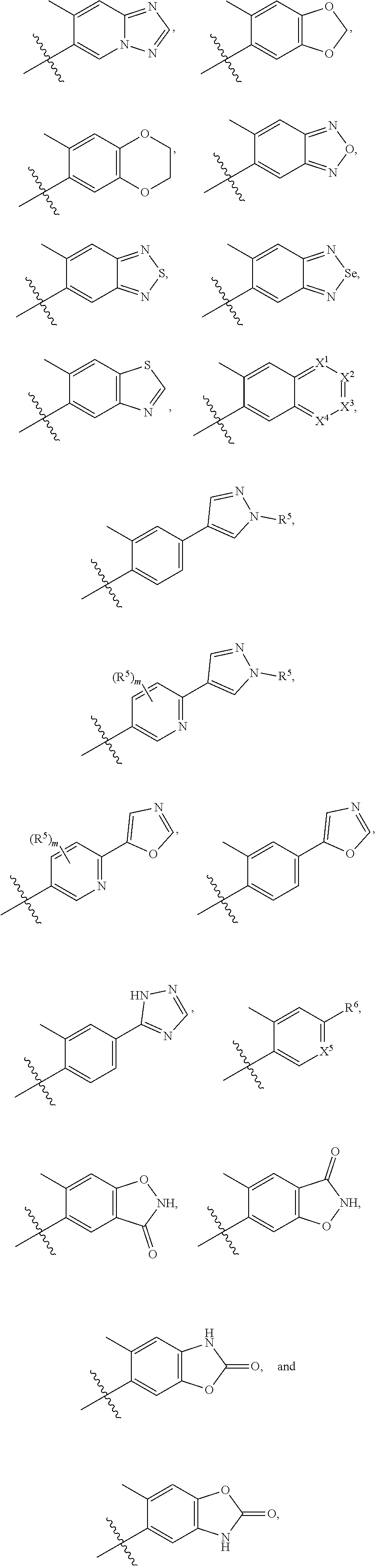

wherein X¹, X², X³, X⁴ and X⁵ are each independently N, CH, or C(R⁵).

In some embodiments, R¹ is:

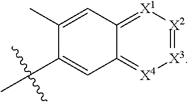

In some embodiments, R¹ is selected from the group consisting of

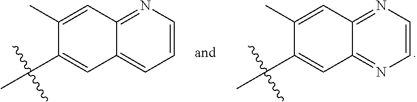 [SMILES: *c1cc2cccnc2cc1C ; *c1cc2nccnc2cc1C]

In some embodiments, A is a 5- or 6-membered heteroaryl or heterocycloalkyl group containing at least one heteroatom selected from the group consisting of N, O, and S, wherein the heteroaryl or heterocycloalkyl is optionally substituted with one or more R⁴. In some embodiments, A is 5- or 6-membered heteroaryl or heterocycloalkyl group containing at least one heteroatom selected from the group consisting of N, O, and S, wherein the heteroaryl or heterocycloalkyl is optionally substituted with one to three R⁴.

In some embodiments, A is a 5-membered heteroaryl or heterocycloalkyl group containing at least one heteroatom selected from the group consisting of N, O, and S, wherein the heteroaryl or heterocycloalkyl is optionally substituted with one or more R⁴. In some embodiments, A is a 5-membered heteroaryl or heterocycloalkyl group containing one or two heteroatoms selected from the group consisting of N, O, and S, wherein the heteroaryl or heterocycloalkyl is optionally substituted with one or more R⁴. In some embodiments, A is a 5-membered heteroaryl or heterocycloalkyl group containing one or two heteroatoms selected from the group consisting of N and O, wherein the heteroaryl or heterocycloalkyl is optionally substituted with one or more R⁴. In some embodiments, A is a 5-membered heteroaryl or heterocycloalkyl group containing two heteroatoms selected from the group consisting of N and O, wherein the heteroaryl or heterocycloalkyl is optionally substituted with one or more R⁴. In some embodiments, A is a 5-membered heteroaryl or heterocycloalkyl group containing one heteroatom selected from the group consisting of N and O, wherein the heteroaryl or heterocycloalkyl is optionally substituted with one or more R⁴.

In some embodiments, A is a 6-membered heteroaryl or heterocycloalkyl group containing at least one heteroatom selected from the group consisting of N, O, and S, wherein the heteroaryl or heterocycloalkyl is optionally substituted with one or more R⁴. In some embodiments, A is a 6-membered heteroaryl or heterocycloalkyl group containing one or two heteroatoms selected from the group consisting of N, O, and S, wherein the heteroaryl or heterocycloalkyl is optionally substituted with one or more R⁴. In some embodiments, A is a 6-membered heteroaryl or heterocycloalkyl group containing one or two heteroatoms selected from the group consisting of N and O, wherein the heteroaryl or heterocycloalkyl is optionally substituted with one or more R⁴. In some embodiments, A is a 6-membered heteroaryl or heterocycloalkyl group containing two heteroatoms selected from the group consisting of N and O, wherein the heteroaryl or heterocycloalkyl is optionally substituted with one or more R⁴. In some embodiments, A is a 6-membered heteroaryl or heterocycloalkyl group containing one heteroatom selected from the group consisting of N and O, wherein the heteroaryl or heterocycloalkyl is optionally substituted with one or more R⁴.

In some embodiments, R⁴ is independently selected from the group consisting of halogen, oxo, thioxo, CD₃, CD₂CD₃, C₁-C₄ alkyl, C₁-C₄ alkoxy, and C₁-C₆ haloalkyl; or two geminal R⁴ together with the intervening geminal carbon atom, form a C₃-C₆ cycloalkyl. In some embodiments, each R⁴ is independently selected from the group consisting of halogen, oxo, thioxo, C₁-C₄ alkyl, C₁-C₄ alkoxy, CD₃, CD₂CD₃, and C₁-C₆ haloalkyl; or two geminal R⁴, together with the intervening geminal carbon atom, form a cyclopropyl. In some embodiments, each R⁴ is oxo, thioxo, CD₃, CD₂CD₃, or methyl, or two geminal R⁴, together with the intervening geminal carbon atom, form a cyclopropyl. In some embodiments, each R⁴ is oxo, thioxo, CD₃, or methyl. In some embodiments, two geminal R⁴, together with the intervening geminal carbon atom, form a cyclopropyl.

In some embodiments, R² is H, halogen, —(CH₂)ₙ—CN, —OH, —(CH₂)ₙ—O—C₁-C₄ alkyl, C₁-C₄ alkyl, C₁-C₄ alkoxy, C₁-C₄ haloalkyl, C₂-C₄ alkenyl, C₂-C₄ alkynyl, or C(O)NH₂. In some embodiments, R² is H. In some embodiments, R² is H, halogen, —(CH₂)ₙ—CN, —OH, —(CH₂)ₙ—O—C₁-C₄ alkyl, C₁-C₄ alkoxy, or C₁-C₄ haloalkyl. In some embodiments, R² is H, fluoro, —CN, —CH₂—CN, —CH₂—O—CH₃, —OH, methoxy, or CF₃. In some embodiments, R² is halogen, —(CH₂)ₙ—CN, —OH, —(CH₂)ₙ—O—C₁-C₄ alkyl, C₁-C₄ alkoxy, or C₁-C₄ haloalkyl. In some embodiments, R² is fluoro, —CN, —CH₂—CN, —CH₂—O—CH₃, —OH, methoxy, or CF₃.

In some embodiments, R³ is selected from the group consisting of H, F, C₂-C₄ alkenyl, C₂-C₄ alkynyl, CN, OH, CH₂OH, NH₂, CH₂NH₂, and C₁-C₄ alkyl. In some embodiments, R³ is H.

In some embodiments, each n is independently an integer from 0 to 4. In some embodiments, n is 0. In some embodiments, n is 1.

In some embodiments, the compounds of Formula (I) have the structure of Formula (Ia-1):

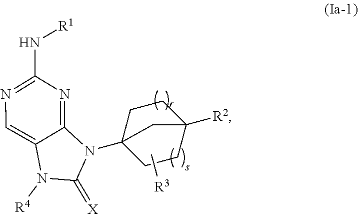

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ia-2):

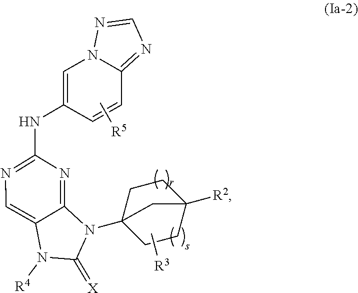

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ia-3):

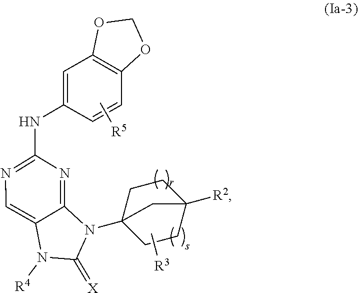

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ia-4):

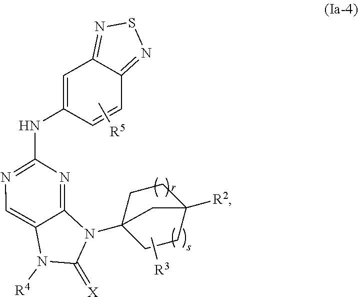

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ia-5):

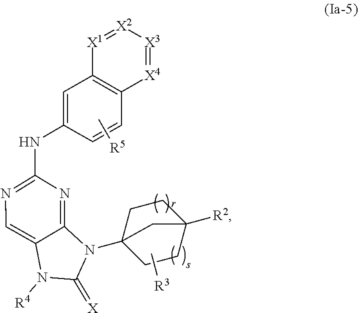

or a pharmaceutically acceptable salt, stereoisomer, solvate, prodrug, or tautomer thereof, wherein X is O, S, NH, or CH₂; and X¹, X², X³, and X⁴ are each independently N, CH, or C(R⁵).

In some embodiments, the compounds of Formula (I) have the structure of Formula (Ia-6):

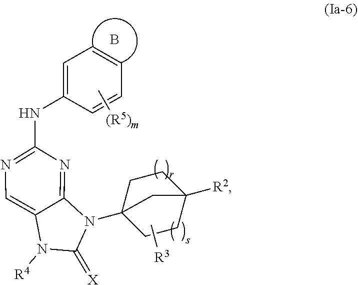

or a pharmaceutically acceptable salt, stereoisomer, solvate, prodrug, or tautomer thereof, wherein X is O, S, NH, or CH₂; B is a 5- or 6-membered aryl or heteroaryl, optionally substituted with one or more R⁵; and m is an integer from 0 to 4. In some embodiments, m is an integer from 1 to 3.

In some embodiments, the compounds of Formula (I) have the structure of Formula (Ia-7):

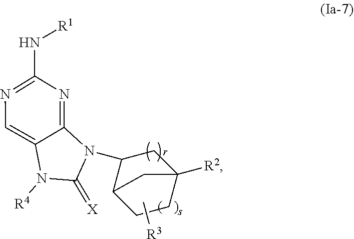

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ib-1):

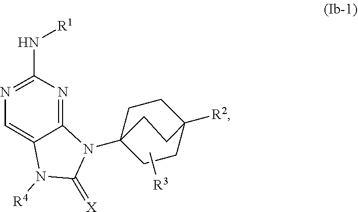

or a pharmaceutically acceptable salt, stereoisomer, solvate, prodrug, or tautomer thereof, wherein X is O, S, NH, or CH₂, provided that when R¹ is

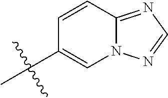 [SMILES: *c1ccc2ncnn2c1]

optionally substituted with one or more R⁵, then X is not O.

In some embodiments, the compounds of Formula (I) have the structure of Formula (Ib-2):

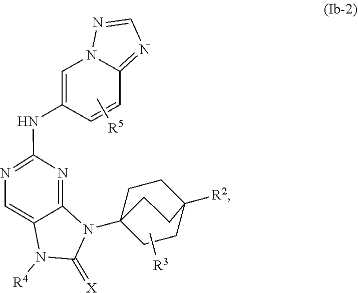

or a pharmaceutically acceptable salt, stereoisomer, solvate, prodrug, or tautomer thereof, wherein X is S, NH, or CH₂.

In some embodiments, the compounds of Formula (I) have the structure of Formula (Ib-3):

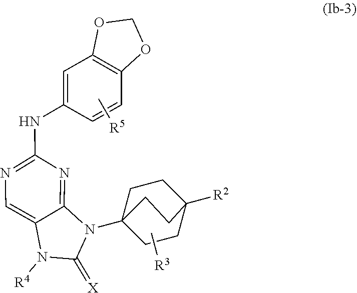

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ib-4):

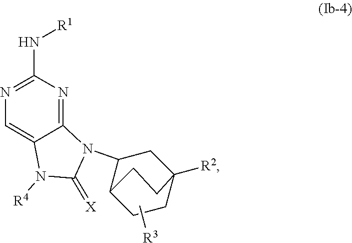

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ic-1):

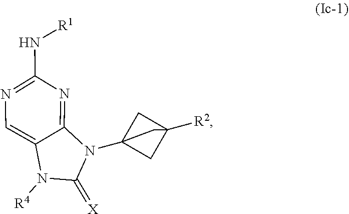

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ic-2):

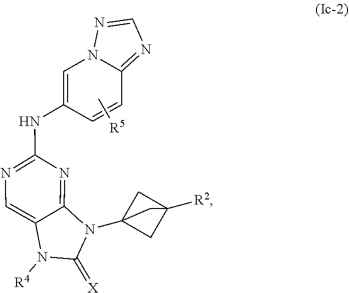

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ic-3):

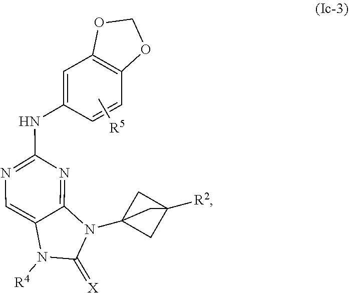

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ic-4):

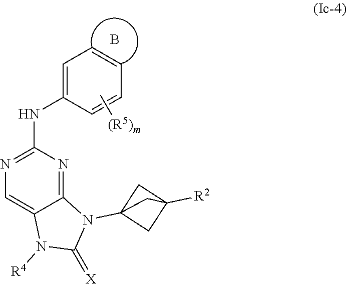

or a pharmaceutically acceptable salt, stereoisomer, solvate, prodrug, or tautomer thereof, wherein X is O, S, NH, or CH₂; B is a 5- or 6-membered aryl or heteroaryl, optionally substituted with one or more R⁵, and m is an integer from 0 to 4. In some embodiments, m is an integer from 1 to 3.

In some embodiments, the compounds of Formula (I) have the structure of Formula (Id-1):

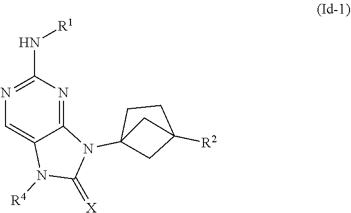

or a pharmaceutically acceptable salt, stereoisomer, solvate, prodrug, or tautomer thereof, wherein X is O, S, NH, or CH₂.

In some embodiments, the compounds of Formula (I) have the structure of Formula (Id-2):

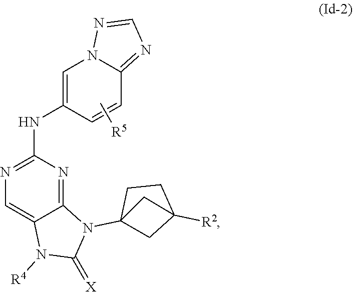

or a pharmaceutically acceptable salt, stereoisomer, solvate, prodrug, or tautomer thereof, wherein X is O, S, NH, or CH₂.

In some embodiments, the compounds of Formula (I) have the structure of Formula (Id-3):

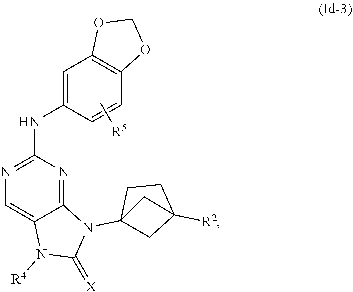

or a pharmaceutically acceptable salt, stereoisomer, solvate, prodrug, or tautomer thereof, wherein X is O, S, NH, or CH₂.

In some embodiments, the compounds of Formula (I) have the structure of Formula (Id-4):

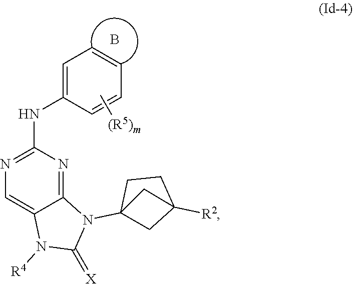

or a pharmaceutically acceptable salt, stereoisomer, solvate, prodrug, or tautomer thereof, wherein X is O, S, NH, or CH₂; B is a 5- or 6-membered aryl or heteroaryl, optionally substituted with one or more R⁵; and m is an integer from 0 to 4. In some embodiments, m is an integer from 1 to 3.

In some embodiments, the compounds of Formula (I) have the structure of Formula (Ie-1):

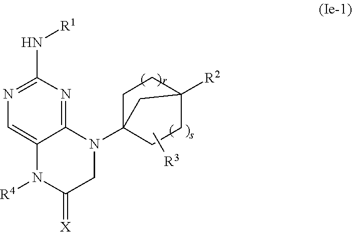

or a pharmaceutically acceptable salt, stereoisomer, solvate, prodrug, or tautomer thereof, wherein X is O, S, NH, or CH₂; and m is an integer from 1 to 3.

In some embodiments, the compounds of Formula (I) have the structure of Formula (Ie-2):

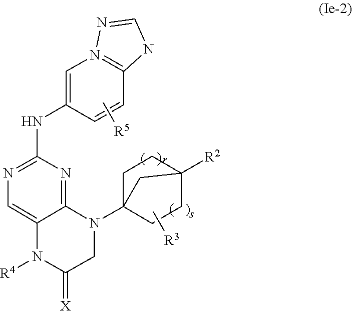

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ie-3):

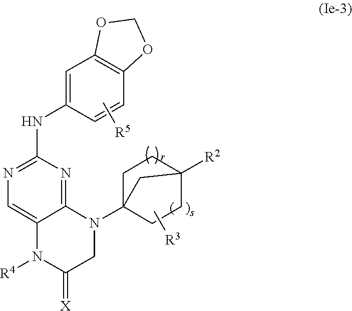

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ie-4):

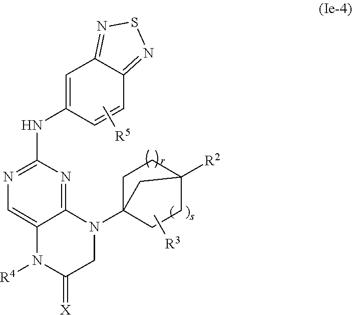

or a pharmaceutically acceptable salt, stereoisomer, solvate, prodrug, or tautomer thereof, wherein X is O, S, NH, or CH₂.

In some embodiments, the compounds of Formula (I) have the structure of Formula (Ie-5):

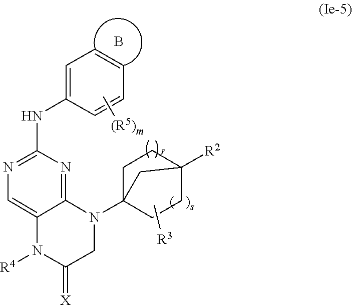

or a pharmaceutically acceptable salt, stereoisomer, solvate, prodrug, or tautomer thereof, wherein X is O, S, NH, or CH₂. B is a 5- or 6-membered aryl or heteroaryl, optionally substituted with one or more R⁵; and m is an integer from 0 to 4. In some embodiments, m is an integer from 1 to 3.

In some embodiments, the compounds of Formula (I) have the structure of Formula (If-1):

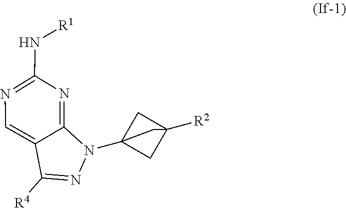

or a pharmaceutically acceptable salt, stereoisomer, solvate, prodrug, or tautomer thereof.

In some embodiments, the compounds of Formula (I) have the structure of Formula (If-2):

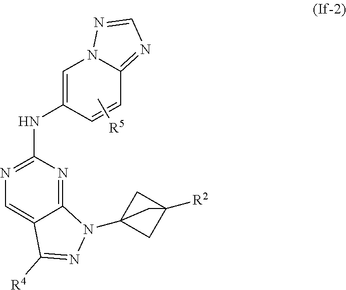

or a pharmaceutically acceptable salt, stereoisomer, solvate, prodrug, or tautomer thereof.

In some embodiments, the compounds of Formula (I) have the structure of Formula

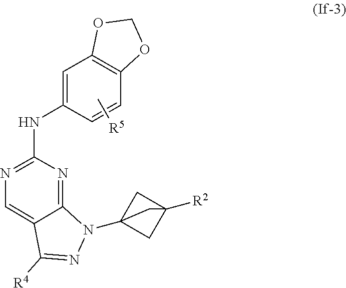

or a pharmaceutically acceptable salt, stereoisomer, solvate, prodrug, or tautomer thereof.

In some embodiments, the compounds of Formula (I) have the structure of Formula (If-4):

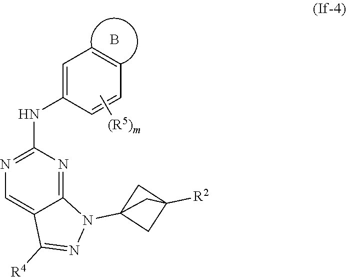

or a pharmaceutically acceptable salt, stereoisomer, solvate, prodrug, or tautomer thereof, wherein B is a 5- or 6-membered aryl or heteroaryl, optionally substituted with one or more R⁵; and m is an integer from 0 to 4. In some embodiments, m is an integer from 1 to 3.

In some embodiments, the compounds of Formula (I) have the structure of Formula (Ig-1):

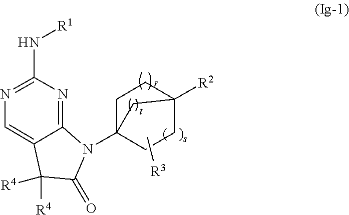

or a pharmaceutically acceptable salt, stereoisomer, solvate, prodrug, or tautomer thereof.

In some embodiments, the compounds of Formula (I) have the structure of Formula (Ig-2):

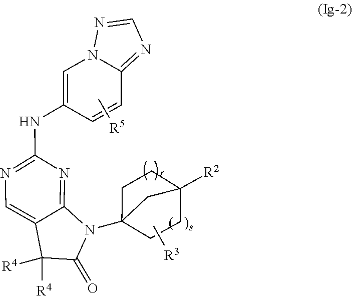

or a pharmaceutically acceptable salt, stereoisomer, solvate, prodrug, or tautomer thereof.

In some embodiments, the compounds of Formula (I) have the structure of Formula (Ig-3):

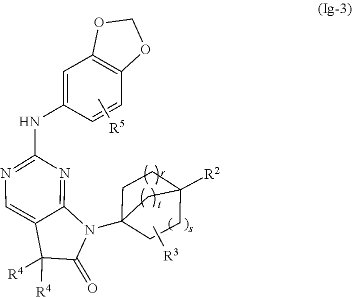

or a pharmaceutically acceptable salt, stereoisomer, solvate, prodrug, or tautomer thereof.

In some embodiments, the compounds of Formula (I) have the structure of Formula (Ig-4):

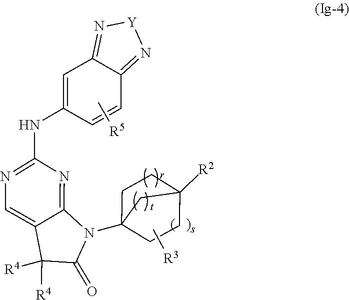

or a pharmaceutically acceptable salt, stereoisomer, solvate, prodrug, or tautomer thereof, wherein Y is O, or S.

In some embodiments, the compounds of Formula (I) have the structure of Formula (Ig-5):

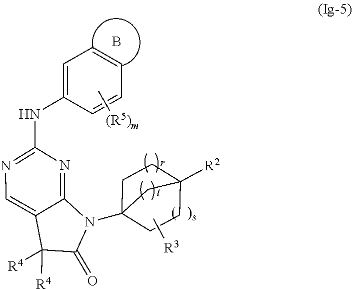

or a pharmaceutically acceptable salt, stereoisomer, solvate, prodrug, or tautomer thereof, wherein B is a 5- or 6-membered aryl or heteroaryl, optionally substituted with one or more R⁵; and m is an integer from 0 to 4. In some embodiments, m is an integer from 1 to 3.

In some embodiments of the foregoing, the compounds of Formula I are compounds or pharmaceutically acceptable salts, stereoisomers, solvates, prodrugs, or tautomers thereof. In some embodiments of the foregoing, the compounds of Formula I are compounds or pharmaceutically acceptable salts, stereoisomers, or tautomers thereof. In some embodiments of the foregoing, the compounds of Formula I are compounds or pharmaceutically acceptable salts or stereoisomers thereof. In some embodiments of the foregoing, the compounds of Formula I are compounds or pharmaceutically acceptable salts thereof.

TABLE 1
Exemplary Compounds

| Compound. | |
|---|---|
| No. | Chemical Structure |
| 1 | 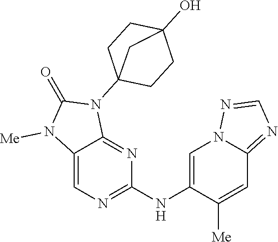 [SMILES: Cc1cc2ncnn2cc1Nc1ncc2c(n1)n(C13CCC(O)(CC1)C3)c(=O)n2C] |
| 2 | 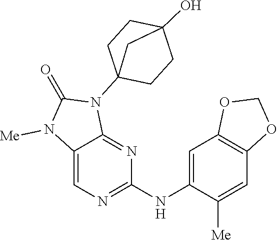 [SMILES: Cc1cc2c(cc1Nc1ncc3c(n1)n(C14CCC(O)(CC1)C4)c(=O)n3C)OCO2] |
| 3 | 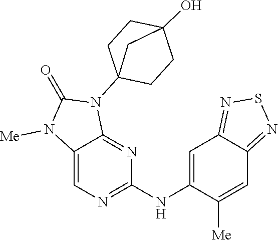 [SMILES: Cc1cc2nsnc2cc1Nc1ncc2c(n1)n(C13CCC(O)(CC1)C3)c(=O)n2C] |
| 4 | 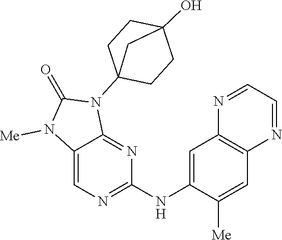 [SMILES: Cc1cc2nccnc2cc1Nc1ncc2c(n1)n(C13CCC(O)(CC1)C3)c(=O)n2C] |
| 5 | 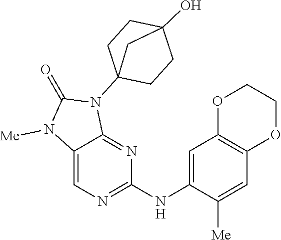 [SMILES: Cc1cc2c(cc1Nc1ncc3c(n1)n(C14CCC(O)(CC1)C4)c(=O)n3C)OCCO2] |
| 6 | 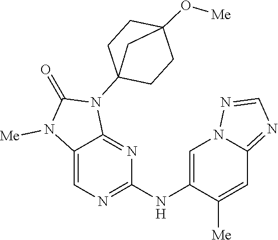 [SMILES: COC12CCC(n3c(=O)n(C)c4cnc(Nc5cn6ncnc6cc5C)nc43)(CC1)C2] |
| 7 | 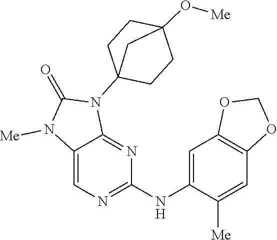 [SMILES: COC12CCC(n3c(=O)n(C)c4cnc(Nc5cc6c(cc5C)OCO6)nc43)(CC1)C2] |
| 8 | 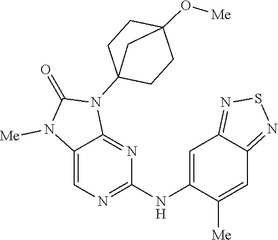 [SMILES: COC12CCC(n3c(=O)n(C)c4cnc(Nc5cc6nsnc6cc5C)nc43)(CC1)C2] |
| 9 | 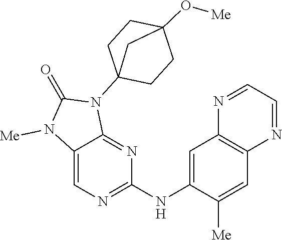 [SMILES: COC12CCC(n3c(=O)n(C)c4cnc(Nc5cc6nccnc6cc5C)nc43)(CC1)C2] |
| 10 | 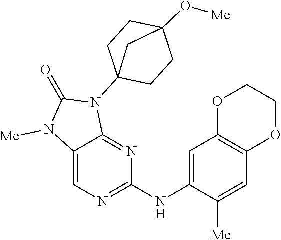 [SMILES: COC12CCC(n3c(=O)n(C)c4cnc(Nc5cc6c(cc5C)OCCO6)nc43)(CC1)C2] |
| 11 | 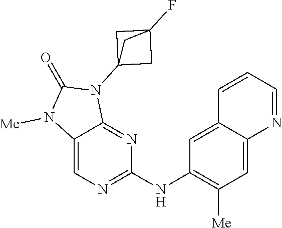 [SMILES: Cc1cc2ncccc2cc1Nc1ncc2c(n1)n(C13CC(F)(C1)C3)c(=O)n2C] |
| 12 | 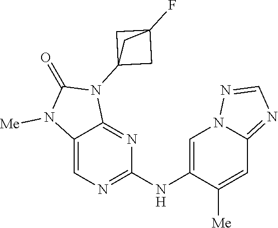 [SMILES: Cc1cc2ncnn2cc1Nc1ncc2c(n1)n(C13CC(F)(C1)C3)c(=O)n2C] |
| 13 | 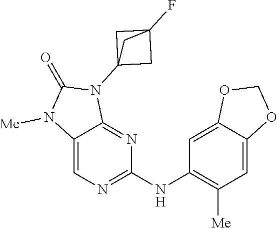 [SMILES: Cc1cc2c(cc1Nc1ncc3c(n1)n(C14CC(F)(C1)C4)c(=O)n3C)OCO2] |
| 14 | 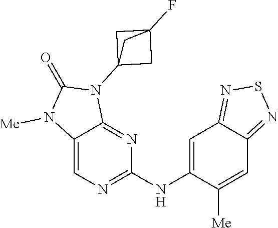 [SMILES: Cc1cc2nsnc2cc1Nc1ncc2c(n1)n(C13CC(F)(C1)C3)c(=O)n2C] |
| 15 | 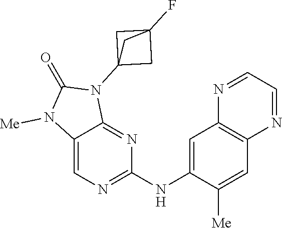 [SMILES: Cc1cc2nccnc2cc1Nc1ncc2c(n1)n(C13CC(F)(C1)C3)c(=O)n2C] |
| 16 | 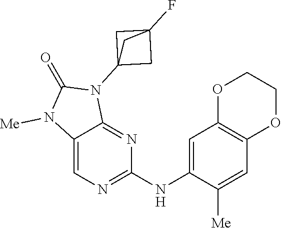 [SMILES: Cc1cc2c(cc1Nc1ncc3c(n1)n(C14CC(F)(C1)C4)c(=O)n3C)OCCO2] |
| 17 | 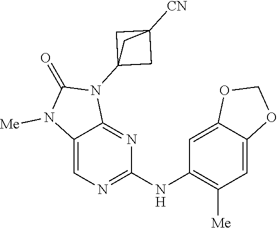 [SMILES: Cc1cc2c(cc1Nc1ncc3c(n1)n(C14CC(C#N)(C1)C4)c(=O)n3C)OCO2] |
| 18 | 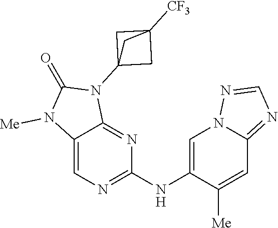 [SMILES: Cc1cc2ncnn2cc1Nc1ncc2c(n1)n(C13CC(C(F)(F)F)(C1)C3)c(=O)n2C] |
| 19 | 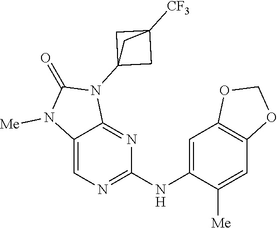 [SMILES: Cc1cc2c(cc1Nc1ncc3c(n1)n(C14CC(C(F)(F)F)(C1)C4)c(=O)n3C)OCO2] |
| 20 | 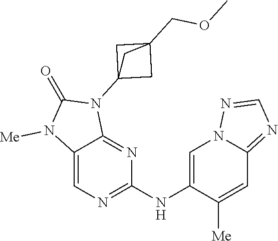 [SMILES: COCC12CC(n3c(=O)n(C)c4cnc(Nc5cn6ncnc6cc5C)nc43)(C1)C2] |
| 21 | 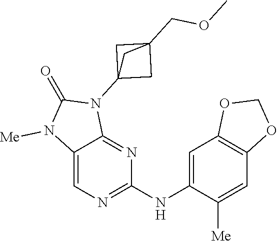 [SMILES: COCC12CC(n3c(=O)n(C)c4cnc(Nc5cc6c(cc5C)OCO6)nc43)(C1)C2] |
| 22 | 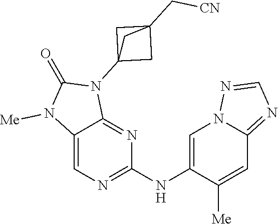 [SMILES: Cc1cc2ncnn2cc1Nc1ncc2c(n1)n(C13CC(CC#N)(C1)C3)c(=O)n2C] |
| 23 | 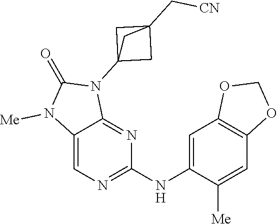 [SMILES: Cc1cc2c(cc1Nc1ncc3c(n1)n(C14CC(CC#N)(C1)C4)c(=O)n3C)OCO2] |
| 24 | 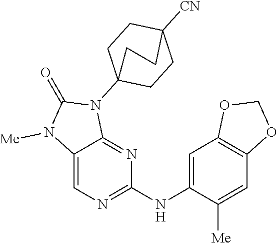 [SMILES: Cc1cc2c(cc1Nc1ncc3c(n1)n(C14CCC(C#N)(CC1)CC4)c(=O)n3C)OCO2] |
| 25 | 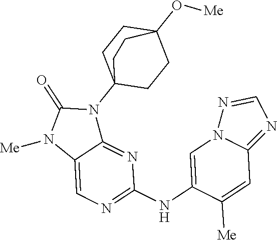 [SMILES: COC12CCC(n3c(=O)n(C)c4cnc(Nc5cn6ncnc6cc5C)nc43)(CC1)CC2] |
| 26 | 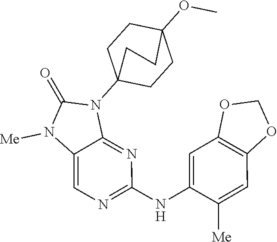 [SMILES: COC12CCC(n3c(=O)n(C)c4cnc(Nc5cc6c(cc5C)OCO6)nc43)(CC1)CC2] |
| 27 | 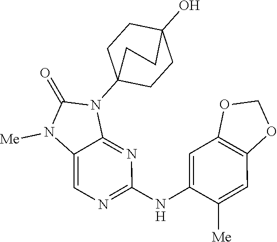 [SMILES: Cc1cc2c(cc1Nc1ncc3c(n1)n(C14CCC(O)(CC1)CC4)c(=O)n3C)OCO2] |
| 28 | 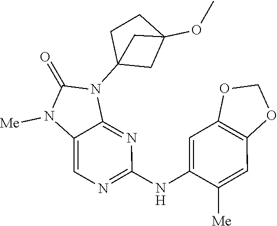 [SMILES: COC12CCC(n3c(=O)n(C)c4cnc(Nc5cc6c(cc5C)OCO6)nc43)(C1)C2] |
| 29 | 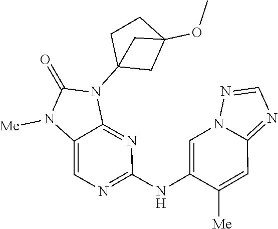 [SMILES: COC12CCC(n3c(=O)n(C)c4cnc(Nc5cn6ncnc6cc5C)nc43)(C1)C2] |
| 30 | 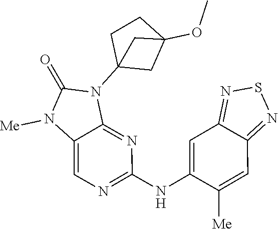 [SMILES: COC12CCC(n3c(=O)n(C)c4cnc(Nc5cc6nsnc6cc5C)nc43)(C1)C2] |
| 31 | 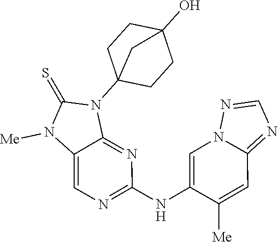 [SMILES: Cc1cc2ncnn2cc1Nc1ncc2c(n1)n(C13CCC(O)(CC1)C3)c(=S)n2C] |
| 32 | 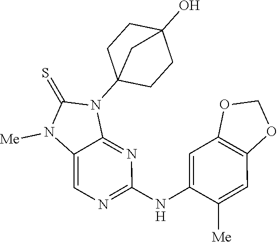 [SMILES: Cc1cc2c(cc1Nc1ncc3c(n1)n(C14CCC(O)(CC1)C4)c(=S)n3C)OCO2] |
| 33 | 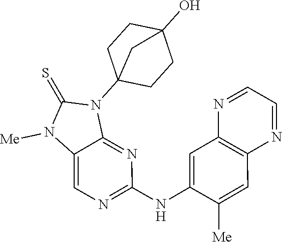 [SMILES: Cc1cc2nccnc2cc1Nc1ncc2c(n1)n(C13CCC(O)(CC1)C3)c(=S)n2C] |
| 34 | 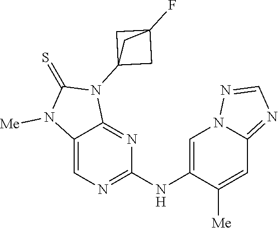 [SMILES: Cc1cc2ncnn2cc1Nc1ncc2c(n1)n(C13CC(F)(C1)C3)c(=S)n2C] |
| 35 | 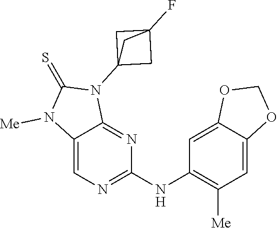 [SMILES: Cc1cc2c(cc1Nc1ncc3c(n1)n(C14CC(F)(C1)C4)c(=S)n3C)OCO2] |
| 36 | 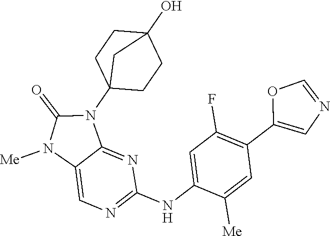 [SMILES: Cc1cc(-c2cnco2)c(F)cc1Nc1ncc2c(n1)n(C13CCC(O)(CC1)C3)c(=O)n2C] |
| 37 | 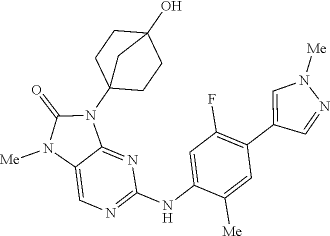 [SMILES: Cc1cc(-c2cnn(C)c2)c(F)cc1Nc1ncc2c(n1)n(C13CCC(O)(CC1)C3)c(=O)n2C] |
| 38 | 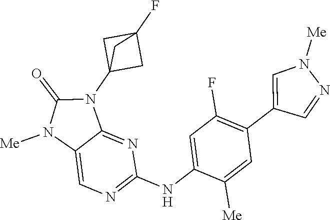 [SMILES: Cc1cc(-c2cnn(C)c2)c(F)cc1Nc1ncc2c(n1)n(C13CC(F)(C1)C3)c(=O)n2C] |
| 39 | 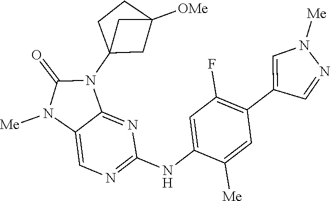 [SMILES: COC12CCC(n3c(=O)n(C)c4cnc(Nc5cc(F)c(-c6cnn(C)c6)cc5C)nc43)(C1)C2] |
| 40 | 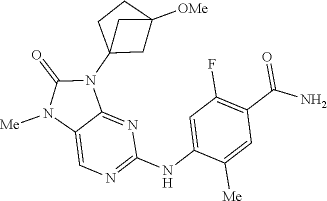 [SMILES: COC12CCC(n3c(=O)n(C)c4cnc(Nc5cc(F)c(C(N)=O)cc5C)nc43)(C1)C2] |
| 41 | 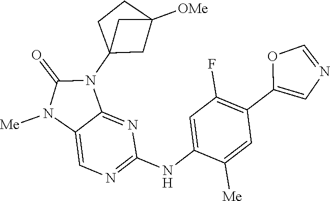 [SMILES: COC12CCC(n3c(=O)n(C)c4cnc(Nc5cc(F)c(-c6cnco6)cc5C)nc43)(C1)C2] |
| 42 | 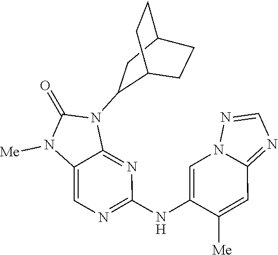 [SMILES: Cc1cc2ncnn2cc1Nc1ncc2c(n1)n(C1CC3CCC1CC3)c(=O)n2C] |
| 43 | 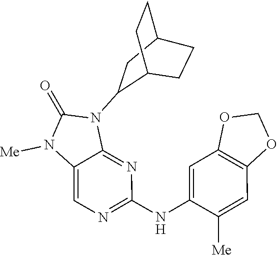 [SMILES: Cc1cc2c(cc1Nc1ncc3c(n1)n(C1CC4CCC1CC4)c(=O)n3C)OCO2] |
| 44 | 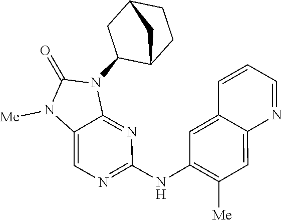 [SMILES: Cc1cc2ncccc2cc1Nc1ncc2c(n1)n([C@H]1C[C@@H]3CC[C@H]1C3)c(=O)n2C] |
| 45 | 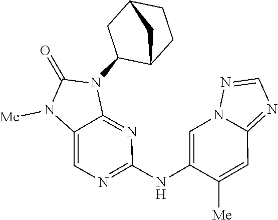 [SMILES: Cc1cc2ncnn2cc1Nc1ncc2c(n1)n([C@H]1C[C@@H]3CC[C@H]1C3)c(=O)n2C] |
| 46 | 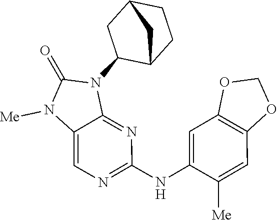 [SMILES: Cc1cc2c(cc1Nc1ncc3c(n1)n([C@H]1C[C@@H]4CC[C@H]1C4)c(=O)n3C)OCO2] |
| 47 | 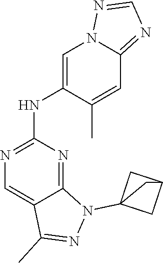 [SMILES: Cc1cc2ncnn2cc1Nc1ncc2c(C)nn(C34CC(C3)C4)c2n1] |
| 48 | 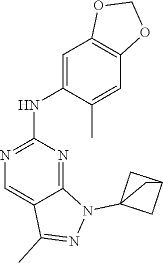 [SMILES: Cc1cc2c(cc1Nc1ncc3c(C)nn(C45CC(C4)C5)c3n1)OCO2] |
| 49 | 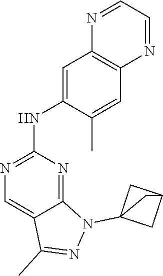 [SMILES: Cc1cc2nccnc2cc1Nc1ncc2c(C)nn(C34CC(C3)C4)c2n1] |
| 50 | 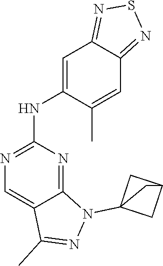 [SMILES: Cc1cc2nsnc2cc1Nc1ncc2c(C)nn(C34CC(C3)C4)c2n1] |
| 51 | 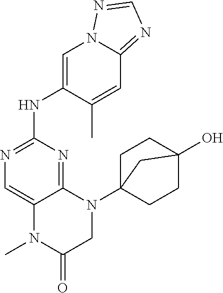 [SMILES: Cc1cc2ncnn2cc1Nc1ncc2c(n1)N(C13CCC(O)(CC1)C3)CC(=O)N2C] |
| 52 | 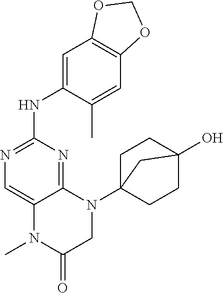 [SMILES: Cc1cc2c(cc1Nc1ncc3c(n1)N(C14CCC(O)(CC1)C4)CC(=O)N3C)OCO2] |
| 53 | 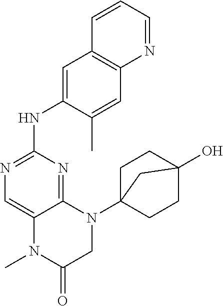 [SMILES: Cc1cc2ncccc2cc1Nc1ncc2c(n1)N(C13CCC(O)(CC1)C3)CC(=O)N2C] |
| 54 | 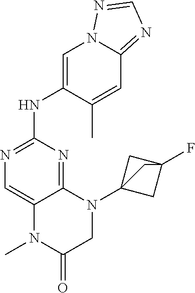 [SMILES: Cc1cc2ncnn2cc1Nc1ncc2c(n1)N(C13CC(F)(C1)C3)CC(=O)N2C] |
| 55 | 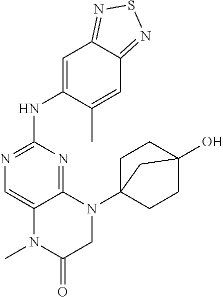 [SMILES: Cc1cc2nsnc2cc1Nc1ncc2c(n1)N(C13CCC(O)(CC1)C3)CC(=O)N2C] |
| 56 | 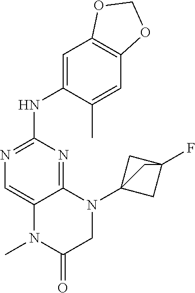 [SMILES: Cc1cc2c(cc1Nc1ncc3c(n1)N(C14CC(F)(C1)C4)CC(=O)N3C)OCO2] |
| 57 | 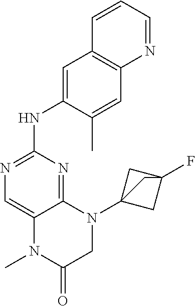 [SMILES: Cc1cc2ncccc2cc1Nc1ncc2c(n1)N(C13CC(F)(C1)C3)CC(=O)N2C] |
| 58 | 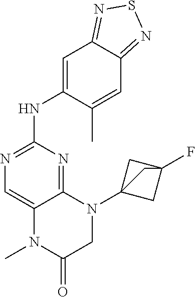 [SMILES: Cc1cc2nsnc2cc1Nc1ncc2c(n1)N(C13CC(F)(C1)C3)CC(=O)N2C] |
| 59 | 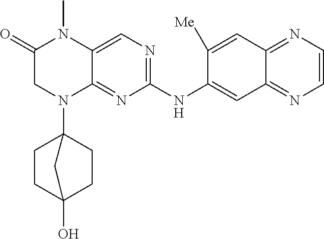 [SMILES: Cc1cc2nccnc2cc1Nc1ncc2c(n1)N(C13CCC(O)(CC1)C3)CC(=O)N2C] |
| 60 | 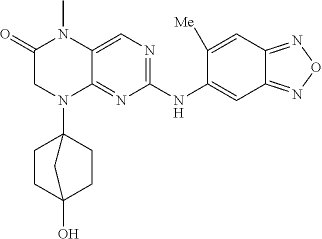 [SMILES: Cc1cc2nonc2cc1Nc1ncc2c(n1)N(C13CCC(O)(CC1)C3)CC(=O)N2C] |
| 61 | 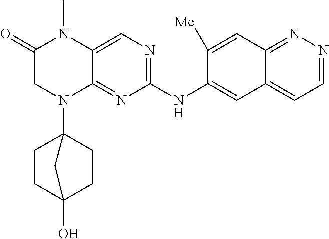 [SMILES: Cc1cc2nnccc2cc1Nc1ncc2c(n1)N(C13CCC(O)(CC1)C3)CC(=O)N2C] |
| 62 | 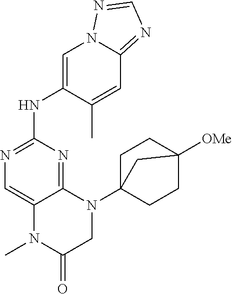 [SMILES: COC12CCC(N3CC(=O)N(C)c4cnc(Nc5cn6ncnc6cc5C)nc43)(CC1)C2] |
| 63 | 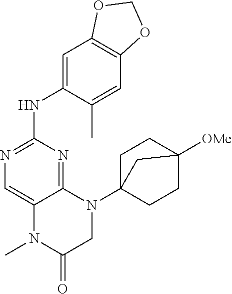 [SMILES: COC12CCC(N3CC(=O)N(C)c4cnc(Nc5cc6c(cc5C)OCO6)nc43)(CC1)C2] |
| 64 | 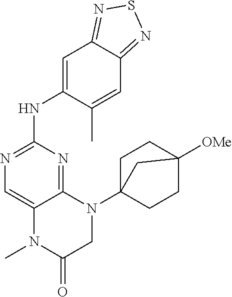 [SMILES: COC12CCC(N3CC(=O)N(C)c4cnc(Nc5cc6nsnc6cc5C)nc43)(CC1)C2] |
| 65 | 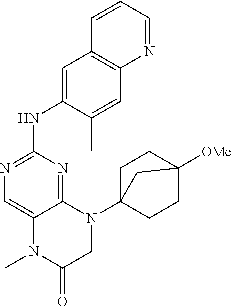 [SMILES: COC12CCC(N3CC(=O)N(C)c4cnc(Nc5cc6cccnc6cc5C)nc43)(CC1)C2] |
| 66 | 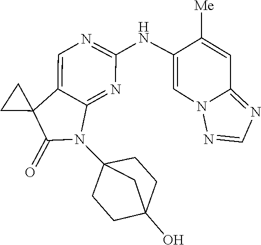 [SMILES: Cc1cc2ncnn2cc1Nc1ncc2c(n1)N(C13CCC(O)(CC1)C3)C(=O)C21CC1] |
| 67 | 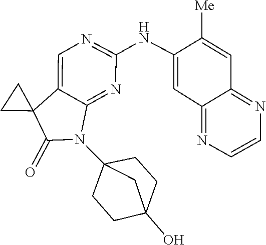 [SMILES: Cc1cc2nccnc2cc1Nc1ncc2c(n1)N(C13CCC(O)(CC1)C3)C(=O)C21CC1] |
| 68 | 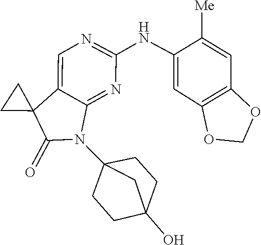 [SMILES: Cc1cc2c(cc1Nc1ncc3c(n1)N(C14CCC(O)(CC1)C4)C(=O)C31CC1)OCO2] |
| 69 | 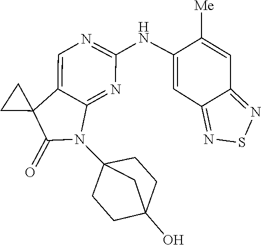 [SMILES: Cc1cc2nsnc2cc1Nc1ncc2c(n1)N(C13CCC(O)(CC1)C3)C(=O)C21CC1] |
| 70 | 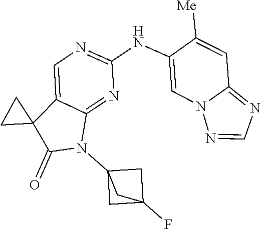 [SMILES: Cc1cc2ncnn2cc1Nc1ncc2c(n1)N(C13CC(F)(C1)C3)C(=O)C21CC1] |
| 71 | 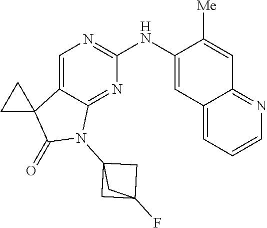 [SMILES: Cc1cc2ncccc2cc1Nc1ncc2c(n1)N(C13CC(F)(C1)C3)C(=O)C21CC1] |
| 72 | 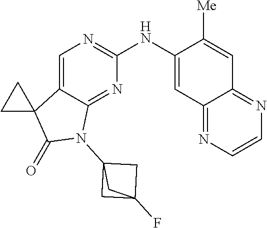 [SMILES: Cc1cc2nccnc2cc1Nc1ncc2c(n1)N(C13CC(F)(C1)C3)C(=O)C21CC1] |
| 73 | 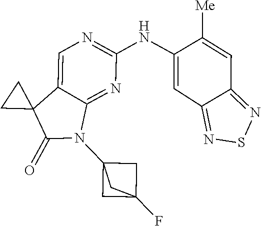 [SMILES: Cc1cc2nsnc2cc1Nc1ncc2c(n1)N(C13CC(F)(C1)C3)C(=O)C21CC1] |
| 74 | 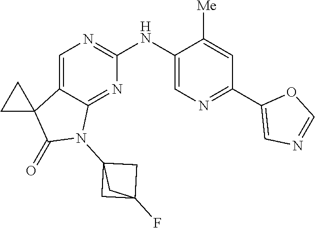 [SMILES: Cc1cc(-c2cnco2)ncc1Nc1ncc2c(n1)N(C13CC(F)(C1)C3)C(=O)C21CC1] |
| 75 | 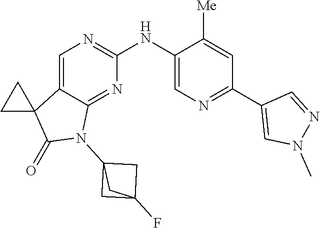 [SMILES: Cc1cc(-c2cnn(C)c2)ncc1Nc1ncc2c(n1)N(C13CC(F)(C1)C3)C(=O)C21CC1] |
| 76 | 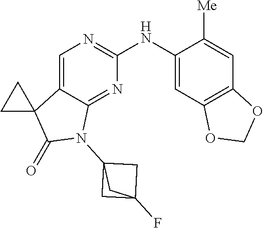 [SMILES: Cc1cc2c(cc1Nc1ncc3c(n1)N(C14CC(F)(C1)C4)C(=O)C31CC1)OCO2] |
| 77 | 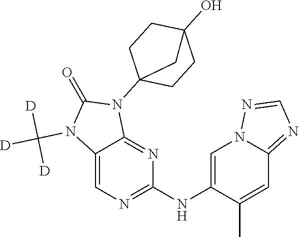 [SMILES: [2H]C([2H])([2H])n1c(=O)n(C23CCC(O)(CC2)C3)c2nc(Nc3cn4ncnc4cc3C)ncc21] |
| 78 | 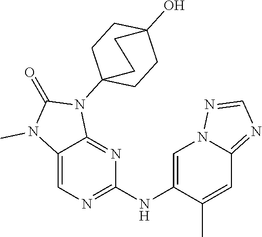 [SMILES: Cc1cc2ncnn2cc1Nc1ncc2c(n1)n(C13CCC(O)(CC1)CC3)c(=O)n2C] |

It should be understood that all isomeric forms are included within the present disclosure, including mixtures thereof. If the compound contains a disubstituted cycloalkyl, the cycloalkyl substituent may have a cis- or trans configuration. All tautomeric forms are also intended to be included.

Compounds of the present disclosure, and pharmaceutically acceptable salts, hydrates, solvates, stereoisomers and prodrugs thereof may exist in their tautomeric form (for example, as an amide or imino ether). All such tautomeric forms are contemplated herein as part of the present disclosure.

The compounds of the present disclosure may contain asymmetric or chiral centers, and, therefore, exist in different stereoisomeric forms. It is intended that all stereoisomeric forms of the compounds of the present disclosure as well as mixtures thereof, including racemic mixtures, form part of the present disclosure. In addition, the present disclosure embraces all geometric and positional isomers. For example, if a compound of the present disclosure incorporates a double bond or a fused ring, both the cis- and trans-forms, as well as mixtures, are embraced within the scope of the present disclosure. Each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the present disclosure may be atropisomers (e.g., substituted biaryls) and are considered as part of this present disclosure. Enantiomers can also be separated by use of a chiral HPLC column.

It is also possible that the compounds of the present disclosure may exist in different tautomeric forms, and all such forms are embraced within the scope of the present disclosure. Also, for example, all keto-enol and imine-enamine forms of the compounds are included in the present disclosure.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this present disclosure, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the present disclosure. Also, for example, all keto-enol and imine-enamine forms of the compounds are included in the present disclosure). Individual stereoisomers of the compounds of the present disclosure may, for example, be substantially free of other stereoisomers, or may be admixed, for example, as racemates or with all other, or other selected, stereoisomers. The chiral centers of the present disclosure can have the S or R configuration as defined by the IUPAC 1974 Recommendations. The use of the terms “salt”, “solvate”, “ester,” “prodrug” and the like, is intended to equally apply to the salt, solvate, ester and prodrug of enantiomers, stereoisomers, rotamers, tautomers, positional isomers, racemates or prodrugs of the inventive compounds.

The compounds of Formula I may form salts which are also within the scope of this present disclosure. Reference to a compound of the Formula herein is understood to include reference to salts thereof, unless otherwise indicated.

The present disclosure is directed to compounds as described herein and pharmaceutically acceptable salts, hydrates, solvates, prodrugs, stereoisomers, or tautomers thereof, and pharmaceutical compositions comprising one or more compounds as described herein, or pharmaceutically acceptable salts, hydrates, solvates, prodrugs, stereoisomers, or tautomers thereof.

### Method of Synthesizing the Compounds

The compounds of the present disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; Greene, T. W., Wuts, P. G. M., Protective Groups in Organic Synthesis, 3rd edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed, Encyclopedia of Reagents for Organic Synthesis, John Wiley and Sons (1995), incorporated by reference herein, are useful and recognised reference textbooks of organic synthesis known to those in the art.

During the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3rd edition. John Wiley & Sons. New York, 1999.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers, however, it will be understood that a given isomer, tautomer, regioisomer or stereoisomer may have a higher level of activity than another isomer, tautomer, regioisomer or stereoisomer.

Methods include but are not limited to those methods described below. Compounds of the present disclosure can be synthesized by following the steps outlined in Schemes G1-G4, which comprise different sequences of assembling intermediates or compounds. Starting materials are either commercially available or made by known procedures in the reported literature or as illustrated below.

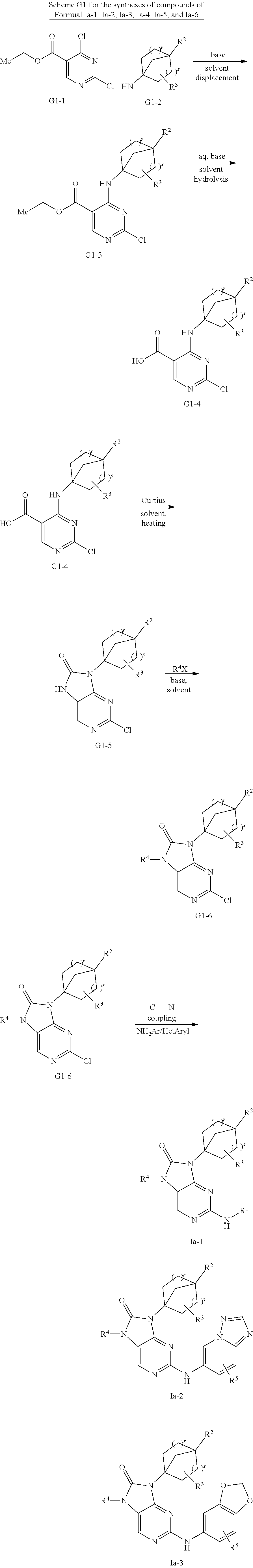

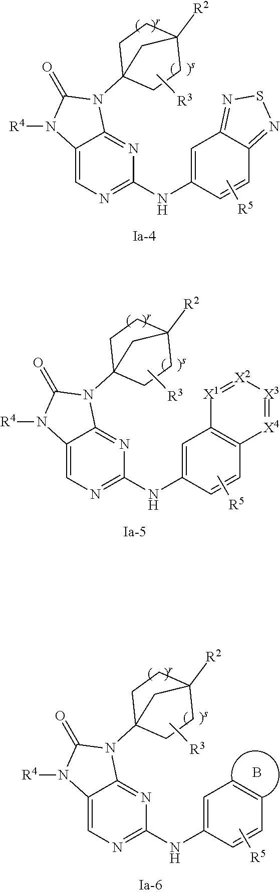

The amine G1-3 can be formed by displacement reactions of amine G1-2 with G1-1 in the presence a base such as DIPEA, Et₃N or K₂CO₃ via nucleophilic substitution. The bicyclic core G1-5 can be formed by hydrolysis of the ester group in G1-3 to the acid G1-3 using an aqueous base such as LiOH or NaOH, followed by Curtius rearrangement and in situ cyclization with DPPA under elevated temperature (such as 40-100° C.). The alkylated G1-6 can be formed from G1-5 with a halide in the presence of a base such as NaOH, NaH, K₂CO₃, etc. Installation of the aniline groups NH₂Ar or NH₂—HetAr can be performed with a Buchwald-Hartwig coupling using catalyst system such as Brett Phos G3 on the chloropyrimidine core G1-6 to synthesize the final product with general formula of Formula Ia-1, Ia-2, Ia-3, Ia-4, Ia-5, and Ia-6 (Scheme G1).

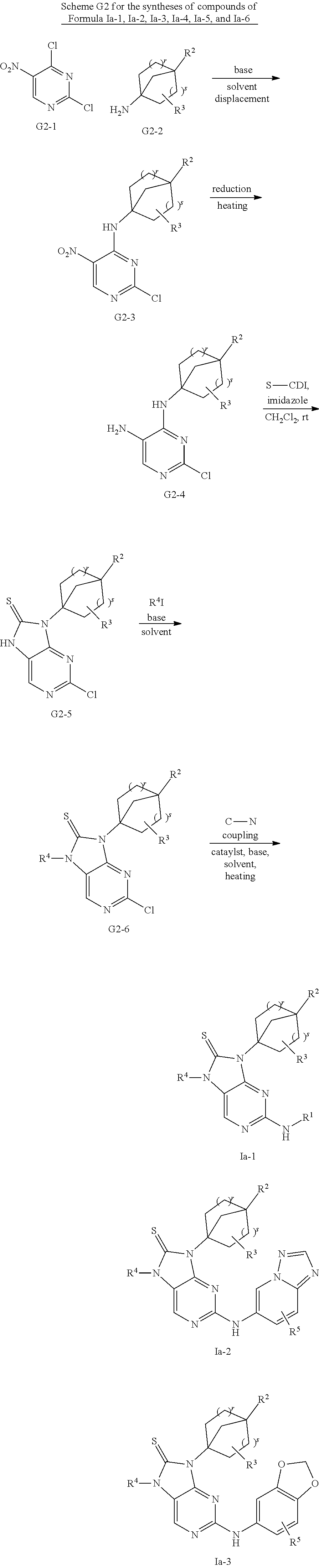

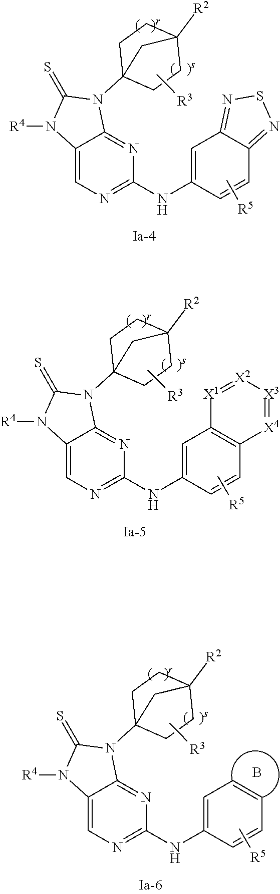

Reduction of NO₂ group in G2-3 to form amine G2-4 can be performed using either Fe or Zn in the presence of HOAc and EtOH under heating. Cyclization of G2-4 with imidazole and 1 1′-thiocarbonyldiimidazole can afford G2-5. The alkylated G2-6 can be formed from G2-5 with a halide in the presence of a base such as NaOH, NaH, K₂CO₃, etc. Installation of the aniline groups NH₂Ar or NH₂—HetAr can be performed with a Buchwald-Hartwig coupling using catalyst system such as Brett Phos G3 on the chloropyrimidine core G2-6 to synthesize the final product with Formula Ta-1, Ta-2, Ta-3, Ta-4, Pa-5, and Ga-6 (Scheme G2).

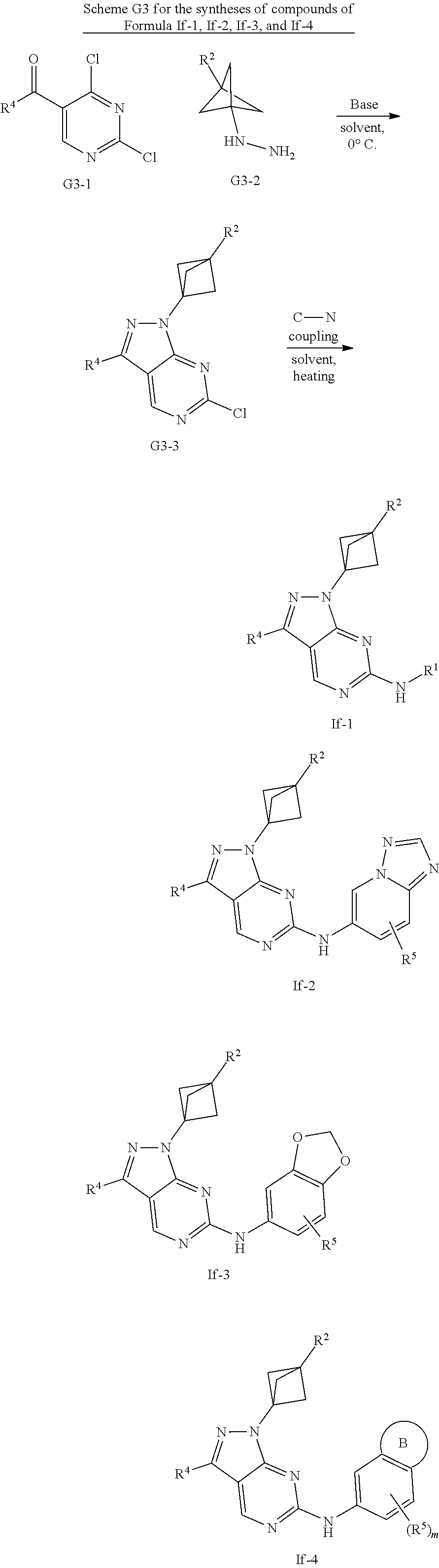

A mixture of base (such as DIPEA), G3-1 and G3-2 in an appropriate solvent (such as THF) at low temperature such as (0° C.) can afford G3-3. C—N coupling reactions of G3-3 with various aryl or heteroaryl amines within the scope of this invention can be achieved in the presence of a suitable catalyst system (such as Brettphos Pd G3) and a base (such as cesium carbonate), which can generate the compounds of Formula If-1, If-2, If-3, and If-4 (Scheme G3).

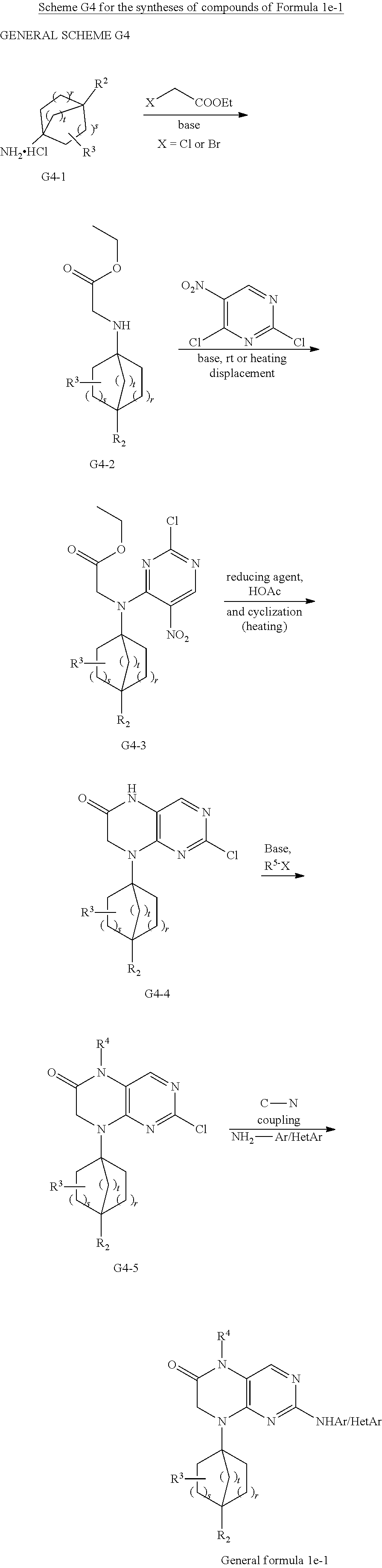

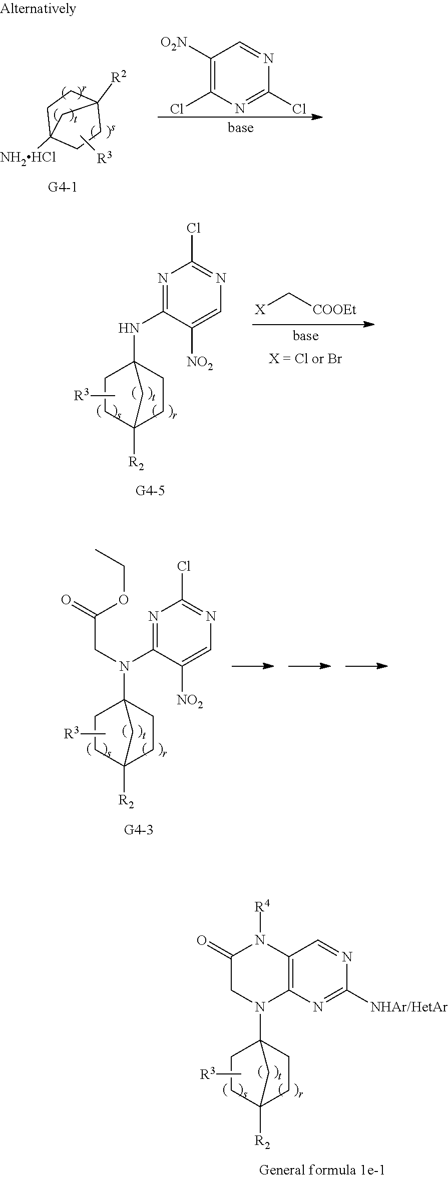

The amine G4-3 can be formed by displacement reactions of amine G4-1 with either 2,4-dichloro-5-nitropyrimidine then ethyl 2-haloacetate 2,4-dichloro-5-nitropyrimidine or with ethyl 2-haloacetate then 2,4-dichloro-5-nitropyrimidine in the presence a base such as Et₃N or K₂CO₃ via nucleophilic substitution. The bicyclic core G4-4 can be formed by reduction of the nitro group to amine using Fe or Zn in the presence of HOAc followed by in situ lactam formation under elevated temperature (such as 40-100° C.). The alkylated G4-5 can be formed from G4-4 with a halide in the presence of a base such as NaOH, NaH, K₂CO₃, etc. Installation of the aniline groups NH₂Ar or NH₂—HetAr can be performed with a Buchwald-Hartwig coupling using catalyst system such as Brett Phos G3 on the chloropyrimidine core G4-5 to synthesize the final product with general formula of 1e-1 (Scheme G4).

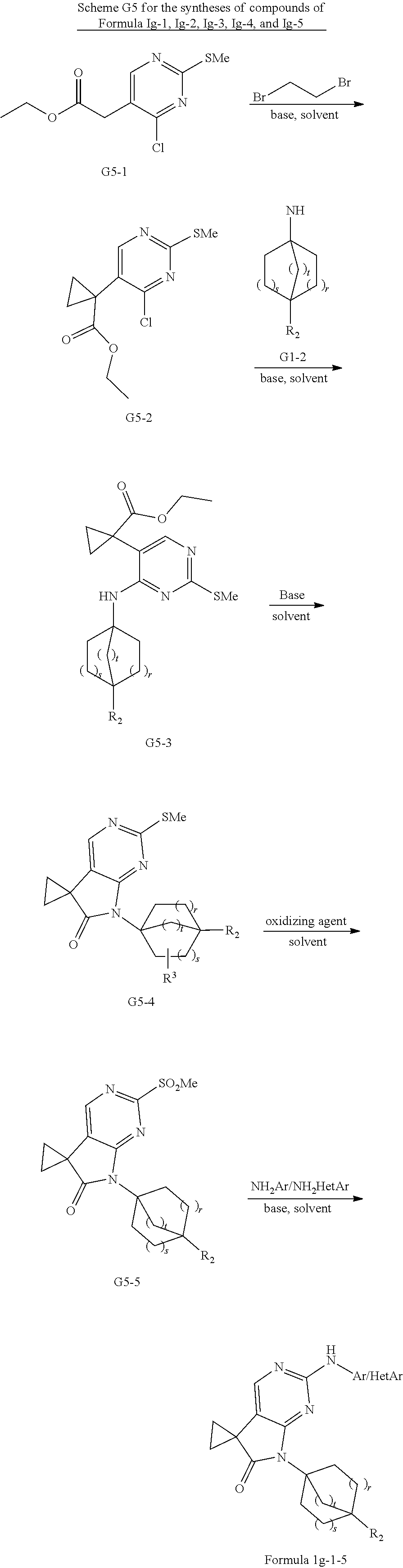

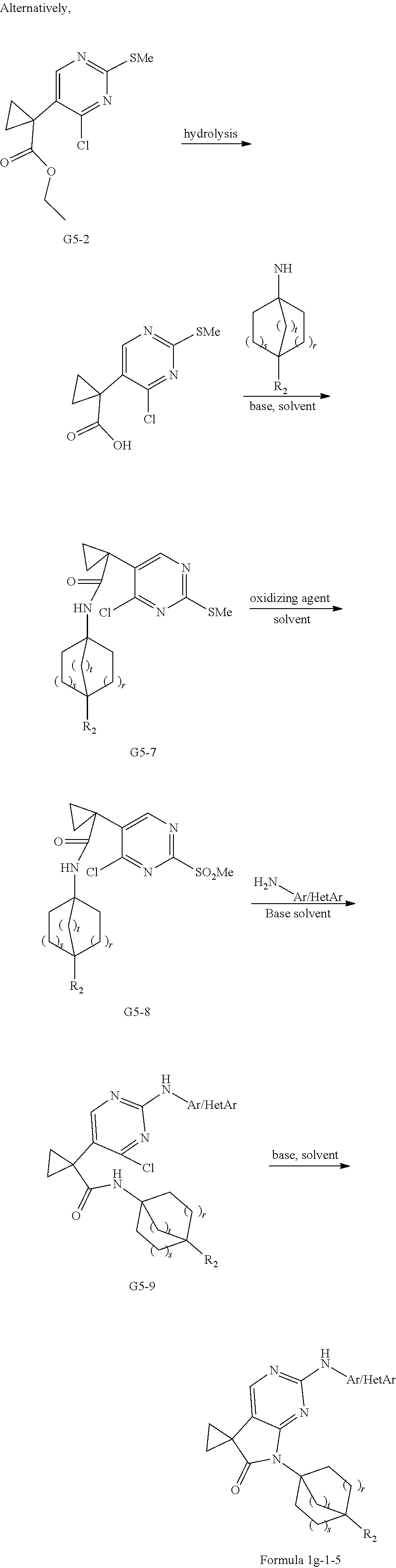

Intermediate G5-2 can be obtained by cyclopropanating G5-1 with dibromoethane in the presence of a base such as NaH in DMF. Amine G5-3 can be formed by displacing amine G1-2 with G5-2 via nucleophilic substitution in the presence a base such as DIPEA, Et₃N or K₂CO₃ via nucleophilic substitution at an elevated temperature such as 100-120° C. in DMA. Treatment of G5-3 with a strong base such as NaH can result in the cyclized intermediate G5-4. Oxidation of the thioether G5-4 to methyl sulfone G5-5 can be achieved with an oxidizing agent such as Oxone.

To synthesize the final product with a general formula of Formula Ig, Ig-2, Ig-3, Ig-4, and Ig-5, the aniline groups NH₂Ar or NH₂—HetAr can be installed through a displacement reaction on the methylsulfone pyrimidine core G5-5 in the presence of a base such as LiHMDS in DMF. Alternatively, the ester G5-2 can be hydrolysized in aqueous base to form the acid G5-6. Amide coupling of G5-6 with amine G1-2 can afford the amide G5-7, which can be treated with an oxidizing agent such as Oxone to form the methyl sulfone analog G5-8. The aniline groups NH₂Ar or NH₂—HetAr can be installed through a displacement reaction on the methylsulfone pyrimidine core G5-8 in the presence of a base such as LiHMDS in THF. The final cyclization reaction of G5-9 can occur in the presence of a strong base such as LiHMDS in THF.

Examples presented herein, unless otherwise stated, are synthesized according to the general procedure presented in Schemes G1-G5.

### Compositions Comprising Compounds of the Disclosure

Another aspect of the present disclosure relates to compositions comprising a (a) a DNA protein kinase inhibitor (DNA-PKI) and (b) a DNA cutting agent, wherein the DNA-PKI is a compound of Formula (I), or a pharmaceutically acceptable salt, hydrate, solvate, prodrug, stereoisomer, or tautomer thereof. In some embodiments, the composition further comprises a cell. In some embodiments, the composition further comprises a donor DNA. In some embodiments, the composition further comprises a cell and a donor DNA.

In some embodiments, the cell is a eukaryotic cell. In some embodiments, the cell is useful in adoptive cell therapy (ACT). In some embodiments, the cell is a stem cell. In some embodiments, the cell is a hematopoietic stem cell (HSC) or an induced pluripotent stem cell (iPSC). In some embodiments, the cell is an immune cell. In some embodiments, the immune cell is a leukocyte, or a lymphocyte (e.g., a T cell, a B cell, or an NK cell). In some embodiments, the immune cell is a lymphocyte. In some embodiments, the lymphocyte is a T cell. In some embodiments, the lymphocyte is a primary T cell. In some embodiments, the lymphocyte is a regulatory T cell. In some embodiments, the lymphocyte is an activated T cell. In some embodiments, the lymphocyte is a non-activated T cell. In some embodiments, the cell is a human cell. In some embodiments, the cell is not a cancer cell.

In some embodiments, the donor DNA comprises a template comprising a sequence encoding a protein, a regulatory sequence, or a sequence encoding structural RNA.

In some embodiments, the DNA cutting agent is selected from a zinc finger nuclease, a TALE effector domain nuclease (TALEN), a CRISPR/Cas nuclease component, and combinations thereof.

In some embodiments, the DNA cutting agent comprises a CRISPR/Cas nuclease component and optionally a guide RNA component. In some embodiments, the CRISPR/Cas nuclease component comprises a Cas nuclease or an mRNA encoding the Cas nuclease. In some embodiments, the CRISPR/Cas nuclease component comprises or encodes a CRISPR/Cas nuclease that generates a double strand DNA break or single strand DNA break. In some embodiments, the CRISPR/Cas nuclease component comprises or encodes a CRISPR/Cas nuclease that generates a single strand DNA break.

In some embodiments, the DNA cutting agent is a CRISPR/Cas nuclease component and a guide RNA component. In some embodiments, the CRISPR/Cas nuclease component comprises a Cas nuclease or an mRNA encoding the Cas nuclease. In some embodiments, the Cas nuclease is a Class 2, Type II Cas nuclease. In some embodiments, the Cas nuclease is a Cas9 nuclease (e.g., a S. pyogenes Cas9 nuclease). In some embodiments, the Cas nuclease is a Class 2, Type V Cas nuclease. In some embodiments, the Cas nuclease is a Cas12a nuclease (e.g., a Acidaminococcus sp. Cas12a nuclease).

In some embodiments, the composition comprises a modified RNA.

In some embodiments, the guide RNA component is a guide RNA nucleic acid. In some embodiments, the guide RNA component is a guide RNA (gRNA). In some embodiments, the guide RNA nucleic acid is or encodes a dual-guide RNA (dgRNA). In some embodiments, the dual-guide RNA is composed of a crRNA and tracrRNA. In some embodiments, the guide RNA nucleic acid is or encodes a single-guide (sgRNA). In some embodiments, the gRNA is a modified gRNA.

In some embodiments, the DNA cutting agent is Cas9 or an mRNA encoding Cas9, and a modified gRNA comprising a modification at one or more of the first five nucleotides at the 5′ end. In some embodiments, the cutting agent is Cas12a or an mRNA encoding Cas12a, and a modified gRNA comprising a DNA/RNA hybrid molecule. In some embodiments, the modified gRNA comprises a modification at one or more of the last five nucleotides at the 3′ end.

In some embodiments, the DNA cutting agent is a Class 2, Type II or Class 2, Type V Cas nuclease and a guide RNA nucleic acid; and the molar ratio of the guide RNA to Cas nuclease is from about 4:1 to 1:4.

In some embodiments, the composition further comprises a vector. In some embodiments, the vector encodes the donor DNA. In some embodiments, the vector is a viral vector (e.g., an AAV). In some embodiments, the vector is a non-viral vector. In some embodiments, the vector is a non-viral vector comprising donor DNA having a linear, close end, circular, single strand, double strand format.

In some embodiments, the composition further comprises an inhibitor of the microhomology mediated end joining (MMEJ) pathway. In some embodiments, the inhibitor of the MMEJ pathway is a DNA polymerase theta (Polθ or POLQ) inhibitor. In some embodiments, the inhibitor of the MMEJ pathway is a FEN1 inhibitor. In some embodiments, the inhibitor of the MMEJ pathway is selected from the group consisting of a PolQ inhibitor selected from the compounds described in J. Med. Chem 2023, 66, 6498 by Pismataro, M. C., et al. and references therein, for example, inhibitor of PolQ is ART558 (Artios Pharma Limited), ART812 (Artios Pharma Limited), novobiocin (Dana-Farber Cancer Institute, Inc.) Compound 23 (Ideaya Biosciences, Inc.), and RP-6685 (Repare Therapeutics), or combinations thereof.

In some embodiments, the concentration of the DNA-PKI in the composition is about 10 μM or less. In some embodiments, the concentration of the DNA-PKI in the composition is from about 0.1 μM to about 10 μM. In some embodiments, the concentration of the DNA-PKI in the composition is from about 0.25 μM to about 5 μM. In some embodiments, the concentration of the DNA-PKI in the composition is from about 0.25 μM to about 10 μM. In some embodiments, the concentration of the DNA-PKI in the composition is from about 0.1 μM to about 5 μM. In some embodiments, the concentration of the DNA-PKI in the composition is from about 0.1 μM to about 0.25 μM.

### Methods of Using the Disclosed Compounds

Another aspect of the present disclosure is directed to a method for targeted genome editing in a cell, comprising contacting the cell with a DNA cutting agent and a DNA-PKI, wherein the DNA-PKI is a compound of Formula (I), or a pharmaceutically acceptable salt, hydrate, solvate, prodrug, stereoisomer, or tautomer thereof.

Another aspect of the present disclosure is directed to a method for repairing a double stranded DNA break in the genome of a cell, comprising contacting the cell with a DNA cutting agent and a DNA-PKI, wherein the DNA-PKI is a compound of Formula (I), or a pharmaceutically acceptable salt, hydrate, solvate, prodrug, stereoisomer, or tautomer thereof. In some embodiments, the double stranded DNA break is a blunt end break. In some embodiments, the double stranded DNA break comprises paired single strand breaks (e.g., made by a combination of nickase nucleases).

Another aspect of the present disclosure is directed to a method for inhibiting or suppressing repair of a DNA break in a cell via a nonhomologous end joining (NHEJ) pathway, comprising contacting the cell with a DNA cutting agent and a DNA-PKI, wherein the DNA-PKI is a compound of Formula (I), or a pharmaceutically acceptable salt, hydrate, solvate, prodrug, stereoisomer, or tautomer thereof. In some embodiments, the method further comprises contacting the cell with an inhibitor of the microhomology mediated end joining (MMEJ) pathway. In some embodiments, the inhibitor of the MMEJ pathway is a DNA polymerase theta (Polθ or POLQ) inhibitor. In some embodiments, the inhibitor of the MMEJ pathway is a FEN1 inhibitor. In some embodiments, the inhibitor of the MMEJ pathway is selected from the group consisting of a PolQ inhibitor selected from the compounds described in J. Med. Chem 2023, 66, 6498 by Pismataro, M. C., et al. and references therein, for example, inhibitor of PolQ is ART558 (Artios Pharma Limited), ART812 (Artios Pharma Limited), novobiocin (Dana-Farber Cancer Institute, Inc.) Compound 23 (Ideaya Biosciences, Inc.), and RP-6685 (Repare Therapeutics), or combinations thereof. Another aspect of the present disclosure is directed to a method for targeted insertion of a donor DNA into the genome of a cell, comprising contacting the cell with a DNA cutting agent, the donor DNA, and a DNA-PKI, wherein the DNA-PKI is a compound of Formula (I), or a pharmaceutically acceptable salt, hydrate, solvate, prodrug, stereoisomer, or tautomer thereof.

In some embodiments of the methods disclosed herein, the method comprises growing the cell in a cell medium free of the DNA-PKI, and adding the DNA-PKI to the cell medium.

In some embodiments of the methods disclosed herein, the method comprises contacting the cell with the DNA cutting agent before contacting the cell with the DNA-PKI. In some embodiments, the method comprises contacting the cell with the DNA-PKI within about six hours of contacting the cell with the DNA cutting agent. In some embodiments, the methods comprise contacting the cell with the DNA-PKI within about three hours of contacting the cell with the DNA cutting agent. In some embodiments, the methods comprise contacting the cell with the DNA-PKI within about two hours of contacting the cell with the DNA cutting agent. In some embodiments, the methods comprise contacting the cell with the DNA-PKI between about 15 minutes and about 45 minutes of contacting the cell with the DNA cutting agent. In some embodiments, the methods comprise contacting the cell with the DNA-PKI about 30 minutes of contacting the cell with the DNA cutting agent.

In some embodiments, the methods comprise contacting the cell with the DNA cutting agent simultaneously with the DNA-PKI.

In some embodiments of the methods disclosed herein, the method comprises growing the cell in a cell medium comprising the DNA-PKI.

In some embodiments, the methods comprise contacting the cell with the DNA cutting agent after contacting the cell with the DNA-PKI. In some embodiments, the methods comprise contacting the cell with the DNA cutting agent within about three hours of contacting the cell with the DNA-PKI.

In some embodiments, contacting the cell with the DNA cutting agent comprises electroporating the cell to allow the DNA cutting agent to enter the cell. In some embodiments, contacting the cell with the DNA cutting agent comprises delivering the DNA cutting agent to the cell using other methods, e.g., microinjection or via a lipid nanoparticle, liposome, exosome, or gold nanoparticle. In some embodiments, the methods comprise contacting the cell with the DNA cutting agent and the donor DNA simultaneously.

In some embodiments of the methods disclosed herein, the method comprises contacting the cell with the DNA cutting agent and the DNA-PKI for at least about one day. In some embodiments, the method comprises contacting the cell with the DNA cutting agent and the DNA-PKI for about one day. In some embodiments, the method comprises contacting the cell with the DNA cutting agent and the DNA-PKI for between about one day and about two weeks. In some embodiments, the method comprises contacting the cell with the DNA cutting agent and the DNA-PKI for about two weeks.

In some embodiments of the methods disclosed herein, the method comprises contacting the cell with the DNA-PKI in a cell medium, wherein the concentration of the DNA-PKI in the cell medium is about 10 μM or less. In some embodiments, the method comprises contacting the cell with the DNA-PKI in a cell medium, wherein the concentration of the DNA-PKI in the cell medium is between about 0.1 μM and about 10 μM. In some embodiments, the method comprises contacting the cell with the DNA-PKI in a cell medium, wherein the concentration of the DNA-PKI in the cell medium is between about 0.25 μM and about 5 μM.

In some embodiments of the methods disclosed herein, the cell is a eukaryotic cell. In some embodiments, the cell is for use in adoptive cell therapy (ACT). In some embodiments, the cell is for use in autologous cell therapy. In some embodiments, the cell is for use in allogenic cell therapy. In some embodiments, the cell is a stem cell. In some embodiments, the cell is a hematopoietic stem cell (HSC) or an induced pluripotent stem cell (iPSC). In some embodiments, the cell is an immune cell. In some embodiments, the immune cell is a leukocyte, or a lymphocyte (e.g., a T cell, a B cell, or an NK cell). In some embodiments, the immune cell is a lymphocyte. In some embodiments, the lymphocyte is a T cell. In some embodiments, the lymphocyte is a primary T cell. In some embodiments, the lymphocyte is a regulatory T cell. In some embodiments, the lymphocyte is an activated T cell. In some embodiments, the lymphocyte is a non-activated T cell. In some embodiments, the cell is a human cell. In some embodiments, the cell is not a cancer cell.

In some embodiments of the methods disclosed herein, the DNA cutting agent is selected from a zinc finger nuclease, a TALE effector domain nuclease (TALEN), a CRISPR/Cas nuclease component, and combinations thereof.

In some embodiments of the methods disclosed herein, the DNA cutting agent comprises a CRISPR/Cas nuclease component and optionally a guide RNA component. In some embodiments, the CRISPR/Cas nuclease component comprises a Cas nuclease or an mRNA encoding the Cas nuclease. In some embodiments, the CRISPR/Cas nuclease component comprises or encodes a CRISPR/Cas nuclease that generates a double strand DNA break or single strand DNA break. In some embodiments, the CRISPR/Cas nuclease component comprises or encodes a CRISPR/Cas nuclease that generates a single strand DNA break.

In some embodiments of the methods disclosed herein, the DNA cutting agent is a CRISPR/Cas nuclease component and a guide RNA component. In some embodiments, the CRISPR/Cas nuclease component comprises a Cas nuclease or an mRNA encoding the Cas nuclease. In some embodiments, the Cas nuclease is a Class 2, Type II Cas nuclease. In some embodiments, the Cas nuclease is a Cas9 nuclease (e.g., a S. pyogenes Cas9 nuclease). In some embodiments, the Cas nuclease is a Class 2, Type V Cas nuclease. In some embodiments, the Cas nuclease is a Cas12a nuclease (e.g., a Acidaminococcus sp. Cas12a nuclease).

In some embodiments of the methods disclosed herein, the methods further comprise contacting the cell with a modified RNA.

In some embodiments of the methods disclosed herein, the methods further comprise contacting the cell with a guide RNA component. In some embodiments, the guide RNA component is a guide RNA nucleic acid. In some embodiments, the guide RNA component is a guide RNA (gRNA). In some embodiments, the guide RNA nucleic acid is or encodes a dual-guide RNA (dgRNA). In some embodiments, the guide RNA nucleic acid is or encodes a single-guide (sgRNA). In some embodiments, the gRNA is a modified gRNA.

In some embodiments of the methods disclosed herein, the DNA cutting agent is Cas9 or an mRNA encoding Cas9, and a modified gRNA comprising a modification at one or more of the first five nucleotides at the 5′ end. In some embodiments, the cutting agent is Cas12a or an mRNA encoding Cas12a, and a modified gRNA comprising a DNA/RNA hybrid molecule. In some embodiments, the modified gRNA comprises a modification at one or more of the last five nucleotides at the 3′ end.

In some embodiments of the methods disclosed herein, the DNA cutting agent is a Class 2, Type II or Class 2, Type V Cas nuclease and a guide RNA nucleic acid; and the molar ratio of the guide RNA to Cas nuclease is from about 4:1 to 1:4.

In some embodiments of the methods disclosed herein, the DNA cutting agent interacts with a target sequence within the TRAC gene of a T cell.

In some embodiments of the methods disclosed herein, the methods comprise contacting the cell with at least two different DNA cutting agents targeting different loci.

In some embodiments, the methods comprise contacting the cell with a vector encoding the DNA cutting agent. In some embodiments, the vector encodes the DNA cutting agent and the donor DNA. In some embodiments, the methods comprise contacting the cell with a vector encoding the DNA cutting agent, and a second vector encoding the donor DNA.

In some embodiments, the vector is a viral vector (e.g., an AAV). In some embodiments, the vector is a non-viral vector. In some embodiments, the vector is a non-viral vector comprising donor DNA having a linear, close end, circular, single strand, double strand format.

In some embodiments of the methods disclosed herein, the DNA cutting agent interacts with a target sequence within the genome of the cell, resulting in a double stranded DNA break (DSB).

In some embodiments of the methods disclosed herein, the methods further comprise contacting the cell with a donor DNA. In some embodiments, the methods comprise contacting the cell with a vector comprising the donor DNA. In some embodiments, the vector encodes the donor DNA. In some embodiments, the donor DNA comprises a template comprising a sequence encoding a protein, a regulatory sequence, or a sequence encoding structural RNA. In some embodiments, the donor DNA comprises a template comprising an exogenous nucleic acid encoding a protein. In some embodiments, the protein is selected from a cytokine, an immunosuppressor, an antibody, a receptor, and an enzyme. In some embodiments, the protein is a receptor. In some embodiments, the receptor is selected from an immunological receptor, a T-cell receptor (TCR), and a chimeric antigen receptor. In some embodiments, the exogenous nucleic acid encodes a TCR chain of a TCR, e.g. a TCR alpha, beta, delta, or gamma chain, or any combination thereof. In some embodiments, the exogenous nucleic acid encodes a TCR alpha and/or TCR beta chain. In some embodiments, the template comprises a first homology arm and a second homology arm that are complementary to sequences located upstream and downstream of the cleavage site, respectively.

In some embodiments of the methods disclosed herein, the method results in a gene knockout. In some embodiments of the methods disclosed herein, the method results in a gene correction. In some embodiments of the methods disclosed herein, the method results in a gene insertion.

In some embodiments, the methods further comprise contacting the cell with an inhibitor of the microhomology mediated end joining (MMEJ) pathway. In some embodiments, the inhibitor of the MMEJ pathway is a DNA polymerase theta (Polθ or POLQ) inhibitor. In some embodiments, the inhibitor of the MMEJ pathway is a FEN1 inhibitor. In some embodiments, the inhibitor of the MMEJ pathway is selected from the group consisting of a PolQ inhibitor selected from the compounds described in J. Med. Chem 2023, 66, 6498 by Pismataro, M. C., et al. and references therein, for example, inhibitor of PolQ is ART558 (Artios Pharma Limited), ART812 (Artios Pharma Limited), novobiocin (Dana-Farber Cancer Institute, Inc.) Compound 23 (Ideaya Biosciences, Inc.), and RP-6685 (Repare Therapeutics), or combinations thereof. Another aspect of the present disclosure relates to compounds of Formula (I), and pharmaceutically acceptable salts, hydrates, solvates, prodrugs, stereoisomers, tautomers, or pharmaceutical compositions thereof, for use in the manufacture of a medicament for cell therapy.

Another aspect of the present disclosure relates to the use of a compound of Formula (I), or a pharmaceutically acceptable salt, hydrate, solvate, prodrug, stereoisomer, tautomer, or pharmaceutical composition thereof, in the treatment of a cell.

In one embodiment, the subject is a mammal.

In one embodiment, the mammal is a human.

Administration of the disclosed compounds may also be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising a compound of the present disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds may be also formulated as a suppository that can be prepared from fatty emulsions or suspensions; using polyalkylene glycols such as propylene glycol, as the carrier.

The disclosed compounds may also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564 which is hereby incorporated by reference in its entirety.

Disclosed compounds may also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the Disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the present disclosure is directed to pharmaceutical compositions comprising a compound of Formula (I) and a pharmaceutically acceptable carrier. The pharmaceutical acceptable carrier may further include an excipient, diluent, or surfactant. In some embodiments, the pharmaceutical composition can further comprise an additional pharmaceutically active agent.

In one embodiment, the pharmaceutical acceptable carrier further comprises an excipient, diluent, surfactant, or any combination thereof.

In one embodiment, the pharmaceutical composition further comprises at least one additional therapeutic agent.

Another aspect of the present disclosure is directed to pharmaceutical compositions for use in cell therapy comprising a compound of Formula (I), or a pharmaceutically acceptable salt thereof.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

In one embodiment, the composition comprises about 1 mg to about 2000 mg of the compound.

In one embodiment, the composition is administered to the subject twice daily, once daily, once every other day, or once weekly.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

The compounds of the present disclosure may be prepared by use of known chemical reactions and procedures. Nevertheless, the following general preparative methods are presented to aid the reader in synthesizing the compounds with specific details provided below in the experimental section to illustrate working examples.

All variable groups of these methods are as described in the generic description if they are not specifically defined below.

It is recognized that compounds of the disclosure with each claimed optional functional group may not be prepared by each of the below-listed methods. Within the scope of each method, optional substituents may appear on reagents or intermediates which may act as protecting or otherwise non-participating groups. Utilizing methods well known to those skilled in the art, these groups are introduced and/or removed during the course of the synthetic schemes which provide the compounds of the present disclosure.

### Acronyms and Abbreviations

Table 2 provides a list of acronyms and abbreviations used in this specification, along with their meanings.

TABLE 2

| ACRONYM OR | |
|---|---|
| ABBREVIATION | MEANING OR DEFINITION |
| Ac | Acetyl |
| ACN, MeCN | Acetonitrile |
| Aq. | Aqueous |
| Boc | t-Butyloxycarbonyl |
| Brettphos Pd G3 | [(2-Di-cyclohexylphosphino-3,6-dimethoxy- |
| | 2′,4′,6′-triisopropyl-1,1′-biphenyl)-2-(2′- |
| | amino-1,1′-biphenyl)]palladium(II) |
| | methanesulfonate |
| | methanesulfonate |
| DCM | Dichloromethane |
| DIPEA, DIEA | N,N-diisopropylethylamine, also known as |
| | Hünig's base |
| DMA | N,N-Dimethylacetamide |
| DMAP | 4-(Dimethylamino)pyridine |
| DMF | N,N-dimethylformamide |
| DMSO | Dimethyl sulfoxide |
| EA, EtOAc | Ethyl acetate |
| h | Hour |
| HCl | Hydrochloric acid |
| HPLC | High pressure liquid chromatography |
| Hunig's base | See DIPEA, DIEA |
| LCMS, LC-MS, | Liquid chromatography-mass spectrometry |
| LC/MS | |
| min | Minute |
| MS | Mass spectrometry |
| MTBE | Methyl tert-butyl ether |
| NMR | Nuclear magnetic resonance |
| PE | Petroleum ether |
| Prep | Preparative |
| RT (in context of | Retention time, in min |
| liquid chromatography) | |
| rt or RT (in the context | Room (ambient) temperature, circa 25° C. |
| of reaction conditions) | |
| Sat. | Saturated |
| Soln | Solution |
| TEA | Triethylamine |
| TFA | Trifluoroacetic acid |
| THF | Tetrahydrofuran |
| TLC | Thin layer chromatography |

### Analytical Procedures

### NMR

The following conditions were used for obtaining proton nuclear magnetic resonance (NMR) spectra: NMR spectra were taken in either 400 MHz or 500 MHz. Bruker instrument using either DMSO-d₆ or CDCl₃ as solvent and internal standard. The crude NMR data was analyzed by using either ACD Spectrus version 2015-01 by ADC Labs or MestReNova software.

Chemical shifts are reported in parts per million (ppm) downfield from internal tetramethylsilane (TMS) or from the position of TMS inferred by the deuterated NMR solvent. Apparent multiplicities are reported as: singlet-s, doublet-d, triplet-t, quartet-q, or multiplet-m. Peaks that exhibit broadening are further denoted as br. Integrations are approximate. It should be noted that integration intensities, peak shapes, chemical shifts and coupling constants can be dependent on solvent, concentration, temperature, pH, and other factors. Further, peaks that overlap with or exchange with water or solvent peaks in the NMR spectrum may not provide reliable integration intensities. In some cases, NMR spectra may be obtained using water peak suppression, which may result in overlapping peaks not being visible or having altered shape and/or integration.

### Liquid Chromatography

The following preparative and/or analytical (LC/MS) liquid chromatography methods were used.

Method A: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+).

Method B: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+).

UHPLC Method C: Column: Waters Acquity BEH C18 2.1×50 mm 1.7 μm particles; Mobile Phase A: 95:5 acetonitrile:water with 0.05% TFA; Mobile Phase B: 95:5 acetonitrile:water with 0.05% TFA; Temperature: 50° C.; Gradient: 0% B to 100% B over 3.00 min, then a 0.50 min hold at 100% B; Flow: 1.0 mL/min; Detection: MS and UV (254 nm).

UPLCMS Method D: Column: Aquity BEH C18 (50×3.0) mm, 1.7 μm, Mobile Phase: A: 5 mm Ammonium formate pH 3.3:ACN (98:02), Mobile Phase B: ACN:Buffer (98:02), Flow Rate: 0.7 ml/min; Time (min)/Grad (% B): 0/20, 1.5/98, 2/98. Detection: MS and UV (254 nm).

UHPLC Method E: Column: Waters Acquity BEH C18 2.1×50 mm 1.7 μm particles; Mobile Phase A: 95:5 acetonitrile:water with 0.05% TFA; Mobile Phase B: 95:5 acetonitrile:water with 0.05% TFA; Temperature: 50° C.; Gradient: 0% B to 100% B over 1.5 min, then a 0.50 min hold at 100% B; Flow: 1.0 mL/min; Detection: MS and UV (254 nm).

LCMS method F: Column: Kinetex XB-C18 (75×3 mm) 2.6 μm; Mobile phase: A: 0.1% TFA in H₂O; Mobile phase: B: 0.1% TFA in ACN; Flow Rate: 1.0 mL/min. Time/Gradient: 5% B to 95% B in 2.5 min, then hold at 95% B for 2 min hold at 95% B.

LCMS Method G: Column: Kinetex XB-C18 (75×3 mm) 2.6 μm; 5 mm Ammonium formate pH 3.3:ACN (98:02); Mobile Phase: B: ACN:Buffer (98:02); Flow Rate: 1.0 mL/min. Time/Gradient: 0% B to 100% B in 4.0 min, then hold at 100% B for 0.6 min (1.5 mL/min). Analytical HPLC A: Column: Kinetex Biphenyl (100×4.6) mm, 2.6 μm, Mobile phase: A: 0.05% TFA in WATER:ACN; Mobile phase: B: 0.05% TFA IN ACN:WATER; Flow: 1.0 mL/min; Time/Gradient (% B): 0/10, 9/60, 11/100, 11.1/100 (1.5 mL/min), 12.5/100 (1.5 mL/min), 13/10, 15/10. Analytical HPLC B: Column: Kinetex EVO C18 (100×4.6) mm, 2.6 μm, Mobile phase: A: 0.05% TFA in water:ACN (95:5), Mobile phase: B: ACN:0.05% TFA in water (95:5); Flow: 1.0 mL/min; Time (min)/Grad (% B): 0/10, 1/30, 9/80, 11/100, 11.1/100 (1.5 mL/min), 12.5/100 (1.5 mL/min), 13/10, 15/10.

### Preparation of 2-chloro-9-(4-hydroxybicyclo[2.2.1]heptan-1-yl)-7-methyl-7,9-dihydro-8H-purin-8-one (Int-1) and 2-chloro-9-(4-methoxybicyclo[2.2.1]heptan-1-yl)-7-methyl-7,9-dihydro-8H-purin-8-one (Int-2) as Shown in Scheme 1

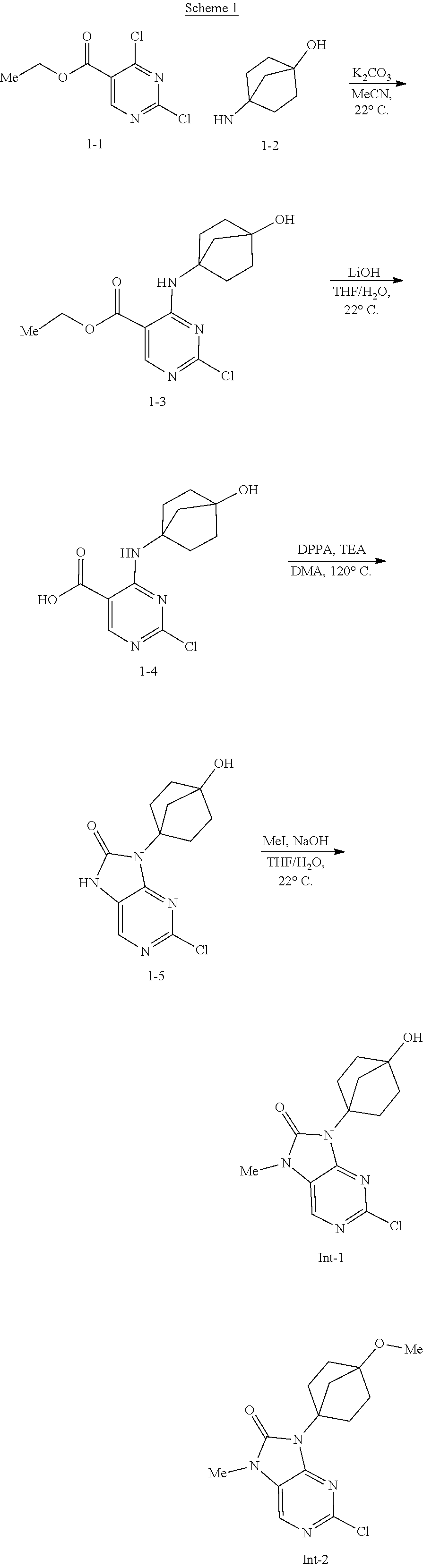

### Step 1: Synthesis of ethyl 2-chloro-4-((4-hydroxybicyclo[2.2.1]heptan-1-yl)amino)pyrimidine-5-carboxylate (1-3)

Potassium carbonate (1.037 g, 7.50 mmol) was added to ethyl 2,4-dichloropyrimidine-5-carboxylate (0.663 g, 3.00 mmol) and 4-aminobicyclo[2.2.1]heptan-1-ol (0.491 g, 3.00 mmol) in acetonitrile (18 mL). The reaction mixture was stirred at RT for 16 h. The precipitate was collected by filtration and washed with EtOAc (10 mL), and the filtrate was removed under reduced pressure to afford the product 1-3 (0.935 g, 3.00 mmol, 100% yield). MS: m/z: Calc'd for C₁₄H₁₈ClN₃O₃ [M+H]⁺ 312, found 312.

### Step 2: Synthesis of 2-chloro-4-((4-hydroxybicyclo[2.2.1]heptan-1-yl)amino)pyrimidine-5-carboxylic acid (1-4)

A solution of LiOH (144 mg, 6.00 mmol) in water (9.0 mL) was added to a stirred solution of ethyl 2-chloro-4-((4-hydroxybicyclo[2.2.1]heptan-1-yl)amino)pyrimidine-5-carboxylate (935 mg, 3.00 mmol) in THF (9.0 mL). The reaction mixture was stirred at 22° C. for 3 h. The organic layers were removed under reduced pressure. The reaction mixture was acidified with 2 M aqueous HCl. The precipitate was collected by filtration, washed with water (8 mL), and dried under vacuum to afford the title compound 1-4 (851 mg, 3.00 mmol, 100% yield) as a white solid. MS: m/z: Calc'd for C₁₂H₁₄ClN₃O₃ [M+H]⁺ 284, found 284.

### Step 3: Synthesis of 2-chloro-9-(4-hydroxybicyclo[2.2.1]heptan-1-yl)-7,9-dihydro-8H-purin-8-one (1-5)

Triethylamine (0.418 mL, 3.00 mmol) was added to 2-chloro-4-((4-hydroxybicyclo[2.2.1]heptan-1-yl)amino)pyrimidine-5-carboxylic acid (850 mg, 3.00 mmol) and diphenylphosphoryl azide (DPPA, 825 mg, 3.00 mmol) in DMA (5.0 mL). The reaction mixture was allowed to stir at 22° C. for 1 h and then at 120° C. for 16 h. The reaction mixture was poured into ice (25 mL), and the precipitate was collected by filtration, washed with water (10 mL), and dried under vacuum to afford the title compound 1-5 (841 mg, 3.00 mmol, 100% yield) as a white solid. MS: m/z: Calc'd for C₁₂H₁₃ClN₄O₂[M+H]⁺ 281, found 281.

### Step 4: Synthesis of 2-chloro-9-(4-hydroxybicyclo[2.2.1]heptan-1-yl)-7-methyl-7,9-dihydro-8H-purin-8-one (Int-1) and 2-chloro-9-(4-methoxybicyclo[2.2.1]heptan-1-yl)-7-methyl-7,9-dihydro-8H-purin-8-one (Int-2)

A solution of NaOH (599 mg, 15.00 mmol) in water (1.0 mL) was added to a stirred solution of 2-chloro-9-(4-hydroxybicyclo[2.2.1]heptan-1-yl)-7,9-dihydro-8H-purin-8-one (841 mg, 3.00 mmol) and Mel (0.937 mL, 15.00 mmol) in THF (6.0 mL). The reaction mixture was stirred at 22° C. for 16 h. The organic layer was removed under reduced pressure. The reaction mixture was diluted with water. The precipitate was collected by filtration, washed with water (30 mL), and dried under vacuum to afford the crude compound. The crude product was purified by column chromatography (0% to 30% EtOAc in Heptane) to afford Int-1 (370 mg, 1.32 mmol, 41.9% yield) as a white solid and Int-2 (90 mg, 0.29 mmol, 9.7% yield) as a white solid.

Int-1: MS: m/z: Calc'd for C₁₃H₁₅ClN₄O₂[M+H]⁺ 295, found 295.

Int-2: MS: m/z: Calc'd for C₁₄H₁₇ClN₄O₂[M+H]⁺ 309, found 309.

### Preparation of 2-chloro-9-(3-fluorobicyclo[1.1.1]pentan-1-yl)-7-methyl-7,9-dihydro-8H-purin-8-one (Int-3)

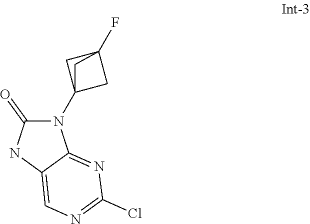 [SMILES: O=c1[nH]c2cnc(Cl)nc2n1C12CC(F)(C1)C2]

The title compound was prepared in 47.15% overall yield as a white solid according to the preparation of Int-1 using 3-fluorobicyclo[1.1.1]pentan-1-amine, HCl in STEP 1. MS: m/z: Calc'd for C₁₁H₁₀ClFN₄O, [M+H]⁺ 269; Found 269.

### Preparation of 3-(2-chloro-7-methyl-8-oxo-7,8-dihydro-9H-purin-9-yl)bicyclo[1.1.1]pentane-1-carbonitrile (Int-4)

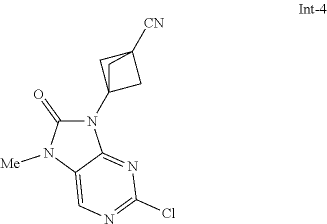 [SMILES: Cn1c(=O)n(C23CC(C#N)(C2)C3)c2nc(Cl)ncc21]

The title compound was prepared in 2.3% overall yield as a white solid according to the preparation of Int-1 using 3-aminobicyclo[1.1.1]pentane-1-carbonitrile in STEP 1. MS: m/z: Calc'd for C₁₂H₁₀ClFN₅O, [M+H]⁺ 276; Found 276.

### Preparation of 2-chloro-7-methyl-9-(3-(trifluoromethyl)bicyclo[1.1.1]pentan-1-yl)-7,9-dihydro-8H-purin-8-one (Int-5)

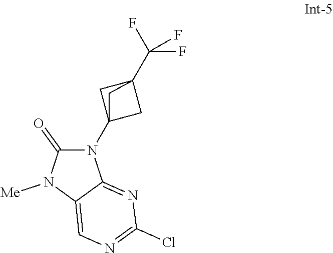 [SMILES: Cn1c(=O)n(C23CC(C(F)(F)F)(C2)C3)c2nc(Cl)ncc21]

The title compound was prepared in 9.4% overall yield as a white solid according to the preparation of Int-1 using 3-(trifluoromethyl)bicyclo[1.1.1]pentan-1-amine, HCl in STEP 1. MS: m/z: Calc'd for C₁₂H₁₀ClF₃N₄O, [M+H]⁺ 319; Found 319.

### Preparation of 2-chloro-9-(3-(methoxymethyl)bicyclo[1.1.1]pentan-1-yl)-7-methyl-7,9-dihydro-8H-purin-8-one (Int-6)

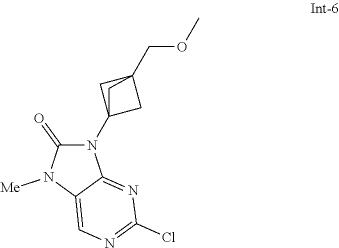 [SMILES: COCC12CC(n3c(=O)n(C)c4cnc(Cl)nc43)(C1)C2]

The title compound was prepared in 52.0% overall yield as a white solid according to the preparation of Int-1 using 3-(methoxymethyl)bicyclo[1.1.1]pentan-1-amine, HCl in STEP 1. MS: m/z: Calc'd for C₁₃H₁₅ClN₄O₂, [M+H]⁺ 295; Found 295.

### Preparation of 2-(3-(2-chloro-7-methyl-8-oxo-7,8-dihydro-9H-purin-9-yl)bicyclo[1.1.1]pentan-1-yl)acetonitrile (Int-7)

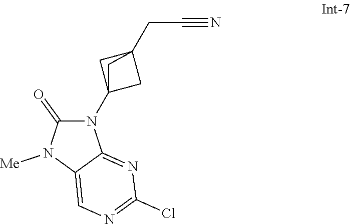 [SMILES: Cn1c(=O)n(C23CC(CC#N)(C2)C3)c2nc(Cl)ncc21]

The title compound was prepared in 47.0% overall yield as a white solid according to the preparation of Int-1 using 2-(3-aminobicyclo[1.1.1]pentan-1-yl)acetonitrile in STEP 1. MS: m/z: Calc'd for C₁₃H₁₂ClN₅O, [M+H]⁺ 290; Found 290.

### Preparation of 2-chloro-9-(3-(hydroxymethyl)bicyclo[1.1.1]pentan-1-yl)-7-methyl-7,9-dihydro-8H-purin-8-one (Int-8)

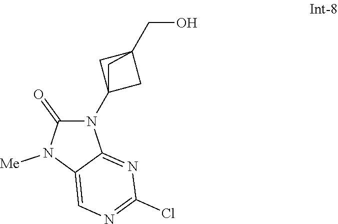 [SMILES: Cn1c(=O)n(C23CC(CO)(C2)C3)c2nc(Cl)ncc21]

The title compound was prepared in 5.1% overall yield as a white solid according to the preparation of Int-1 using (3-aminobicyclo[1.1.1]pentan-1-yl)methanol, HCl in STEP 1. MS: m/z: Calc'd for C₁₃H₁₂ClN₅O, [M+H]⁺ 281; Found 281.

### Preparation of 4-(2-chloro-7-methyl-8-oxo-7,8-dihydro-9H-purin-9-yl)bicyclo[2.2.2]octane-1-carbonitrile (Int-9)

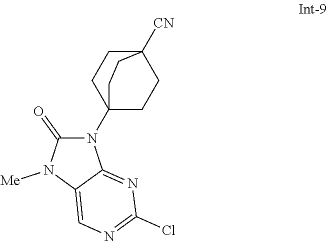 [SMILES: Cn1c(=O)n(C23CCC(C#N)(CC2)CC3)c2nc(Cl)ncc21]

The title compound was prepared in 37.8% overall yield as a white solid according to the preparation of Int-1 using 4-aminobicyclo[2.2.2]octane-1-carbonitrile in STEP 1. MS: m/z: Calc'd for C₁₅H₁₆ClN₅O, [M+H]⁺ 318; Found 318.

### Preparation of 2-chloro-9-(4-methoxybicyclo[2.2.2]octan-1-yl)-7-methyl-7,9-dihydro-8H-purin-8-one (Int-10)

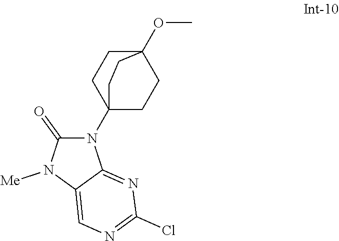 [SMILES: COC12CCC(n3c(=O)n(C)c4cnc(Cl)nc43)(CC1)CC2]

The title compound was prepared in 62.4% overall yield as a white solid according to the preparation of Int-1 using 4-methoxybicyclo[2.2.2]octan-1-amine, HCl in STEP 1. MS: m/z: Calc'd for C₁₅H₁₉ClN₄O₂, [M+H]⁺ 323; Found 323.

### Preparation of 2-chloro-9-(4-hydroxybicyclo[2.2.2]octan-1-yl)-7-methyl-7,9-dihydro-8H-purin-8-one (Int-11)

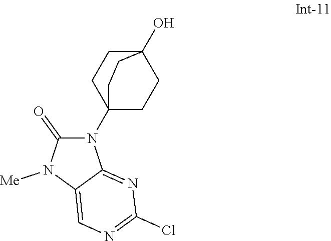 [SMILES: Cn1c(=O)n(C23CCC(O)(CC2)CC3)c2nc(Cl)ncc21]

The title compound was prepared in 53.9% overall yield as a white solid according to the preparation of Int-1 using 4-aminobicyclo[2.2.2]octan-1-ol, HCl in STEP 1. MS: m/z: Calc'd for C₁₄H₁₇ClN₄O₂, [M+H]⁺ 309; Found 309.

### Preparation of 2-chloro-9-(4-methoxybicyclo[2.1.1]hexan-1-yl)-7-methyl-7,9-dihydro-8H-purin-8-one (Int-12)

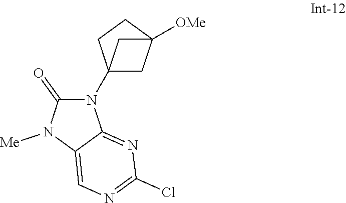 [SMILES: COC12CCC(n3c(=O)n(C)c4cnc(Cl)nc43)(C1)C2]

The title compound was prepared in 27.0% overall yield as a white solid according to the preparation of Int-2 using 4-aminobicyclo[2.1.1]hexan-1-ol, HCl in STEP 1. MS: m/z: Calc'd for C₁₃H₁₅ClN₄O₂, [M+H]⁺ 295; Found 295.

### Preparation of 2-chloro-9-(4-hydroxybicyclo[2.2.1]heptan-1-yl)-7-methyl-7,9-dihydro-8H-purine-8-thione (Int-13) as Shown in Scheme 2

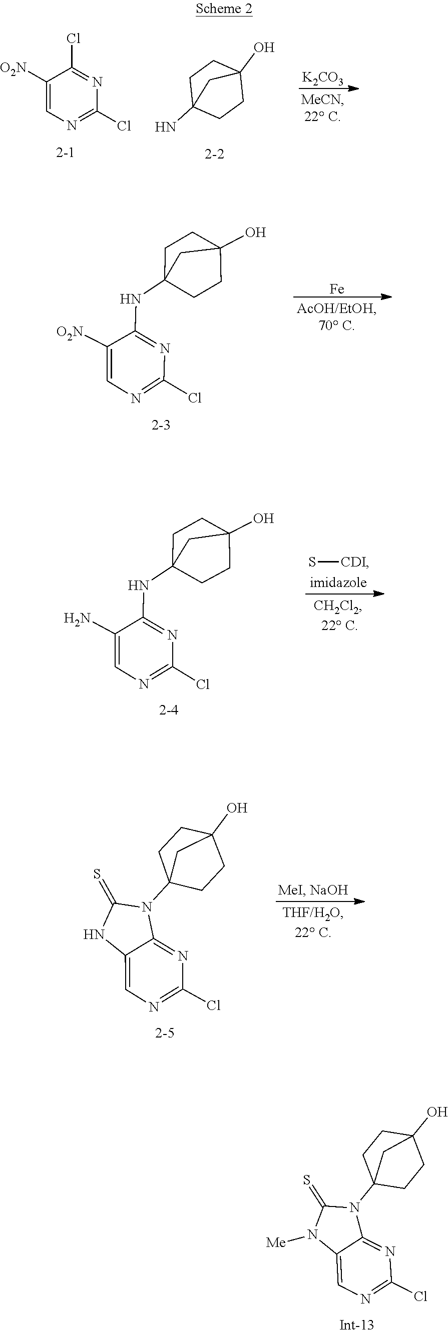

### Step 1: Synthesis of 4-((2-chloro-5-nitropyrimidin-4-yl)amino)bicyclo[2.2.1]heptan-1-ol (2-3)

Potassium carbonate (1.04 g, 7.50 mmol) was added to 2,4-dichloro-5-nitropyrimidine (582 mg, 3.00 mmol) and 4-aminobicyclo[2.2.1]heptan-1-ol (491 mg, 3.00 mmol) in acetonitrile (18.0 mL). The reaction mixture was stirred at 22° C. for 16 h. The precipitate was collected by filtration and washed with EtOAc (10 mL), and the filtrate was removed under reduced pressure. The crude product was purified by column chromatography (0% to 80% EtOAc in Heptane) to afford the title compound (170 mg, 0.60 mmol, 19.9% yield) as a pale-yellow solid. MS: m/z: Calc'd for C₁H₁₃ClN₄O₃, [M+H]⁺ 285; Found 285.

### Step 2: Synthesis of 4-((5-amino-2-chloropyrimidin-4-yl)amino)bicyclo[2.2.1]heptan-1-ol (2-4)

To a vial containing 4-((2-chloro-5-nitropyrimidin-4-yl)amino)bicyclo[2.2.1]heptan-1-ol (0.60 mmol, 170 mg) was added iron (167 mg, 2.99 mmol), EtOH (0.5 mL), H₂O (0.5 mL) and AcOH (0.5 mL). The resulting mixture was allowed to warm up to 70° C. and stir at the same temperature for 1 h. The resulting suspension was filtered and filter cake was washed with DCM/MeOH. The filtrate was concentrated and purified by column chromatography (50% to 100% EtOAc in Heptane) to afford the product (152 mg, 0.60 mmol, 100% yield) as a solid. MS: m/z: Calc'd for C₁₁H₁₅ClN₄O, [M+H]⁺ 255; Found 255.

### Step 3: Synthesis of 2-chloro-9-(4-hydroxybicyclo[2.2.1]heptan-1-yl)-7,9-dihydro-8H-purine-8-thione (2-5)

To a solution of 4-((5-amino-2-chloropyrimidin-4-yl)amino)bicyclo[2.2.1]heptan-1-ol (152 mg, 0.60 mmol) in CH₂Cl₂ (3.0 mL) was added imidazole (81 mg, 1.19 mmol) and 1 1′-thiocarbonyldiimidazole (319 mg, 1.79 mmol). The resulting solution was allowed to stir at 22° C. for 1 h. The mixture was acidified by 1 M HCl to PH=5 and the solid crashed out. The precipitate was collected and dried under vacuum to afford the product (177 mg, 0.59 mmol, 98.3% yield) as a white solid. MS: m/z: Calc'd for C₁₂H₁₃ClN₄OS, [M+H]⁺ 297; Found 297.

### Step 4: Synthesis of 2-chloro-9-(4-hydroxybicyclo[2.2.1]heptan-1-yl)-7-methyl-7,9-dihydro-8H-purine-8-thione (Int-13)

A solution of NaOH (71.6 mg, 1.79 mmol) in water (1.5 mL) was added to a stirred solution of 2-chloro-9-(4-hydroxybicyclo[2.2.2]octan-1-yl)-7,9-dihydro-8H-purine-8-thione (177 mg, 0.59 mmol) and Mel (0.112 mL, 1.79 mmol) in THF (3.0 mL). The reaction mixture was stirred at 22° C. for 16 h. The organic layer was removed under reduced pressure. The reaction mixture was diluted with water. The precipitate was collected by filtration, washed with water (3.0 mL), and dried under vacuum to afford the title compound (116 mg, 0.37 mmol, 62.6% yield) as a white solid. MS: m/z: Calc'd for C₁₃H₁₅ClN₄OS, [M+H]⁺ 311; Found 311.

### Preparation of 2-chloro-9-(3-fluorobicyclo[1.1.1]pentan-1-yl)-7-methyl-7,9-dihydro-8H-purine-8-thione (Int-14)

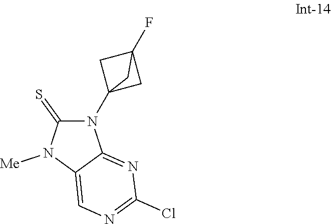 [SMILES: Cn1c(=S)n(C23CC(F)(C2)C3)c2nc(Cl)ncc21]

The title compound was prepared in 15.1% overall yield as a white solid according to the preparation of Int-13 using 3-fluorobicyclo[1.1.1]pentan-1-amine, HCl in STEP 1. MS: m/z: Calc'd for C₁₁H₁₀ClFN₄S, [M+H]⁺ 285; Found 285.

### Preparation of 9-((1s,4s)-bicyclo[2.2.2]octan-2-yl)-2-chloro-7-methyl-7,9-dihydro-8H-purin-8-one (Int-15)

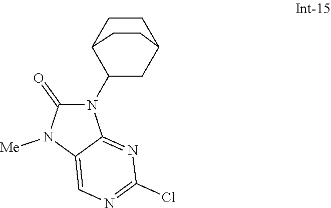 [SMILES: Cn1c(=O)n(C2CC3CCC2CC3)c2nc(Cl)ncc21]

The title compound was prepared in 38.4% overall yield as a white solid according to the preparation of Int-1 using (1s,4s)-bicyclo[2.2.2]octan-2-amine, HCl in STEP 1. MS: m/z: Calc'd for C₁₄H₁₇ClN₄O, [M+H]⁺ 293; Found 293.

### Preparation of 9-((1S,2S,4R)-bicyclo[2.2.1]heptan-2-yl)-2-chloro-7-methyl-7,9-dihydro-8H-purin-8-one (Int-16)

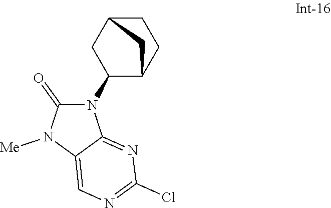 [SMILES: Cn1c(=O)n([C@H]2C[C@@H]3CC[C@H]2C3)c2nc(Cl)ncc21]

The title compound was prepared in 23.9% overall yield as a white solid according to the preparation of Int-1 using (1S,2S,4R)-bicyclo[2.2.1]heptan-2-amine in STEP 1. MS: m/z: Calc'd for C₁₃H₁₅ClN₄O, [M+H]⁺ 279; Found 279.

### Preparation of 1-(bicyclo[1.1.1]pentan-1-yl)-6-chloro-3-methyl-1H-pyrazolo[3,4-d]pyrimidine (Int-17) as Shown in Scheme 3

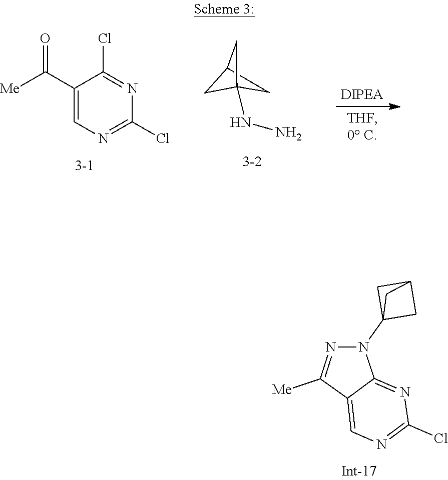 [SMILES: CC(=O)c1cnc(Cl)nc1Cl ; NNC12CC(C1)C2 ; Cc1nn(C23CC(C2)C3)c2nc(Cl)ncc12]

A mixture of DIPEA (0.21 mL, 1.20 mmol), bicyclo[1.1.1]pentan-1-ylhydrazine, 2 HCl (0.056 g, 0.33 mmol) and 1-(2,4-dichloropyrimidin-5-yl)ethanone (0.057 g, 0.30 mmol) in THF (3.00 mL) was stirred for 3 h at 0° C. under nitrogen atmosphere. The resulting mixture was concentrated under reduced pressure. The residue was purified by MPLC (hexane/EtOAc 1:1) to afford the product (18.7 mg, 0.08 mmol, 26.7% yield) as a white solid. MS: m/z: Calc'd for C₁₁H₁₁ClN₄, [M+H]⁺ 235; Found 235.

### Preparation of 1-(bicyclo[1.1.1]pentan-1-yl)-6-chloro-3-methyl-1H-pyrazolo[3,4-d]pyrimidine (Int-18) as Shown in Scheme 4

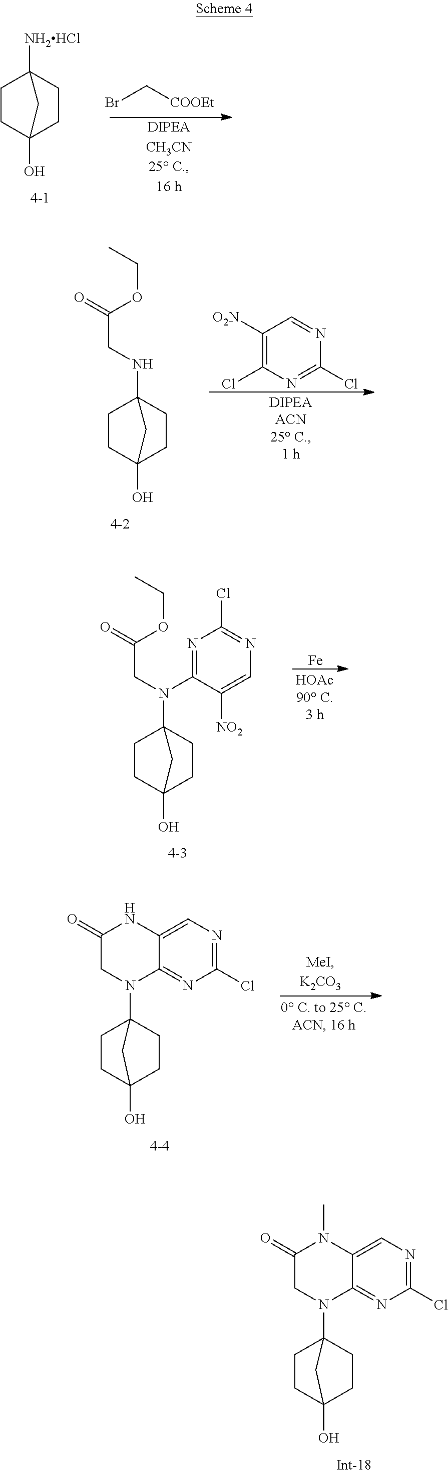

### Step 1. Ethyl (4-hydroxybicyclo[2.2.1]heptan-1-yl)glycinate

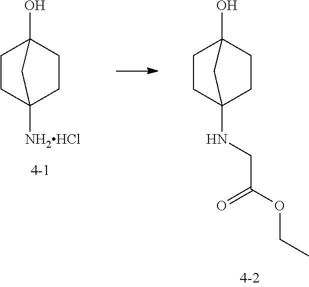

To a stirred solution of 4-aminobicyclo[2.2.1]heptan-1-ol hydrochloride (1.0 g, 6.11 mmol) in acetonitrile (20 mL) were added DIPEA (2.67 mL, 15.28 mmol) and ethyl bromoacetate (1.021 g, 6.11 mmol) at ambient temperature. Then, it was stirred at 25° C. for 16 h. The reaction was monitored by GCMS. The reaction mixture was diluted with water (100 mL). The crude product was extracted with ethyl acetate (2×100 mL). The combined organic layers were washed with brine, dried with anhydrous sodium sulphate, and concentrated under reduced pressure to give the product, ethyl (4-hydroxybicyclo[2.2.1]heptan-1-yl)glycinate (1.1 g, 2.73 mmol, 44.7% yield). GCMS m/z (M, M+H)⁺; 215.2, 216.2; Rt=6.019 min.

Column: Kinetex XB—C18 (75×30) mm, 2.6 μm Mobile Phase A: 5 mm Ammonium formate pH 3.3:ACN (98:02), Flow Rate: 1.0 ml/min.

### Step 2. Ethyl N-(2-chloro-5-nitropyrimidin-4-yl)-N-(4-hydroxybicyclo[2.2.1]heptan-1-yl)glycinate

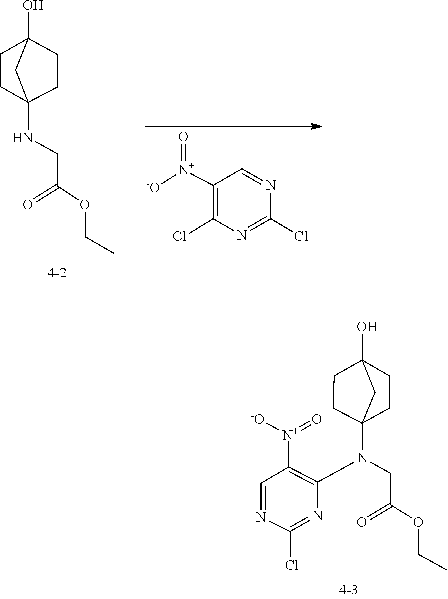

To a stirred solution of ethyl (4-hydroxybicyclo[2.2.1]heptan-1-yl)glycinate (550 mg, 2.58 mmol) in acetonitrile (4 mL) were added DIPEA (1.126 mL, 6.45 mmol) and 2,4-dichloro-5-nitropyrimidine (500 mg, 2.58 mmol) at 0-25° C. Then, it was stirred at 25° C. for 1 h. The reaction was monitored by TLC and LCMS. The reaction mixture was diluted with water (100 mL). The crude product was extracted with ethyl acetate (2×100 mL). The combined organic layer was washed with brine, dried with anhydrous sodium sulphate, and concentrated under reduced pressure. The concentrated product was purified by RP-column purification in (0.1% ammonium acetate:ACN) to obtain ethyl N-(2-chloro-5-nitropyrimidin-4-yl)-N-(4-hydroxybicyclo[2.2.1]heptan-1-yl)glycinate (700 mg, 1.718 mmol, 66.6% yield) as a brown gum.

Column: Kinetex XB—C18 (75×30) mm, 2.6 μm Mobile Phase A: 5 mm Ammonium formate pH 3.3:ACN (98:02) Mobile Phase A: 5 mm Ammonium formate pH 3.3:ACN (98:02) Flow Rate: 1.0 ml/min.

### Step 3. 2-Chloro-8-(4-hydroxybicyclo[2.2.1]heptan-1-yl)-7,8-dihydropteridin-6(5H)-one

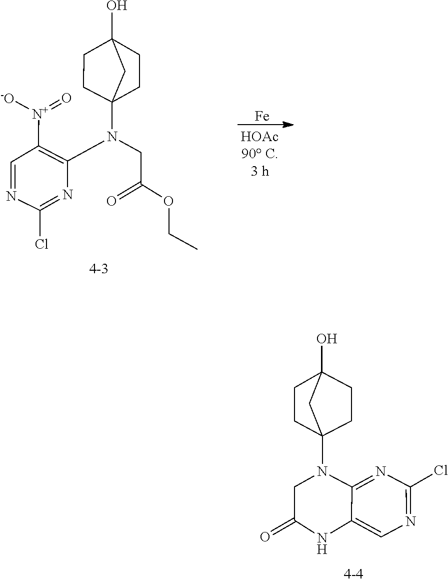

To a stirred solution of ethyl N-(2-chloro-5-nitropyrimidin-4-yl)-N-(4-hydroxybicyclo[2.2.1]heptan-1-yl)glycinate (700 mg, 1.888 mmol) in acetic Acid (10 mL) were added Iron (316 mg, 5.66 mmol) at 25° C. Then, it was stirred at 70° C. for 2 h. The reaction was monitored by TLC and LCMS. The reaction mixture was concentrated under vacuum, diluted with water (70 mL), and neutralized with 10% NaHCO₃ solution (pH-8), and filtered through cilite. The crude product was extracted with ethyl acetate (2×80 mL). The combined organic layer was washed with brine, dried with anhydrous sodium sulphate, and concentrated under reduced pressure. The concentrated product was purified by RP-column purification in (0.1% ammonium acetate:ACN) to obtain 2-chloro-8-(4-hydroxybicyclo[2.2.1]heptan-1-yl)-7,8-dihydropteridin-6(5H)-one (280 mg, 0.893 mmol, 47.3% yield) as an off-white solid.

LCMS m/z (M, M+H)⁺; 295.2, 296.2; Rt: 1.66-1.69

Column: Kinetex XB—C18 (75×30) mm, 2.6 μm Mobile Phase A: 5 mm Ammonium formate pH 3.3:ACN (98:02) Mobile Phase A: 5 mm Ammonium formate pH 3.3:ACN (98:02) Flow Rate: 1.0 ml/min.

### Step 4. 2-Chloro-8-(4-hydroxybicyclo[2.2.1]heptan-1-yl)-5-methyl-7,8-dihydropteridin-6(5H)-one (Int-18)

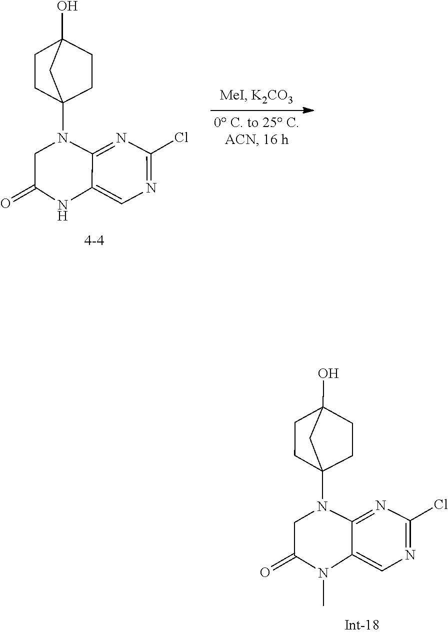 [SMILES: O=C1CN(C23CCC(O)(CC2)C3)c2nc(Cl)ncc2N1 ; CN1C(=O)CN(C23CCC(O)(CC2)C3)c2nc(Cl)ncc21]

To a stirred solution of 2-chloro-8-(4-hydroxybicyclo[2.2.1]heptan-1-yl)-7,8-dihydropteridin-6(5H)-one (150 mg, 0.509 mmol) in N,N-Dimethylformamide (3 mL) were added potassium carbonate (211 mg, 1.527 mmol) and Mel (0.064 mL, 1.018 mmol) at 0° C. Then, it was stirred at 0-25° C. for 16 hr. The reaction was monitored and LCMS. The reaction mixture was diluted with water (30 mL). The crude product was extracted with ethyl acetate (2×50 mL). The combined organic layer was washed with brine, dried with anhydrous sodium sulphate, and concentrated under reduced pressure. The concentrated product was purified by RP-column purification in (0.1% ammonium acetate:ACN) to obtain 2-chloro-8-(4-hydroxybicyclo[2.2.1]heptan-1-yl)-5-methyl-7,8-dihydropteridin-6(5H)-one (60 mg, 0.190 mmol, 37.4% yield) as an off-white solid.

LCMS m/z (M, M+H)⁺; 309.2, 310.2; Rt: 0.842-0.88 min.

Column: Kinetex XB—C18 (75×30) mm, 2.6 μm Mobile Phase A: 5 mm Ammonium formate pH 3.3:ACN (98:02) Mobile Phase A: 5 mm Ammonium formate pH 3.3:ACN (98:02) Flow Rate: 1.0 ml/min.

### Example 1: 9-(4-hydroxybicyclo[2.2.1]heptan-1-yl)-7-methyl-2-((7-methyl-[1,2,4]triazolo[1,5-a]pyridin-6-yl)amino)-7,9-dihydro-8H-purin-8-one (Compound 1)

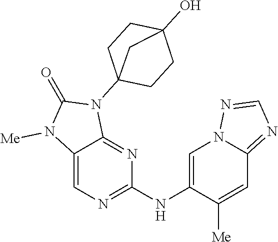 [SMILES: Cc1cc2ncnn2cc1Nc1ncc2c(n1)n(C13CCC(O)(CC1)C3)c(=O)n2C]

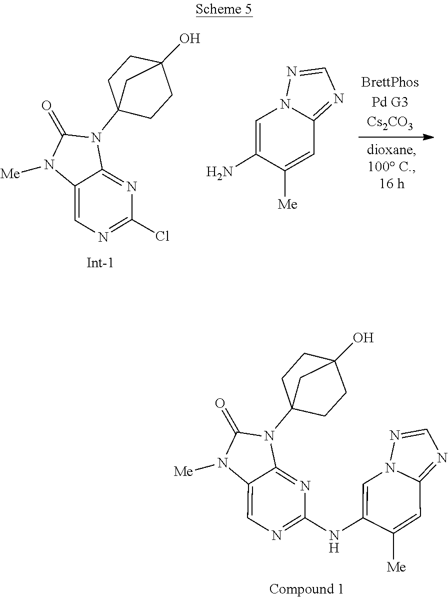 [SMILES: Cn1c(=O)n(C23CCC(O)(CC2)C3)c2nc(Cl)ncc21 ; Cc1cc2ncnn2cc1N ; Cc1cc2ncnn2cc1Nc1ncc2c(n1)n(C13CCC(O)(CC1)C3)c(=O)n2C]

1,4-Dioxane (0.5 mL) was added to the vial with 2-chloro-9-(1,1-dimethylsilolan-3-yl)-7-methyl-7,9-dihydro-8H-purin-8-one (15 mg, 0.05 mmol) and 7-methyl-[1,2,4]triazolo[1,5-a]pyridin-6-amine (7.4 mg, 0.05 mmol), Brettphos Pd G3 (9.1 mg, 0.01 mmol) and Cesium carbonate (33 mg, 0.10 mmol). The resulting suspension was purged with N₂ for 10 mins and allowed to stir vigorously at 100° C. for 16 h. The mixture was cooled to 22° C., filtered through celite and washed with 10% MeOH in DCM. The filtrate was taken, and the solvent was removed in vacuo. The resulting residue was purified by reversed-phase chromatography (0.05% NH₄OAc in H₂O and ACN) to obtain the title compound (14.5 mg, 72.5% yield) as a white solid. MS: m/z: Calc'd for C₂₀H₂₂N₈O₂ [M+H]⁺ 407; Found 407. ¹H NMR (500 MHz, DMSO-d₆) δ 9.10 (s, 1H), 8.54 (s, 1H), 8.37 (s, 1H), 8.06 (s, 1H), 7.70 (s, 1H), 5.05-5.01 (m, 1H), 3.40-3.34 (m, 3H), 2.39 (s, 5H), 2.31 (s, 2H), 1.99-1.91 (m, 2H), 1.70 (br t, J=8.7 Hz, 2H), 1.58-1.51 (m, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 407, retention time 1.16 min, purity: 100%; LCMS 2 with TFA: [M+H]⁺ found 407, retention time 1.03 min, purity: 97.2%.

Compound 1 was also prepared according to the following synthetic protocol.

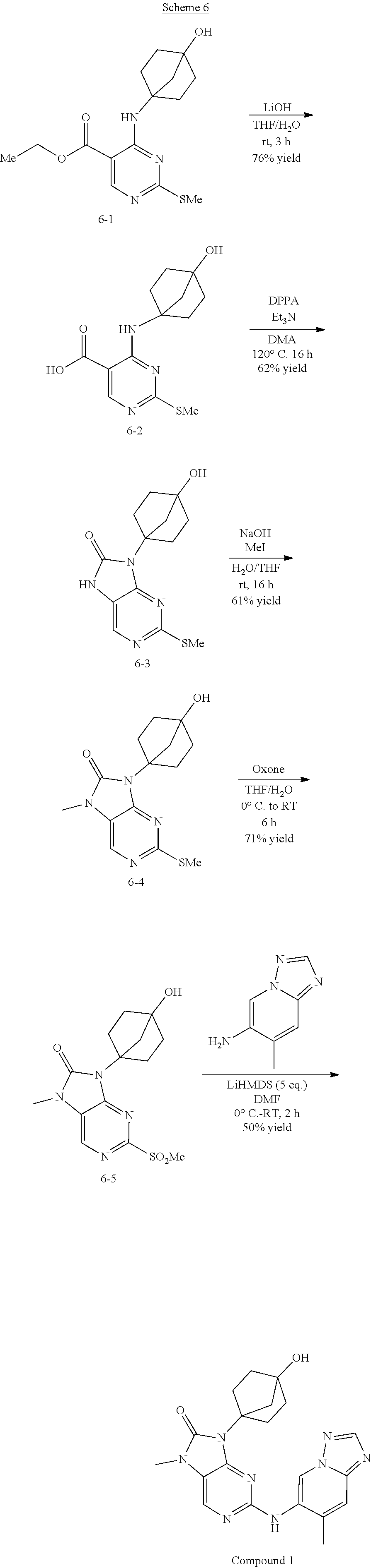

### Step 1′. 4-((4-hydroxybicyclo[2.2.1]heptan-1-yl)amino)-2-(methylthio)pyrimidine-5-carboxylic acid (6-2)

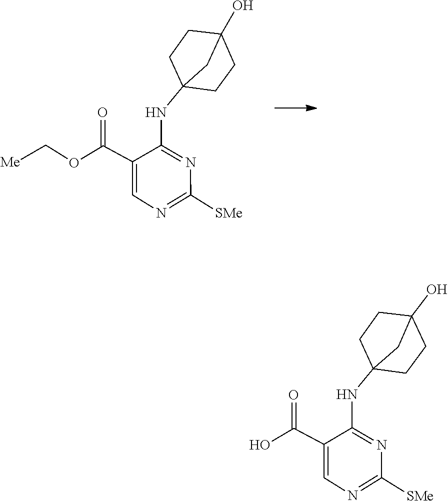 [SMILES: CCOC(=O)c1cnc(SC)nc1NC12CCC(O)(CC1)C2 ; CSc1ncc(C(=O)O)c(NC23CCC(O)(CC2)C3)n1]

In a 20-ml glass vial, ethyl 4-((4-hydroxybicyclo[2.2.1]heptan-1-yl)amino)-2-(methylthio)pyrimidine-5-carboxylate (450 mg, 1.391 mmol) was taken. To the reaction mixture, THF (6 mL) and Water (6 mL) was added followed by Lithium hydroxide monohydrate (234 mg, 5.57 mmol). The reaction mixture was stirred for 3 h at 25° C. The reaction mixture was concentrated under vacuum to remove the organic volatile and acidified with 1.5N HCl. Precipitation was not observed. The reaction mixture was further concentrated under vacuum (volume of aqueous layer was reduced to half). White precipitate was obtained. The white solid was filtered through buchner funnel and dried under vacuum to afford 4-((4-hydroxybicyclo[2.2.1]heptan-1-yl)amino)-2-(methylthio)pyrimidine-5-carboxylic acid (380 mg, 1.055 mmol, 76% yield) as a white solid. UPLC-MS: 296.0 [M+H]⁺.

### Step 2′. 9-(4-hydroxybicyclo[2.2.1]heptan-1-yl)-2-(methylthio)-7,9-dihydro-8H-purin-8-one (6-3)

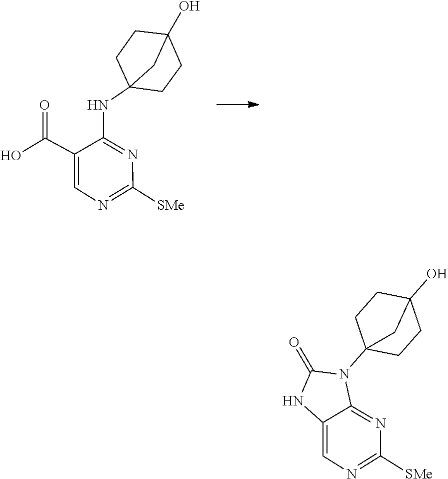 [SMILES: CSc1ncc(C(=O)O)c(NC23CCC(O)(CC2)C3)n1 ; CSc1ncc2[nH]c(=O)n(C34CCC(O)(CC3)C4)c2n1]

In a 8-ml glass vial 4-((4-hydroxybicyclo[2.2.1]heptan-1-yl)amino)-2-(methylthio)pyrimidine-5-carboxylic acid (380 mg, 1.287 mmol) was taken. To it 1,4-Dioxane (15 mL) and DMA (1.2 mL) was added. To it then triethylamine (0.197 mL, 1.415 mmol) was added followed by diphenyl phosphoryl azide (389 mg, 1.415 mmol). The reaction mixture was stirred for 1 h at 25° C. The reaction mixture was stirred at 100° C. over a period of 16 h. Brown reaction mixture was concentrated under vacuum. Crude was purified by silica gel column chromatography using silica gel (60-120 mesh) and 0-10% MeOH/DCM to give 9-(4-hydroxybicyclo[2.2.1]heptan-1-yl)-2-(methylthio)-7,9-dihydro-8H-purin-8-one (250 mg, 0.803 mmol, 62.4% yield). LCMS: m/z 293.2 [M+H]⁺.

### Step 3′. 9-(4-hydroxybicyclo[2.2.1]heptan-1-yl)-7-methyl-2-(methylthio)-7,9-dihydro-8H-purin-8-one (6-4)

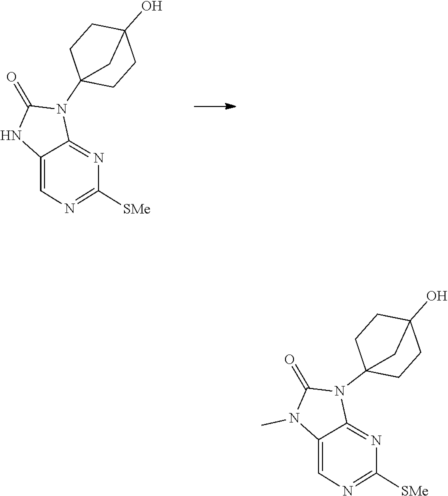 [SMILES: CSc1ncc2[nH]c(=O)n(C34CCC(O)(CC3)C4)c2n1 ; CSc1ncc2c(n1)n(C13CCC(O)(CC1)C3)c(=O)n2C]

In a 20 ml glass vial 9-(4-hydroxybicyclo[2.2.1]heptan-1-yl)-2-(methylthio)-7,9-dihydro-8H-purin-8-one (125 mg, 0.428 mmol) was taken and to it THF (3 mL) and Water (1 mL) was added. To it then sodium hydroxide (68.4 mg, 1.710 mmol) was added followed by iodomethane (0.053 mL, 0.855 mmol). The reaction mixture was stirred at 25° C. over a period of 5 h. After 5 h UPLCMS and TLC was checked it showed that SM was not consumed. Reaction mixture was stirred at 25° C. over a period of 11 h. Heterogeneous reaction mixture. The reaction mixture was concentrated under vacuum. Crude was purified with silica gel (60-120 mesh) and eluted with 0-10% MeOH/DCM to give 9-(4-hydroxybicyclo[2.2.1]heptan-1-yl)-7-methyl-2-(methylthio)-7,9-dihydro-8H-purin-8-one (124 mg, 76% yield). LC-MS: m/z 307 [M+H]⁺, RT=1.32 min.

### Step 4′. 9-(4-hydroxybicyclo[2.2.1]heptan-1-yl)-7-methyl-2-(methylsulfonyl)-7,9-dihydro-8H-purin-8-one (6-5)

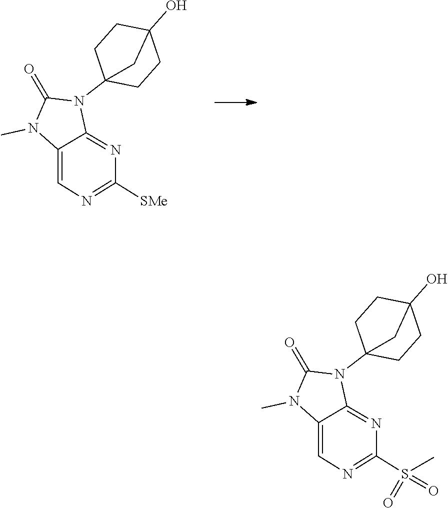

In a 20-ml glass vial 9-(4-hydroxybicyclo[2.2.1]heptan-1-yl)-7-methyl-2-(methylthio)-7,9-dihydro-8H-purin-8-one (100 mg, 0.326 mmol) was taken and to it THF (3 mL) and Water (3 mL) was added. The reaction mixture was cooled to 0° C. and to it OXONE, monopersulfate (602 mg, 0.979 mmol) was added. The reaction mixture was stirred for 6 h at 25° C. Reaction was monitored by UPLC-MS and TLC. The reaction mixture was filtered through buchner funnel. The solid obtained was washed with 5% MeOH/DCM. The combined filtrate was concentrated under vacuum and purified through RP purification. Pure fraction after concentration gave the desired compound (90 mg, 81% yield). LC-MS: m/z 339 [M+H]⁺.

### Step 5′. 9-(4-hydroxybicyclo[2.2.1]heptan-1-yl)-7-methyl-2-((7-methyl-[1,2,4]triazolo[1,5-a]pyridin-6-yl)amino)-7,9-dihydro-8H-purin-8-one (Compound 1)

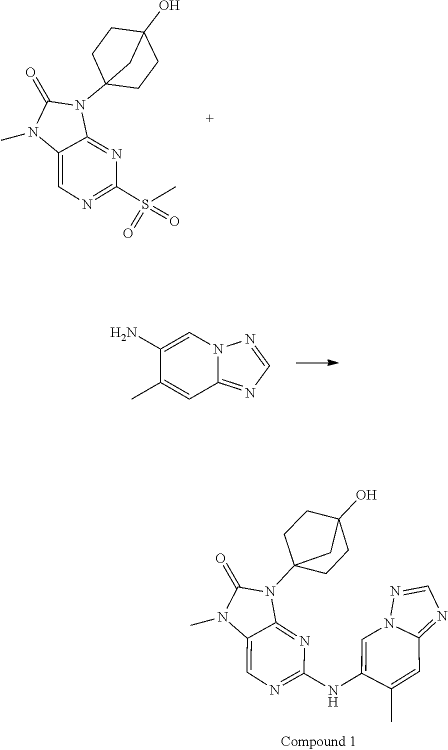

In a 8-ml glass vial 9-(4-hydroxybicyclo[2.2.1]heptan-1-yl)-7-methyl-2-(methylsulfonyl)-7,9-dihydro-8H-purin-8-on e (35 mg, 0.103 mmol) was taken and to it 7-methyl-[1,2,4]triazolo[1,5-a]pyridin-6-amine (22.99 mg, 0.155 mmol) and DMF (0.5 mL) were added. To the mixture Lithium bis(trimethylsilyl)amide solution in THF (0.517 mL, 0.517 mmol) was added. The reaction mixture was stirred for 2 h at 25° C. After worked up the crude was purified by Prep HPLC: Diluent: THF:ACN:water (50:30:20), Column: X Select C18 (150×19) mm, 5 micron, Temperature: Ambient, Mobile phase A: 5 mM Ammonium Formate, Mobile phase B: ACN, Flow: 15 mL/min, Time (min)/Grad: 0/10, 12/50) to give the desired product as a white solid (21.2 mg, 50.1%). LCMS: m/z: 407.3 (M+H)+ LCMS (purity)=99.38%, RT: 1.453 min; HPLC (purity)=99.29%, RT: 5.85 min (HPLC Method B; ¹H NMR (400 MHz, DMSO-d6): δ 9.10 (s, 1H), 8.53 (s, 1H), 8.37 (s, 1H), 8.06 (s, 1H), 7.71 (s, 1H), 5.01 (s, 1H), 3.27 (s, 3H), 2.39-2.42 (m, 5H), 2.32-2.34 (m, 2H), 1.92-1.99 (m, 2H), 1.67-1.74 (m, 2H), 1.53-1.58 (m, 2H).

### Example 2: 9-(4-hydroxybicyclo[2.2.1]heptan-1-yl)-7-methyl-2-((6-methylbenzo[d][1,3]dioxol-5-yl)amino)-7,9-dihydro-8H-purin-8-one (Compound 2)

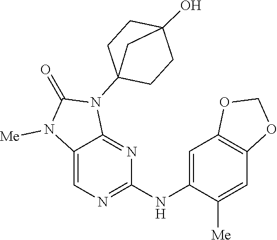 [SMILES: Cc1cc2c(cc1Nc1ncc3c(n1)n(C14CCC(O)(CC1)C4)c(=O)n3C)OCO2]

The title compound was prepared in 68.0% overall yield as a white solid according to the preparation of EXAMPLE 1 using 6-methylbenzo[d][1,3]dioxol-5-amine. MS: m/z: Calc'd for C₂₁H₂₃N₅O₄ [M+H]⁺ 410; Found 410. ¹H NMR (500 MHz, DMSO-d₆) δ 8.14 (s, 1H), 7.95 (s, 1H), 7.01 (s, 1H), 6.78 (s, 1H), 5.95 (s, 2H), 5.05-5.01 (m, 1H), 3.43-3.20 (m, 3H), 2.39 (br s, 2H), 2.30 (s, 2H), 2.11 (s, 3H), 1.97-1.88 (m, 2H), 1.74-1.66 (m, 2H), 1.59-1.52 (m, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 410, retention time 1.46 min, purity: 100%; LCMS 2 with TFA: [M+H]⁺ found 410, retention time 1.17 min, purity: 100%.

### Example 3: 9-(4-hydroxybicyclo[2.2.1]heptan-1-yl)-7-methyl-2-((6-methylbenzo[c][1,2,5]thiadiazol-5-yl)amino)-7,9-dihydro-8H-purin-8-one (Compound 3)

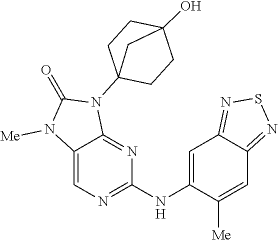 [SMILES: Cc1cc2nsnc2cc1Nc1ncc2c(n1)n(C13CCC(O)(CC1)C3)c(=O)n2C]

The title compound was prepared in 51.5% overall yield as a white solid according to the preparation of EXAMPLE 1 using 6-methylbenzo[c][1,2,5]thiadiazol-5-amine. MS: m/z: Calc'd for C₂₁H₂₃N₅O₄ [M+H]⁺ 424; Found 424. ¹H NMR (500 MHz, DMSO-d₆) δ 8.57 (s, 1H), 8.40 (s, 1H), 8.20 (s, 1H), 7.89 (s, 1H), 5.10-5.06 (m, 1H), 3.34-3.15 (m, 3H), 2.54 (s, 3H), 2.50-2.46 (m, 2H), 2.38 (s, 2H), 2.05-1.98 (m, 2H), 1.75 (br t, J=8.3 Hz, 2H), 1.63-1.56 (m, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 424, retention time 1.48 min, purity: 100%; LCMS 2 with TFA: [M+H]⁺ found 424, retention time 1.19 min, purity: 100%.

### Example 4: 9-(4-hydroxybicyclo[2.2.1]heptan-1-yl)-7-methyl-2-((7-methylquinoxalin-6-yl)amino)-7,9-dihydro-8H-purin-8-one (Compound 4)

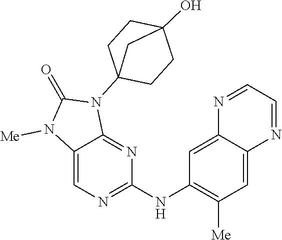 [SMILES: Cc1cc2nccnc2cc1Nc1ncc2c(n1)n(C13CCC(O)(CC1)C3)c(=O)n2C]

The title compound was prepared in 69.2% overall yield as a white solid according to the preparation of EXAMPLE 1 using 7-methylquinoxalin-6-amine. MS: m/z: Calc'd for C₂₂H₂₃N₇O₂ [M+H]⁺ 418; Found 418. ¹H NMR (500 MHz, DMSO-d₆) δ 8.79 (d, J=1.7 Hz, 1H), 8.72 (d, J=1.7 Hz, 1H), 8.60 (s, 1H), 8.53 (s, 1H), 8.19 (s, 1H), 7.90 (s, 1H), 5.02 (s, 1H), 3.43-3.33 (m, 3H), 2.57 (s, 3H), 2.48 (br d, J=0.9 Hz, 2H), 2.36 (s, 2H), 2.00 (br t, J=10.9 Hz, 2H), 1.74 (br t, J=9.2 Hz, 2H), 1.62-1.53 (m, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 418, retention time 1.22 min, purity: 100%; LCMS 2 with TFA: [M+H]⁺ found 418, retention time 1.00 min, purity: 100%.

### Example 5: 9-(4-hydroxybicyclo[2.2.1]heptan-1-yl)-7-methyl-2-((7-methyl-2,3-dihydrobenzo[b][1,4]diox in-6-yl)amino)-7,9-dihydro-8H-purin-8-one (Compound 5)

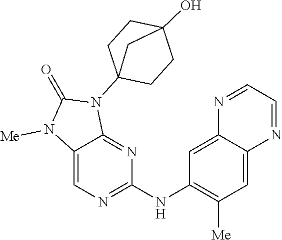 [SMILES: Cc1cc2nccnc2cc1Nc1ncc2c(n1)n(C13CCC(O)(CC1)C3)c(=O)n2C]

The title compound was prepared in 59.2% overall yield as a white solid according to the preparation of EXAMPLE 1 using 7-methyl-2,3-dihydrobenzo[b][1,4]dioxin-6-amine, HCl. MS: m/z: Calc'd for C₂₁H₂₃N₅O₄ [M+H]⁺ 424; Found 424. ¹H NMR (500 MHz, DMSO-d₆) δ 8.08 (s, 1H), 7.95 (s, 1H), 6.99 (s, 1H), 6.67 (s, 1H), 5.04-5.01 (m, 1H), 4.19 (s, 4H), 3.32-3.14 (m, 3H), 2.41 (br s, 2H), 2.31 (s, 2H), 2.08 (s, 3H), 1.96-1.89 (m, 2H), 1.75-1.67 (m, 2H), 1.55 (br s, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 424, retention time 1.36 min, purity: 98.8%; LCMS 2 with TFA: [M+H]⁺ found 424, retention time 1.07 min, purity: 98.9%.

### Example 6: 9-(4-methoxybicyclo[2.2.1]heptan-1-yl)-7-methyl-2-((7-methyl-[1,2,4]triazolo[1,5-a]pyridin-6-yl)amino)-7,9-dihydro-8H-purin-8-one (Compound 6)

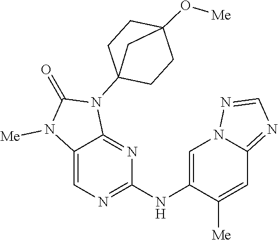 [SMILES: COC12CCC(n3c(=O)n(C)c4cnc(Nc5cn6ncnc6cc5C)nc43)(CC1)C2]

The title compound was prepared in 53.8% overall yield as a white solid according to the preparation of EXAMPLE 1 using 2-chloro-9-(4-methoxybicyclo[2.2.1]heptan-1-yl)-7-methyl-7,9-dihydro-8H-purin-8-one (Int-2) and 7-methyl-[1,2,4]triazolo[1,5-a]pyridin-6-amine. MS: m/z: Calc'd for C₂₁H₂₃N₅O₄ [M+H]⁺ 421; Found 421. ¹H NMR (500 MHz, DMSO-d₆) δ 9.03 (s, 1H), 8.54 (s, 1H), 8.31 (s, 1H), 8.03 (s, 1H), 7.65 (s, 1H), 3.37-3.20 (m, 3H), 3.06 (s, 3H), 2.40-2.32 (m, 6H), 2.28 (s, 2H), 1.95-1.87 (m, 2H), 1.78-1.71 (m, 1H), 1.49 (br s, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 421, retention time 1.36 min, purity: 100%; LCMS 2 with TFA: [M+H]⁺ found 421, retention time 1.11 min, purity: 100%.

### Example 7: 9-(4-methoxybicyclo[2.2.1]heptan-1-yl)-7-methyl-2-((6-methylbenzo[d][1,3]dioxol-5-yl)amino)-7,9-dihydro-8H-purin-8-one (Compound 7)

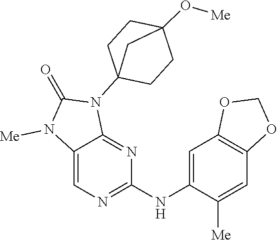 [SMILES: COC12CCC(n3c(=O)n(C)c4cnc(Nc5cc6c(cc5C)OCO6)nc43)(CC1)C2]

The title compound was prepared in 43.6% overall yield as a di-TFA salt according to the preparation of EXAMPLE 1 using 2-chloro-9-(4-methoxybicyclo[2.2.1]heptan-1-yl)-7-methyl-7,9-dihydro-8H-purin-8-one (Int-2) and 6-methylbenzo[d][1,3]dioxol-5-amine. MS: m/z: Calc'd for C₂₂H₂₅N₅O₄ [M+H]⁺ 424; Found 424. ¹H NMR (500 MHz, DMSO-d₆) δ 7.99 (s, 1H), 7.22 (s, 1H), 7.12 (s, 1H), 7.01 (d, J=5.6 Hz, 2H), 6.84 (s, 1H), 5.97 (s, 2H), 3.25 (s, 3H), 3.21 (s, 3H), 2.41 (br s, 2H), 2.33 (s, 2H), 2.12 (s, 3H), 1.99-1.91 (m, 2H), 1.81 (br s, 2H), 1.61-1.52 (m, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 424, retention time 1.75 min, purity: 100%; LCMS 2 with TFA: [M+H]⁺ found 424, retention time 1.41 min, purity: 100%.

### Example 8: 9-(4-methoxybicyclo[2.2.1]heptan-1-yl)-7-methyl-2-((6-methylbenzo[c][1,2,5]thiadiazol-5-yl)amino)-7,9-dihydro-8H-purin-8-one (Compound 8)

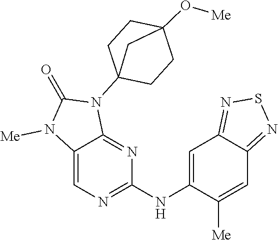 [SMILES: COC12CCC(n3c(=O)n(C)c4cnc(Nc5cc6nsnc6cc5C)nc43)(CC1)C2]

The title compound was prepared in 81.8% overall yield as a white solid according to the preparation of EXAMPLE 1 using 2-chloro-9-(4-methoxybicyclo[2.2.1]heptan-1-yl)-7-methyl-7,9-dihydro-8H-purin-8-one (Int-2) and 6-methylbenzo[c][1,2,5]thiadiazol-5-amine. MS: m/z: Calc'd for C₂₁H₂₃N₇O₂S [M+H]⁺ 438; Found 438. ¹H NMR (500 MHz, DMSO-d₆) δ 8.54 (s, 1H), 8.46 (s, 1H), 8.24-8.19 (m, 1H), 7.90 (s, 1H), 3.40-3.25 (m, 3H), 3.19 (s, 3H), 2.54 (s, 5H), 2.46 (s, 2H), 2.08-1.99 (m, 2H), 1.87 (br t, J=13.0 Hz, 2H), 1.65-1.55 (m, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 438, retention time 1.85 min, purity: 100%; LCMS 2 with TFA: [M+H]⁺ found 438, retention time 1.39 min, purity: 100%.

### Example 9: 9-(4-methoxybicyclo[2.2.1]heptan-1-yl)-7-methyl-2-((7-methylquinoxalin-6-yl)amino)-7,9-dihydro-8H-purin-8-one (Compound 9)

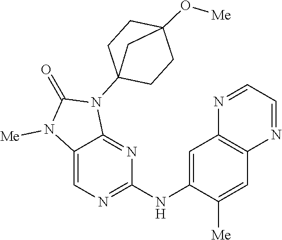 [SMILES: COC12CCC(n3c(=O)n(C)c4cnc(Nc5cc6nccnc6cc5C)nc43)(CC1)C2]

The title compound was prepared in 73.8% overall yield as a white solid according to the preparation of EXAMPLE 1 using 2-chloro-9-(4-methoxybicyclo[2.2.1]heptan-1-yl)-7-methyl-7,9-dihydro-8H-purin-8-one (Int-2) and 7-methylquinoxalin-6-amine. MS: m/z: Calc'd for C₂₃H₂₅N₇O₂ [M+H]⁺ 432; Found 432. ¹H NMR (500 MHz, DMSO-d₆) δ 8.78 (d, J=1.9 Hz, 1H), 8.72 (d, J=1.9 Hz, 1H), 8.59-8.56 (m, 2H), 8.21 (s, 1H), 7.90 (s, 1H), 3.39 (s, 3H), 3.16 (s, 3H), 2.57 (s, 3H), 2.45 (s, 4H), 2.06-1.98 (m, 2H), 1.90-1.82 (m, 2H), 1.57 (br s, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 432, retention time 1.49 min, purity: 98.8%; LCMS 2 with TFA: [M+H]⁺ found 432, retention time 1.31 min, purity: 100%.

### Example 10: 9-(4-methoxybicyclo[2.2.1]heptan-1-yl)-7-methyl-2-((7-methyl-2,3-dihydrobenzo[b][1,4]dioxin-6-yl)amino)-7,9-dihydro-8H-purin-8-one (Compound 10)

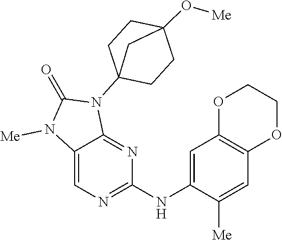 [SMILES: COC12CCC(n3c(=O)n(C)c4cnc(Nc5cc6c(cc5C)OCCO6)nc43)(CC1)C2]

The title compound was prepared in 66.7% overall yield as a white solid according to the preparation of EXAMPLE 1 using 2-chloro-9-(4-methoxybicyclo[2.2.1]heptan-1-yl)-7-methyl-7,9-dihydro-8H-purin-8-one (Int-2) and 7-methyl-2,3-dihydrobenzo[b][1,4]dioxin-6-amine, HCl. MS: m/z: Calc'd for C₂₃H₂₇N₅O₄ [M+H]⁺ 438; Found 438. ¹H NMR (500 MHz, DMSO-d₆) δ 8.13 (s, 1H), 7.98 (s, 1H), 7.00 (s, 1H), 6.67 (s, 1H), 4.18 (s, 4H), 3.24 (s, 3H), 3.22 (s, 3H), 2.43 (br s, 2H), 2.39-2.33 (m, 2H), 2.09 (s, 3H), 1.99-1.90 (m, 2H), 1.82 (br t, J=11.6 Hz, 2H), 1.55 (br s, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 438, retention time 1.66 min, purity: 98.1%; LCMS 2 with TFA: [M+H]⁺ found 438, retention time 1.33 min, purity: 96.5%.

### Example 11: 9-(3-fluorobicyclo[1.1.1]pentan-1-yl)-7-methyl-2-((7-methylquinolin-6-yl)amino)-7,9-dihydro-8H-purin-8-one (Compound 11)

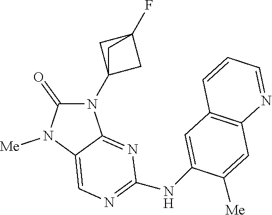 [SMILES: Cc1cc2ncccc2cc1Nc1ncc2c(n1)n(C13CC(F)(C1)C3)c(=O)n2C]

The title compound was prepared in 30.1% overall yield as a white solid according to the preparation of EXAMPLE 1 using 2-chloro-9-(3-fluorobicyclo[1.1.1]pentan-1-yl)-7-methyl-7,9-dihydro-8H-purin-8-one (Int-3) and 7-methylquinolin-6-amine. MS: m/z: Calc'd for C₂₁H₁₉FN₆O [M+H]⁺ 391; Found 391. ¹H NMR (500 MHz, DMSO-d₆) δ 8.74 (d, J=3.8 Hz, 1H), 8.64 (s, 1H), 8.28 (s, 1H), 8.21-8.18 (m, 2H), 7.86 (s, 1H), 7.44 (dd, J=8.2, 4.2 Hz, 1H), 3.18 (s, 1H), 3.43-3.15 (m, 2H), 2.72 (d, J=1.7 Hz, 6H), 2.50 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 391, retention time 1.69 min, purity: 100%; LCMS 2 with TFA: [M+H]⁺ found 391, retention time 1.25 min, purity: 100%.

### Example 12: 9-(3-fluorobicyclo[1.1.1]pentan-1-yl)-7-methyl-2-((7-methyl-[1,2,4]triazolo[1,5-a]pyridin-6-yl)amino)-7,9-dihydro-8H-purin-8-one (Compound 12)

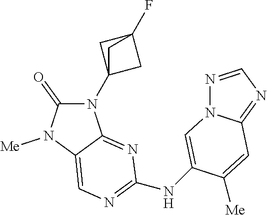 [SMILES: Cc1cc2ncnn2cc1Nc1ncc2c(n1)n(C13CC(F)(C1)C3)c(=O)n2C]

The title compound was prepared in 61.2% overall yield as a white solid according to the preparation of EXAMPLE 1 using 2-chloro-9-(3-fluorobicyclo[1.1.1]pentan-1-yl)-7-methyl-7,9-dihydro-8H-purin-8-one (Int-3) and 7-methyl-[1,2,4]triazolo[1,5-a]pyridin-6-amine. MS: m/z: Calc'd for C₁₈H₁₇FN₈O [M+H]⁺ 381; Found 381. ¹H NMR (500 MHz, DMSO-d₆) δ 9.15 (s, 1H), 8.69 (s, 1H), 8.38 (s, 1H), 8.14 (s, 1H), 7.72 (s, 1H), 3.38-3.17 (m, 3H), 2.69 (d, J=2.0 Hz, 6H), 2.40 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 381, retention time 1.43 min, purity: 100%; LCMS 2 with TFA: [M+H]⁺ found 381, retention time 1.25 min, purity: 100%.

### Example 13: 9-(3-fluorobicyclo[1.1.1]pentan-1-yl)-7-methyl-2-((6-methylbenzo[d][1,3]dioxol-5-yl)amino)-7,9-dihydro-8H-purin-8-one (Compound 13)

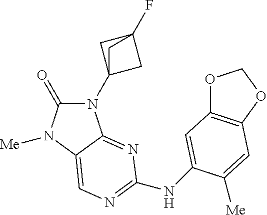 [SMILES: Cc1cc2c(cc1Nc1ncc3c(n1)n(C14CC(F)(C1)C4)c(=O)n3C)OCO2]

The title compound was prepared in 79.4% overall yield as a TFA salt according to the preparation of EXAMPLE 1 using 2-chloro-9-(3-fluorobicyclo[1.1.1]pentan-1-yl)-7-methyl-7,9-dihydro-8H-purin-8-one (Int-3) and 6-methylbenzo[d][1,3]dioxol-5-amine. MS: m/z: Calc'd for C₁₉H₁₈FN₅O₃ [M+H]⁺ 384; Found 384. ¹H NMR (500 MHz, DMSO-d₆) δ 8.03 (s, 1H), 7.24-6.97 (m, 3H), 6.81 (s, 1H), 5.97 (s, 2H), 3.28-3.17 (m, 3H), 2.67 (d, J=2.1 Hz, 6H), 2.12 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 384, retention time 1.82 min, purity: 99.3%; LCMS 2 with TFA: [M+H]⁺ found 384, retention time 1.49 min, purity: 99.2%.

### Example 14: 9-(3-fluorobicyclo[1.1.1]pentan-1-yl)-7-methyl-2-((6-methylbenzo[c][1,2,5]thiadiazol-5-yl)amino)-7,9-dihydro-8H-purin-8-one (Compound 14)

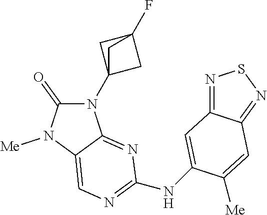 [SMILES: Cc1cc2nsnc2cc1Nc1ncc2c(n1)n(C13CC(F)(C1)C3)c(=O)n2C]

The title compound was prepared in 82.3% overall yield as a white solid according to the preparation of EXAMPLE 1 using 2-chloro-9-(3-fluorobicyclo[1.1.1]pentan-1-yl)-7-methyl-7,9-dihydro-8H-purin-8-one (Int-3) and 6-methylbenzo[c][1,2,5]thiadiazol-5-amine. MS: m/z: Calc'd for C₁₈H₁₆FN₇OS [M+H]⁺ 398; Found 398. ¹H NMR (500 MHz, DMSO-d₆) δ 8.62 (s, 1H), 8.55 (s, 1H), 8.27 (s, 1H), 7.91 (s, 1H), 3.33 (s, 3H), 2.78 (d, J=2.1 Hz, 6H), 2.55 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 398, retention time 1.89 min, purity: 100%; LCMS 2 with TFA: [M+H]⁺ found 398, retention time 1.63 min, purity: 100%.

### Example 15: 9-(3-fluorobicyclo[1.1.1]pentan-1-yl)-7-methyl-2-((7-methylquinoxalin-6-yl)amino)-7,9-dihydro-8H-purin-8-one (Compound 15)

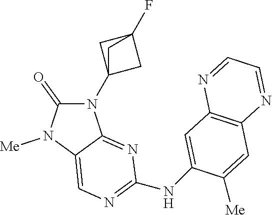 [SMILES: Cc1cc2nccnc2cc1Nc1ncc2c(n1)n(C13CC(F)(C1)C3)c(=O)n2C]

The title compound was prepared in 84.1% overall yield as a white solid according to the preparation of EXAMPLE 1 using 2-chloro-9-(3-fluorobicyclo[1.1.1]pentan-1-yl)-7-methyl-7,9-dihydro-8H-purin-8-one (Int-3) and 7-methylquinoxalin-6-amine. MS: m/z: Calc'd for C₂₀H₁₈FN₇O [M+H]⁺ 392; Found 392. ¹H NMR (500 MHz, DMSO-d₆) δ 8.82 (s, 1H), 8.74 (d, J=1.7 Hz, 1H), 8.71 (s, 1H), 8.66 (s, 1H), 8.26 (s, 1H), 7.92 (s, 1H), 3.33 (s, 3H), 2.79-2.73 (m, 6H), 2.58 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 392, retention time 1.54 min, purity: 100%; LCMS 2 with TFA: [M+H]⁺ found 392, retention time 1.31 min, purity: 100%.

### Example 16: 9-(3-fluorobicyclo[1.1.1]pentan-1-yl)-7-methyl-2-((7-methyl-2,3-dihydrobenzo[b][1,4]dioxin-6-yl)amino)-7,9-dihydro-8H-purin-8-one (Compound 16)

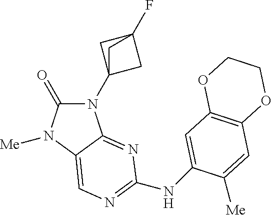 [SMILES: Cc1cc2c(cc1Nc1ncc3c(n1)n(C14CC(F)(C1)C4)c(=O)n3C)OCCO2]

The title compound was prepared in 74.7% overall yield as a white solid according to the preparation of EXAMPLE 1 using 2-chloro-9-(3-fluorobicyclo[1.1.1]pentan-1-yl)-7-methyl-7,9-dihydro-8H-purin-8-one (Int-3) and 7-methyl-2,3-dihydrobenzo[b][1,4]dioxin-6-amine, HCl. MS: m/z: Calc'd for C₂₀H₂₀FN₅O₃ [M+H]⁺ 398; Found 398. ¹H NMR (500 MHz, DMSO-d₆) δ 8.51-8.05 (m, 1H), 8.03-8.01 (m, 1H), 7.02 (s, 1H), 6.69 (s, 1H), 4.20 (s, 4H), 3.26 (s, 3H), 2.68 (d, J=1.3 Hz, 6H), 2.09 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 398, retention time 1.73 min, purity: 93.7%; LCMS 2 with TFA: [M+H]⁺ found 398, retention time 1.37 min, purity: 100%.

### Example 17: 3-(7-methyl-2-((6-methylbenzo[d][1,3]dioxol-5-yl)amino)-8-oxo-7,8-dihydro-9H-purin-9-yl) bicyclo[1.1.1]pentane-1-carbonitrile (Compound 17)

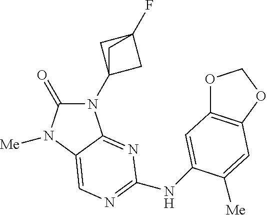 [SMILES: Cc1cc2c(cc1Nc1ncc3c(n1)n(C14CC(F)(C1)C4)c(=O)n3C)OCO2]

The title compound was prepared in 40.7% overall yield as a white solid according to the preparation of EXAMPLE 1 using 3-(2-chloro-7-methyl-8-oxo-7,8-dihydro-9H-purin-9-yl)bicyclo[1.1.1]pentane-1-carbonitrile (Int-4) and 6-methylbenzo[d][1,3]dioxol-5-amine. MS: m/z: Calc'd for C₂₀H₁₈FN₆O₃ [M+H]⁺ 391; Found 391. ¹H NMR (500 MHz, DMSO-d₆) δ 8.30 (s, 1H), 8.02 (s, 1H), 7.02 (s, 1H), 6.80 (s, 1H), 5.97 (s, 2H), 3.35-3.15 (m, 3H), 2.82 (s, 6H), 2.12 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 391, retention time 1.63 min, purity: 98.9%; LCMS 2 with TFA: [M+H]⁺ found 391, retention time 1.29 min, purity: 96.4%.

### Example 18: 7-methyl-2-((7-methyl-[1,2,4]triazolo[1,5-a]pyridin-6-yl)amino)-9-(3-(trifluoromethyl)bicyclo[1.1.1]pentan-1-yl)-7,9-dihydro-8H-purin-8-one (Compound 18)

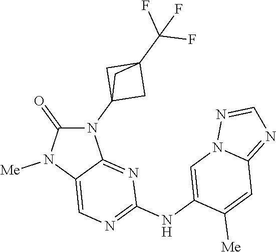 [SMILES: Cc1cc2ncnn2cc1Nc1ncc2c(n1)n(C13CC(C(F)(F)F)(C1)C3)c(=O)n2C]

The title compound was prepared in 38.9% overall yield as a white solid according to the preparation of EXAMPLE 1 using 2-chloro-7-methyl-9-(3-(trifluoromethyl)bicyclo[1.1.1]pentan-1-yl)-7,9-dihydro-8H-purin-8-one (Int-5) and 7-methyl-[1,2,4]triazolo[1,5-a]pyridin-6-amine. MS: m/z: Calc'd for C₁₉H₁₇F₃N₈O [M+H]⁺ 431; Found 431. ¹H NMR (500 MHz, DMSO-d₆) δ 9.15 (s, 1H), 8.68 (s, 1H), 8.37 (s, 1H), 8.14 (s, 1H), 7.72 (s, 1H), 3.28 (s, 3H), 2.62 (s, 6H), 2.40 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 431, retention time 1.58 min, purity: 100%; LCMS 2 with TFA: [M+H]⁺ found 431, retention time 1.35 min, purity: 100%.

### Example 19: 7-methyl-2-((6-methylbenzo[d][1,3]dioxol-5-yl)amino)-9-(3-(trifluoromethyl)bicyclo[1.1.1]pentan-1-yl)-7,9-dihydro-8H-purin-8-one (Compound 19)

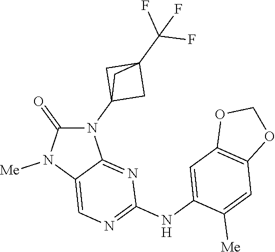 [SMILES: Cc1cc2c(cc1Nc1ncc3c(n1)n(C14CC(C(F)(F)F)(C1)C4)c(=O)n3C)OCO2]

The title compound was prepared in 42.3% overall yield as a di-TFA salt according to the preparation of EXAMPLE 1 using 2-chloro-7-methyl-9-(3-(trifluoromethyl)bicyclo[1.1.1]pentan-1-yl)-7,9-dihydro-8H-purin-8-one (Int-5) and 6-methylbenzo[d][1,3]dioxol-5-amine. MS: m/z: Calc'd for C₂₀H₁₈F₃N₅O₃ [M+H]⁺ 434; Found 434. ¹H NMR (500 MHz, DMSO-d₆) δ 8.03 (s, 1H), 7.23-6.99 (m, 4H), 6.82 (s, 1H), 5.96 (s, 2H), 3.26 (s, 3H), 2.60 (s, 6H), 2.12 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 434, retention time 2.01 min, purity: 100%; LCMS 2 with TFA: [M+H]⁺ found 434, retention time 1.64 min, purity: 99.2%.

### Example 20: 9-(3-(methoxymethyl)bicyclo[1.1.1]pentan-1-yl)-7-methyl-2-((7-methyl-[1,2,4]triazolo[1,5-a]pyridin-6-yl)amino)-7,9-dihydro-8H-purin-8-one (Compound 20)

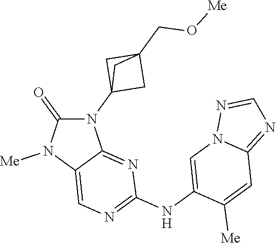 [SMILES: COCC12CC(n3c(=O)n(C)c4cnc(Nc5cn6ncnc6cc5C)nc43)(C1)C2]

The title compound was prepared in 58.2% overall yield as a white solid according to the preparation of EXAMPLE 1 using 2-chloro-9-(3-(methoxymethyl)bicyclo[1.1.1]pentan-1-yl)-7-methyl-7,9-dihydro-8H-purin-8-one (Int-6) and 7-methyl-[1,2,4]triazolo[1,5-a]pyridin-6-amine. MS: m/z: Calc'd for C₂₀H₂₂N₈O₂ [M+H]⁺ 407; Found 407. ¹H NMR (500 MHz, DMSO-d₆) δ 9.15 (s, 1H), 8.60 (s, 1H), 8.37 (s, 1H), 8.10 (s, 1H), 7.71 (s, 1H), 3.46 (s, 2H), 3.31-3.21 (m, 3H), 2.57-2.52 (m, 3H), 2.40 (s, 3H), 2.27 (s, 6H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 407, retention time 1.29 min, purity: 100%; LCMS 2 with TFA: [M+H]⁺ found 407, retention time 1.16 min, purity: 100%.

### Example 21: 9-(3-(methoxymethyl)bicyclo[1.1.1]pentan-1-yl)-7-methyl-2-((6-methylbenzo[d][1,3]dioxol-5-yl)amino)-7,9-dihydro-8H-purin-8-one (Compound 21)

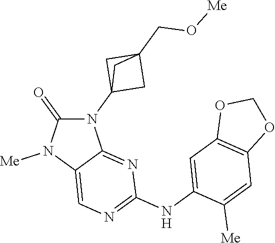 [SMILES: COCC12CC(n3c(=O)n(C)c4cnc(Nc5cc6c(cc5C)OCO6)nc43)(C1)C2]

The title compound was prepared in 54.5% overall yield as a white solid according to the preparation of EXAMPLE 1 using 2-chloro-9-(3-(methoxymethyl)bicyclo[1.1.1]pentan-1-yl)-7-methyl-7,9-dihydro-8H-purin-8-one (Int-6) and 6-methylbenzo[d][1,3]dioxol-5-amine. MS: m/z: Calc'd for C₂₀H₂₂N₈O₂ [M+H]⁺ 410; Found 410. ¹H NMR (500 MHz, DMSO-d₆) δ 8.25 (br s, 1H), 7.99 (s, 1H), 7.05 (s, 1H), 6.79 (s, 1H), 5.95 (s, 2H), 3.47 (s, 3H), 3.28 (s, 2H), 3.24 (s, 3H), 2.25 (s, 6H), 2.12 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 410, retention time 1.69 min, purity: 100%; LCMS 2 with TFA: [M+H]⁺ found 410, retention time 1.37 min, purity: 99.3%.

### Example 22: 2-(3-(7-methyl-2-((7-methyl-[1,2,4]triazolo[1,5-a]pyridin-6-yl)amino)-8-oxo-7,8-dihydro-9H-purin-9-yl)bicyclo[1.1.1]pentan-1-yl)acetonitrile (Compound 22)

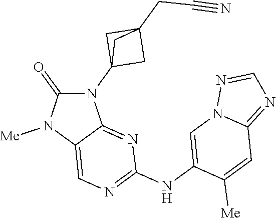 [SMILES: Cc1cc2ncnn2cc1Nc1ncc2c(n1)n(C13CC(CC#N)(C1)C3)c(=O)n2C]

The title compound was prepared in 60.0% overall yield as a white solid according to the preparation of EXAMPLE 1 using 2-(3-(2-chloro-7-methyl-8-oxo-7,8-dihydro-9H-purin-9-yl)bicyclo[1.1.1]pentan-1-yl)acetonitrile (Int-7) and 7-methyl-[1,2,4]triazolo[1,5-a]pyridin-6-amine. MS: m/z: Calc'd for C₂₀H₁₉N₉O [M+H]⁺ 402; Found 402. ¹H NMR (500 MHz, DMSO-d₆) δ 9.11 (s, 1H), 8.63 (s, 1H), 8.37 (s, 1H), 8.10 (s, 1H), 7.70 (s, 1H), 3.27 (s, 2H), 2.98 (s, 3H), 2.40 (s, 3H), 2.35 (s, 6H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 402, retention time 1.26 min, purity: 100%; LCMS 2 with TFA: [M+H]⁺ found 402, retention time 1.12 min, purity: 99.3%.

### Example 23: 2-(3-(7-methyl-2-((6-methylbenzo[d][1,3]dioxol-5-yl)amino)-8-oxo-7,8-dihydro-9H-purin-9-yl)bicyclo[1.1.1]pentan-1-yl)acetonitrile (Compound 23)

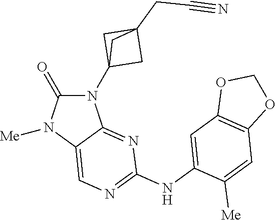 [SMILES: Cc1cc2c(cc1Nc1ncc3c(n1)n(C14CC(CC#N)(C1)C4)c(=O)n3C)OCO2]

The title compound was prepared in 59.5% overall yield as a white solid according to the preparation of EXAMPLE 1 using 2-(3-(2-chloro-7-methyl-8-oxo-7,8-dihydro-9H-purin-9-yl)bicyclo[1.1.1]pentan-1-yl)acetonitrile (Int-7) and 6-methylbenzo[d][1,3]dioxol-5-amine. MS: m/z: Calc'd for C₂₁H₂₀N₆O₃ [M+H]⁺ 405; Found 405. ¹H NMR (500 MHz, DMSO-d₆) δ 8.27 (s, 1H), 7.99 (s, 1H), 7.02 (s, 1H), 6.78 (s, 1H), 5.95 (s, 2H), 3.24 (s, 2H), 2.98 (s, 3H), 2.33 (s, 6H), 2.12 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 405, retention time 1.61 min, purity: 100%; LCMS 2 with TFA: [M+H]⁺ found 405, retention time 1.30 min, purity: 100%.

### Example 24: 4-(7-methyl-2-((6-methylbenzo[d][1,3]dioxol-5-yl)amino)-8-oxo-7,8-dihydro-9H-purin-9-yl) bicyclo[2.2.2]octane-1-carbonitrile (Compound 24)

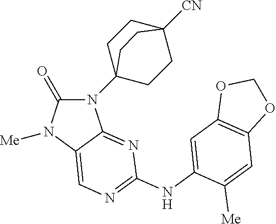 [SMILES: Cc1cc2c(cc1Nc1ncc3c(n1)n(C14CCC(C#N)(CC1)CC4)c(=O)n3C)OCO2]

The title compound was prepared in 78.5% overall yield as a white solid according to the preparation of EXAMPLE 1 using 4-(2-chloro-7-methyl-8-oxo-7,8-dihydro-9H-purin-9-yl)bicyclo[2.2.2]octane-1-carbonitrile (Int-9) and 6-methylbenzo[d][1,3]dioxol-5-amine. MS: m/z: Calc'd for C₂₂H₂₃N₉O [M+H]⁺ 433; Found 433. ¹H NMR (500 MHz, DMSO-d₆) δ 7.98 (s, 1H), 7.24-6.98 (m, 2H), 6.85 (s, 1H), 5.99 (s, 2H), 3.23 (s, 3H), 2.46-2.38 (m, 6H), 2.11 (s, 3H), 2.04-1.99 (m, 6H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 433, retention time 1.74 min, purity: 99.3%; LCMS 2 with TFA: [M+H]⁺ found 433, retention time 1.34 min, purity: 98.5%.

### Example 25: 9-(4-methoxybicyclo[2.2.2]octan-1-yl)-7-methyl-2-((7-methyl-[1,2,4]triazolo[1,5-a]pyridin-6-yl)amino)-7,9-dihydro-8H-purin-8-one (Compound 25)

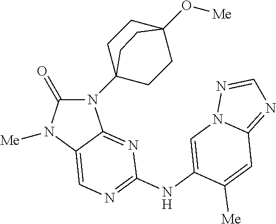 [SMILES: COC12CCC(n3c(=O)n(C)c4cnc(Nc5cn6ncnc6cc5C)nc43)(CC1)CC2]

The title compound was prepared in 79.2% overall yield as a white solid according to the preparation of EXAMPLE 1 using 2-chloro-9-(4-methoxybicyclo[2.2.2]octan-1-yl)-7-methyl-7,9-dihydro-8H-purin-8-one (Int-10) and 7-methyl-[1,2,4]triazolo[1,5-a]pyridin-6-amine. MS: m/z: Calc'd for C₂₂H₂₆N₈O₂ [M+H]⁺ 435; Found 435. ¹H NMR (500 MHz, DMSO-d₆) δ 9.14 (s, 1H), 8.57 (s, 1H), 8.38 (s, 1H), 8.08 (s, 1H), 7.71 (s, 1H), 3.06 (s, 3H), 2.48 (br d, J=8.3 Hz, 6H), 2.40 (s, 3H), 1.72-1.65 (m, 6H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 435, retention time 1.31 min, purity: 100%; LCMS 2 with TFA: [M+H]⁺ found 435, retention time 1.06 min, purity: 100%.

### Example 26: 9-(4-methoxybicyclo[2.2.2]octan-1-yl)-7-methyl-2-((6-methylbenzo[d][1,3]dioxol-5-yl)amino)-7,9-dihydro-8H-purin-8-one (Compound 26)

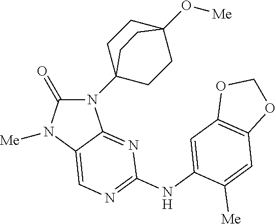 [SMILES: COC12CCC(n3c(=O)n(C)c4cnc(Nc5cc6c(cc5C)OCO6)nc43)(CC1)CC2]

The title compound was prepared in 93.9% overall yield as a white solid according to the preparation of EXAMPLE 1 using 2-chloro-9-(4-methoxybicyclo[2.2.2]octan-1-yl)-7-methyl-7,9-dihydro-8H-purin-8-one (Int-10) and 6-methylbenzo[d][1,3]dioxol-5-amine. MS: m/z: Calc'd for C₂₃H₂₇N₅O₄ [M+H]⁺ 438; Found 438. ¹H NMR (500 MHz, DMSO-d₆) δ 7.97 (s, 1H), 7.27-6.97 (m, 2H), 6.86 (s, 1H), 5.99 (s, 2H), 3.23 (s, 3H), 3.07 (s, 3H), 2.48-2.43 (m, 6H), 2.12 (s, 3H), 1.72-1.67 (m, 6H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 438, retention time 1.73 min, purity: 100%; LCMS 2 with TFA: [M+H]⁺ found 438, retention time 1.34 min, purity: 100%.

### Example 27: 9-(4-hydroxybicyclo[2.2.2]octan-1-yl)-7-methyl-2-((6-methylbenzo[d][1,3]dioxol-5-yl)amino)-7,9-dihydro-8H-purin-8-one (Compound 27)

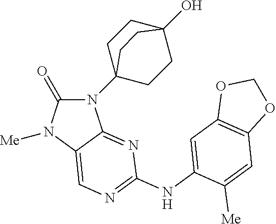 [SMILES: Cc1cc2c(cc1Nc1ncc3c(n1)n(C14CCC(O)(CC1)CC4)c(=O)n3C)OCO2]

The title compound was prepared in 77.2% overall yield as a white solid according to the preparation of EXAMPLE 1 using 2-chloro-9-(4-hydroxybicyclo[2.2.2]octan-1-yl)-7-methyl-7,9-dihydro-8H-purin-8-one (Int-11) and 6-methylbenzo[d][1,3]dioxol-5-amine. MS: m/z: Calc'd for C₂₂H₂₅N₅O₄ [M+H]⁺ 424; Found 424. ¹H NMR (500 MHz, DMSO-d₆) δ 7.95 (s, 1H), 7.03 (s, 1H), 6.80 (s, 1H), 5.96 (s, 2H), 3.21 (s, 2H), 2.47-2.42 (m, 6H), 2.12 (s, 3H), 1.66-1.61 (m, 6H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 424, retention time 1.40 min, purity: 100%; LCMS 2 with TFA: [M+H]⁺ found 424, retention time 1.05 min, purity: 100%.

### Example 28: 9-(4-methoxybicyclo[2.1.1]hexan-1-yl)-7-methyl-2-((6-methylbenzo[d][1,3]dioxol-5-yl)amino)-7,9-dihydro-8H-purin-8-one (Compound 28)

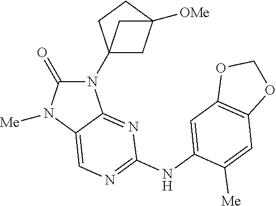 [SMILES: COC12CCC(n3c(=O)n(C)c4cnc(Nc5cc6c(cc5C)OCO6)nc43)(C1)C2]

The title compound was prepared in 29.6% overall yield as a white solid according to the preparation of EXAMPLE 1 using 2-chloro-9-(4-methoxybicyclo[2.1.1]hexan-1-yl)-7-methyl-7,9-dihydro-8H-purin-8-one (Int-12) and 6-methylbenzo[d][1,3]dioxol-5-amine. MS: m/z: Calc'd for C₂₁H₂₃N₅O₄ [M+H]⁺ 410; Found 410. ¹H NMR (500 MHz, DMSO-d₆) δ 7.99 (s, 1H), 7.0-7.0 (m, 1H), 6.83 (s, 1H), 5.97 (s, 2H), 3.3-3.3 (m, 2H), 2.1-2.2 (m, 7H), 1.9-2.0 (m, 2H), 1.7-1.8 (m, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 410, retention time 1.63 min, purity: 98.3%; LCMS 2 with TFA: [M+H]⁺ found 410, retention time 1.30 min, purity: 96.8%.

### Example 29: 9-(4-methoxybicyclo[2.1.1]hexan-1-yl)-7-methyl-2-((7-methyl-[1,2,4]triazolo[1,5-a]pyridin-6-yl)amino)-7,9-dihydro-8H-purin-8-one (Compound 29)

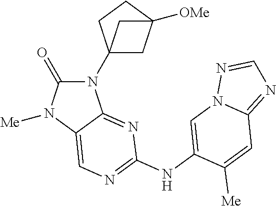 [SMILES: COC12CCC(n3c(=O)n(C)c4cnc(Nc5cn6ncnc6cc5C)nc43)(C1)C2]

The title compound was prepared in 31.8% overall yield as a white solid according to the preparation of EXAMPLE 1 using 2-chloro-9-(4-methoxybicyclo[2.1.1]hexan-1-yl)-7-methyl-7,9-dihydro-8H-purin-8-one (Int-12) and 7-methyl-[1,2,4]triazolo[1,5-a]pyridin-6-amine. MS: m/z: Calc'd for C₂₀H₂₂N₈O₂ [M+H]⁺ 407; Found 407. ¹H NMR (500 MHz, DMSO-d₆) δ 9.16 (s, 1H), 8.59 (s, 1H), 8.36 (s, 1H), 8.11 (s, 1H), 7.70 (s, 1H), 3.4-3.28 (s, 2H), 3.23 (m, 1H), 2.40 (s, 3H), 2.13 (br s, 4H), 2.01-1.98 (m, 2H), 1.74-1.71 (m, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 407, retention time 1.21 min, purity: 100%; LCMS 2 with TFA: [M+H]⁺ found 407, retention time 1.03 min, purity: 100%.

### Example 30: 9-(4-methoxybicyclo[2.1.1]hexan-1-yl)-7-methyl-2-((6-methylbenzo[c][1,2,5]thiadiazol-5-yl)amino)-7,9-dihydro-8H-purin-8-one (Compound 30)

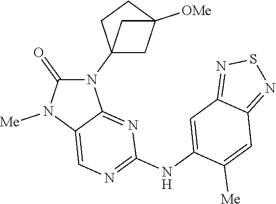 [SMILES: COC12CCC(n3c(=O)n(C)c4cnc(Nc5cc6nsnc6cc5C)nc43)(C1)C2]

The title compound was prepared in 33.6% overall yield as a white solid according to the preparation of EXAMPLE 1 using 2-chloro-9-(4-methoxybicyclo[2.1.1]hexan-1-yl)-7-methyl-7,9-dihydro-8H-purin-8-one (Int-12) and 6-methylbenzo[c][1,2,5]thiadiazol-5-amine. MS: m/z: Calc'd for C₂₀H₂₁N₇O₂S [M+H]⁺ 424; Found 424. ¹H NMR (500 MHz, DMSO-d₆) δ 8.63 (s, 1H), 8.49 (s, 1H), 8.24 (s, 1H), 7.90 (s, 1H), 3.43 (s, 3H), 3.33 (s, 2H), 2.27-2.19 (m, 4H), 2.08-2.02 (m, 2H), 1.78 (br dd, J=5.1, 7.1 Hz, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 424, retention time 1.79 min, purity: 100%; LCMS 2 with TFA: [M+H]⁺ found 424, retention time 1.79 min, purity: 100%.

### Example 31: 9-(4-hydroxybicyclo[2.2.1]heptan-1-yl)-7-methyl-2-((7-methyl-[1,2,4]triazolo[1,5-a]pyridin-6-yl)amino)-7,9-dihydro-8H-purine-8-thione (Compound 31)

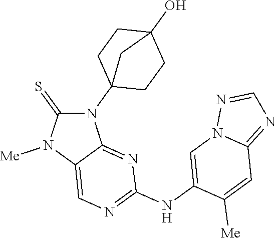 [SMILES: Cc1cc2ncnn2cc1Nc1ncc2c(n1)n(C13CCC(O)(CC1)C3)c(=S)n2C]

The title compound was prepared in 60.6% overall yield as a white solid according to the preparation of EXAMPLE 1 using 2-chloro-9-(4-hydroxybicyclo[2.2.1]heptan-1-yl)-7-methyl-7,9-dihydro-8H-purine-8-thione (Int-13) and 7-methyl-[1,2,4]triazolo[1,5-a]pyridin-6-amine. MS: m/z: Calc'd for C₂₀H₂₂N₈OS [M+H]⁺ 423; Found 423. ¹H NMR (500 MHz, DMSO-d₆) δ 9.13 (s, 1H), 8.76 (s, 1H), 8.60-8.58 (m, 1H), 8.38 (s, 1H), 7.72 (s, 1H), 5.19-5.15 (m, 1H), 2.68 (s, 3H), 2.58 (br s, 2H), 2.46-2.42 (m, 2H), 2.40 (s, 3H), 1.98-1.90 (m, 2H), 1.76 (br s, 2H), 1.60 (br s, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 423, retention time 1.26 min, purity: 100%; LCMS 2 with TFA: [M+H]⁺ found 423, retention time 1.08 min, purity: 100%.

### Example 32: 9-(4-hydroxybicyclo[2.2.1]heptan-1-yl)-7-methyl-2-((6-methylbenzo[d][1,3]dioxol-5-yl)amino)-7,9-dihydro-8H-purine-8-thione (Compound 32)

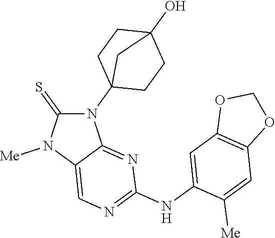 [SMILES: Cc1cc2c(cc1Nc1ncc3c(n1)n(C14CCC(O)(CC1)C4)c(=S)n3C)OCO2]

The title compound was prepared in 74.8% overall yield as a white solid according to the preparation of EXAMPLE 1 using 2-chloro-9-(4-hydroxybicyclo[2.2.1]heptan-1-yl)-7-methyl-7,9-dihydro-8H-purine-8-thione (Int-13) and 6-methylbenzo[d][1,3]dioxol-5-amine. MS: m/z: Calc'd for C₂₁H₂₃N₅O₃S [M+H]⁺ 426; Found 426. ¹H NMR (500 MHz, DMSO-d₆) δ 8.51 (s, 1H), 7.05 (s, 1H), 7.25-6.98 (m, 1H), 6.82 (s, 1H), 5.97 (s, 2H), 2.67 (s, 3H), 2.60-2.53 (m, 2H), 2.42 (s, 2H), 2.13 (s, 3H), 1.92 (br s, 2H), 1.76 (br s, 2H), 1.61 (br s, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 426, retention time 1.58 min, purity: 98.3%; LCMS 2 with TFA: [M+H]⁺ found 426, retention time 1.23 min, purity: 99.2%.

### Example 33: 9-(4-hydroxybicyclo[2.2.1]heptan-1-yl)-7-methyl-2-((7-methylquinoxalin-6-yl)amino)-7,9-dihydro-8H-purine-8-thione (Compound 33)

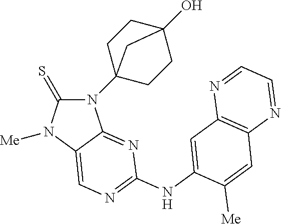 [SMILES: Cc1cc2nccnc2cc1Nc1ncc2c(n1)n(C13CCC(O)(CC1)C3)c(=S)n2C]

The title compound was prepared in 71.4% overall yield as a white solid according to the preparation of EXAMPLE 1 using 2-chloro-9-(4-hydroxybicyclo[2.2.1]heptan-1-yl)-7-methyl-7,9-dihydro-8H-purine-8-thione (Int-13) and 7-methylquinoxalin-6-amine. MS: m/z: Calc'd for C₂₂H₂₃N₇OS [M+H]⁺ 434; Found 434. ¹H NMR (500 MHz, DMSO-d₆) δ 8.83-8.77 (m, 2H), 8.74 (d, J=1.7 Hz, 1H), 8.71 (s, 1H), 8.64 (s, 1H), 7.92 (s, 1H), 2.70 (s, 5H), 2.58 (s, 3H), 2.55 (s, 1H), 2.49 (br s, 2H), 2.04-1.96 (m, 2H), 1.84-1.76 (m, 2H), 1.67-1.60 (m, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 434, retention time 1.49 min, purity: 100%; LCMS 2 with TFA: [M+H]⁺ found 434, retention time 1.26 min, purity: 99.4%.

### Example 34: 9-(3-fluorobicyclo[1.1.1]pentan-1-yl)-7-methyl-2-((7-methyl-[1,2,4]triazolo[1,5-a]pyridin-6-yl)amino)-7,9-dihydro-8H-purine-8-thione (Compound 34)

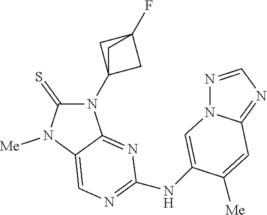 [SMILES: Cc1cc2ncnn2cc1Nc1ncc2c(n1)n(C13CC(F)(C1)C3)c(=S)n2C]

The title compound was prepared in 77.0% overall yield as a white solid according to the preparation of EXAMPLE 1 using 2-chloro-9-(3-fluorobicyclo[1.1.1]pentan-1-yl)-7-methyl-7,9-dihydro-8H-purine-8-thione (Int-14) and 7-methyl-[1,2,4]triazolo[1,5-a]pyridin-6-amine. MS: m/z: Calc'd for C₁₈H₁₇FN₈S [M+H]⁺ 397; Found 397. ¹H NMR (500 MHz, DMSO-d₆) δ 9.23 (s, 1H), 8.96 (s, 1H), 8.66 (s, 1H), 8.43 (s, 1H), 7.75 (s, 1H), 2.84 (d, J=1.9 Hz, 6H), 2.71 (s, 3H), 2.42 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 397, retention time 1.56 min, purity: 98.3%; LCMS 2 with TFA: [M+H]⁺ found 397, retention time 1.36 min, purity: 100%.

### Example 35: 9-(3-fluorobicyclo[1.1.1]pentan-1-yl)-7-methyl-2-((6-methylbenzo[d][1,3]dioxol-5-yl)amino)-7,9-dihydro-8H-purine-8-thione (Compound 35)

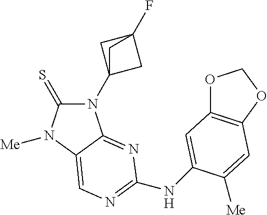 [SMILES: Cc1cc2c(cc1Nc1ncc3c(n1)n(C14CC(F)(C1)C4)c(=S)n3C)OCO2]

The title compound was prepared in 71.7% overall yield as a white solid according to the preparation of EXAMPLE 1 using 2-chloro-9-(3-fluorobicyclo[1.1.1]pentan-1-yl)-7-methyl-7,9-dihydro-8H-purine-8-thione (Int-14) and 6-methylbenzo[d][1,3]dioxol-5-amine. MS: m/z: Calc'd for C₁₉H₁₈FN₅O₂S [M+H]⁺ 400; Found 400. ¹H NMR (500 MHz, DMSO-d₆) δ 8.54 (s, 2H), 7.11 (s, 1H), 6.81 (s, 1H), 5.97 (s, 2H), 2.81 (d, J=1.9 Hz, 6H), 2.69 (s, 3H), 2.14 (s, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 400, retention time 1.99 min, purity: 98.6%; LCMS 2 with TFA: [M+H]⁺ found 400, retention time 1.61 min, purity: 97.4%.

### Example 36: 2-((5-fluoro-2-methyl-4-(oxazol-5-yl)phenyl)amino)-9-(4-hydroxybicyclo[2.2.1]heptan-1-yl)-7-methyl-7,9-dihydro-8H-purin-8-one (Compound 36)

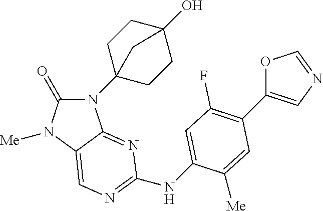 [SMILES: Cc1cc(-c2cnco2)c(F)cc1Nc1ncc2c(n1)n(C13CCC(O)(CC1)C3)c(=O)n2C]

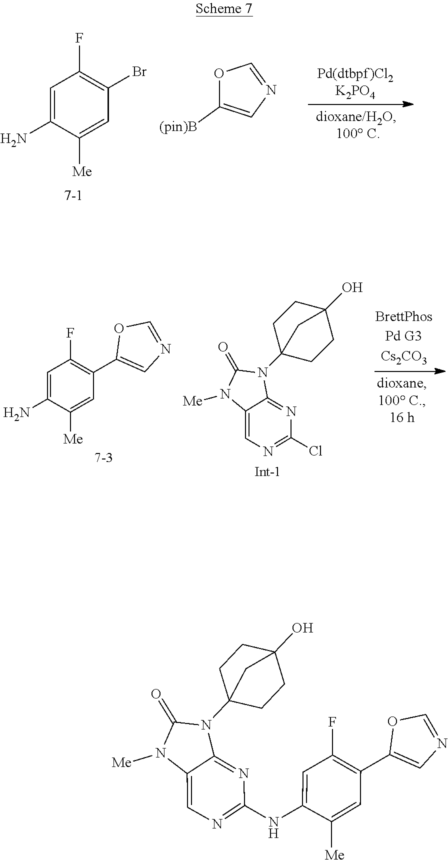

### Step 1: Synthesis of 5-fluoro-2-methyl-4-(oxazol-5-yl)aniline

To a 2-dram vial containing oxazole-5-boronic acid pinacol ester (5 mg, 0.18 mmol), 4-bromo-5-fluoro-2-methylaniline (31 mg, 0.15 mmol), [1,1′-Bis(di-tert-butylphosphino)ferrocene]dichloropalladium(II) (9.8 mg, 0.015 mmol) and potassium phosphate tribasic (64 mg, 0.30 mmol) was added dioxane (1.0 mL) and water (0.2 mL). The resulting mixture was allowed to stir at 100° C. for over night. The resulting suspension was concentrated to dryness, dissolved in EA and filtered to afford the target compound (29 mg, 0.015 mmol, 100% yield) as a dark oil.

### Step 2: Synthesis of 2-((5-fluoro-2-methyl-4-(oxazol-5-yl)phenyl)amino)-9-(4-hydroxybicyclo[2.2.1]heptan-1-yl)-7-methyl-7,9-dihydro-8H-purin-8-one

The title compound was prepared in 28.9% overall yield as a white solid according to the preparation of EXAMPLE 1 using 2-chloro-9-(4-hydroxybicyclo[2.2.1]heptan-1-yl)-7-methyl-7,9-dihydro-8H-purin-8-one (Int-1) and 5-fluoro-2-methyl-4-(oxazol-5-yl)aniline. MS: m/z: Calc'd for C₂₃H₂₃FN₆O₃ [M+H]⁺ 451; Found 451. ¹H NMR (500 MHz, DMSO-d₆) δ 8.48-8.39 (m, 2H), 8.15-8.10 (m, 1H), 7.95 (d, J=14.5 Hz, 1H), 7.61-7.55 (m, 1H), 7.41 (d, J=3.2 Hz, 1H), 3.28 (s, 3H), 3.17 (s, 1H), 2.45 (br d, J=1.3 Hz, 2H), 2.36 (s, 2H), 2.32 (s, 3H), 2.03-1.93 (m, 2H), 1.79-1.69 (m, 2H), 1.64-1.54 (m, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 451, retention time 1.52 min, purity: 91.2%; LCMS 2 with TFA: [M+H]⁺ found 451, retention time 1.24 min, purity: 98.1%.

### Example 37: 2-((5-fluoro-2-methyl-4-(1-methyl-1H-pyrazol-4-yl)phenyl)amino)-9-(4-hydroxybicyclo[2.2.1]heptan-1-yl)-7-methyl-7,9-dihydro-8H-purin-8-one (Compound 37)

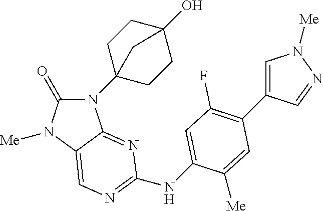 [SMILES: Cc1cc(-c2cnn(C)c2)c(F)cc1Nc1ncc2c(n1)n(C13CCC(O)(CC1)C3)c(=O)n2C]

The title compound was prepared in 54.8% overall yield as a white solid according to the preparation of EXAMPLE 36 using 1-methyl-4-(4,4,5,5-tetramethyl-1,3,2-dioxaborolan-2-yl)-1H-pyrazole in Step 1. MS: m/z: Calc'd for C₂₄H₂₆FN₇O₂[M+H]⁺ 464; Found 464. ¹H NMR (500 MHz, DMSO-d₆) δ 8.27 (s, 1H), 8.09-8.05 (m, 1H), 8.05-8.02 (m, 1H), 7.82 (s, 1H), 7.71 (d, J=13.8 Hz, 1H), 7.49 (br d, J=9.0 Hz, 1H), 3.90-3.86 (m, 3H), 3.41-3.21 (m, 3H), 2.44 (br s, 2H), 2.35 (s, 2H), 2.26 (s, 3H), 2.01-1.93 (m, 2H), 1.73 (br s, 2H), 1.57 (br s, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 464, retention time 1.46 min, purity: 98.1%; LCMS 2 with TFA: [M+H]⁺ found 464, retention time 1.19 min, purity: 97.3%.

### Example 38: 2-((5-fluoro-2-methyl-4-(1-methyl-1H-pyrazol-4-yl)phenyl)amino)-9-(3-fluorobicyclo[1.1.1]pentan-1-yl)-7-methyl-7,9-dihydro-8H-purin-8-one (Compound 38)

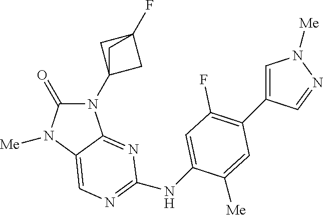 [SMILES: Cc1cc(-c2cnn(C)c2)c(F)cc1Nc1ncc2c(n1)n(C13CC(F)(C1)C3)c(=O)n2C]

The title compound was prepared in 58.9% overall yield as a white solid according to the preparation of EXAMPLE 36 using 1-methyl-4-(4,4,5,5-tetramethyl-1,3,2-dioxaborolan-2-yl)-1H-pyrazole in Step 1 and 2-chloro-9-(3-fluorobicyclo[1.1.1]pentan-1-yl)-7-methyl-7,9-dihydro-8H-purin-8-one (Int-3) in Step 2. MS: m/z: Calc'd for C₂₂H₂₁F₂N₇O [M+H]⁺ 438; Found 438. ¹H NMR (500 MHz, DMSO-d₆) δ 8.49 (s, 1H), 8.17-8.11 (m, 1H), 8.06 (br s, 1H), 7.84 (s, 1H), 7.73 (br d, J=14.0 Hz, 1H), 7.52 (d, J=8.7 Hz, 1H), 3.91-3.85 (m, 3H), 3.43-3.26 (m, 3H), 2.75-2.70 (m, 6H), 2.29-2.24 (m, 3H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 438, retention time 1.77 min, purity: 95.8%; LCMS 2 with TFA: [M+H]⁺ found 438, retention time 1.48 min, purity: 100%.

### Example 39: 2-((5-fluoro-2-methyl-4-(1-methyl-1H-pyrazol-4-yl)phenyl)amino)-9-(4-methoxybicyclo[2.1.1]hexan-1-yl)-7-methyl-7,9-dihydro-8H-purin-8-one (Compound 39)

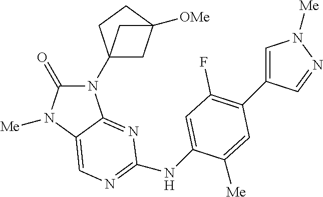 [SMILES: COC12CCC(n3c(=O)n(C)c4cnc(Nc5cc(F)c(-c6cnn(C)c6)cc5C)nc43)(C1)C2]

The title compound was prepared in 48.1% overall yield as a white solid according to the preparation of EXAMPLE 36 using 1-methyl-4-(4,4,5,5-tetramethyl-1,3,2-dioxaborolan-2-yl)-1H-pyrazole in Step 1 and 2-chloro-9-(4-methoxybicyclo[2.1.1]hexan-1-yl)-7-methyl-7,9-dihydro-8H-purin-8-one (Int-12) in Step 2. MS: m/z: Calc'd for C₂₄H₂₆FN₇O [M+H]⁺ 464; Found 464. ¹H NMR (500 MHz, DMSO-d₆) δ 8.46 (s, 1H), 8.12 (s, 1H), 8.05 (s, 1H), 7.83 (s, 1H), 7.74 (d, J=14.0 Hz, 1H), 7.51 (d, J=8.5 Hz, 1H), 3.91-3.87 (m, 3H), 3.42 (br s, 1H), 3.31-3.24 (m, 5H), 3.17 (s, 1H), 2.37 (s, 1H), 2.27 (s, 3H), 2.18 (s, 4H), 2.01 (br dd, J=6.2, 7.3 Hz, 2H), 1.75 (br dd, J=5.4, 6.7 Hz, 2H), 1.25-1.12 (m, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 464, retention time 1.68 min, purity: 91.5%; LCMS 2 with TFA: [M+H]⁺ found 464, retention time 1.35 min, purity: 96.4%.

### Example 40: 2-fluoro-4-((9-(4-methoxybicyclo[2.1.1]hexan-1-yl)-7-methyl-8-oxo-8,9-dihydro-7H-purin-2-yl)amino)-5-methylbenzamide (Compound 40)

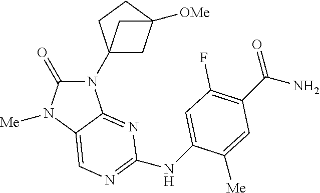 [SMILES: COC12CCC(n3c(=O)n(C)c4cnc(Nc5cc(F)c(C(N)=O)cc5C)nc43)(C1)C2]

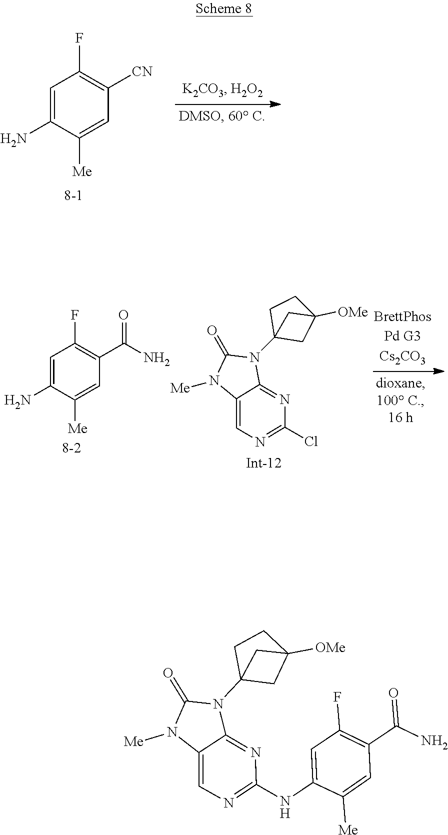 [SMILES: Cc1cc(C#N)c(F)cc1N ; Cc1cc(C(N)=O)c(F)cc1N ; COC12CCC(n3c(=O)n(C)c4cnc(Cl)nc43)(C1)C2 ; COC12CCC(n3c(=O)n(C)c4cnc(Nc5cc(F)c(C(N)=O)cc5C)nc43)(C1)C2]

### Step 1: Synthesis of 4-amino-2-fluoro-5-methylbenzamide

4-Amino-2-fluoro-5-methylbenzonitrile (150 mg, 0.999 mmol) was stirred in DMSO (0.7 mL) cooled in an ice bath. H₂O₂ (0.153 mL, 4.99 mmol) was added. The reaction was stirred at 60° C. for 1.5 h before H₂O (3 mL) was added. The mixture was filtered and the solids were washed with water, collected and dried under vacuum to afford the product (115 mg, 0.68 mmol, 68.4% yield) as a white solid.

### Step 2: Synthesis of 2-fluoro-4-((9-(4-methoxybicyclo[2.1.1]hexan-1-yl)-7-methyl-8-oxo-8,9-dihydro-7H-purin-2-yl)amino)-5-methylbenzamide

The title compound was prepared in 21.2% overall yield as a white solid according to the preparation of EXAMPLE 1 using 2-chloro-9-(4-methoxybicyclo[2.1.1]hexan-1-yl)-7-methyl-7,9-dihydro-8H-purin-8-one (Int-12) and 4-amino-2-fluoro-5-methylbenzamide. MS: m/z: Calc'd for C₂₁H₂₃FN₆O₃ [M+H]⁺ 427; Found 427. ¹H NMR (500 MHz, DMSO-d₆) δ 8.54-8.40 (m, 1H), 8.25-8.11 (m, 1H), 7.94 (d, J=14.5 Hz, 1H), 7.55 (br d, J=8.8 Hz, 1H), 7.48-7.36 (m, 2H), 3.34-3.30 (m, 1H), 3.30-3.26 (m, 2H), 2.29 (s, 3H), 2.22 (br s, 4H), 2.02 (br d, J=6.7 Hz, 2H), 1.77 (br d, J=5.2 Hz, 2H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 427, retention time 1.36 min, purity: 913.4%; LCMS 2 with TFA: [M+H]⁺ found 427, retention time 1.19 min, purity: 93.3%.

### Example 41: 2-((5-fluoro-2-methyl-4-(oxazol-5-yl)phenyl)amino)-9-(4-methoxybicyclo[2.1.1]hexan-1-yl)-7-methyl-7,9-dihydro-8H-purin-8-one (Compound 41)

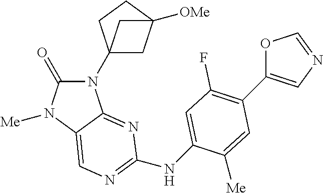 [SMILES: COC12CCC(n3c(=O)n(C)c4cnc(Nc5cc(F)c(-c6cnco6)cc5C)nc43)(C1)C2]

The title compound was prepared in 24.2% overall yield as a white solid according to the preparation of EXAMPLE 36 using 2-chloro-9-(4-methoxybicyclo[2.1.1]hexan-1-yl)-7-methyl-7,9-dihydro-8H-purin-8-one (Int-12) in Step 2. MS: m/z: Calc'd for C₂₃H₂₃FN₆O₃ [M+H]⁺ 451; Found 451. ¹H NMR (500 MHz, DMSO-d₆) δ 8.54 (s, 1H), 8.47 (s, 1H), 8.18 (s, 1H), 8.00 (d, J=13.9 Hz, 1H), 7.58 (d, J=8.5 Hz, 1H), 7.43 (d, J=3.2 Hz, 1H), 3.32-3.24 (m, 4H), 2.33 (s, 3H), 2.20 (br s, 4H), 2.09-1.99 (m, 2H), 1.80-1.73 (m, 2H), 1.13 (d, J=13.9 Hz, 1H).

Purification conditions: Column: XBridge C18, 19 mm×200 mm, 5 μm particles; Flow Rate: 20 mL/min; Column Temperature: 25° C. Fraction collection was triggered by UV (220 nm) and MS (ESI+). LCMS Method A: [M+H]⁺ found 451, retention time 1.76 min, purity: 95.4%; LCMS 2 with TFA: [M+H]⁺ found 451, retention time 1.51 min, purity: 96.5%.

### Example 42: 9-((1s,4s)-bicyclo[2.2.2]octan-2-yl)-7-methyl-2-((7-methyl-[1,2,4]triazolo[1,5-a]pyridin-6-yl)amino)-7,9-dihydro-8H-purin-8-one (Compound 42)

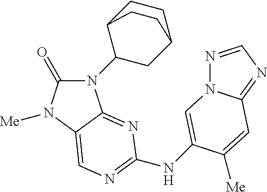 [SMILES: Cc1cc2ncnn2cc1Nc1ncc2c(n1)n(C1CC3CCC1CC3)c(=O)n2C]

The title compound was prepared in 52.9% overall yield as a white solid according to the preparation of EXAMPLE 1 using 9-((1s,4s)-bicyclo[2.2.2]octan-2-yl)-2-chloro-7-methyl-7,9-dihydro-8H-purin-8-one (Int-15) and 7-methyl-[1,2,4]triazolo[1,5-a]pyridin-6-amine. MS: m/z: Calc'd for C₂₁H₂₄N₆O [M+H]⁺ 405; Found 405. ¹H NMR (500 MHz, DMSO-d₆) δ 9.03 (s, 1H), 8.62 (s, 1H), 8.37 (s, 1H), 8.11 (s, 1H), 7.71 (s, 1H), 4.48-4.43 (m, 1H), 3.39-3.36 (m, 3H), 2.75 (br d, J=19.3 Hz, 1H), 2.36 (s, 3H), 1.83 (br s, 1H), 1.81-1.73 (m, 1H), 1.64 (br s, 1H), 1.57 (br s, 1H), 1.53-1.42 (m, 4H), 1.33 (br s, 1H), 1.23 (br t, J=11.9 Hz, 1H), 1.08 (br s, 1H).

### Example 43: 9-((1s,4s)-bicyclo[2.2.2]octan-2-yl)-7-methyl-2-((6-methylbenzo[d][1,3]dioxol-5-yl)amino)-7,9-dihydro-8H-purin-8-one (Compound 43)

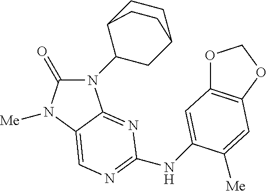 [SMILES: Cc1cc2c(cc1Nc1ncc3c(n1)n(C1CC4CCC1CC4)c(=O)n3C)OCO2]

The title compound was prepared in 59.8% overall yield as a white solid according to the preparation of EXAMPLE 1 using 9-((1s,4s)-bicyclo[2.2.2]octan-2-yl)-2-chloro-7-methyl-7,9-dihydro-8H-purin-8-one (Int-15) and 6-methylbenzo[d][1,3]dioxol-5-amine. MS: m/z: Calc'd for C₂₂H₂₅N₅O₃ [M+H]⁺ 408; Found 408. ¹H NMR (500 MHz, DMSO-d₆) δ 8.23 (s, 1H), 8.01 (s, 1H), 6.93 (s, 1H), 6.78 (s, 1H), 5.93 (d, J=2.7 Hz, 2H), 4.46-4.40 (m, 1H), 3.47-3.30 (m, 3H), 2.84-2.78 (m, 1H), 2.10 (s, 3H), 1.93-1.84 (m, 1H), 1.75 (br t, J=12.4 Hz, 1H), 1.64 (br s, 1H), 1.60-1.49 (m, 6H), 1.26 (br s, 2H).

### Example 44: 9-((1S,2S,4R)-bicyclo[2.2.1]heptan-2-yl)-7-methyl-2-((7-methylquinolin-6-yl)amino)-7,9-dihydro-8H-purin-8-one (Compound 44)

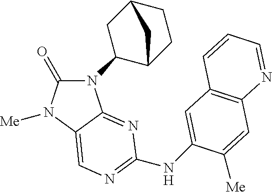 [SMILES: Cc1cc2ncccc2cc1Nc1ncc2c(n1)n([C@H]1C[C@@H]3CC[C@H]1C3)c(=O)n2C]

The title compound was prepared in 72.3% overall yield as a white solid according to the preparation of EXAMPLE 1 using 9-((1S,2S,4R)-bicyclo[2.2.1]heptan-2-yl)-2-chloro-7-methyl-7,9-dihydro-8H-purin-8-one (Int-16) and 7-methylquinolin-6-amine. MS: m/z: Calc'd for C₂₃H₂₄N₆O [M+H]⁺ 401; Found 401. ¹H NMR (500 MHz, DMSO-d₆) δ 8.95 (br d, J=3.5 Hz, 1H), 8.61 (br d, J=10.1 Hz, 1H), 8.47 (br s, 1H), 8.19 (s, 1H), 7.98 (s, 1H), 7.77 (br s, 1H), 7.29-6.96 (m, 4H), 4.27-4.21 (m, 1H), 3.33 (s, 3H), 2.58 (s, 3H), 2.41 (br s, 2H), 2.25-2.17 (m, 2H), 1.72-1.64 (m, 1H), 1.55-1.42 (m, 2H), 1.25 (br d, J=10.2 Hz, 1H), 1.22-1.14 (m, 1H), 1.02 (br d, J=8.9 Hz, 1H).

### Example 45: 9-((1S,2S,4R)-bicyclo[2.2.1]heptan-2-yl)-7-methyl-2-((7-methyl-[1,2,4]triazolo[1,5-a]pyridin-6-yl)amino)-7,9-dihydro-8H-purin-8-one (Compound 45)

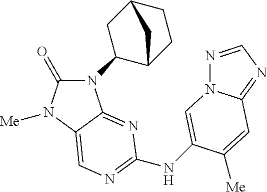 [SMILES: Cc1cc2ncnn2cc1Nc1ncc2c(n1)n([C@H]1C[C@@H]3CC[C@H]1C3)c(=O)n2C]

The title compound was prepared in 70.6% overall yield as a white solid according to the preparation of EXAMPLE 1 using 9-((1S,2S,4R)-bicyclo[2.2.1]heptan-2-yl)-2-chloro-7-methyl-7,9-dihydro-8H-purin-8-one (Int-16) and 7-methyl-[1,2,4]triazolo[1,5-a]pyridin-6-amine. MS: m/z: Calc'd for C₂₀H₂₂N₈O [M+H]⁺ 401; Found 401. ¹H NMR (500 MHz, DMSO-d₆) δ 8.88 (s, 1H), 8.18 (d, J=1.5 Hz, 1H), 7.87 (s, 1H), 7.50 (s, 1H), 7.05-6.72 (m, 1H), 3.94 (br s, 1H), 3.06 (s, 3H), 2.21-2.12 (m, 4H), 2.09 (br s, 1H), 1.99 (br s, 1H), 1.87 (br s, 1H), 1.39 (br t, J=10.5 Hz, 1H), 1.22 (br t, J=10.9 Hz, 2H), 1.03-0.95 (m, 1H), 0.92 (br d, J=8.9 Hz, 1H), 0.73 (br d, J=10.2 Hz, 1H).

### Example 46: 9-((1S,2S,4R)-bicyclo[2.2.1]heptan-2-yl)-7-methyl-2-((6-methylbenzo[d][1,3]dioxol-5-yl)amino)-7,9-dihydro-8H-purin-8-one (Compound 46)

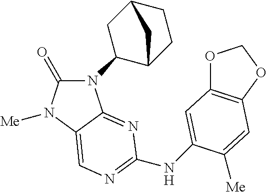 [SMILES: Cc1cc2c(cc1Nc1ncc3c(n1)n([C@H]1C[C@@H]4CC[C@H]1C4)c(=O)n3C)OCO2]

The title compound was prepared in 73.6% overall yield as a white solid according to the preparation of EXAMPLE 1 using 9-((1S,2S,4R)-bicyclo[2.2.1]heptan-2-yl)-2-chloro-7-methyl-7,9-dihydro-8H-purin-8-one (Int-16) and 6-methylbenzo[d][1,3]dioxol-5-amine. MS: m/z: Calc'd for C₂₁H₂₃N₅O₃ [M+H]⁺ 394; Found 394. ¹H NMR (500 MHz, DMSO-d₆) δ 8.01 (s, 1H), 7.30-7.01 (m, 1H), 7.01-6.99 (m, 1H), 6.84 (s, 1H), 5.98 (d, J=1.6 Hz, 2H), 4.15 (br d, J=5.6 Hz, 1H), 3.64-2.87 (m, 3H), 2.41 (br s, 1H), 2.33 (br s, 1H), 2.28 (br s, 1H), 2.17-2.09 (m, 4H), 1.66-1.60 (m, 1H), 1.49 (br s, 2H), 1.26-1.20 (m, 1H), 1.16 (br d, J=11.0 Hz, 1H), 1.07-1.02 (m, 1H).

### Example 47: 1-(bicyclo[1.1.1]pentan-1-yl)-3-methyl-N-(7-methyl-[1,2,4]triazolo[1,5-a]pyridin-6-yl)-1H-pyrazolo[3,4-d]pyrimidin-6-amine (Compound 47)

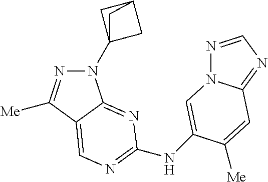 [SMILES: Cc1cc2ncnn2cc1Nc1ncc2c(C)nn(C34CC(C3)C4)c2n1]

The title compound was prepared in 87.0% overall yield as a white solid according to the preparation of EXAMPLE 1 using 1-(bicyclo[1.1.1]pentan-1-yl)-6-chloro-3-methyl-1H-pyrazolo[3,4-d]pyrimidine (Int-17) and 7-methyl-[1,2,4]triazolo[1,5-a]pyridin-6-amine. MS: m/z: Calc'd for C₁₈H₁₈N₈ [M+H]⁺ 347; Found 347. ¹H NMR (500 MHz, DMSO-d₆) δ 9.36 (s, 1H), 9.19 (s, 1H), 8.97 (s, 1H), 8.41 (s, 1H), 7.74 (s, 1H), 2.63 (s, 1H), 2.44 (s, 6H), 2.32 (s, 6H).

### Example 48: 1-(bicyclo[1.1.1]pentan-1-yl)-3-methyl-N-(6-methylbenzo[d][1,3]dioxol-5-yl)-1H-pyrazolo[3,4-d]pyrimidin-6-amine (Compound 48)

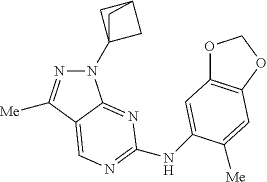 [SMILES: Cc1cc2c(cc1Nc1ncc3c(C)nn(C45CC(C4)C5)c3n1)OCO2]

The title compound was prepared in 42.9% overall yield as a white solid according to the preparation of EXAMPLE 1 using 1-(bicyclo[1.1.1]pentan-1-yl)-6-chloro-3-methyl-1H-pyrazolo[3,4-d]pyrimidine (Int-17) and 6-methylbenzo[d][1,3]dioxol-5-amine. MS: m/z: Calc'd for C₁₉H₁₉N₅O₂ [M+H]⁺ 350; Found 350. ¹H NMR (500 MHz, DMSO-d₆) δ 8.84 (s, 1H), 8.79 (s, 1H), 7.21 (s, 1H), 6.81 (s, 1H), 5.98 (s, 2H), 2.61 (s, 1H), 2.40 (s, 3H), 2.28 (s, 6H), 2.15 (s, 3H).

### Example 49: N-(1-(bicyclo[1.1.1]pentan-1-yl)-3-methyl-1H-pyrazolo[3,4-d]pyrimidin-6-yl)-7-methylquinoxalin-6-amine (Compound 49)

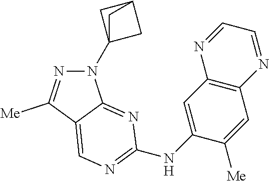 [SMILES: Cc1cc2nccnc2cc1Nc1ncc2c(C)nn(C34CC(C3)C4)c2n1]

The title compound was prepared in 71.3% overall yield as a white solid according to the preparation of EXAMPLE 1 using 1-(bicyclo[1.1.1]pentan-1-yl)-6-chloro-3-methyl-1H-pyrazolo[3,4-d]pyrimidine (Int-17) and 7-methylquinoxalin-6-amine. MS: m/z: Calc'd for C₂₀H₁₉N₇[M+H]⁺ 358; Found 358. ¹H NMR (500 MHz, DMSO-d₆) δ 9.12 (s, 1H), 9.05 (s, 1H), 8.90 (s, 1H), 8.85 (s, 1H), 8.78 (s, 1H), 7.95 (s, 1H), 2.68 (s, 1H), 2.61 (s, 3H), 2.48 (s, 3H), 2.41 (s, 6H).

### Example 50: N-(1-(bicyclo[1.1.1]pentan-1-yl)-3-methyl-1H-pyrazolo[3,4-d]pyrimidin-6-yl)-6-methylbenzo[c][1,2,5]thiadiazol-5-amine (Compound 50)

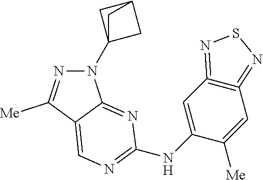

The title compound was prepared in 52.1% overall yield as a white solid according to the preparation of EXAMPLE 1 using 1-(bicyclo[1.1.1]pentan-1-yl)-6-chloro-3-methyl-1H-pyrazolo[3,4-d]pyrimidine (Int-17) and 6-methylbenzo[c][1,2,5]thiadiazol-5-amine. MS: m/z: Calc'd for C₁₈H₁₇N₇S [M+H]⁺ 364; Found 364. ¹H NMR (500 MHz, DMSO-d₆) δ 9.06 (s, 1H), 9.04-8.98 (m, 1H), 8.86 (s, 1H), 7.94 (s, 1H), 2.70 (s, 1H), 2.58 (s, 3H), 2.48 (s, 3H), 2.42 (s, 6H).

### Example 51: 8-{4-Hydroxybicyclo[2.2.1]heptan-1-yl}-5-methyl-2-({7-methyl-[1,2,4]triazolo[1,5-a]pyridin-6-yl}amino)-5,6,7,8-tetrahydropteridin-6-one (Compound 51)

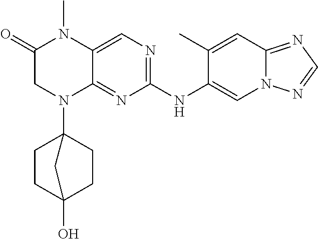 [SMILES: Cc1cc2ncnn2cc1Nc1ncc2c(n1)N(C13CCC(O)(CC1)C3)CC(=O)N2C]

To a stirred solution of 2-chloro-8-(4-hydroxybicyclo[2.2.1]heptan-1-yl)-5-methyl-7,8-dihydropteridin-6(5H)-one (Int-18, 40 mg, 0.130 mmol) in dioxane (5 mL) were added Cs₂CO₃ (127 mg, 0.389 mmol) and 7-methyl-[1,2,4]triazolo[1,5-a]pyridin-6-amine (26.9 mg, 0.181 mmol) at ambient temperature. The mixture was degassed for 5 min. 1,1′-bis(dicyclohexylphosphino)ferrocene (14.99 mg, 0.026 mmol) and Pd₂(dba)₃ (23.73 mg, 0.026 mmol) were added to the reaction mixture and degassed for 5 min. It was heated at 100° C. for 2 h under microwave. The reaction was monitored by TLC and LCMS. The reaction mixture was diluted with water (50 mL). The crude product was extracted with ethyl acetate (2×50 mL). The combined organic layer was washed with brine, dried with anhydrous sodium sulphate, and concentrated under reduced pressure. The crude was purified by RP-HPLC to give the titled compound (26 mg, 46.9%).

Column: Kinetex XB—C18 (75×30) mm, 2.6 μm Mobile Phase A: 5 mm Ammonium formate pH 3.3:ACN (98:02) Mobile Phase A: 5 mm Ammonium formate pH 3.3:ACN (98:02) Flow Rate: 1.0 ml/min. ¹H NMR (400 MHz, DMSO-d6): δ 8.92 (s, 1H), 8.39 (s, 1H), 8.35 (s, 1H), 7.72 (s, 1H), 7.70 (s, 1H), 4.84 (s, 1H), 4.05 (s, 2H), 3.17 (s, 3H), 2.32 (s, 3H), 2.16-2.20 (m, 2H), 1.72 (s, 2H), 1.48-1.60 (m, 4H), 1.14-1.18 (m, 2H). HPLC (purity)=98.32%. Column: X-select CSH C18 (150×4.6) mm, 3.5 μm, Mobile phase: A: 10 mM Ammonium acetate in water, Mobile phase: B: CAN, Flow: 1.0 mL/min. Time/Grad (% B): 0/10, 9/60, 11/100, 11.1/100 (1.5 mL/min), 12.5/100 (1.5 mL/min), 13/10, 15/10. RT: 6.063 min, LCMS m/z: 421.2 [M+H]⁺, RT=1.26 min, Column: Kinetec Biphenyl (100×4.6 mm) 2.6 μm, Mobile phase: A: 0.1% TFA in H₂O, Mobile phase: B: 0.1% TFA in AC, Flow Rate: 1.0 mL/min. Time/Grad (% B): 0/10, 7/60, 7.1/100, 8/100, 8.1/10, 10/10.

Using procedures similar to Example 51, the following Examples 52-56 were obtained.

### Example 52: 8-{4-Hydroxybicyclo[2.2.1]heptan-1-yl}-5-methyl-2-[(6-methyl-2H-1,3-benzodioxol-5-yl)amino]-5,6,7,8-tetrahydropteridin-6-one (Compound 52)

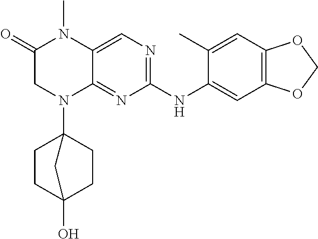 [SMILES: Cc1cc2c(cc1Nc1ncc3c(n1)N(C14CCC(O)(CC1)C4)CC(=O)N3C)OCO2]

LCMS: m/z 424.2 [M+H]⁺, RT=2.51 min (conditions: Column: XSELECT CSH C18(50×4.6 mm) 3.5 μm Mobile phase: A: 10 mm AA in H₂O. Mobile phase: B: CAN. Flow Rate: 1 mL/min. Time (min)/Grad (% B): 0/5, 2.5/95, 2.51/95, 4.5/95). HPLC purity: 99.06%, RT: 4.33 min (conditions: Column: Kinetex Biphenyl (100×4.6) mm, 2.6 μm Mobile phase: A: 0.05% TFA in water:ACN (95:5). Mobile phase: B: ACN:0.05% TFA in water (95:5). Flow: 1.0 mL/min. Time (min)/Grad (% B): 0/10, 9/60, 11/100, 11.1/100 (1.5 mL/min), 12.5/100 (1.5 mL/min), 13/10, 15/10). ¹H NMR (400 MHz, DMSO-d6): δ 8.15 (s, 1H), 7.98 (s, 1H), 7.62 (s, 1H), 6.80 (d, J=9.20 Hz, 2H), 5.94 (s, 2H), 4.01 (s, 2H), 3.15 (s, 3H), 2.24-2.27 (m, 2H), 2.06 (s, 3H), 1.73 (s, 2H), 1.57-1.58 (m, 4H), 1.36-1.38 (m, 2H).

### Example 53: 8-{4-Hydroxybicyclo[2.2.1]heptan-1-yl}-5-methyl-2-[(7-methylquinolin-6-yl)amino]-5,6,7,8-tetrahydropteridin-6-one (Compound 53)

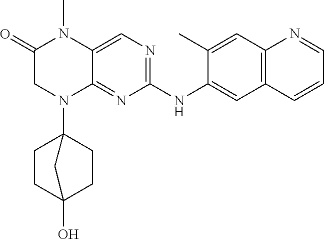 [SMILES: Cc1cc2ncccc2cc1Nc1ncc2c(n1)N(C13CCC(O)(CC1)C3)CC(=O)N2C]

LCMS: m/z 431.1 [M+H]⁺, RT=3.94 min (conditions: Column: Kinetec Biphenyl (100×4.6 mm) 2.6 μm. Mobile phase: A: 0.1% TFA in H2O; B: 0.1% TFA in CAN. Flow Rate: 1.0 mL/min. Time (min)/Grad (% B): 0/10, 7/60, 7.1/100, 8/100, 8.1/10, 10/10). HPLC purity: 86.2%, RT: 7.84 min (conditions: Column: X-select CSH C18 (150×4.6) mm, 3.5 μm Mobile phase: A: 10 mM Ammonium acetate in water Mobile phase: B: CAN. Flow: 1.0 mL/min. Time (min)/Grad (% B): 0/10, 9/60, 11/100, 11.1/100 (1.5 mL/min), 12.5/100 (1.5 mL/min), 13/10, 15/10). ¹H NMR (400 MHz, DMSO-d6): δ 8.75-8.77 (m, 1H), 8.3 (s, 1H), 8.17 (d, J=7.60 Hz, 1H), 8.04 (s, 1H), 7.86 (s, 1H), 7.74 (s, 1H), 7.41-7.44 (m, 1H), 4.70-4.90 (m, 1H), 4.07 (s, 2H), 3.19 (s, 3H), 2.45 (s, 3H), 2.31-2.34 (m, 2H), 1.74 (s, 2H), 1.53-1.62 (m, 4H), 1.13-1.5 (m, 2H).

### Example 54: 8-{3-Fluorobicyclo[1.1.1]pentan-1-yl}-5-methyl-2-({7-methyl-[1,2,4]triazolo[1,5-a]pyridin-6-yl}amino)-5,6,7,8-tetrahydropteridin-6-one (Compound 54)

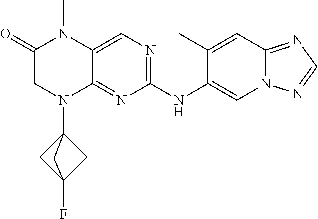 [SMILES: Cc1cc2ncnn2cc1Nc1ncc2c(n1)N(C13CC(F)(C1)C3)CC(=O)N2C]

LCMS: m/z 395.0 [M+H]⁺, RT=1.09 min (conditions: Column: Kinetex XB—C18 (75×3.0) mm, 2.6 μm. Mobile Phase: A: 5 mm Ammonium formate pH 3.3:ACN (98:02); B: ACN:Buffer (98:02). Flow Rate: 1.0 ml/min. Time (min)/Grad (% B): 0/20, 4/100, 4.1/100 (1.5 ml/min), 4.6/100 (1.5 ml/min), 4.7/20, 5/20). HPLC purity: 98.3%, RT: 4.15 min (conditions: Column: Kinetex Biphenyl (100×4.6) mm, 2.6 micron. Mobile phase A: 0.05% TFA in water:Acetonitrile (95:5), Mobile phase B: 0.05% TFA in water:Acetonitrile. Flow: 1.0 mL/min. Time (min)/Grad (% B): 0/10, 9/60, 11/100, 11.1/100 (1.5 mL/min), 12.5/100 (1.5 mL/min), 13/10, 15/10). ¹H NMR (400 MHz, DMSO-d₆): δ 8.97 (s, 1H), 8.48 (s, 1H), 8.39 (s, 1H), 7.8 (s, 1H), 7.74 (s, 1H), 4.05 (s, 2H), 3.19 (s, 3H), 2.34-2.35 (m, 9H).

### Example 55: 8-{4-Hydroxybicyclo[2.2.1]heptan-1-yl}-5-methyl-2-[(6-methyl-2,1,3-benzothiadiazol-5-yl)amino]-5,6,7,8-tetrahydropteridin-6-one (Compound 55)

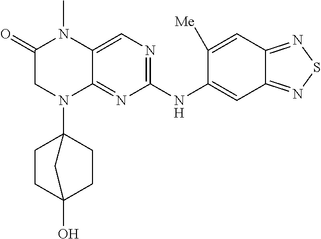 [SMILES: Cc1cc2nsnc2cc1Nc1ncc2c(n1)N(C13CCC(O)(CC1)C3)CC(=O)N2C]

LCMS: m/z 438.0 [M+H]⁺, RT=0.92 min (conditions: Column: Column: Kinetex XB—C18 (75×3.0) mm, 2.6 μm. Mobile Phase A: 5 mm Ammonium formate pH 3.3:ACN (98:02), Mobile Phase: B: ACN:Buffer (98:02). Flow Rate: 1.0 mL/min. Time (min)/Grad (% B): 0/20, 4/100, 4.1/100 (1.5 mL/min), 4.6/100 (1.5 mL/min), 4.7/20, 5/20). HPLC purity: 93.9%, RT: 4.26 min (conditions: Column: Kinetex Biphenyl (100×4.6) mm, 2.6 micron. Mobile phase A: 0.05% TFA in water:Acetonitrile (95:5), Mobile phase B: 0.05% TFA in water:Acetonitrile (5:95). Flow: 1.0 mL/min. Time (min)/Grad (% B): 0/10, 9/60, 11/100, 11.1/100 (1.5 mL/min), 12.5/100 (1.5 mL/min), 13/10, 15/10). ¹H NMR (400 MHz, DMSO-d₆): δ 8.39 (s, 1H), 8.21 (s, 1H), 7.90 (s, 1H), 7.81 (s, 1H), 4.89 (s, 1H), 4.12 (s, 2H), 3.21 (s, 3H), 1.81 (s, 2H), 1.62-1.69 (m, 4H), 1.30-1.40 (m, 2H). One CH₃ and —CH₂ peak are merged with the solvent residue at 2.5 ppm.

### Example 56: 8-{3-Fluorobicyclo[1.1.1]pentan-1-yl}-5-methyl-2-[(6-methyl-2H-1,3-benzodioxol-5-yl)amino]-5,6,7,8-tetrahydropteridin-6-one (Compound 56)

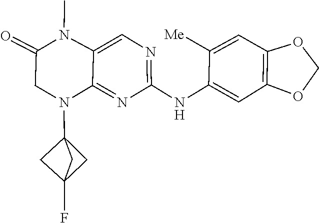 [SMILES: Cc1cc2c(cc1Nc1ncc3c(n1)N(C14CC(F)(C1)C4)CC(=O)N3C)OCO2]

LCMS: m/z 398.0 [M+H]⁺, RT=1.77 min (conditions: Kinetex XB—C18 (75×3.0) mm, 2.6 μm. Mobile Phase A: 5 mm Ammonium formate pH 3.3:ACN (98:02), Mobile Phase: B: ACN:Buffer (98:02). Flow Rate: 1.0 ml/min. Time/Grad (% B): 0/20, 4/100, 4.1/100 (1.5 mL/min), 4.6/100 (1.5 mL/min), 4.7/20, 5/20). HPLC purity: 98.0%, RT: 6.81 min (conditions: Column: Kinetex Biphenyl (100×4.6) mm, 2.6 micron. Mobile phase A: 0.05% TFA in water:Acetonitrile (95:5), Mobile phase B: 0.05% TFA in water:Acetonitrile. Flow: 1.0 mL/min. Time (min)/Grad (% B): 0/10, 9/60, 11/100, 11.1/100 (1.5 mL/min), 12.5/100 (1.5 mL/min), 13/10, 15/10). ¹H NMR (400 MHz, DMSO-d₆): δ 8.17 (d, J=8.80 Hz, 1H), 7.72 (s, 1H), 6.83 (d, J=10.00 Hz, 2H), 5.95 (s, 1H), 4.01 (s, 2H), 3.18 (s, 3H), 2.35 (d, J=2.00 Hz, 6H), 2.09 (s, 3H).

The following Examples were prepared in a methodology analogous to the Example 51 with suitable reagents, precursors and starting materials.

### Example 57: 8-(3-fluorobicyclo[1.1.1]pentan-1-yl)-5-methyl-2-((7-methylquinolin-6-yl)amino)-7,8-dihydropteridin-6(5H)-one (Compound 57)

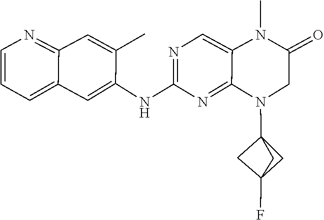 [SMILES: Cc1cc2ncccc2cc1Nc1ncc2c(n1)N(C13CC(F)(C1)C3)CC(=O)N2C]

LC-MS: m/z: 405.2 [M+H]⁺; RT=1.20 min (LCMS method G). HPLC purity: 95.39%, RT=7.41 min. (HPLC Method A). ¹H NMR (400 MHz, DMSO-d6): δ 8.74-8.76 (m, 1H), 8.47 (s, 1H), 8.19 (d, J=8.40 Hz, 1H), 8.04 (s, 1H), 7.87 (s, 1H), 7.84 (s, 1H), 7.40-7.43 (m, 1H), 4.05 (s, 2H), 3.21 (s, 3H), 2.46 (s, 3H), 2.37 (d, J=2.40 Hz, 6H).

### Example 58: 8-(3-fluorobicyclo[1.1.1]pentan-1-yl)-5-methyl-2-((6-methylbenzo[c][1,2,5]thiadiazol-5-yl)amino)-7,8-dihydropteridin-6(5H)-one (Compound 58)

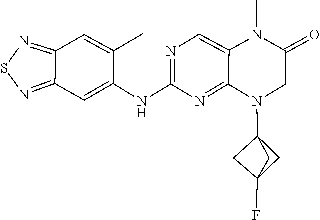 [SMILES: Cc1cc2nsnc2cc1Nc1ncc2c(n1)N(C13CC(F)(C1)C3)CC(=O)N2C]

LC-MS: m/z: 412.0 [M+H]⁺; RT=2.32 min (LCMS method G). HPLC purity: 96.73%, RT=4.79 min. (HPLC Method B). ¹H NMR (400 MHz, DMSO-d₆): δ 8.84 (s, 1H), 7.82 (s, 2H), 6.84 (s, 1H), 4.13 (s, 2H), 3.37 (s, 3H), 2.63 (d, J=2.00 Hz, 6H), 2.56 (d, J=0.80 Hz, 3H)

### Example 59: 8-(4-hydroxybicyclo[2.2.1]heptan-1-yl)-5-methyl-2-((7-methylquinoxalin-6-yl)amino)-7,8-dihydropteridin-6(5H)-one (Compound 59)

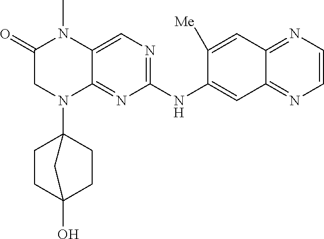 [SMILES: Cc1cc2nccnc2cc1Nc1ncc2c(n1)N(C13CCC(O)(CC1)C3)CC(=O)N2C]

LC-MS: m/z: 432.0 [M+H]⁺; RT=1.48 min (LCMS method F). HPLC purity: 93.64%, RT=7.54 min. (HPLC Method A). ¹H NMR (400 MHz, DMSO-d₆): δ 8.80 (d, J=2.00 Hz, 1H), 8.74 (d, J=2 Hz, 1H), 8.42 (s, 1H), 8.33 (s, 1H), 7.90 (s, 1H), 7.80 (s, 1H), 4.86 (s, 1H), 4.11 (s, 2H), 3.21 (s, 3H), 2.67 (s, 3H), 1.79 (s, 2H), 1.59-1.66 (m, 4H), 1.30-1.40 (m, 2H), two protons are merged with the solvent peak at 2.5 ppm.

### Example 60: 8-(4-hydroxybicyclo[2.2.1]heptan-1-yl)-5-methyl-2-((6-methylbenzo[c][1,2,5]oxadiazol-5-yl)amino)-7,8-dihydropteridin-6(5H)-one (Compound 60)

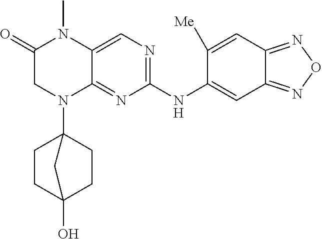 [SMILES: Cc1cc2nonc2cc1Nc1ncc2c(n1)N(C13CCC(O)(CC1)C3)CC(=O)N2C]

LC-MS: m/z: 422.0 [M+H]⁺, RT=1.532 min (LCMS method G). HPLC purity: 98.44%, RT=3.28 min. (HPLC Method B). ¹H NMR (400 MHz, DMSO-d6): δ 8.34 (s, 1H), 8.2 (s, 1H), 7.86 (s, 2H), 4.95 (s, 1H), 4.16 (s, 2H), 3.22 (s, 3H), 2.57-2.62 (m, 2H), 2.49 (s, 3H), 1.87 (s, 2H), 1.66-1.78 (m, 4H), 1.46-1.51 (m, 2H).

### Example 61: 8-(4-hydroxybicyclo[2.2.1]heptan-1-yl)-5-methyl-2-((7-methylcinnolin-6-yl)amino)-7,8-dihydropteridin-6(5H)-one (Compound 61)

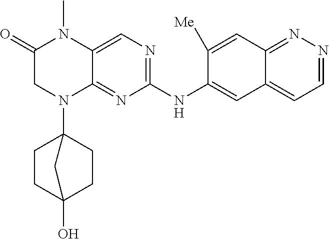 [SMILES: Cc1cc2nnccc2cc1Nc1ncc2c(n1)N(C13CCC(O)(CC1)C3)CC(=O)N2C]

LC-MS: m/z: 432.2 [M+H]⁺, RT=2.76 min (LCMS method G). HPLC purity: 93.24%, RT=2.81 min (HPLC Method A). ¹H NMR (400 MHz, DMSO-d₆): δ 9.16 (d, J=6.00 Hz, 1H), 8.32-8.37 (m, 2H), 8.25 (s, 1H), 7.91 (d, J=6.00 Hz, 1H), 7.83 (s, 1H), 5.00 (s, 1H), 4.14 (s, 2H), 3.22 (s, 3H), 2.54-2.58 (m, 4H), 1.83 (s, 2H), 1.60-1.71 (m, 4H), 1.34-1.38 (m, 2H), one proton is merged with the solvent peak at 2.5 ppm.

### Example 62: 8-(4-methoxybicyclo[2.2.1]heptan-1-yl)-5-methyl-2-((7-methyl-[1,2,4]triazolo[1,5-a]pyridin-6-yl)amino)-7,8-dihydropteridin-6(5H)-one (Compound 62)

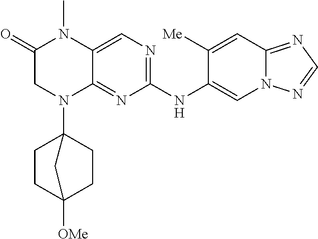 [SMILES: COC12CCC(N3CC(=O)N(C)c4cnc(Nc5cn6ncnc6cc5C)nc43)(CC1)C2]

LC-MS: m/z: 435.2 [M+H]⁺, RT=2.29 min (LCMS method G). HPLC purity: 95.85%, RT=5.82 min (HPLC Method B). ¹H NMR (400 MHz, DMSO-d₆): δ 8.91 (s, 1H), 8.38 (s, 2H), 7.72 (s, 2H), 4.05 (s, 2H), 3.21 (s, 3H), 3.03 (s, 3H), 2.32 (s, 3H), 2.14-2.18 (m, 2H), 1.82 (s, 2H), 1.60-1.63 (m, 4H), 1.16-1.24 (m, 2H).

### Example 63: 8-(4-methoxybicyclo[2.2.1]heptan-1-yl)-5-methyl-2-((6-methylbenzo[d][1,3]dioxol-5-yl)amino)-7,8-dihydropteridin-6(5H)-one (Compound 63)

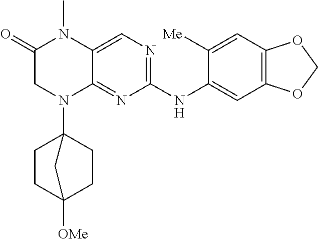 [SMILES: COC12CCC(N3CC(=O)N(C)c4cnc(Nc5cc6c(cc5C)OCO6)nc43)(CC1)C2]

LC-MS: m/z: 438.2 [M+H]⁺, RT=1.48 min (LCMS method G). HPLC purity: 95.31%, RT=6.19 min (HPLC Method A). ¹H NMR (400 MHz, DMSO-d6): δ 8.03 (s, 1H), 7.65 (s, 1H), 6.81 (d, J=4.80 Hz, 2H), 5.95 (s, 2H), 4.02 (s, 2H), 3.15 (d, J=7.20 Hz, 6H), 2.20-2.24 (m, 2H), 2.07 (s, 3H), 1.86 (s, 2H), 1.67-1.68 (m, 4H), 1.36-1.40 (m, 2H).

### Example 64: 8-(4-methoxybicyclo[2.2.1]heptan-1-yl)-5-methyl-2-((6-methylbenzo[c][1,2,5]thiadiazol-5-yl)amino)-7,8-dihydropteridin-6(5H)-one (Compound 64)

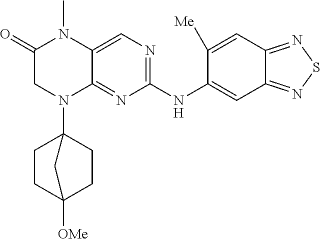 [SMILES: COC12CCC(N3CC(=O)N(C)c4cnc(Nc5cc6nsnc6cc5C)nc43)(CC1)C2]

LC-MS: m/z: 452.7 [M+H]⁺, RT=1.96 min (LCMS method F). HPLC purity: 96.76%, RT=5.92 min (HPLC Method A). ¹H NMR (400 MHz, DMSO-d6): δ 8.37 (s, 1H), 8.28 (s, 1H), 7.91 (s, 1H), 7.83 (s, 1H), 4.13 (s, 2H), 3.22 (s, 3H), 3.08 (s, 3H), 1.93 (s, 2H), 1.73-1.76 (m, 4H), 1.37-1.41 (m, 2H). Three CH₃ and two CH₂ protons are merged in solvent peak.

### Example 65: 8-(4-methoxybicyclo[2.2.1]heptan-1-yl)-5-methyl-2-((7-methylquinolin-6-yl)amino)-7,8-dihydropteridin-6(5H)-one (Compound 65)

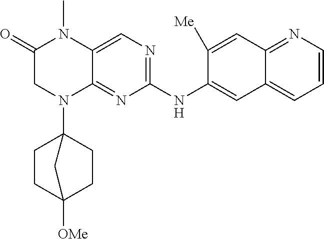 [SMILES: COC12CCC(N3CC(=O)N(C)c4cnc(Nc5cc6cccnc6cc5C)nc43)(CC1)C2]

LC-MS: m/z: 445.8 [M+H]⁺, RT=1.45 min (LCMS method F). HPLC purity: 95.01%, RT=8.44 min (Conditions: Column: X-Bridge C8(150×4.6) mm, 3.5 μm, Mobile Phase A: 10 mm Ammonium Acetate in Water, Mobile Phase B: ACN:100%, Flow: 1.0 mL/min, Time (min)/Grad (% B): 0/10, 9/60, 11/100, 11.1/100 (1.5 mL/min), 12.5/100 (1.5 mL/min), 13/10, 15/10). ¹H NMR (400 MHz, DMSO-d6): δ 8.76 (d, J=2.80 Hz, 1H), 8.36 (s, 1H), 8.19 (d, J=7.20 Hz, 1H), 8.02 (s, 1H), 7.86 (s, 1H), 7.76 (s, 1H), 7.41-7.44 (m, 1H), 4.07 (s, 2H), 3.20 (s, 3H), 3.00 (s, 3H), 2.45 (s, 3H), 2.20-2.40 (m, 2H), 0.00 (s, 2H), 1.61-1.66 (m, 4H).

### Example 66. 7′-(4-hydroxybicyclo[2.2.1]heptan-1-yl)-2′-((7-methyl-[1,2,4]triazolo[1,5-a]pyridin-6-yl)amino)spiro[cyclopropane-1,5′-pyrrolo[2,3-d]pyrimidin]-6′(7′H)-one (Compound 66)

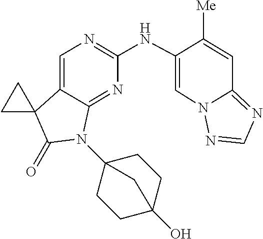 [SMILES: Cc1cc2ncnn2cc1Nc1ncc2c(n1)N(C13CCC(O)(CC1)C3)C(=O)C21CC1]

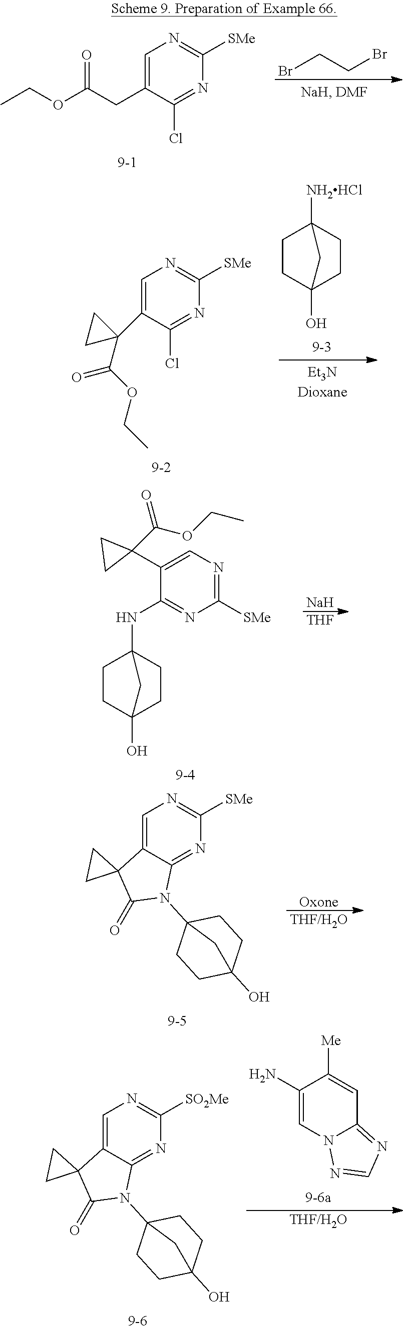

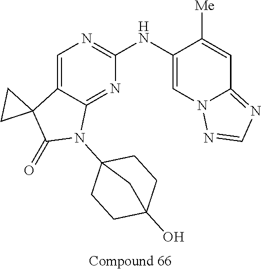 [SMILES: Cc1cc2ncnn2cc1Nc1ncc2c(n1)N(C13CCC(O)(CC1)C3)C(=O)C21CC1]

### Step 1. Ethyl 1-(4-chloro-2-(methylthio)pyrimidin-5-yl)cyclopropane-1-carboxylate (9-2)

To a stirred solution of NaH (1.75 g, 73.0 mmol) in DMF (70 mL) was added ethyl 2-(4-chloro-2-(methylthio)pyrimidin-5-yl)acetate (7.20 g, 29.2 mmol). To it then 1,2-dibromoethane (8.22 g, 43.8 mmol) was added at 0° C. The resulting reaction mixture was stirred at 25° C. for 16 hr. It was quenched with 30 mL of water at 0° C. and extracted with ethyl acetate (2×80 mL). The combined organic layers were washed with brine, dried with anhydrous sodium sulphate, and concentrated under reduced pressure to afford the crude product, which was purified by silica gel column chromatography eluted in 30-40% EtOAc/petroleum ether to afford ethyl 1-(4-chloro-2-(methylthio)pyrimidin-5-yl) cyclopropane-1-carboxylate (3.5 g, 12.32 mmol, 42.2% yield) as a brown gum. LCMS: m/z: 273.2 [M+H]⁺ RT=2.65 min (LCMS method F).

### Step 2. Ethyl 1-(4-((4-hydroxybicyclo[2.2.1]heptan-1-yl)amino)-2-(methylthio)pyrimidin-5-yl)cyclopropane-1-carboxylate (9-4)

To a stirred mixture of ethyl 1-(4-chloro-2-(methylthio)pyrimidin-5-yl)cyclopropane-1-carboxylate (500 mg, 1.833 mmol) in N,N-Dimethylacetamide (10 mL) were added 4-aminobicyclo[2.2.1]heptan-1-ol hydrochloride (450 mg, 2.75 mmol) and sodium bicarbonate (308 mg, 3.67 mmol). The reaction mixture was stirred at 130° C. over a period of 16 h. The resulting reaction mixture was diluted with water (50 mL) and extracted with EtOAc (2×100 mL). The combined organic layer was dried over sodium sulfate, filtered and concentrated under reduced pressure to afford the crude. The crude compound was purified by RP purification [Diluent: THF:Water:ACN (50:20:30), Column: Redisep 40 gm C18, 20-40 micron, Mobile phase A: 5 mM Ammonium formate in water; Mobile phase B: Acetonitrile. Compound elution (%): 35% Acetonitrile/5 mM Ammonium formate in water Flow Rate 40 ml/min] to afford ethyl 1-(4-((4-hydroxybicyclo[2.2.1]heptan-1-yl)amino)-2-(methylthio)pyrimidin-5-yl)cyclopropane-1-carboxylate (220 mg, 0.381 mmol, 20.80% yield) as off-white solid. LCMS: m/z: 364.6 [M+H]⁺, RT=0.36 min (LCMS method F).

### Step 3. 7′-(4-hydroxybicyclo[2.2.1]heptan-1-yl)⁻²′-(methylthio)spiro[cyclopropane-1,5′-pyrrolo[2,3-d]pyrimidin]-6′(7′H)-one (9-5)

A stirred solution of ethyl 1-(4-((4-hydroxybicyclo[2.2.1]heptan-1-yl)amino)-2-(methylthio)pyrimidin-5-yl)cyclopropane-1-carboxylate (60 mg, 0.165 mmol) in THF (3 mL) was cooled to 0° C. Then to it NaH (19.81 mg, 0.495 mmol) was added at 0° C. The reaction mixture was stirred at 60° C. for 1 h. Then the reaction mixture was cooled to 0° C., quenched with 50 mL of water and extracted with ethyl acetate (2×80 mL). The combined organic layer was washed with brine, filtered and dried with anhydrous sodium sulphate and concentrated under reduced pressure to afford 70 mg of crude compound (mixture of compound 3-acid and desired compound 4). The crude compound was dissolved in DMF (5 mL) and to it HATU (94 mg, 0.248 mmol) and DIPEA (0.072 mL, 0.413 mmol) were added. The reaction mixture was stirred at 25° C. over a period of 16 h. The resulting reaction mixture was diluted with water (50 mL) and extracted with EtOAc (2×100 mL). The combined organic layer was dried over sodium sulfate, filtered and concentrated under reduced pressure to afford the crude. The crude compound was purified by RP purification [Diluent: THF:Water:ACN (50:20:30) Column: Redisep 40 gm C18, 20-40 micron, Mobile phase A: 5 mM Ammonium formate in water; Mobile phase B: Acetonitrile. Compound elution (%): 35% Acetonitrile/5 mM Ammonium formate in water, Flow Rate: 40 ml/min] to afford 7′-(4-hydroxybicyclo[2.2.1]heptan-1-yl)-2′-(methylthio)spiro[cyclopropane-1,5′-pyrrolo[2,3-d]pyrimidin]-6′(7′H)-one (60 mg, 0.157 mmol, 95% yield). LCMS: m/z: 318.2 [M+H]⁺, RT=1.70 min (LCMS method G).

### Step 4. 7′-(4-hydroxybicyclo[2.2.1]heptan-1-yl)-2′-(methylsulfonyl)spiro[cyclopropane-1,5′-pyrrolo[2,3-d]pyrimidin]-6′(7′H)-one (9-6)

A stirred solution of 7′-(4-hydroxybicyclo[2.2.1]heptan-1-yl)-2′-(methylthio)spiro[cyclopropane-1,5′-pyrrolo[2,3-d]pyrimidin]-6′(7′H)-one (60 mg, 0.189 mmol) in THF (3 mL) and H₂O (3 mL) was cooled to 0° C. To it then oxone (291 mg, 0.473 mmol) was added at 0° C. The resulting reaction mixture was stirred at 25° C. for 16 h. The reaction mixture was quenched with 3 mL of aq. solution of sodium bisulphite and diluted with 50 mL of water. The reaction mixture was extracted with ethyl acetate (2×60 mL). The combined organic fraction was washed with brine, dried over sodium sulfate, filtered and concentrated under reduced pressure to afford the crude product. The crude compound was purified by RP column purification [Diluent: THF:Water:ACN (50:20:30) Column: Redisep 40 gm C18, 20-40 micron, Mobile phase A: 5 mM Ammonium formate in water; Mobile phase B: Acetonitrile; Instrument 1D: Teledyne Isco-Combi flash. Compound elution (%): 30% Acetonitrile/5 mM Ammonium formate in water Flow Rate: 30 ml/min] to afford 7′-(4-hydroxybicyclo[2.2.1]heptan-1-yl)-2′-(methylsulfonyl)spiro[cyclopropane-1,5′-pyrrolo[2,3-d]pyrimidin]-6′(7′H)-one (60 mg, 0.137 mmol, 72.7% yield) as off white solid. LCMS: m/z: 350.0 [M+H]⁺, RT=0.74 min (UHPLC Method D).

### Step 5. 7′-(4-hydroxybicyclo[2.2.1]heptan-1-yl)-2′-((7-methyl-[1,2,4]triazolo[1,5-a]pyridin-6-yl)amino)spiro[cyclopropane-1,5′-pyrrolo[2,3-d]pyrimidin]-6′(7′H)-one (Compound 66)

To a stirred solution of 7′-(4-hydroxybicyclo[2.2.1]heptan-1-yl)-2′-(methylsulfonyl)spiro[cyclopropane-1,5′-pyrrolo[2,3-d]pyrimidin]-6′(7′H)-one (30 mg, 0.086 mmol) and 7-methyl-[1,2,4]triazolo[1,5-a]pyridin-6-amine (16.54 mg, 0.112 mmol) in THF (4 mL) was added LHMDS (0.343 mL, 0.343 mmol). The resulting reaction mixture was stirred at 25° C. for 1 h. The reaction mixture was diluted with 80 mL of water and extracted with ethyl acetate (2×90 mL). The combined organic layer was washed with brine, dried with anhydrous sodium sulphate, filtered, and concentrated under reduced pressure. The crude compound was purified by prep HPLC purification. Prep HPLC condition: Diluent: water:THF:ACN (30:30:40), Column: X-Bridge C18 (150×19) mm, 5 micron, Temperature: Ambient, Mobile phase A: 5 mM Ammonium formate, Mobile phase B: acetonitrile, Flow: 15 mL/min, Time/Grad: 0/30, 7/70. The pure fractions were collected and lyophilized to afford 7′-(4-hydroxybicyclo[2.2.1]heptan-1-yl)-2′-((7-methyl-[1,2,4]triazolo[1,5-a]pyridin-6yl)amino)spiro[cyclopropane-1,5′-pyrrolo[2,3-d]pyrimidin]-6′(7′H)-one (4.5 mg, 10.50 μmol, 12.23% yield) as off white solid. LCMS: m/z 418.0 [M+H]⁺, RT=1.32 min (LCMS method G). HPLC (purity)=97.43%, RT=5.22 min (HPLC Method B). ¹H NMR (400 MHz, DMSO-d₆): δ 9.02 (s, 1H), 8.81 (s, 1H), 8.39 (s, 1H), 7.85 (s, 1H), 7.72 (s, 1H), 4.94 (s, 1H), 2.33-2.49 (m, 5H), 2.26 (s, 2H), 1.75-1.82 (m, 2H), 1.58-1.63 (m, 4H), 1.45-1.47 (m, 4H).

The following Examples were prepared in a methodology analogous to the Example 66 with suitable reagents, precursors and starting materials.

### Example 67. 7′-(4-hydroxybicyclo[2.2.1]heptan-1-yl)-2′-((7-methylquinoxalin-6-yl)amino)spiro[cyclopropane-1,5′-pyrrolo[2,3-d]pyrimidin]-6′(7′H)-one (Compound 67)

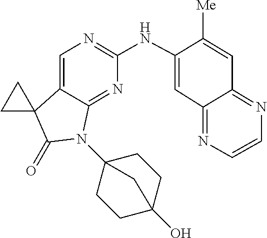 [SMILES: Cc1cc2nccnc2cc1Nc1ncc2c(n1)N(C13CCC(O)(CC1)C3)C(=O)C21CC1]

LC-MS: m/z: 429.3 [M+H]⁺; RT=1.70 min (LCMS method F). HPLC purity: 93.03%, RT=3.54 min (HPLC Method B). ¹H NMR (400 MHz, DMSO-d6): δ 8.81-8.83 (m, 2H), 8.76 (d, J=1.82 Hz, 1H), 8.50 (s, 1H), 7.98 (s, 1H), 7.93 (s, 1H), 2.55-2.62 (m, 6H), 2.31 (s, 2H), 1.84-1.90 (m, 2H), 1.65-1.71 (m, 4H), 1.47-1.52 (m, 4H).

### Example 68. 7′-(4-hydroxybicyclo[2.2.1]heptan-1-yl)-2′-((6-methylbenzo[d][1,3]dioxol-5-yl)amino)spiro[cyclopropane-1,5′-pyrrolo[2,3-d]pyrimidin]-6′(7′H)-one (Compound 68)

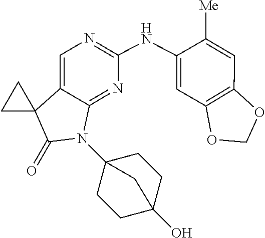 [SMILES: Cc1cc2c(cc1Nc1ncc3c(n1)N(C14CCC(O)(CC1)C4)C(=O)C31CC1)OCO2]

LC-MS: m/z: 421.1 [M+H]⁺; RT=1.86 min (LCMS method F). HPLC purity: 99.38%, RT=5.01 min (HPLC Method A). ¹H NMR (400 MHz, DMSO-d6): δ 8.46 (s, 1H), 7.76 (s, 1H), 6.93 (s, 1H), 6.79 (s, 1H), 5.96 (s, 2H), 4.95 (s, 1H), 2.42-2.45 (m, 2H), 2.25-2.30 (m, 2H), 2.11 (s, 3H), 1.78-1.84 (m, 2H), 1.62-1.69 (m, 2H), 1.54-1.57 (m, 2H), 1.48-1.51 (m, 2H), 1.10-1.30 (m, 2H).

### Example 69. 7′-(4-hydroxybicyclo[2.2.1]heptan-1-yl)-2′-((6-methylbenzo[c][1,2,5]thiadiazol-5-yl)amino)spiro[cyclopropane-1,5′-pyrrolo[2,3-d]pyrimidin]-6′(7′H)-one (Compound 69)

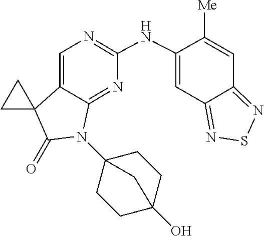 [SMILES: Cc1cc2nsnc2cc1Nc1ncc2c(n1)N(C13CCC(O)(CC1)C3)C(=O)C21CC1]

LC-MS: m/z: 435.0 [M+H]⁺; RT=1.90 min (LCMS method F). HPLC purity: 94.30%, RT=4.87 min (HPLC Method B). ¹H NMR (400 MHz, DMSO-d6): δ 8.71 (s, 1H), 8.454 (s, 1H), 7.99 (s, 1H), 7.93 (s, 1H), 4.96 (s, 1H), 2.53 (s, 3H), 2.30-2.32 (m, 2H), 1.86-1.92 (m, 2H), 1.66-1.71 (m, 4H), 1.50-1.52 (m, 4H). One CH2 group (two protons) is merged with the solvent peak.

### Example 70. 7′-(3-fluorobicyclo[1.1.1]pentan-1-yl)-2′-((7-methyl-[1,2,4]triazolo[1,5-a]pyridin-6-yl)amino)spiro[cyclopropane-1,5′-pyrrolo[2,3-d]pyrimidin]-6′(7′H)-one (Compound 70)

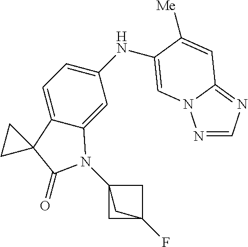 [SMILES: Cc1cc2ncnn2cc1Nc1ccc2c(c1)N(C13CC(F)(C1)C3)C(=O)C21CC1]

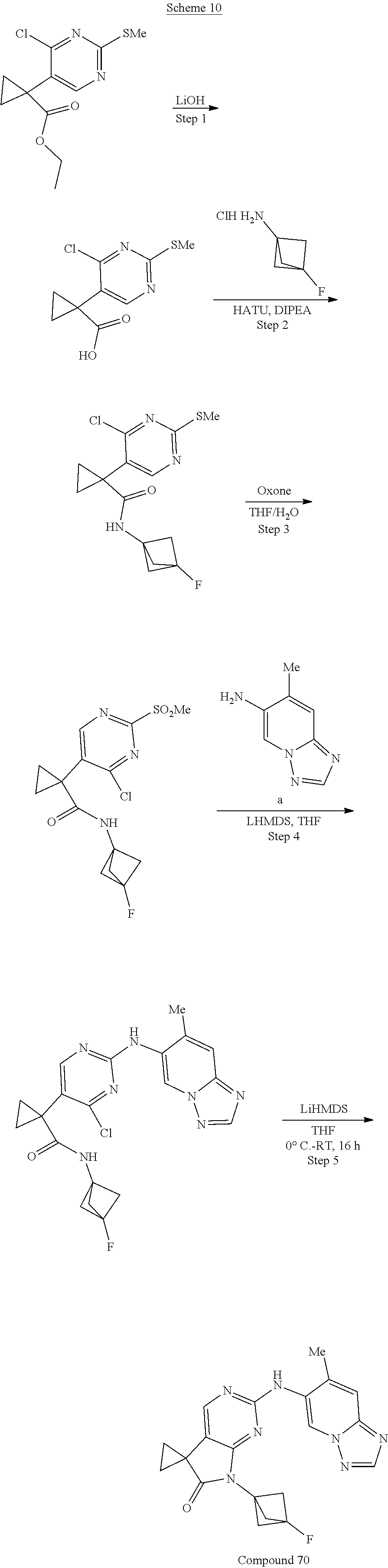

### Step 1. 1-(4-Chloro-2-(methylthio)pyrimidin-5-yl)cyclopropane-1-carboxylic acid

To a stirred solution of ethyl 1-(4-chloro-2-(methylthio)pyrimidin-5-yl)cyclopropane-1-carboxylate (2.0 g, 7.33 mmol) in THF (20 mL) and H₂O (20 mL) were added LiOH (0.878 g, 36.7 mmol) at ambient temperature. Then, it was stirred at ambient temperature for 6 h. The reaction was monitored by LCMS. The reaction mixture was diluted with water (50 mL) and acidified with 1.5N HCl solution (pH ˜6). The crude product was extracted with ethyl acetate (2×100 mL). The combined organic layer was washed with brine, dried with anhydrous sodium sulfate and concentrated under reduced pressure. The reaction mixture was purified by RP-column purification in 0.1 ammonium formate in acetonitrile to 1-(4-chloro-2-(methylthio)pyrimidin-5-yl)cyclopropane-1-carboxylic acid (1.55 g, 5.89 mmol, 80% yield) as an off-white solid. LCMS m/z 245.0 [M−H]⁺, RT=1.00 min (UHPLC Method D).

### Step 2. 1-(4-chloro-2-(methylthio)pyrimidin-5-yl)-N-(3-fluorobicyclo[1.1.1]pentan-1-yl)cyclopropane-1-carboxamide

To a stirred solution of 1-(4-chloro-2-(methylthio)pyrimidin-5-yl)cyclopropane-1-carboxylic acid (1500 mg, 6.13 mmol) in DMF (15 mL) were added DIPEA (3.21 mL, 18.39 mmol) and 3-fluorobicyclo[1.1.1]pentan-1-amine, HCl (1265 mg, 9.20 mmol) followed by N,N,N′,N′-Tetramethylchloroformamidinium hexafluorophosphate (3440 mg, 12.26 mmol) at ambient temperature. It was then stirred at 0-25° C. for 6 hr. The reaction was monitored by LCMS. The reaction mixture was diluted with water (70 mL). The crude product was extracted with ethyl acetate (2×110 mL). The combined organic layer was washed with brine, dried with anhydrous sodium sulphate and concentrated under reduced pressure. The reaction mixture was purified by RP-column purification in 0.1 ammonium formate in acetonitrile to afford 1-(4-chloro-2-(methylthio)pyrimidin-5-yl)-N-(3-fluorobicyclo[1.1.1]pentan-1-yl)cyclopropane-1-carboxamide (700 mg, 2.022 mmol, 33.0% yield) as an off-white solid. LCMS m/z 329.2 [M−H]⁺; RT=2.82 min (LCMS method G).

### Step 3. 1-(4-chloro-2-(methylsulfonyl)pyrimidin-5-yl)-N-(3-fluorobicyclo[1.1.1]pentan-1-yl)cyclopropane-1-carboxamide

To a stirred solution of 1-(4-chloro-2-(methylthio)pyrimidin-5-yl)-N-(3-fluorobicyclo[1.1.1]pentan-1-yl)cyclopropane-1-carboxamide (140 mg, 0.427 mmol) in THF (9 mL) and H₂O (9 mL) were added oxone (788 mg, 1.281 mmol) at 0° C. The resultant reaction mixture was heated at 0-25° C. for 16 h. The reaction was monitored by LCMS. The reaction mixture was diluted with sodium meta sulfate (8 mL) and water (60 mL). The crude product was extracted with ethyl acetate (2×80 mL). The combined organic layer was washed with brine, dried with anhydrous sodium sulphate and concentrated under reduced pressure. The concentrated product was purified by RP-column purification in (0.1% ammonium acetate:ACN) to obtain 1-(4-chloro-2-(methylsulfonyl)pyrimidin-5-yl)-N-(3-fluorobicyclo[1.1.1]pentan-1-yl)cyclopropane-1-carboxamide (140 mg, 0.370 mmol, 87% yield) as a off white solid. LCMS m/z 360.0 [M+H]⁺; RT=2.23-2.45 min (LCMS method G).

### Step 4. 1-(4-chloro-2-((7-methyl-[1,2,4]triazolo[1,5-a]pyridin-6-yl)amino)pyrimidin-5-yl)-N-(3-fluorobicyclo[1.1.1]pentan-1-yl)cyclopropane-1-carboxamide

To a stirred solution of 1-(4-chloro-2-(methylsulfonyl)pyrimidin-5-yl)-N-(3-fluorobicyclo[1.1.1]pentan-1-yl)cyclopropane-1-carboxamide (100 mg, 0.278 mmol) and 7-methyl-[1,2,4]triazolo[1,5-a]pyridin-6-amine (41.2 mg, 0.278 mmol) in THF (6 mL) were added LHMDS (1.112 mL, 1.112 mmol) at 0° C. The resultant reaction mixture was heated at 0-25° C. for 1 h. The reaction mixture was diluted with water (20 mL). The crude product was extracted with ethyl acetate (2×50 mL), The combined organic layer was washed with brine, dried with anhydrous sodium sulphate and concentrated under reduced pressure. The concentrated product was purified by trituration with EtOAc:pet ether (5:95) to obtain 1-(4-chloro-2-((7-methyl-[1,2,4]triazolo[1,5-a]pyridin-6-yl)amino)pyrimidin-5-yl)-N-(3-fluorobicyclo[1.1.1]pentan-1-yl)cyclopropane-1-carboxamide (100 mg, 0.140 mmol, 50.5% yield) as a brownish gum.

LC-MS: m/z: 428.2 [M+H]⁺, RT=1.06 min (UHPLC Method D).

### Step 5. 7′-(3-fluorobicyclo[1.1.1]pentan-1-yl)-2′-((7-methyl-[1,2,4]triazolo[1,5-a]pyridin-6-yl)amino)spiro[cyclopropane-1,5′-pyrrolo[2,3-d]pyrimidin]-6′(7′H)-one

To a stirred solution of 1-(4-chloro-2-((7-methyl-[1,2,4]triazolo[1,5-a]pyridin-6-yl)amino)pyrimidin-5-yl)-N-(3-fluorobicyclo[1.1.1]pentan-1-yl)cyclopropane-1-carboxamide (100 mg, 0.234 mmol) in THF (6 mL) were added LHMDS (0.935 mL, 0.935 mmol) at 0° C. The resultant reaction mixture was heated at 0-25° C. for 1 h. The reaction mixture was diluted with water (20 mL). The crude product was extracted with ethyl acetate (2×50 mL). The combined organic layer was washed with brine, dried with anhydrous sodium sulphate and concentrated under reduced pressure. The concentrated product was purified by RP-prep purification in 0.1 ammonium formate in ACN to obtain 7′-(3-fluorobicyclo[1.1.1]pentan-1-yl)-2′-((7-methyl-[1,2,4]triazolo[1,5-a]pyridin-6-yl)amino)spiro[cyclopropane-1,5′-pyrrolo[2,3-d]pyrimidin]-6′(7′H)-one (28 mg, 0.071 mmol, 30.4% yield) as an-off white solid. LC-MS: m/z: 392.0 [M+H]⁺; RT=1.89 min (LCMS method F). HPLC purity: 99.39%, RT=6.05 min (HPLC Method A). ¹H NMR (400 MHz, DMSO-d6): δ 9.07 (s, 1H), 8.52 (s, 1H), 8.40 (s, 1H), 7.94 (s, 1H), 7.74 (s, 1H), 2.62 (d, J=2.40 Hz, 6H), 2.38 (s, 3H), 1.66-1.69 (m, 2H), 1.50-1.53 (m, 2H).

The following Examples were prepared in a methodology analogous to Example 66 with suitable reagents, precursors and starting materials.

### Example 71. 7′-(3-fluorobicyclo[1.1.1]pentan-1-yl)-2′-((7-methylquinolin-6-yl)amino)spiro[cyclopropane-1,5′-pyrrolo[2,3-d]pyrimidin]-6′(7′H)-one (Compound 71)

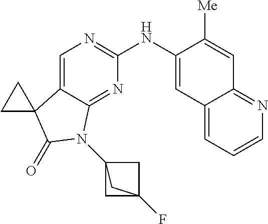 [SMILES: Cc1cc2ncccc2cc1Nc1ncc2c(n1)N(C13CC(F)(C1)C3)C(=O)C21CC1]

LC-MS: m/z: 402.0 [M+H]⁺; RT=1.87 min (LCMS method F). HPLC purity: 97.72%, RT=4.15 min (HPLC Method B). ¹H NMR (400 MHz, DMSO-d6): δ 8.95 (s, 1H), 8.78 (dd, J=1.7, 4.2 Hz, 1H), 8.23 (d, J=8.4 Hz, 1H), 8.16 (s, 1H), 7.99 (s, 1H), 7.89 (s, 1H), 7.45 (dd, J=4.3, 8.3 Hz, 1H), 2.64 (d, J=2.4 Hz, 6H), 1.71-1.68 (m, 2H), 1.54-1.51 (m, 2H).

### Example 72. 7′-(3-fluorobicyclo[1.1.1]pentan-1-yl)-2′-((7-methylquinoxalin-6-yl)amino)spiro[cyclopropane-1,5′-pyrrolo[2,3-d]pyrimidin]-6′(7′H)-one (Compound 72)

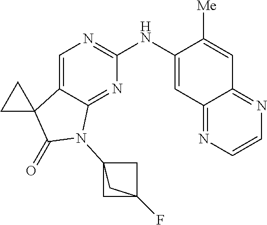 [SMILES: Cc1cc2nccnc2cc1Nc1ncc2c(n1)N(C13CC(F)(C1)C3)C(=O)C21CC1]

LC-MS: m/z: 403.2 [M+H]⁺; RT=2.81 min (LCMS method G). HPLC purity: 99.35%, RT=6.05 min (HPLC Method B). ¹H NMR (400 MHz, DMSO-d6): δ 8.95 (s, 1H), 8.82 (d, J=1.6 Hz, 1H), 8.77 (d, J=2.00 Hz, 1H), 8.52 (s, 1H), 8.06 (s, 1H), 7.94 (d, J=0.80 Hz, 1H), 2.71 (d, J=2.40 Hz, 4H), 2.57 (s, 3H), 1.72-1.74 (m, 2H), 1.55-1.57 (m, 2H).

### Example 73. 7′-(3-fluorobicyclo[1.1.1]pentan-1-yl)-2′-((6-methylbenzo[c][1,2,5]thiadiazol-5-yl)amino)spiro[cyclopropane-1,5′-pyrrolo[2,3-d]pyrimidin]-6′(7′H)-one (Compound 73)

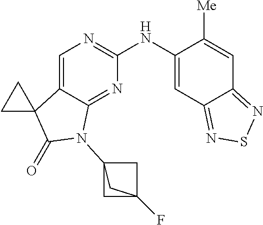 [SMILES: Cc1cc2nsnc2cc1Nc1ncc2c(n1)N(C13CC(F)(C1)C3)C(=O)C21CC1]

LC-MS: m/z: 409.0 [M+H]⁺; RT=3.29 min (conditions: Column: Kinetex XB—C18 (75×3) mm, 2.6 μm, Mobile Phase: A: 5 mm Ammonium formate pH 3.3:ACN (98:02), Mobile Phase: B: ACN:Buffer (98:02), Flow Rate: 1.0 ml/min, Time/Grad (% B): 0/40, 3.5/100, 3.51/100 (1.5 ml/min), 4.5/100 (1.5 ml/min), 4.7/40, 5/40). HPLC purity: 99.10%, RT=7.91 min (HPLC Method B). ¹H NMR (400 MHz, DMSO-d6): δ 8.84 (s, 1H), 8.48 (s, 1H), 8.07 (s, 1H), 7.94 (s, 1H), 2.73 (d, J=2.00 Hz, 6H), 2.54 (d, J=0.80 Hz, 3H), 1.73-1.76 (m, 2H), 1.56-1.58 (m, 2H).

### Example 74. 7′-(3-fluorobicyclo[1.1.1]pentan-1-yl)-2′-((4-methyl-6-(oxazol-5-yl)pyridin-3-yl)amino)spiro[cyclopropane-1,5′-pyrrolo[2,3-d]pyrimidin]-6′(7′H)-one (Compound 74)

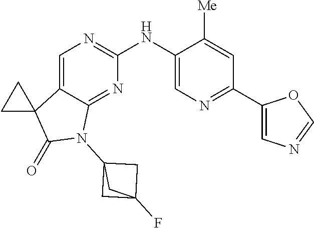 [SMILES: Cc1cc(-c2cnco2)ncc1Nc1ncc2c(n1)N(C13CC(F)(C1)C3)C(=O)C21CC1]

LC-MS: m/z: 419.0 [M+H]⁺; RT=2.46 min (LCMS method G). HPLC purity: 99.39%, RT=4.76 min (HPLC Method B). ¹H NMR (400 MHz, DMSO-d6): δ 8.84 (s, 1H), 8.48 (s, 1H), 8.07 (s, 1H), 7.94 (s, 1H), 2.73 (d, J=2.00 Hz, 6H), 2.54 (d, J=0.80 Hz, 3H), 1.73-1.76 (m, 2H), 1.56-1.58 (m, 2H).

### Example 75. 7′-(3-fluorobicyclo[1.1.1]pentan-1-yl)-2′-((4-methyl-6-(1-methyl-1H-pyrazol-4-yl)pyridin-3-yl)amino)spiro[cyclopropane-1,5′-pyrrolo[2,3-d]pyrimidin]-6′(7′H)-one (Compound 75)

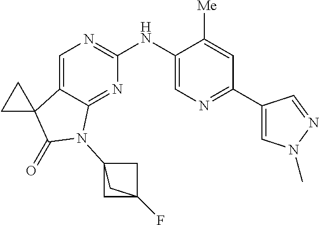 [SMILES: Cc1cc(-c2cnn(C)c2)ncc1Nc1ncc2c(n1)N(C13CC(F)(C1)C3)C(=O)C21CC1]

LC-MS: m/z: 432.0 [M+H]⁺; RT=2.14 min (LCMS method G). HPLC purity: 98.67%, RT=4.60 min (HPLC Method B). ¹H NMR (400 MHz, DMSO-d6): δ 8.89 (s, 1H), 8.48 (s, 1H), 8.22 (s, 1H), 7.95 (d, J=0.80 Hz, 1H), 7.89 (s, 1H), 7.56 (s, 1H), 3.89 (s, 3H), 2.63 (d, J=2.00 Hz, 6H), 2.25 (s, 3H), 1.65-1.68 (m, 2H), 1.49-1.52 (m, 2H).

### Example 76. 7′-(3-fluorobicyclo[1.1.1]pentan-1-yl)-2′-((6-methylbenzo[d][1,3]dioxol-5-yl)amino)spiro[cyclopropane-1,5′-pyrrolo[2,3-d]pyrimidin]-6′(7′H)-one (Compound 76)

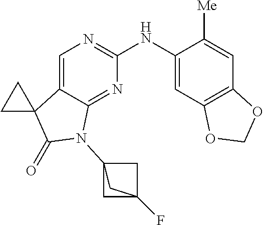 [SMILES: Cc1cc2c(cc1Nc1ncc3c(n1)N(C14CC(F)(C1)C4)C(=O)C31CC1)OCO2]

LC-MS: m/z: 395.0 [M+H]⁺; RT=3.02 min (LCMS method G). HPLC purity: 98.77%, RT=5.76 min (HPLC Method B). ¹H NMR (400 MHz, DMSO-d6): δ 8.59 (s, 1H), 7.84 (s, 1H), 6.96 (s, 1H), 6.82 (s, 1H), 5.97 (s, 2H), 2.63 (d, J=2.00 Hz, 6H), 2.12 (s, 3H), 1.62-1.65 (m, 2H), 1.47-1.50 (m, 2H).

### Example 77

### 9-(4-hydroxybicyclo[2.2.1]heptan-1-yl)-7-(methyl-d3)-2-((7-methyl-[1,2,4]triazolo[1,5-a]pyridin-6-yl)amino)-7,9-dihydro-8H-purin-8-one (Compound 77)

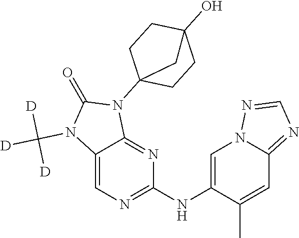 [SMILES: [2H]C([2H])([2H])n1c(=O)n(C23CCC(O)(CC2)C3)c2nc(Nc3cn4ncnc4cc3C)ncc21]

Example 77 was prepared in a synthetic sequence analogous to Example 1 using CD₃I instead of CH₃I in the alkylation step. LC-MS: m/z: 410.1 [M+H]⁺. HPLC purity: 100%, RT=1.08 min (LCMS Method A). ¹H NMR (400 MHz, DMSO-d6): δ 9.10 (s, 1H), 8.52 (s, 1H), 8.37 (s, 1H), 7.70 (s, 1H), 5.06 (s, 1H), 2.39 (m, 5H), 2.31 (s, 2H), 1.99-1.89 (m, 2H), 1.70 (br, t, J=8.6 Hz, 2H), 1.61-1.49 (m, 2H).

### Example 78

### 9-(4-hydroxybicyclo[2.2.2]octan-1-yl)-7-methyl-2-((7-methyl-[1,2,4]triazolo[1,5-a]pyridin-6-yl)amino)-7,9-dihydro-8H-purin-8-one (Compound 78)

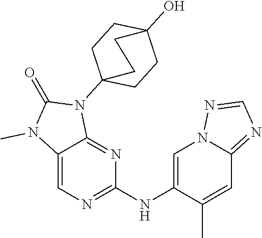 [SMILES: Cc1cc2ncnn2cc1Nc1ncc2c(n1)n(C13CCC(O)(CC1)CC3)c(=O)n2C]

(9-(4-hydroxybicyclo[2.2.2]octan-1-yl)-7-methyl-2-((7-methyl-[1,2,4]triazolo[1,5-a]pyridin-6-yl)amino)-7,9-dihydro-8H-purin-8-one (Compound 78) previously disclosed in PCT publication No.: WO2022017368A1.

### Biological Examples and Activities

Various compounds of the instant disclosure including a control DNA-PK inhibitor herein referenced as Compound 78 (see structure below) were evaluated in DNA-PK biochemical assays and one or more of the kinase counter-screen assays shown above, for example, ATM, PI3Kα, PI3Kβ, and PI3Kδ biochemical Assays.

### DNA-PK Biochemical Assay Using Reaction Biology's HotSpot Kinase Assay Protocol:

Reagent: Base Reaction buffer; 20 mM Hepes (pH 7.5), 10 mM MgCl₂, 1 mM EGTA, 0.01% Brij35, 0.02 mg/ml BSA, 0.1 mM Na₃VO₄, 2 mM DTT, 1% DMSO Required cofactors are added individually to each kinase reaction.

Reaction Procedure: 1. Prepare substrate in freshly prepared Reaction Buffer; 2. Deliver any required cofactors to the substrate solution above; 3. Deliver kinase into the substrate solution and gently mix; 4. Deliver compounds in 100% DMSO into the kinase reaction mixture by Acoustic technology (Echo550; nanoliter range), incubate for 20 min at room temperature; 5. Deliver 33P-ATP into the reaction mixture to initiate the reaction; 6. Incubate for 2 hours at room temperature; 7. Detect kinase activity by P81 filter-binding method.

### Ataxia-Telangiectasia Mutated (ATM) Biochemical Assay Using Reaction Biology's HTRF Kinase Assay Protocol:

### Assay Format:

The assay detects kinase activity by antibody based HTRF assay. The GST-tagged p53 protein serves as substrate and Eu-labeled-antibody with specific phosphorylation site binds upon phosphorylation which is monitored by HTRF from the anti-GST-d2.

Reagent: Reaction buffer; 50 mM Hepes (pH 7.5), 150 mM NaCl, 10 mM MnCl2, 1% Glycerol, 0.01% Brij35, 1 mM DTT, 1% DMSO.

Substrate: Human p53 (aa2-393), RBC produced in E. coli, N-terminal GST-Tag, MW=70.42 kDa.

Detection; MAb Anti-p53-pS15-Eu cryptate, CisBio cat #61P08KAZ; MAb Anti GST-d2

Reaction Procedure: 1. Deliver 2× Enzyme in wells of reaction plate except No E control wells. Add buffer instead; 2. Deliver compounds in 100% DMSO into the kinase reaction mixture by Acoustic technology (Echo550; nanoliter range), incubate for 30 min at room temperature; 3. Deliver 2× substrate+ATP mixture into the reaction mixture to initiate the reaction; 4. Incubate for 2 hours at room temperature.

HTRF Detection: 1. Deliver 2× Stop/Detection mixture and incubate for 2 hours to overnight in the dark. 2. HTRF measurement (Ex/Em1/Em2=320/615/665 nm) in EnVision.

Phosphoinositide 3-kinase R (PI3KP) Biochemical Assay using Reaction Biology's ADP-Glo Assay Platform:

Step 1: Lipid kinases: Reaction Biology Corporation (RBC) currently offers lipid kinases in ADP-Glo format

Step 2: Assay Description Assay principle: The kinase reactions utilize ATP and produce ADP as a byproduct. The ADP production is quantified by ADP-Glo luminescence detection. This is a 3-step reaction: First, the kinase reaction with lipid substrate is carried out in the presence of ATP, and the reaction is quenched and depleted remaining ATP with ADP-Glo™ reagent, and then finally ADP is converted to ATP which is measured using a luciferase/luciferin reaction.

Step 3: Assay Procedure: 1. Prepare kinase in freshly prepared Reaction Buffer, kinase without substrate is delivered to background wells; 2. Deliver substrate into the kinase solution and gently mix, deliver to assay wells; 3. Deliver compounds in 100% DMSO into the kinase reaction mixture by Acoustic technology (Echo550; nanoliter range), incubate for 20 min at room temperature; 4. Deliver ATP into the reaction mixture to initiate the reaction; 5. Incubate for 60 min at 30° C.; 6. Quench the reaction with ADP-Glo reagent and incubate for 40 min; 7. Add Detection Mixture and incubate for 30 min; 8. Measure luminescence.

Step 4: Data Analysis: The luminescence is converted into M ADP production based on ADP standard curves. The nonlinear regression to obtain the standard curve and IC₅₀ values are performed using Graphpad Prism software.

Phosphoinositide 3-kinase α (PI3Kα) Biochemical Assay: A solution was prepared containing 0.2 nM Anti-HIS-Terbium (Cisbio, 64CUSTAZU), 3.7 nM designed-probe and 1.0 nM His-PIK3CA/PIK3R1 (p110 alpha/p85 alpha) in FRET Buffer (20 mM HEPES, 10 mM MgCl2, 0.015% Brij-35, 4 mM DTT, 0.05 mg/mL BSA). Using Formulatrix Tempest for liquid handling, the detection antibody/enzyme/probe solution (2 uL per well) was dispensed into wells of a 1536 plate (Black Low Binding Polystyrene 1536 Plate (Corning, 3724) containing 10 nL of compounds of interest at appropriate concentration in DMSO. The plate was incubated at room temperature for 1 h. FRET was measured using the EnVision plate reader (Excitation: 340 nM, Emission: 520 nM/495 nM). Total signal (0% inhibition) was calculated from wells containing 10 nL DMSO only. Blank signal (100% inhibition) calculated from wells containing 10 nL of 15 nM staurosporine and internal controls.

Phosphoinositide 3-kinase (PI3K) Biochemical Assay: A solution was prepared containing 0.2 nM Anti-HIS-Terbium (Cisbio, 64CUSTAZU), 40 nM designed-probe and 1.0 nM His-PIK3CD/PIK3R1 (p110 delta/p85 alpha) in FRET Buffer (20 mM HEPES, 10 mM MgCl2, 0.015% Brij-35, 4 mM DTT, 0.05 mg/mL BSA). Using Formulatrix Tempest for liquid handling, the detection antibody/enzyme/probe solution (2 uL per well) was dispensed into wells of a 1536 plate (Black Low Binding Polystyrene 1536 Plate (Corning, 3724) containing 10 nL of compounds of interest at appropriate concentration in DMSO. The plate was incubated at room temperature for 1 h. FRET was measured using the EnVision plate reader (Excitation: 340 nM, Emission: 520 nM/495 nM). Total signal (0% inhibition) was calculated from wells containing 10 nL DMSO only. Blank signal (100% inhibition) calculated from wells containing 10 nL of 15 nM staurosporine and internal controls.

### Biological Data

### Table 3. Biological Data

### Legend:

| Legend for Table 3 | Biochemical Assay IC₅₀ (nM) |
|---|---|
| A | IC₅₀ < 1 nM |
| B | 1 nM ≤ IC₅₀ < 10 nM |
| C | 10 nM ≤ IC₅₀ < 100 nM |
| D | 100 nM ≤ IC₅₀ < 1 μM |
| E | IC₅₀ ≥ 1 μM |

| | DNA-PK | ATM | PI3Kβ | | |
|---|---|---|---|---|---|
| Com- | HotSpot 10 | HTRF 10 | ADP | PI3Kδ | |
| pound | μM ATP | μM ATP | Glo | HTRF | PI3KαHTRF |
| Number | IC₅₀ (nM) | IC₅₀ (nM) | IC₅₀ (nM) | IC₅₀ (nM) | IC₅₀ (nM) |
| 1 | A, 0.07 | C, 88 | D, 658 | E, 1828 | D, 128 |
| 2 | A | D | E | E | E |
| 3 | A | B | | E | C |
| 4 | A | C | | D | D |
| 5 | A | C | | E | D |
| 6 | A | C | D | E | D |
| 7 | A | D | E | E | D |
| 8 | A | C | | D | C |
| 9 | A | C | | D | C |
| 10 | A | D | | E | C |
| 11 | A | C | D | E | D |
| 12 | A | D | E | E | E |
| 13 | B | E | E | E | E |
| 14 | A | D | | E | D |
| 15 | B | D | | E | D |
| 16 | C | D | | E | E |
| 17 | B | D | | E | E |
| 18 | B | B | | E | E |
| 19 | B | D | | E | E |
| 20 | B | B | | E | D |
| 21 | B | C | | E | E |
| 22 | B | B | | E | D |
| 23 | B | C | | E | D |
| 24 | A | C | | E | D |
| 25 | A | B | | D | D |
| 26 | A | C | | E | D |
| 27 | A | C | | E | C |
| 28 | B | E | | E | |
| 29 | B | D | | E | E |
| 30 | A | D | | E | D |
| 31 | B | D | | C | D |
| 32 | B | E | | D | E |
| 33 | B | D | | C | D |
| 34 | C | E | | D | E |
| 35 | D | E | | E | E |
| 36 | A | A | | E | D |
| 37 | A | B | | E | D |
| 38 | C | C | | E | |
| 39 | C | C | | E | D |
| 40 | B | D | | E | D |
| 41 | B | B | | E | D |
| 42 | A | C | D | E | D |
| 43 | A | D | E | E | E |
| 44 | A | B | D | D | C |
| 45 | A | C | D | E | D |
| 46 | A | D | E | E | E |
| 47 | B | D | | E | E |
| 48 | B | D | | E | E |
| 49 | A | C | | D | D |
| 50 | A | C | | E | E |
| 51 | B | D | | D | D |
| 52 | C | E | | E | E |
| 53 | A | C | | C | |
| 54 | B | E | | E | E |
| 55 | A | B | | D | C |
| 56 | C | E | | E | E |
| 57 | B | C | | D | D |
| 58 | A | D | | | E |
| 59 | A | C | | D | D |
| 60 | A | C | | D | D |
| 61 | A | B | | D | C |
| 62 | A | C | | E | E |
| 63 | B | D | | D | E |
| 64 | A | B | | D | C |
| 65 | A | B | | D | C |
| 66 | A | D | | D | C |
| 67 | A | D | | D | C |
| 68 | A | E | | D | D |
| 69 | A | C | | C | C |
| 70 | A | D | | E | E |
| 71 | A | D | | E | D |
| 72 | A | D | | E | D |
| 73 | A | E | | E | D |
| 74 | B | C | | E | D |
| 75 | B | C | | E | D |
| 76 | B | E | | E | E |
| 77 | A | C | | D | D |
| 78 | A, 0.71 | C, 36 | | D, 214 | D, 152 |

T Cell Cytotoxicity Assay—CD3 Glo Proliferation Assay: Frozen vials of CD3 T cells were thawed in assay media (RPMI 1640, 5% HI FBS, 1× L/G, 1×NEAA, 1× Sodium Pyruvate, 1×P/S (Gibco, Waltham MA), and viable cells counted using the Moxi V cell counter (Orflo, Ketchum ID). Thawed T cells were collected by centrifugation at 1600 rpm for 10 mins at room temperature. T cell pellets were resuspended in 10 ml of assay media in 50 ml conical tube and allowed to rest in 37° C. incubator for 1 hour. T cell suspension was removed from 37° C. incubator and diluted to 5e5 cells per ml. T cells were then stimulated using an anti-CD3/anti-CD28 stimulatory reagent prepared using an oligomeric streptavidin mutein reagent produced as described in WO 2018/197949 (see also Postoak et al., Scientific Reports (2020)) at 4 μg per 1 million T cells for 24 hours at 37° C. The next day, compound plates were prepared by ten, 3-fold serial dilutions in 100% DMSO with top concentration at 3 mM. All compound dilutions were done on Echo-qualified 384w plates (Beckman, Indianapolis IN) before 200 nanoliters were acoustically transferred to Corning 384w tissue culture treated plates (Catalog #353988, Corning, Tewksbury MA) using an ECHO 650 acoustical liquid handler (Beckman, Indianapolis IN). To neutralize anti-CD3/anti-CD28 stimulatory reagent-mediated T cell activation, 50 mM D-Biotin was added to anti-CD3/anti-CD28 stimulatory reagent-activated T cell cultures at 1:50 dilution and incubated for 10 minutes at 37° C. Forty microliters of neutralized T cell culture or assay media control was dispensed per well on coming assay plates pre-printed with 200 nanoliters of compound in DMSO using a multidrop liquid handler (ThermoFisher, Waltham MA). Plates were incubated for 3 days at which point 10 ul per well of Celltiter Glo reagent (Promega, Madison WI) was added, incubated at room temperature for 10 minutes then read on an Envision plate reader (Perkin-Elmer, Waltham MA). The concentration where 50% reduction in Celltiter Glo signal from well containing only DMSO (CC₅₀) was calculated using a four-parameter logistic equation.

### T Cell Media and Thaw

CD4+ and CD8+ T cells were isolated from healthy donor T cells and combined at a 1:1 ratio of CD4 to CD8 T cells in a serum free T cell media (TCM) containing recombinant cytokines as follows: 100 IU/mL IL-2, 1500 IU/mL IL-7, 19 IU/mL IL-15.

### T Cell Activation

For T cell stimulation, an anti-CD3/anti-CD28 stimulatory reagent was prepared using an oligomeric streptavidin mutein reagent produced as described in WO 2018/197949 (see also Poltorak et al., Scientific Reports (2020)). The oligomeric streptavidin mutein reagent had an average hydrodynamic radius of 90-120 nm and contained an average of 2000-2800 tetramers of a streptavidin mutein (Strep-Tactin® m2, SEQ ID NO: 6). The oligomeric streptavidin mutein reagent was mixed at room temperature with (i) an anti-CD3 Fab fragment individually fused at the carboxy-terminus of its heavy chain to a streptavidin-binding peptide sequence (Twin-Strep-tag®, SEQ ID NO: 16) and (ii) an anti-CD28 Fab fragment also individually fused at the carboxy-terminus of its heavy chain to a streptavidin-binding peptide sequence (Twin-Strep-tag®, SEQ ID NO: 16). The peptide-tagged Fab fragments were recombinantly produced (see International Patent App. Pub. Nos. WO 2013/011011 and WO 2013/124474). The anti-CD3 Fab fragment was derived from the CD3 binding monoclonal antibody produced by the hybridoma cell line OKT3 (ATCC® CRL-8001™; see also U.S. Pat. No. 4,361,549) and contained the heavy chain variable domain (SEQ ID NO: 31) and light chain variable domain (SEQ ID NO: 32) of the anti-CD3 antibody OKT3 described in Arakawa et al., J. Biochem. 120, 657-662 (1996). The anti-CD28 Fab fragment was derived from antibody CD28.3 (deposited as a synthetic single chain Fv construct under GenBank Accession No. AF451974.1; see also Vanhove et al., BLOOD, 15 Jul. 2003, Vol. 102, No. 2, pages 564-570) and contained the heavy chain variable domain (SEQ ID NO: 33) and the light chain variable domain (SEQ ID NO: 34) of the anti-CD28 antibody CD28.3. To prepare the anti-CD3/anti-CD28 stimulatory reagent, 0.3 mg of oligomeric streptavidin mutein reagent, 0.5 μg of peptide-tagged anti-CD3 Fab fragments, and 0.5 μg of peptide-tagged anti-CD28 Fab fragment was used.

Isolated T cells described above were suspended at a density of about 3×10⁶ cells/mL and the anti-CD3/anti-CD28 stimulatory reagent was added to the cells in a media supplemented with 100 IU/mL IL-2, 1500 IU/mL IL-7, 19 IU/mL IL-15. Cells were cultured in 6 well plates (Corning 351146) and incubated at 37° C. for 48 hours.

### T Cell Engineering

48 hours post-activation, T cells were counted and resuspended in buffer at a density of 5×10⁷ cells/mL.

For introducing a genetic disruption at the endogenous TCRα constant region (TRAC) locus by CRISPR/Cas9-mediated gene editing, ribonucleoprotein (RNP) composed of Cas9 protein (Aldevron) and TRAC-targeted single guide RNA (sgRNA) with targeting domain sequence GAGAAUCAAAAUCGGUGAAU (SEQ ID NO:28; targeting within exon 1 of the endogenous TRAC gene) was added to the resuspended T cells to achieve a final concentration of 2 μM of RNP. The T cell/RNP solution was transferred into electroporation cuvettes, 100 μL/cuvette (Lonza P3 Primary Cell 4D-Nucleofector X Kit L V4XP-3024) and electroporated using a Lonza 4D-Nucleofector X Unit (Lonza) with pulse code DN-100/P3. Immediately following electroporation, 600 μL of TCM was added per electroporation cuvette and cells were rested in cuvettes at 37 C for 15 minutes. Electroporated cells were pooled and transferred into 96 well flat bottom recovery plates (Corning 351172) containing AAV encoding an exemplary homology directed repair template for insertion of an exemplary anti-BCMA CAR into the TRAC locus at a MOI of 5×10³ viral genomes/cell, DNA-PK inhibitors at the indicated concentrations, 1 mM d-biotin, 100 IU/mL IL-2, 1500 IU/mL IL-7, and 19 IU/mL IL-15 in a final volume of 210 μL TCM/well and a final cell density (based on pre-electroporation counts) of 5×10⁵ T cells/well. The anti-BCMA CAR is described in WO2019/090003.

The exemplary anti-BCMA CAR (SEQ ID NO: 198, encoded by SEQ ID NO: 197) included a human IgG-kappa signaling sequence, a human anti-BCMA scFv (Table 4); a modified IgG4-hinge CH2-CH3 (SEQ ID NO:184, encoded by SEQ ID NO:183) spacer (which spacer may in some instances be referred to as “LS”; a human CD28 transmembrane domain (SEQ ID NO: 186, encoded by SEQ ID NO: 185); a human 4-1BB-derived intracellular co-signaling sequence (SEQ ID NO: 188, encoded by SEQ ID NO: 187); and a human CD3-zeta derived intracellular signaling domain (SEQ ID NO: 190, encoded by SEQ ID NO: 189).

The exemplary human anti-BCMA scFv contained an scFv with the following sequences:

TABLE 4
Sequence identifier (SEQ ID NO) for Exemplary scFv

| Antigen- | | | | | | | | | |
|---|---|---|---|---|---|---|---|---|---|
| binding | CDR- | CDR- | CDR- | CDR- | CDR- | CDR- | | | |
| domain | H1 | H2 | H3 | L1 | L2 | L3 | V_{H} | V_{L} | scFv |
| BCMA-55 | 56 | 57 | 58 | 59 | 60 | 61 | 36 | 37 | 180 |

The general structure of the exemplary homology directed repair template polynucleotide was as follows: [5′ homology arm (SEQ ID NO:191)]-[promoter (SEQ ID NO: 187)]-[transgene sequence encoding the anti-BCMA CAR ((SEQ ID NO: 193)]-[3′ homology arm (SEQ ID NO: 192)]. The homology arms included approximately 600 bp of nucleic acid sequences homologous to sequences surrounding the target integration site in exon 1 of the human TCRα constant region (TRAC) gene. The sequence of the entire homology directed repair template polynucleotide that was used is given in SEQ ID NO: 194.

Control samples were engineered as described above with the omission of DNA-PK inhibitor treatment (untreated) or AAV (TRAC KO only) in the corresponding wells of the recovery plate.

### T Cell Expansion

24 hours after electroporation, T cells were transferred into 24 well GREX plates (Wilson Wolf 80192M) in a final volume of 3 mL TCM/well supplemented with 100 IU/mL IL-2, 1500 IU/mL IL-7, and 19 IU/mL IL-15. Cells were expanded in GREX plates for a total of 5 days post-electroporation with cytokine replenishment every 2-3 days (final volume of 4 mL/well at 5 days post-electroporation). At day 5 post-electroporation the cell viability and count were measured using AOPI staining (Nexcelom CS2-0106) and the CellacaMX automated cell counter (Nexcelom Bioscience). CAR knock-in efficiency was measured by flow cytometry as described below.

### Flow Cytometry

Following T cell engineering and 5 days of expansion, cells were characterized for TRAC knock-out and CAR knock-in by flow cytometry. Briefly, 2-5×105 cells/well were transferred to 96 well U-bottom plates (Corning 351177) for staining with LIVE/DEAD Fixable near-IR (ThermoFisher L34993) according to the manufacturers protocol, followed by a cocktail of antibodies targeting CD3 (BioLegend, UCHT1), CD4 (BioLegend, OKT4), CD8 (BD Horizon, RPA-T8), and anti-idiotypic antibody (which binds to the extracellular portion of the exemplary anti-BCMA CAR; see WO2021/113776) diluted in cell staining buffer (BioLegend D5RE-01386-1) for 30 minutes at 4 C. Following staining, cells were washed, resuspended in 100 uL cell staining buffer/well, and analyzed using a FACSymphony A5 cytometer (BD Biosciences) using the high throughput plate reader to collect 20,000 live cells/well. Data analysis was performed using FlowJo 10.8.1 (BD Biosciences) and JMP 15.2.0 (SAS Institute Inc.). Total CAR+ T cells were calculated by multiplying % CAR+ by total cell count, and then normalized to the untreated condition by dividing total CAR+ T cells of DNA PK inhibitor treated conditions by the average of three untreated conditions, subtracting 1, and multiplying by 100 to get the total CAR+ cell yield as a percent change relative to the untreated control.

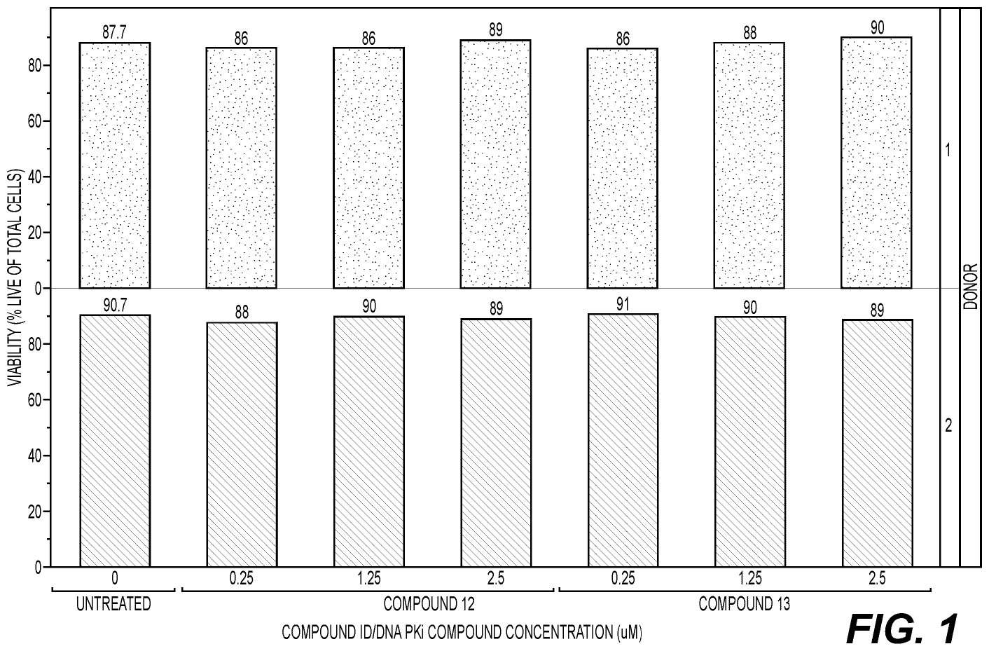

FIG. 1 shows the effect of DNA-PK inhibitor compounds (e.g. Compound 12 and Compound 13) at 0.25 μM, 1.25 μM, and 2.5 μM on cell viability 5 days after electroporation with two donors (donor 1 shown in dark grey bar and donor 2 in light grey bar). Live cells are shown as a percentage of total cells.

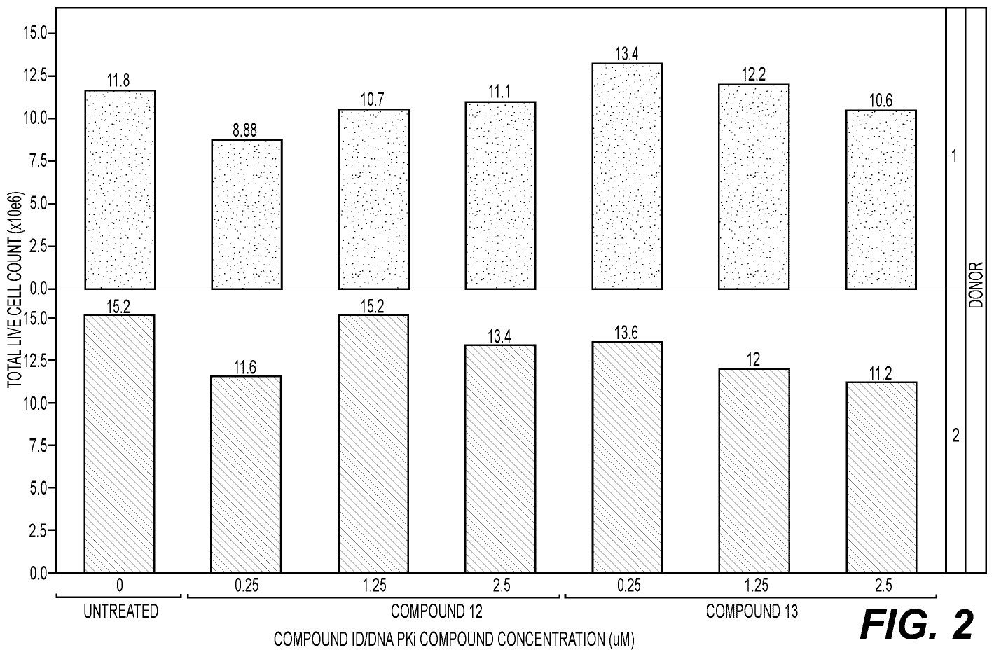

FIG. 2 shows the effect of DNA-PK inhibitor compounds on T cell proliferation 5 days after electroporation. Total live cell counts (×10e6) are shown.

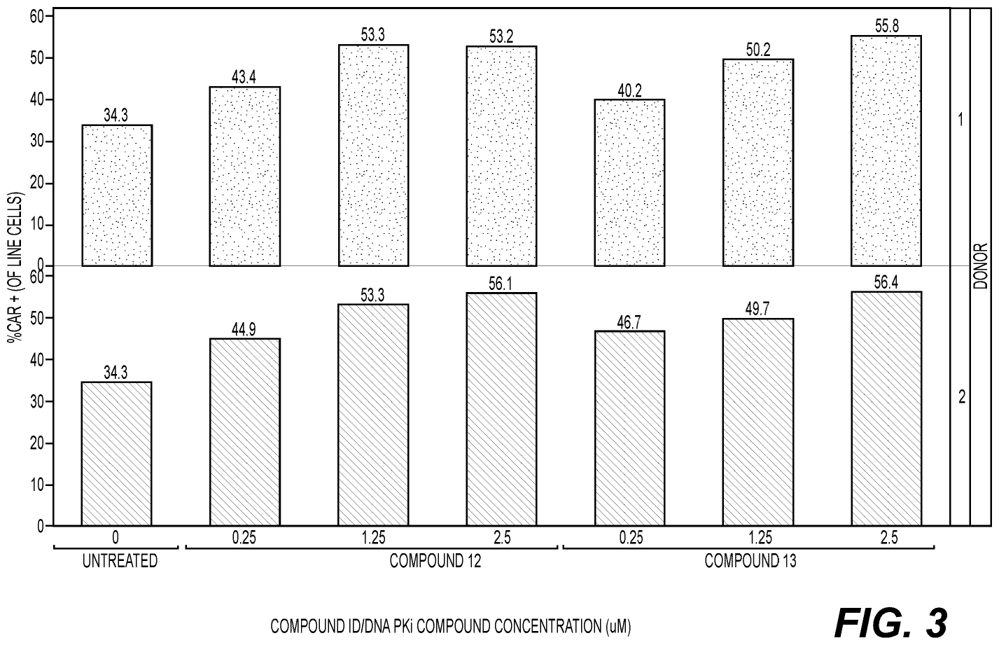

FIG. 3 shows the effect of DNA-PK inhibitor compounds on CAR insertion into the TRAC locus 5 days after electroporation. Frequency of CAR+ T cells is shown as a percentage of total live cells.

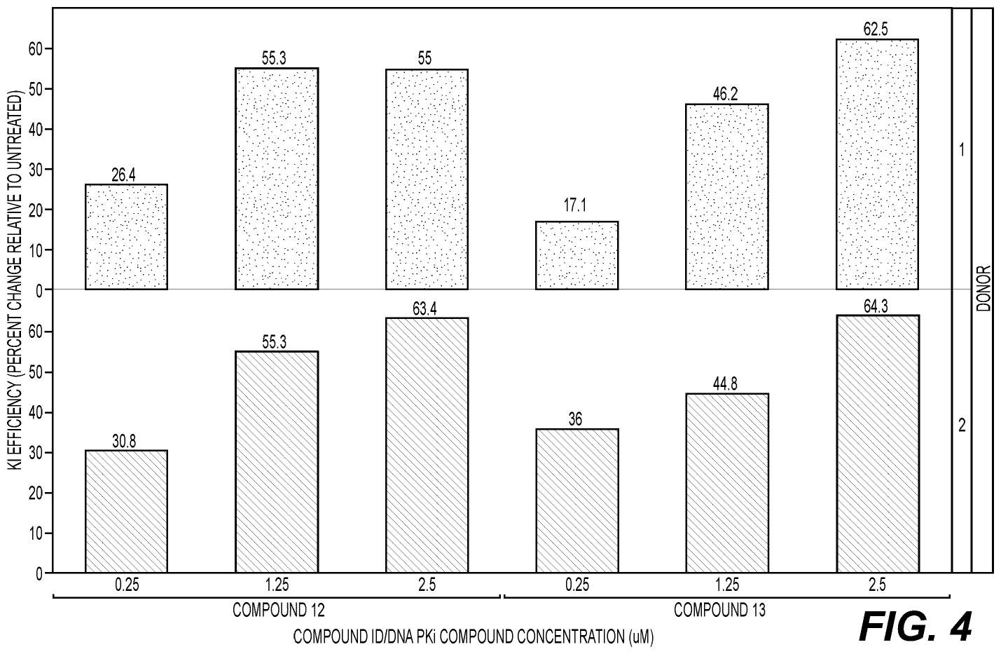

FIG. 4 shows the effect of DNA-PK inhibitor compounds on CAR insertion into the TRAC locus 5 days after electroporation. KI efficiency is shown as percentage change over the untreated control condition (calculated by dividing the % CAR+ of DNA-PKi treated by untreated, subtracting 1, and multiplying by 100).

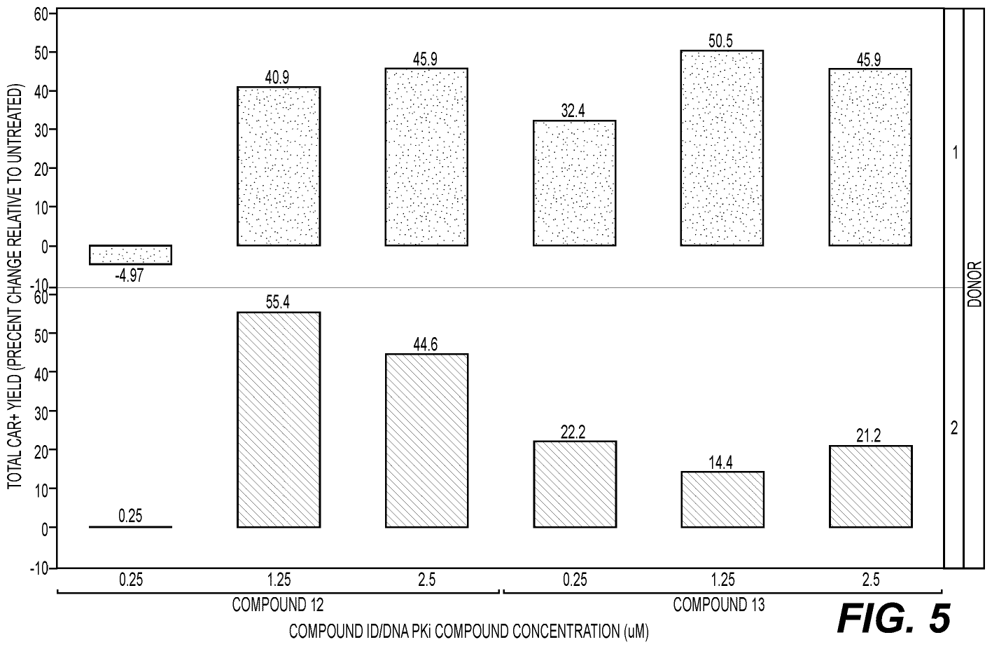

FIG. 5 shows the effect of DNA-PK inhibitor compounds on total CAR+ cell yields 5 days after electroporation. Relative CAR+ yield is shown as percentage change over the untreated control condition (calculated by dividing the number of CAR+ cells in DNA-PKi treated condition by untreated, subtracting 1, and multiplying by 100).

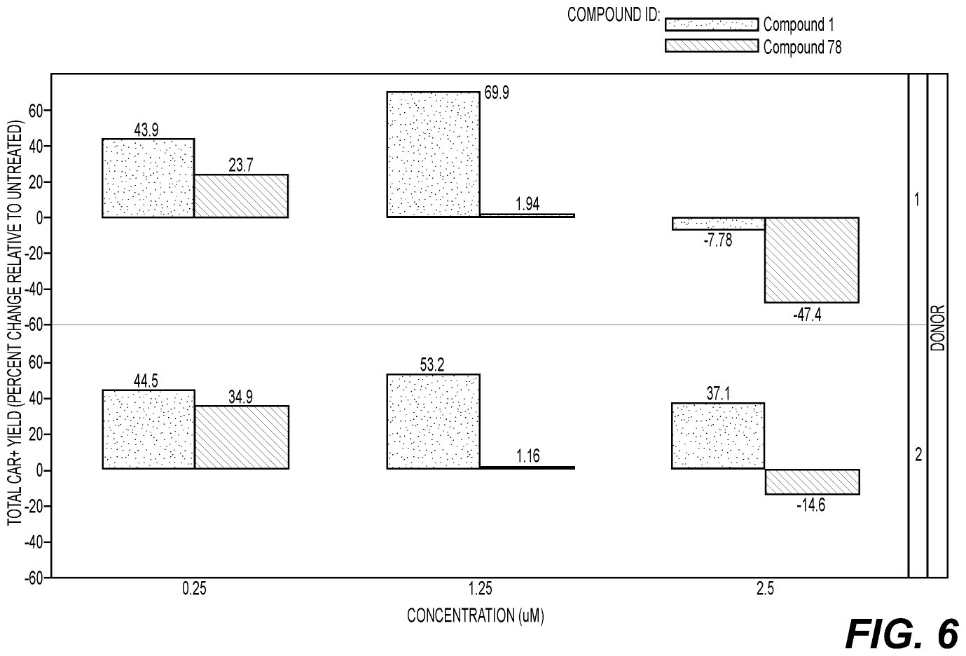

FIG. 6 shows the effect of DNA-PK inhibitor compounds (Compound 1 and Compound 78) on total CAR+ cell yields 5 days after electroporation. Relative CAR+ yield is shown as percentage change over the untreated control condition (calculated by dividing the number of CAR+ cells in DNA-PKi treated condition by untreated, subtracting 1, and multiplying by 100).

TABLE 5
Sequences

| SEQ | | |
|---|---|---|
| ID | | |
| NO: | Sequence | Description |
| 6 | MEAGITGTWYNQLGSTFIVTAGADGALTGTYIGARGNAESRYV | streptavidin mutein |
| | LTGRYDSAPATDGSGTALGWTVAWKNNYRNAHSATTWSGQY | (Strep-Tactin ® |
| | VGGAEARINTQWLLTSGTTEANAWKSTLVGHDTFTKVKPSAAS | m2) |
| 16 | SAWSHPQFEKGGGSGGGSGGSAWSHPQFEK | streptavidin- |
| | | binding peptide |
| | | sequence |
| | | (Twin-Strep-tag ®) |
| | | (aa) |
| 29 | ESKYGPPCPPCPAPPVAGPSVFLFPPKPKDTLMISRTPEVTCVVV | Hinge-CH2—CH3 |
| | DVSQEDPEVQFNWYVDGVEVHNAKTKPREEQFQSTYRVVSVLT | spacer |
| | VLHQDWLNGKEYKCKVSNKGLPSSIEKTISKAKGQPREPQVYTL | Homo sapiens (aa) |
| | PPSQEEMTKNQVSLTCLVKGFYPSDIAVEWESNGQPENNYKTTP | |
| | PVLDSDGSFFLYSRLTVDKSRWQEGNVFSCSVMHEALHNHYTQ | |
| | KSLSLSLGK | |
| 31 | QVQLQQSGAELARPGASVKMSCKASGYTFTRYTMHWVKQRPG | anti-CD3 antibody |
| | QGLEWIGYINPSRGYTNYNQKFKDKATLTTDKSSSTAYMQLSSL | OKT3 heavy chain |
| | TSEDSAVYYCARYYDDHYCLDYWGQGTTLTVSS | (aa) |
| 32 | QIVLTQSPAIMSASPGEKVTMTCSASSSVSYMNWYQQKSGTSPK | anti-CD3 antibody |
| | RWIYDTSKLASGVPAHFRGSGSGTSYSLTISGMEAEDAATYYCQ | OKT3 light chain |
| | QWSSNPFTFGSGTKLEIN | (aa) |
| 33 | LQQSGAELVKPGASVRLSCKASGYTFTEYIIHWIKLRSGQGLEWI | heavy chain |
| | GWFYPGSNDIQYNAKFKGKATLTADKSSSTVYMELTGLTSEDS | variable domain of |
| | AVYFCARRDDFSGYDALPYWGQGTMVTV | anti-CD28 |
| | | antibody CD28.3 |
| | | (aa) |
| 34 | DIQMTQSPASLSVSVGETVTITCRTNENIYSNLAWYQQKQGKSP | light chain variable |
| | QLLIYAATHLVEGVPSRFSGSGSGTQYSLKITSLQSEDFGNYYCQ | domain of |
| | HFWGTPCTFGGGTKLEIKR | anti-CD28 |
| | | antibody CD28.3 |
| | | (aa) |
| 36 | EVQLVQSGAEMKKPGASLKLSCKASGYTFIDYYVYWMRQAPG | Variable heavy |
| | QGLESMGWINPNSGGTNYAQKFQGRVTMTRDTSISTAYMELSR | (VH) Anti-BCMA |
| | LRSDDTAMYYCARSQRDGYMDYWGQGTLVTVSS | (aa) |
| 37 | QSALTQPASVSASPGQSIAISCTGTSSDVGWYQQHPGKAPKLMIY | Variable light (VL) |
| | EDSKRPSGVSNRFSGSKSGNTASLTISGLQAEDEADYYCSSNTRS | Anti-BCMA (aa) |
| | STLVFGGGTKLTVLG | |
| 56 | DYYVY | BCMA-55 |
| | | CDR-H1 |
| | | (aa)-Kabat |
| | | numbering |
| 57 | WINPNSGGTNYAQKFQG | BCMA-55 |
| | | CDR-H2 |
| | | (aa)-Kabat |
| | | numbering |
| 58 | SQRDGYMDY | BCMA-55 |
| | | CDR-H3 |
| | | (aa)-Kabat, |
| | | Chothia, and AbM |
| | | numbering |
| 59 | TGTSSDVG | BCMA-55 |
| | | CDR-L1 |
| | | (aa)-Kabat, |
| | | Chothia, and AbM |
| | | numbering |
| 60 | EDSKRPS | BCMA-55 |
| | | CDR-L2 |
| | | (aa)-Kabat, |
| | | Chothia, and AbM |
| | | numbering |
| 61 | SSNTRSSTLV | BCMA-55 |
| | | CDR-L3 |
| | | (aa)-Kabat, |
| | | Chothia, and AbM |
| | | numbering |
| 179 | gaatctaagtacggaccgccctgccctccctgccctgctcctcctgtggctggaccaagcgtgttcctg | Modified IgG4 |
| | tttccacctaagcctaaagataccctgatgatttcccgcacacctgaagtgacttgcgtggtcgtggacg | hinge-IgG2/IgG4 |
| | tgagccaggaggatccagaagtgcagttcaactggtacgtggacggcgtggaagtccacaatgctaa | CH2-IgG4 CH3 |
| | gactaaaccccgagaggaacagtttcagtcaacttaccgggtcgtgagcgtgctgaccgtcctgcatc | spacer (nt) |
| | aggattggctgaacgggaaggagtataagtgcaaagtgtctaataagggactgcctagctccatcgag | |
| | aaaacaattagtaaggcaaaagggcagcctcgagaaccacaggtgtataccctgccccctagccag | |
| | gaggaaatgaccaagaaccaggtgtccctgacatgtctggtcaaaggcttctatccaagtgacatcgc | |
| | cgtggagtgggaatcaaatgggcagcccgagaacaattacaagaccacaccacccgtgctggactct | |
| | gatggaagtttctttctgtattccaggctgaccgtggataaatctcgctggcaggagggcaacgtgttct | |
| | cttgcagtgtcatgcacgaagccctgcacaatcattatacacagaagtcactgagcctgtccctgggca | |
| | aa | |
| 180 | QSALTQPASVSASPGQSIAISCTGTSSDVGWYQQHPGKAPKLMIY | BCMA-55 scFv |
| | EDSKRPSGVSNRFSGSKSGNTASLTISGLQAEDEADYYCSSNTRS | (aa) |
| | STLVFGGGTKLTVLGSRGGGGSGGGGSGGGGSLEMAEVQLVQS | |
| | GAEMKKPGASLKLSCKASGYTFIDYYVYWMRQAPGQGLESMG | |
| | WINPNSGGTNYAQKFQGRVTMTRDTSISTAYMELSRLRSDDTA | |
| | MYYCARSQRDGYMDYWGQGTLVTVSS | |
| 183 | gagtctaaatacggaccgccttgtcctccttgtcccgctcctcctgttgccggaccttccgtgttcctgttt | optimized SSE |
| | cctccaaagcctaaggacaccctgatgatcagcaggacccctgaagtgacctgcgtggtggtggatg | modified IgG4 |
| | tgtcccaagaggatcccgaggtgcagttcaactggtatgtggacggcgtggaagtgcacaacgccaa | hinge-IgG2/IgG4 |
| | gaccaagcctagagaggaacagttccagagcacctacagagtggtgtccgtgctgacagtgctgcac | C_{H}2-IgG4 C_{H}3 |
| | caggattggctgaacggcaaagagtacaagtgcaaggtgtccaacaagggcctgcctagcagcatc | spacer (nt) |
| | gagaaaaccatctccaaggccaagggccagccaagagagccccaggtttacacactgcctccaagc | |
| | caagaggaaatgaccaagaatcaggtgtccctgacatgcctggtcaagggcttctacccctccgatat | |
| | cgccgtggaatgggagagcaatggccagcctgagaacaactacaagaccacacctcctgtgctgga | |
| | cagcgacggcagtttcttcctgtatagtagactcaccgtggataaatcaagatggcaagagggcaacg | |
| | tgttcagctgcagcgtgatgcacgaggccctgcacaaccactacacccagaaaagcctgagcctgtct | |
| | ctgggcaag | |
| 184 | ESKYGPPCPPCPAPPVAGPSVFLFPPKPKDTLMISRTPEVTCVVV | optimized SSE |
| | DVSQEDPEVQFNWYVDGVEVHNAKTKPREEQFQSTYRVVSVLT | modified IgG4 |
| | VLHQDWLNGKEYKCKVSNKGLPSSIEKTISKAKGQPREPQVYTL | hinge-IgG2/IgG4 |
| | PPSQEEMTKNQVSLTCLVKGFYPSDIAVEWESNGQPENNYKTTP | C_{H}2-IgG4 C_{H}3 |
| | PVLDSDGSFFLYSRLTVDKSRWQEGNVFSCSVMHEALHNHYTQ | spacer (aa) |
| | KSLSLSLGK | |
| 185 | atgttctgggtgctcgtggtcgttggcggagtgctggcctgttacagcctgctggttaccgtggccttcat | CD28 |
| | catcttttgggtc | transmembrane |
| | | domain (nt) |
| 186 | MFWVLVVVGGVLACYSLLVTVAFIIFWV | CD28 |
| | | transmembrane |
| | | domain (aa) |
| 187 | aagcggggcagaaagaagctgctctacatcttcaagcagcccttcatgcggcccgtgcagaccacac | 4-1BB-derived |
| | aagaggaagatggctgctcctgcagattccccgaggaagaagaaggcggctgcgagctg | intracellular |
| | | co-signaling |
| | | sequence (nt) |
| 188 | KRGRKKLLYIFKQPFMRPVQTTQEEDGCSCRFPEEEEGGCEL | 4-1BB-derived |
| | | intracellular |
| | | co-signaling |
| | | sequence (aa) |
| 189 | agagtgaagttcagcagatccgccgacgctccagcctatcagcagggccaaaaccagctgtacaac | CD3-zeta derived |
| | gagctgaacctggggagaagagaagagtacgacgtgctggataagcggagaggcagagatcctga | intracellular |
| | aatgggcggcaagcccagacggaagaatcctcaagagggcctgtataatgagctgcagaaagacaa | signaling domain |
| | gatggccgaggcctacagcgagatcggaatgaagggcgagcgcagaagaggcaagggacacgat | (nt) |
| | ggactgtaccagggcctgagcaccgccaccaaggatacctatgacgcactgcacatgcaggccctg | |
| | ccacctaga | |
| 190 | RVKFSRSADAPAYQQGQNQLYNELNLGRREEYDVLDKRRGRDP | CD3-zeta derived |
| | EMGGKPRRKNPQEGLYNELQKDKMAEAYSEIGMKGERRRGKG | intracellular |
| | HDGLYQGLSTATKDTYDALHMQALPPR | signaling domain |
| | | (aa) |
| 191 | ggatctgcgatcgctccggtgcccgtcagtgggcagagcgcacatcgcccacagtccccgagaagtt | EF1 alpha promoter |
| | ggggggaggggtcggcaattgaaccggtgcctagagaaggtggcgcggggtaaactgggaaagt | with HTLV1 |
| | gatgtcgtgtactggctccgcctttttcccgaggggggggagaaccgtatataagtgcagtagtcgcc | enhancer |
| | gtgaacgttctttttcgcaacgggtttgccgccagaacacagctgaagcttcgaggggctcgcatctct | |
| | ccttcacgcgcccgccgccctacctgaggccgccatccacgccggttgagtegcgttctgccgcctc | |
| | ccgcctgtggtgcctcctgaactgcgtccgccgtctaggtaagtttaaagctcaggtcgagaccgggc | |
| | ctttgtccggcgctcccttggagcctacctagactcagccggctctccacgctttgcctgaccctgcttg | |
| | ctcaactctacgtctttgtttcgttttctgttctgcgccgttacagatccaagctgtgaccggegcctac | |
| 192 | atggtgctgcagacccaggtgttcatcagcctgctgctgtggatctctggcgcctatgga | Human IgG-kappa |
| | | signal sequence |
| | | (nt) |
| 193 | GGGGSGGGGSGGGGS | (4GS)₃ linker (aa) |
| 194 | tgctttatttgtgaaatttgtgatgctattgctttatttgtaaccattataagctgcaataaacaagttaacaac | polyA tail (nt) |
| | aacaattgcattcattttatgtttcaggttcagggggaggtgtgggaggttttttaaa | |
| 195 | ctctatcaatgagagagcaatctcctggtaatgtgatagatttcccaacttaatgccaacataccataaac | TRAC 5′ HA (nt) |
| | ctcccattctgctaatgcccagcctaagttggggagaccactccagattccaagatgtacagtttgctttg | |
| | ctgggcctttttcccatgcctgcctttactctgccagagttatattgctggggttttgaagaagatcctatta | |
| | aataaaagaataagcagtattattaagtagccctgcatttcaggtttccttgagtggcaggccaggcctg | |
| | gccgtgaacgttcactgaaatcatggcctcttggccaagattgatagcttgtgcctgtccctgagtccca | |
| | gtccatcacgagcagctggtttctaagatgctatttcccgtataaagcatgagaccgtgacttgccagcc | |
| | ccacagagccccgcccttgtccatcactggcatctggactccagcctgggttggggcaaagagggaa | |
| | atgagatcatgtcctaaccctgatcctcttgtcccacagatatccagaaccctgaccctgccgtgtacca | |
| | gctgagagactctaaatccagtgacaagtctgtctgcctattcaccgat | |
| 196 | tttgattctcaaacaaatgtgtcacaaagtaaggattctgatgtgtatatcacagacaaaactgtgctaga | TRAC 3′ HA (nt) |
| | catgaggtctatggacttcaagagcaacagtgctgtggcctggagcaacaaatctgactttgcatgtgc | |
| | aaacgccttcaacaacagcattattccagaagacaccttcttccccagcccaggtaagggcagctttgg | |
| | tgccttcgcaggctgtttccttgcttcaggaatggccaggttctgcccagagctctggtcaatgatgtcta | |
| | aaactcctctgattggtggtctcggccttatccattgccaccaaaaccctctttttactaagaaacagtga | |
| | gccttgttctggcagtccagagaatgacacgggaaaaaagcagatgaagagaaggtggcaggaga | |
| | gggcacgtggcccagcctcagtctctccaactgagttcctgcctgcctgcctttgctcagactgtttgcc | |
| | ccttactgctcttctaggcctcattctaagccccttctccaagttgcctctccttatttctccctgtctgccaa | |
| | aaaatctttcccagctcactaagtcagtctcacgcagtcactcattaacccaccaatcactgattgtg | |
| 197 | atggtgctgcagacccaggtgttcatcagcctgctgctgtggatctctggcgcctatggacagtctgcc | anti-BCMA CAR |
| | ctgacacagcctgccagcgttagtgctagtcccggacagtctatcgccatcagctgtaccggcaccag | (nt) |
| | ctctgacgttggctggtatcagcagcaccctggcaaggcccctaagctgatgatctacgaggacagca | |
| | agaggcccagcggcgtgtccaatagattcagcggcagcaagagcggcaacaccgccagcctgaca | |
| | attagcggactgcaggccgaggacgaggccgattactactgcagcagcaacacccggtccagcaca | |
| | ctggtttttggcggaggcaccaagctgacagtgctgggatctagaggtggcggaggatctggcggcg | |
| | gaggaagcggaggcggcggatctcttgaaatggctgaagtgcagctggtgcagtctggcgccgaga | |
| | tgaagaaacctggcgcctctctgaagctgagctgcaaggccagcggctacaccttcatcgactactac | |
| | gtgtactggatgcggcaggcccctggacagggactcgaatctatgggctggatcaaccccaatagcg | |
| | gcggcaccaattacgcccagaaattccagggcagagtgaccatgaccagagacaccagcatcagca | |
| | ccgcctacatggaactgagccggctgagatccgacgacaccgccatgtactactgcgccagatctca | |
| | gcgcgacggctacatggattattggggccagggaaccctggtcaccgtgtccagcgagtctaaatac | |
| | ggaccgccttgtcctccttgtcccgctcctcctgttgccggaccttccgtgttcctgtttcctccaaagcct | |
| | aaggacaccctgatgatcagcaggacccctgaagtgacctgcgtggtggtggatgtgtcccaagagg | |
| | atcccgaggtgcagttcaactggtatgtggacggcgtggaagtgcacaacgccaagaccaagcctag | |
| | agaggaacagttccagagcacctacagagtggtgtccgtgctgacagtgctgcaccaggattggctg | |
| | aacggcaaagagtacaagtgcaaggtgtccaacaagggcctgcctagcagcatcgagaaaaccatc | |
| | tccaaggccaagggccagccaagagagccccaggtttacacactgcctccaagccaagaggaaatg | |
| | accaagaatcaggtgtccctgacatgcctggtcaagggcttctacccctccgatatcgccgtggaatg | |
| | ggagagcaatggccagcctgagaacaactacaagaccacacctcctgtgctggacagcgacggca | |
| | gtttcttcctgtatagtagactcaccgtggataaatcaagatggcaagagggcaacgtgttcagctgca | |
| | gcgtgatgcacgaggccctgcacaaccactacacccagaaaagcctgagcctgtctctgggcaagat | |
| | gttctgggtgctcgtggtcgttggcggagtgctggcctgttacagcctgctggttaccgtggccttcatc | |
| | atcttttgggtcaagcggggcagaaagaagctgctctacatcttcaagcagcccttcatgcggcccgtg | |
| | cagaccacacaagaggaagatggctgctcctgcagattccccgaggaagaagaaggcggctgcga | |
| | gctgagagtgaagttcagcagatccgccgacgctccagcctatcagcagggccaaaaccagctgtac | |
| | aacgagctgaacctggggagaagagaagagtacgacgtgctggataagcggagaggcagagatcc | |
| | tgaaatgggcggcaagcccagacggaagaatcctcaagagggcctgtataatgagctgcagaaaga | |
| | caagatggccgaggcctacagcgagatcggaatgaagggcgagcgcagaagaggcaagggaca | |
| | cgatggactgtaccagggcctgagcaccgccaccaaggatacctatgacgcactgcacatgcaggc | |
| | cctgccacctagataa | |
| 198 | MVLQTQVFISLLLWISGAYGQSALTQPASVSASPGQSIAISCTGTS | anti-BCMA CAR |
| | SDVGWYQQHPGKAPKLMIYEDSKRPSGVSNRFSGSKSGNTASLT | (aa) |
| | ISGLQAEDEADYYCSSNTRSSTLVFGGGTKLTVLGSRGGGGSGG | |
| | GGSGGGGSLEMAEVQLVQSGAEMKKPGASLKLSCKASGYTFID | |
| | YYVYWMRQAPGQGLESMGWINPNSGGTNYAQKFQGRVTMTR | |
| | DTSISTAYMELSRLRSDDTAMYYCARSQRDGYMDYWGQGTLV | |
| | TVSSESKYGPPCPPCPAPPVAGPSVFLFPPKPKDTLMISRTPEVTC | |
| | VVVDVSQEDPEVQFNWYVDGVEVHNAKTKPREEQFQSTYRVV | |
| | SVLTVLHQDWLNGKEYKCKVSNKGLPSSIEKTISKAKGQPREPQ | |
| | VYTLPPSQEEMTKNQVSLTCLVKGFYPSDIAVEWESNGQPENNY | |
| | KTTPPVLDSDGSFFLYSRLTVDKSRWQEGNVFSCSVMHEALHN | |
| | HYTQKSLSLSLGKMFWVLVVVGGVLACYSLLVTVAFIIFWVKR | |
| | GRKKLLYIFKQPFMRPVQTTQEEDGCSCRFPEEEEGGCELRVKFS | |
| | RSADAPAYQQGQNQLYNELNLGRREEYDVLDKRRGRDPEMGG | |
| | KPRRKNPQEGLYNELQKDKMAEAYSEIGMKGERRRGKGHDGL | |
| | YQGLSTATKDTYDALHMQALPPR | |
| 199 | ctctatcaatgagagagcaatctcctggtaatgtgatagatttcccaacttaatgccaacataccataaac | Entire homology |
| | ctcccattctgctaatgcccagcctaagttggggagaccactccagattccaagatgtacagtttgctttg | directed repair |
| | ctgggcctttttcccatgcctgcctttactctgccagagttatattgctggggttttgaagaagatcctatta | template, including |
| | aataaaagaataagcagtattattaagtagccctgcatttcaggtttccttgagtggcaggccaggcctg | homology arms |
| | gccgtgaacgttcactgaaatcatggcctcttggccaagattgatagcttgtgcctgtccctgagtccca | (nt) |
| | gtccatcacgagcagctggtttctaagatgctatttcccgtataaagcatgagaccgtgacttgccagcc | |
| | ccacagagccccgcccttgtccatcactggcatctggactccagcctgggttggggcaaagagggaa | |
| | atgagatcatgtcctaaccctgatcctcttgtcccacagatatccagaaccctgaccctgccgtgtacca | |
| | gctgagagactctaaatccagtgacaagtctgtctgcctattcaccgatggatccggatctgcgatcgct | |
| | ccggtgcccgtcagtgggcagagcgcacatcgcccacagtccccgagaagttggggggaggggtc | |
| | ggcaattgaaccggtgcctagagaaggtggcgcggggtaaactgggaaagtgatgtcgtgtactggc | |
| | tccgcctttttcccgaggggggggagaaccgtatataagtgcagtagtcgccgtgaacgttctttttcg | |
| | caacgggtttgccgccagaacacagctgaagcttcgaggggctcgcatctctccttcacgcgcccgc | |
| | cgccctacctgaggccgccatccacgccggttgagtcgcgttctgccgcctcccgcctgtggtgcctc | |
| | ctgaactgcgtccgccgtctaggtaagtttaaagctcaggtcgagaccgggcctttgtccggcgctccc | |
| | ttggagcctacctagactcagccggctctccacgctttgcctgaccctgcttgctcaactctacgtctttgt | |
| | ttcgttttctgttctgcgccgttacagatccaagctgtgaccggcgcctacggctagcgccaccatggtg | |
| | ctgcagacccaggtgttcatcagcctgctgctgtggatctctggcgcctatggacagtctgccctgaca | |
| | cagcctgccagcgttagtgctagtcccggacagtctatcgccatcagctgtaccggcaccagctctga | |
| | cgttggctggtatcagcagcaccctggcaaggcccctaagctgatgatctacgaggacagcaagagg | |
| | cccagcggcgtgtccaatagattcagcggcagcaagagcggcaacaccgccagcctgacaattagc | |
| | ggactgcaggccgaggacgaggccgattactactgcagcagcaacacccggtccagcacactggtt | |
| | tttggcggaggcaccaagctgacagtgctgggatctagaggtggcggaggatctggcggcggagg | |
| | aagcggaggcggcggatctcttgaaatggctgaagtgcagctggtgcagtctggcgccgagatgaa | |
| | gaaacctggcgcctctctgaagctgagctgcaaggccagcggctacaccttcatcgactactacgtgt | |
| | actggatgcggcaggcccctggacagggactcgaatctatgggctggatcaaccccaatagcggcg | |
| | gcaccaattacgcccagaaattccagggcagagtgaccatgaccagagacaccagcatcagcaccg | |
| | cctacatggaactgagccggctgagatccgacgacaccgccatgtactactgcgccagatctcagcg | |
| | cgacggctacatggattattggggccagggaaccctggtcaccgtgtccagcgagtctaaatacgga | |
| | ccgccttgtcctccttgtcccgctcctcctgttgccggaccttccgtgttcctgtttcctccaaagcctaag | |
| | gacaccctgatgatcagcaggacccctgaagtgacctgcgtggtggtggatgtgtcccaagaggatc | |
| | ccgaggtgcagttcaactggtatgtggacggcgtggaagtgcacaacgccaagaccaagcctagag | |
| | aggaacagttccagagcacctacagagtggtgtccgtgctgacagtgctgcaccaggattggctgaa | |
| | cggcaaagagtacaagtgcaaggtgtccaacaagggcctgcctagcagcatcgagaaaaccatctc | |
| | caaggccaagggccagccaagagagccccaggtttacacactgcctccaagccaagaggaaatga | |
| | ccaagaatcaggtgtccctgacatgcctggtcaagggcttctacccctccgatatcgccgtggaatgg | |
| | gagagcaatggccagcctgagaacaactacaagaccacacctcctgtgctggacagcgacggcagt | |
| | ttcttcctgtatagtagactcaccgtggataaatcaagatggcaagagggcaacgtgttcagctgcagc | |
| | gtgatgcacgaggccctgcacaaccactacacccagaaaagcctgagcctgtctctgggcaagatgt | |
| | tctgggtgctcgtggtcgttggcggagtgctggcctgttacagcctgctggttaccgtggccttcatcat | |
| | cttttgggtcaagcggggcagaaagaagctgctctacatcttcaagcagcccttcatgcggcccgtgc | |
| | agaccacacaagaggaagatggctgctcctgcagattccccgaggaagaagaaggcggctgcgag | |
| | ctgagagtgaagttcagcagatccgccgacgctccagcctatcagcagggccaaaaccagctgtaca | |
| | acgagctgaacctggggagaagagaagagtacgacgtgctggataagcggagaggcagagatcct | |
| | gaaatgggcggcaagcccagacggaagaatcctcaagagggcctgtataatgagctgcagaaaga | |
| | caagatggccgaggcctacagcgagatcggaatgaagggcgagcgcagaagaggcaagggaca | |
| | cgatggactgtaccagggcctgagcaccgccaccaaggatacctatgacgcactgcacatgcaggc | |
| | cctgccacctagataaactagtgtcgactgctttatttgtgaaatttgtgatgctattgctttatttgtaaccat | |
| | tataagctgcaataaacaagttaacaacaacaattgcattcattttatgtttcaggttcagggggaggtgt | |
| | gggaggttttttaaagggccctttgattctcaaacaaatgtgtcacaaagtaaggattctgatgtgtatatc | |
| | acagacaaaactgtgctagacatgaggtctatggacttcaagagcaacagtgctgtggcctggagcaa | |
| | caaatctgactttgcatgtgcaaacgccttcaacaacagcattattccagaagacaccttcttccccagc | |
| | ccaggtaagggcagctttggtgccttcgcaggctgtttccttgcttcaggaatggccaggttctgccca | |
| | gagctctggtcaatgatgtctaaaactcctctgattggtggtctcggccttatccattgccaccaaaaccc | |
| | tctttttactaagaaacagtgagccttgttctggcagtccagagaatgacacgggaaaaaagcagatga | |
| | agagaaggtggcaggagagggcacgtggcccagcctcagtctctccaactgagttcctgcctgcctg | |
| | cctttgctcagactgtttgccccttactgctcttctaggcctcattctaagccccttctccaagttgcctctcc | |
| | ttatttctccctgtctgccaaaaaatctttcccagctcactaagtcagtctcacgcagtcactcattaaccc | |
| | accaatcactgattgtg | |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

### Enumerated Embodiments

Enumerated Embodiment 1. A compound of Formula (I):

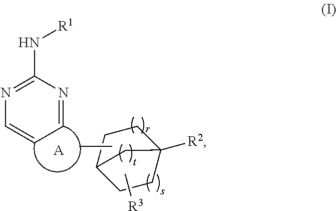

or a pharmaceutically acceptable salt, stereoisomer, solvate, prodrug, or tautomer thereof,
wherein:
A is a 5- or 6-membered heteroaryl or heterocycloalkyl group containing at least one heteroatom selected from the group consisting of N, O, and S, wherein the heteroaryl or heterocycloalkyl is optionally substituted with one or more R⁴.
R¹ is an aryl or heteroaryl containing at least one heteroatom selected from the group consisting of N, O, S, and Se, wherein the aryl or heteroaryl is optionally substituted with one or more R⁵.
R² is H, halogen, —(CH₂)ₙ—CN, —OH, —(CH₂)ₙ—O—C₁-C₄ alkyl, C₁-C₄ alkyl, C₁-C₄ alkoxy, C₁-C₄ haloalkyl, C₂-C₄ alkenyl, C₂-C₄ alkynyl, or C(O)NH₂;
R³ is selected from the group consisting of H, F, C₂-C₄ alkenyl, C₂-C₄ alkynyl, CN, OH, CH₂OH, NH₂, CH₂NH₂, and C₁-C₄ alkyl;
each R⁴ is independently selected from the group consisting of halogen, oxo, thioxo, C₁-C₄ alkyl, C₁-C₄ alkoxy, CD₃, CD₂CD₃, and C₁-C₆ haloalkyl; or
two geminal R⁴ together with the intervening geminal carbon atom, form a C₃-C₆ cycloalkyl;
each R⁵ is independently selected from the group consisting of halogen, NH₂, OH, —CN, C(O)NH₂, C(O)NHR⁷, C1-C₄ alkyl, C1-C₄ alkyl, C₂-C₄ alkenyl, C₂-C₄ alkynyl, CD₃, CD₂CD₃, C₁-C₆ alkoxy, C₁-C₆ haloalkyl, C₃-C₆ cycloalkyl, heterocycloalkyl, heteroaryl, and aryl, wherein the alkyl, alkoxy, haloalkyl, cycloalkyl, heterocycloalkyl, heteroaryl, or aryl is optionally substituted with one or more R⁶.
each R⁶ is independently selected from the group consisting of halogen, OH, oxo, NH₂, CHO, C₁-C₄ alkyl, and C₁-C₆ alkoxy;
each R⁷ is independently selected from H and C₁-C₄ alkyl;
each n is independently an integer from 0-4;
r is an integer from 0 to 2;
s is an integer from 0 to 2; and
t is an integer from 1 to 2,
provided that when A is a 5-membered heterocycle containing N—C(O)—N in the ring and R¹ is

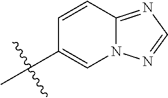 [SMILES: *c1ccc2ncnn2c1]

optionally substituted with one or more R⁵, then

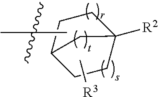

is not

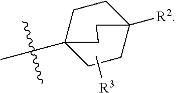

Enumerated Embodiment 2. The compound of enumerated embodiment 1, or a pharmaceutically acceptable salt, stereoisomer, solvate, prodrug, or tautomer thereof, wherein R¹ is

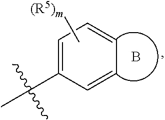

wherein B is a 5- or 6-membered aryl or heteroaryl, optionally substituted with one or more R⁵; and m is an integer from 0-4.

Enumerated Embodiment 3. The compound of enumerated embodiment 1, or a pharmaceutically acceptable salt, stereoisomer, solvate, prodrug, or tautomer thereof, wherein R¹ is selected from the group consisting of:

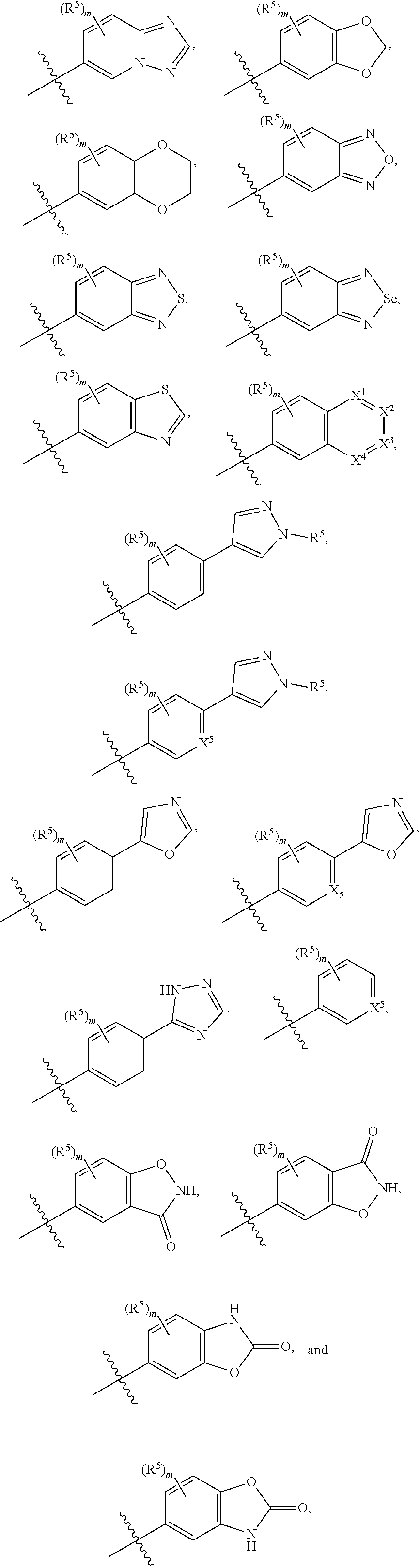

wherein:
X¹, X², X³, X⁴ and X⁵ are each independently N, CH, or C(R⁵); and
m is an integer from 1 to 3.

Enumerated Embodiment 4. The compound of any one of enumerated embodiments 1-3, wherein the compound is of Formula (Ia-1):

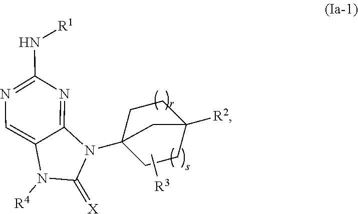

or a pharmaceutically acceptable salt, stereoisomer, solvate, prodrug, or tautomer thereof, wherein X is O, S, NH, or CH₂.

Enumerated Embodiment 5. The compound of any one of enumerated embodiments 1, 3, or 4, wherein the compound is of Formula (Ia-2):

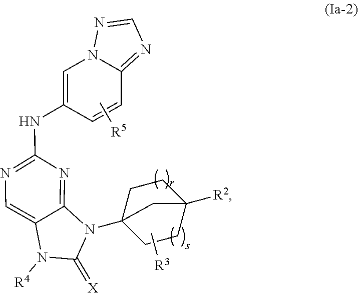

or a pharmaceutically acceptable salt, stereoisomer, solvate, prodrug, or tautomer thereof, wherein X is O, S, NH, or CH₂.

Enumerated Embodiment 6. The compound of any one of enumerated embodiments 1-4, wherein the compound is of Formula (Ia-3):

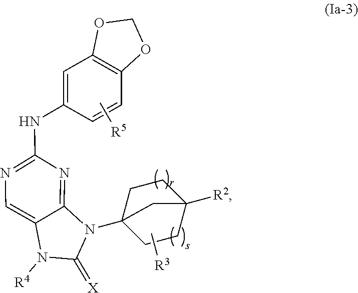

or a pharmaceutically acceptable salt, stereoisomer, solvate, prodrug, or tautomer thereof, wherein X is O, S, NH, or CH₂.

Enumerated Embodiment 7. The compound of any one of enumerated embodiments 1-4, wherein the compound is of Formula (Ia-4):

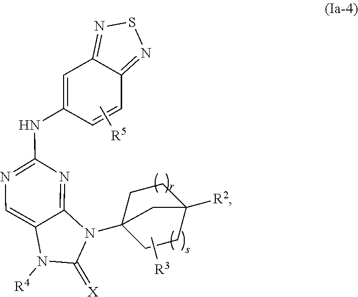

or a pharmaceutically acceptable salt, stereoisomer, solvate, prodrug, or tautomer thereof, wherein X is O, S, NH, or CH₂.

Enumerated Embodiment 8. The compound of any one of enumerated embodiments 1-4, wherein the compound is of Formula (Ia-5):

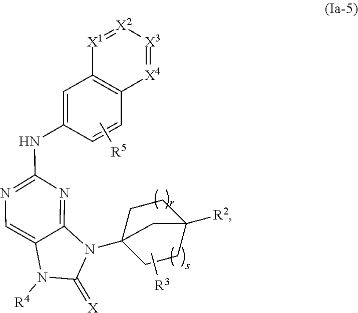

or a pharmaceutically acceptable salt, stereoisomer, solvate, prodrug, or tautomer thereof, wherein
X is O, S, NH, or CH₂; and
X¹, X², X³, and X⁴ are each independently N, CH, or C(R⁵).

Enumerated Embodiment 9. The compound of any one of enumerated embodiments 1-4, wherein the compound is of Formula (Ia-6):

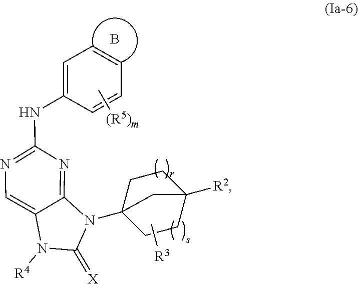

or a pharmaceutically acceptable salt, stereoisomer, solvate, prodrug, or tautomer thereof, wherein X is O, S, NH, or CH₂;
B is a 5- or 6-membered aryl or heteroaryl, optionally substituted with one or more R⁵; and
m is an integer from 0-4.

Enumerated Embodiment 10. The compound of any one of enumerated embodiments 1-Error! Reference source not found., wherein the compound is of Formula (Ia-7):

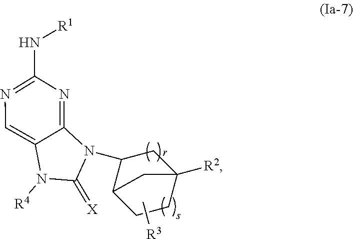

or a pharmaceutically acceptable salt, stereoisomer, solvate, prodrug, or tautomer thereof, wherein X is O, S, NH, or CH₂.

Enumerated Embodiment 11. The compound of any one of enumerated embodiments 1-3, wherein the compound is of Formula (Ib-1):

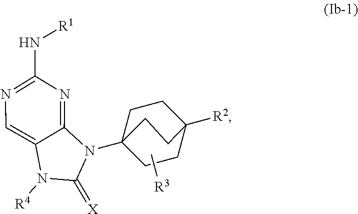

or a pharmaceutically acceptable salt, stereoisomer, solvate, prodrug, or tautomer thereof, wherein X is O, S, NH, or CH₂, provided that when R¹ is

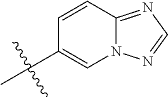 [SMILES: *c1ccc2ncnn2c1]

optionally substituted with one or more R⁵, then X is not O.

Enumerated Embodiment 12. The compound of any one of enumerated embodiments 1, 3 or 11, wherein the compound is of Formula (Ib-2):

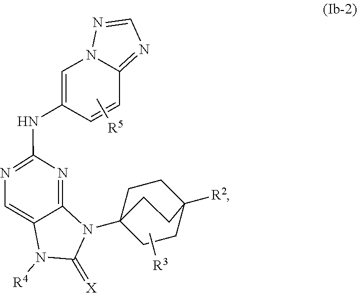

or a pharmaceutically acceptable salt, stereoisomer, solvate, prodrug, or tautomer thereof, wherein X is S, NH, or CH₂.

Enumerated Embodiment 13. The compound of any one of enumerated embodiments 1-3, or 11, wherein the compound is of Formula (Ib-3):

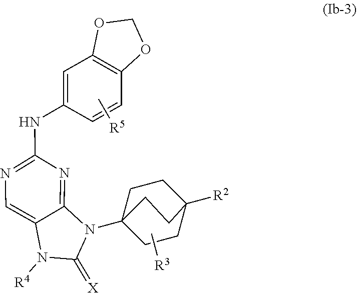

or a pharmaceutically acceptable salt, stereoisomer, solvate, prodrug, or tautomer thereof, wherein X is O, S, NH, or CH₂.

Enumerated Embodiment 14. The compound of any one of enumerated embodiments 1-3, or 11, wherein the compound is of Formula (Ib-4):

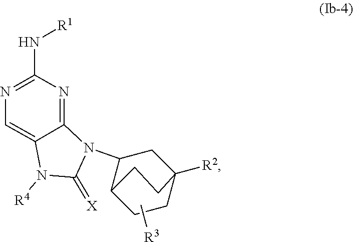

or a pharmaceutically acceptable salt, stereoisomer, solvate, prodrug, or tautomer thereof, wherein X is O, S, NH, or CH₂.

Enumerated Embodiment 15. The compound of any one of enumerated embodiments 1-3, wherein the compound is of Formula (Ic-1):

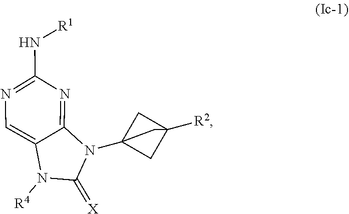

or a pharmaceutically acceptable salt, stereoisomer, solvate, prodrug, or tautomer thereof, wherein X is O, S, NH, or CH₂.

Enumerated Embodiment 16. The compound of any one of enumerated embodiments 1, 3, or 15, wherein the compound is of Formula (Ic-2):

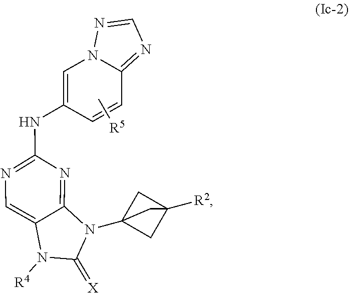

or a pharmaceutically acceptable salt, stereoisomer, solvate, prodrug, or tautomer thereof, wherein X is O, S, NH, or CH₂.

Enumerated Embodiment 17. The compound of any one of enumerated embodiments 1-3, or 15, wherein the compound is of Formula (Ic-3):

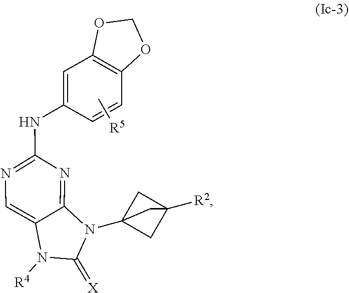

or a pharmaceutically acceptable salt, stereoisomer, solvate, prodrug, or tautomer thereof, wherein X is O, S, NH, or CH₂.

Enumerated Embodiment 18. The compound of any one of enumerated embodiments 1-3, or 15, wherein the compound is of Formula (Ic-4):

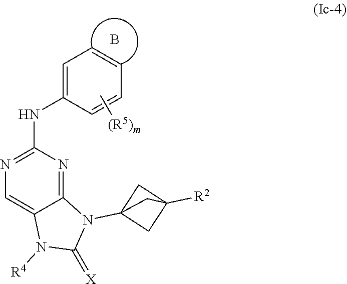

or a pharmaceutically acceptable salt, stereoisomer, solvate, prodrug, or tautomer thereof, wherein
X is O, S, NH, or CH₂; B is a 5- or 6-membered aryl or heteroaryl, optionally substituted with one or more R⁵; and
m is an integer from 1 to 3.

Enumerated Embodiment 19. The compound of any one of enumerated embodiments 1-3, wherein the compound is of Formula (Id-1):

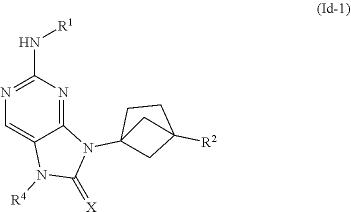

or a pharmaceutically acceptable salt, stereoisomer, solvate, prodrug, or tautomer thereof, wherein X is O, S, NH, or CH₂.

Enumerated Embodiment 20. The compound of any one of enumerated embodiments 1, 3, or 19, wherein the compound is of Formula (Id-2):

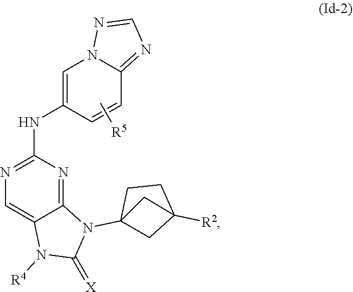

or a pharmaceutically acceptable salt, stereoisomer, solvate, prodrug, or tautomer thereof, wherein X is O, S, NH, or CH₂.

Enumerated Embodiment 21. The compound of any one of enumerated embodiments 1-3, or 19, wherein the compound is of Formula (Id-3):

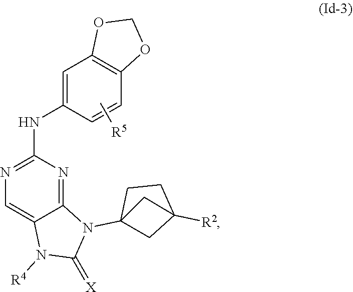

or a pharmaceutically acceptable salt, stereoisomer, solvate, prodrug, or tautomer thereof, wherein X is O, S, NH, or CH₂.

Enumerated Embodiment 22. The compound of any one of enumerated embodiments 1-3, or 19, wherein the compound is of Formula (Id-4):

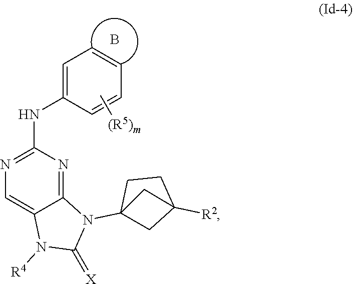

or a pharmaceutically acceptable salt, stereoisomer, solvate, prodrug, or tautomer thereof, wherein X is O, S, NH, or CH₂; and
m is an integer from 1 to 3.

Enumerated Embodiment 23. The compound of any one of enumerated embodiments 1-3, wherein the compound is of Formula (Ie-1):

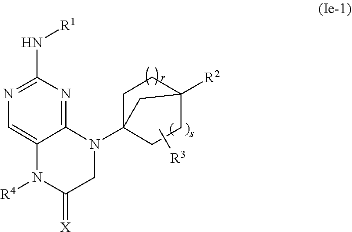

or a pharmaceutically acceptable salt, stereoisomer, solvate, prodrug, or tautomer thereof, wherein X is O, S, NH, or CH₂.

Enumerated Embodiment 24. The compound of any one of enumerated embodiments 1, 3, or 19, wherein the compound is of Formula (Ie-2):

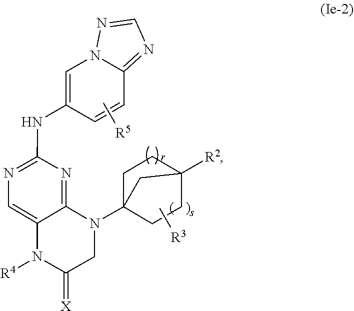

or a pharmaceutically acceptable salt, stereoisomer, solvate, prodrug, or tautomer thereof, wherein X is O, S, NH, or CH₂.

Enumerated Embodiment 25. The compound of any one of enumerated embodiments 1-3, or 23, wherein the compound is of Formula (Ie-3):

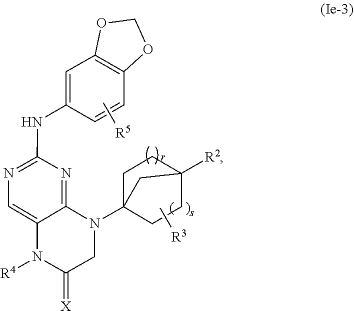

or a pharmaceutically acceptable salt, stereoisomer, solvate, prodrug, or tautomer thereof, wherein X is O, S, NH, or CH₂.

Enumerated Embodiment 26. The compound of any one of enumerated embodiments 1-3, or 23, wherein the compound is of Formula (Ie-4):

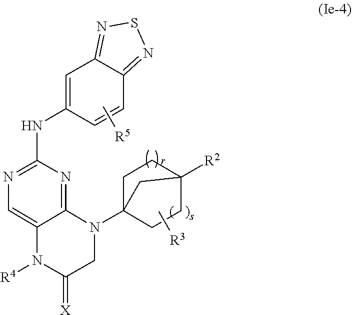

or a pharmaceutically acceptable salt, stereoisomer, solvate, prodrug, or tautomer thereof, wherein
X is O, S, NH, or CH₂.

Enumerated Embodiment 27. The compound of any one of enumerated embodiments 1-3, or 23, wherein the compound is of Formula (Ie-5):

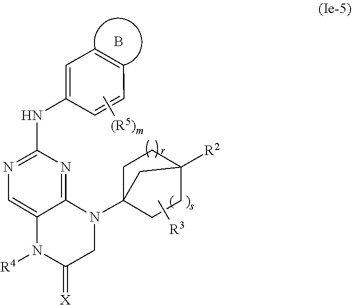

or a pharmaceutically acceptable salt, stereoisomer, solvate, prodrug, or tautomer thereof, wherein
X is O, S, NH, or CH₂;
B is a 5- or 6-membered aryl or heteroaryl, optionally substituted with one or more R⁵; and
m is an integer from 1 to 3.

Enumerated Embodiment 28. The compound of any one of enumerated embodiments 1-3, wherein the compound is of Formula (If-1):

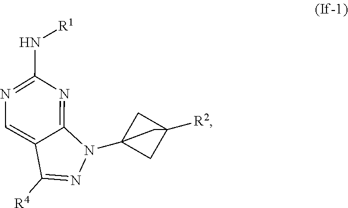

or a pharmaceutically acceptable salt, stereoisomer, solvate, prodrug, or tautomer thereof.

Enumerated Embodiment 29. The compound of any one of enumerated embodiments 1, 3, or 28, wherein the compound is of Formula (If-2):

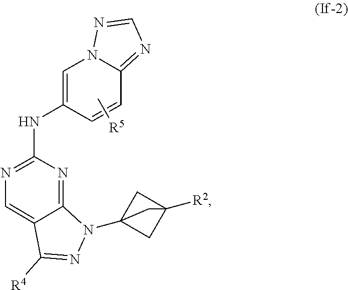

or a pharmaceutically acceptable salt, stereoisomer, solvate, prodrug, or tautomer thereof.

Enumerated Embodiment 30. The compound of any one of enumerated embodiments 1-3, or 28, wherein the compound is of Formula (If-3):

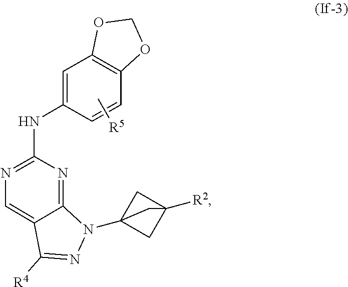

or a pharmaceutically acceptable salt, stereoisomer, solvate, prodrug, or tautomer thereof.

Enumerated Embodiment 31. The compound of any one of enumerated embodiments 1-3, or 28, wherein the compound is of Formula (If-4):

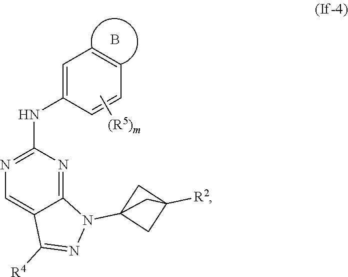

or a pharmaceutically acceptable salt, stereoisomer, solvate, prodrug, or tautomer thereof, wherein
B is a 5- or 6-membered aryl or heteroaryl, optionally substituted with one or more R⁵; and
m is an integer from 1 to 3.

Enumerated Embodiment 32. The compounds of any one of enumerated embodiments 1-3, wherein the compound is of Formula (I) (Ig-1):

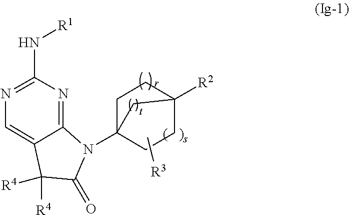

or a pharmaceutically acceptable salt, stereoisomer, solvate, prodrug, or tautomer thereof.

Enumerated Embodiment 33. The compounds of any one of enumerated embodiments 1-3, wherein the compound is of Formula (I) (Ig-2)

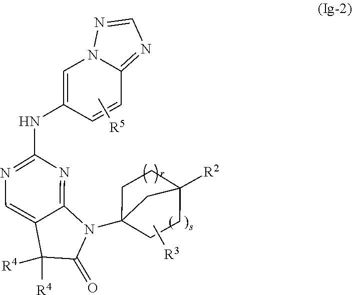

or a pharmaceutically acceptable salt, stereoisomer, solvate, prodrug, or tautomer thereof.

Enumerated Embodiment 34. The compounds of any one of enumerated embodiments 1-3, wherein the compound is of Formula I (Ig-3):

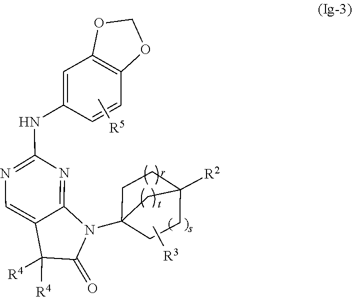

or a pharmaceutically acceptable salt, stereoisomer, solvate, prodrug, or tautomer thereof.

Enumerated Embodiment 35. The compounds of any one of enumerated embodiments 1-3, wherein the compound is of Formula (I) (Ig-4):

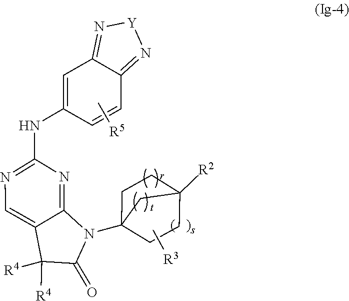

or a pharmaceutically acceptable salt, stereoisomer, solvate, prodrug, or tautomer thereof, wherein Y is O, or S.

Enumerated Embodiment 36. The compounds of any one of enumerated embodiments 1-3, wherein the compound is of Formula (I) (Ig-5):

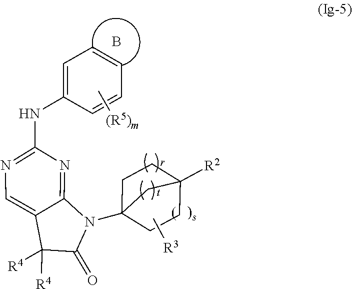

or a pharmaceutically acceptable salt, stereoisomer, solvate, prodrug, or tautomer thereof, wherein B is a 5- or 6-membered aryl or heteroaryl, optionally substituted with one or more R⁵; and m is an integer from 0 to 4. In some embodiments, m is an integer from 1 to 3.

Enumerated Embodiment 37. The compound of enumerated embodiment 1 or 3, wherein the compound is selected from the group consisting of:

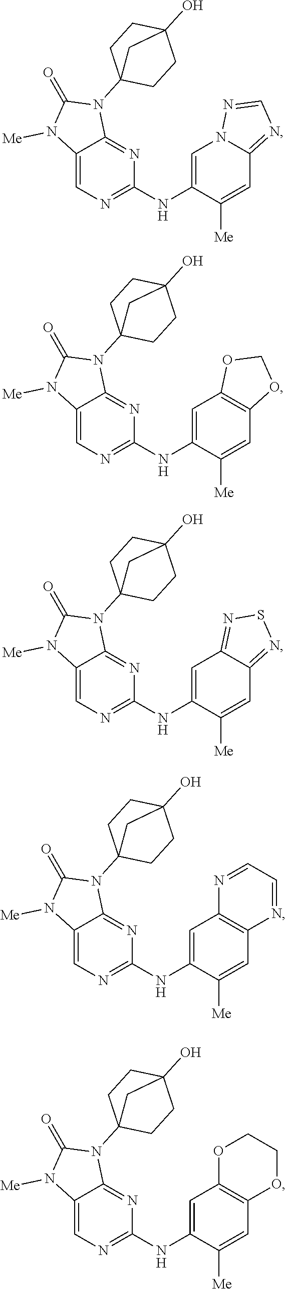

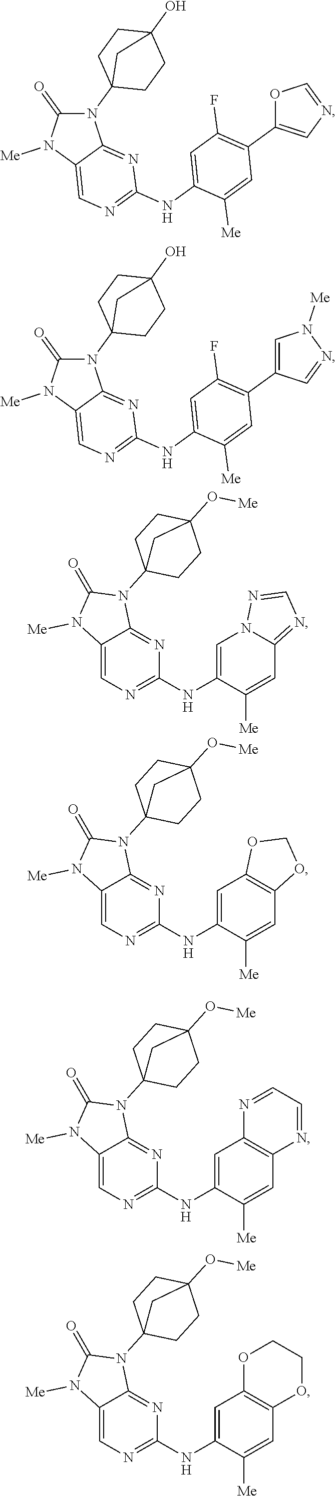

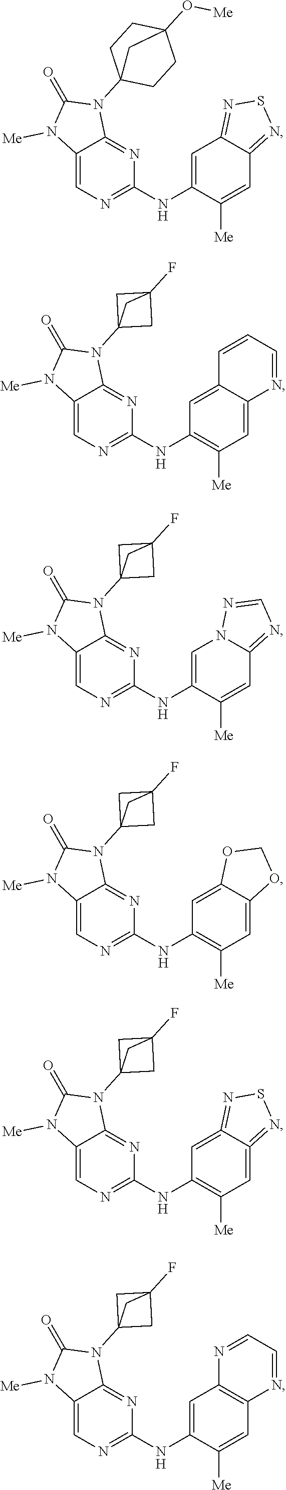

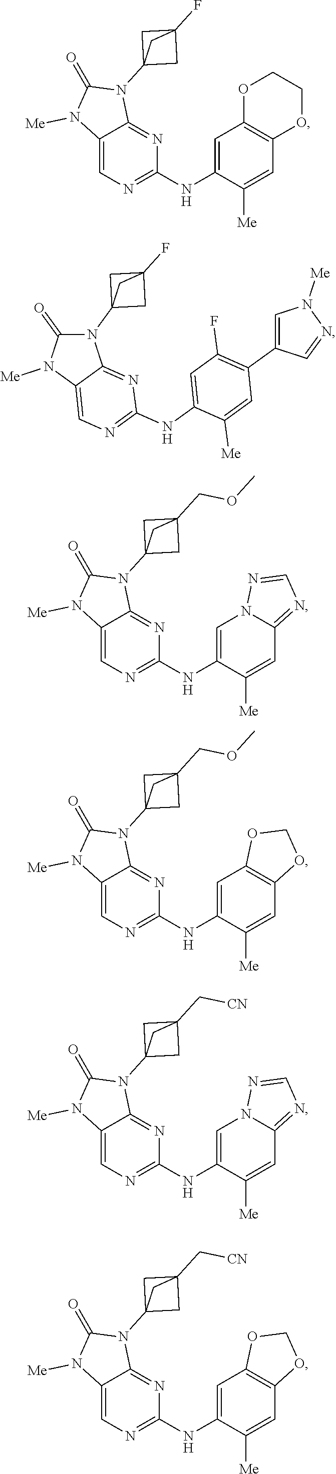

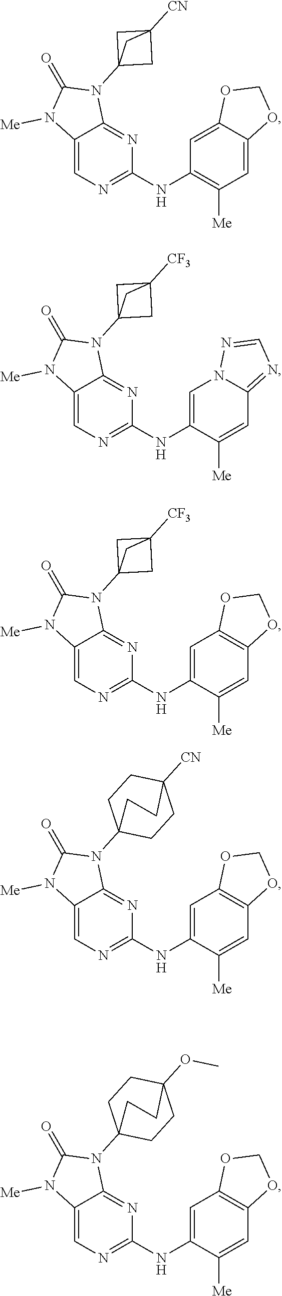

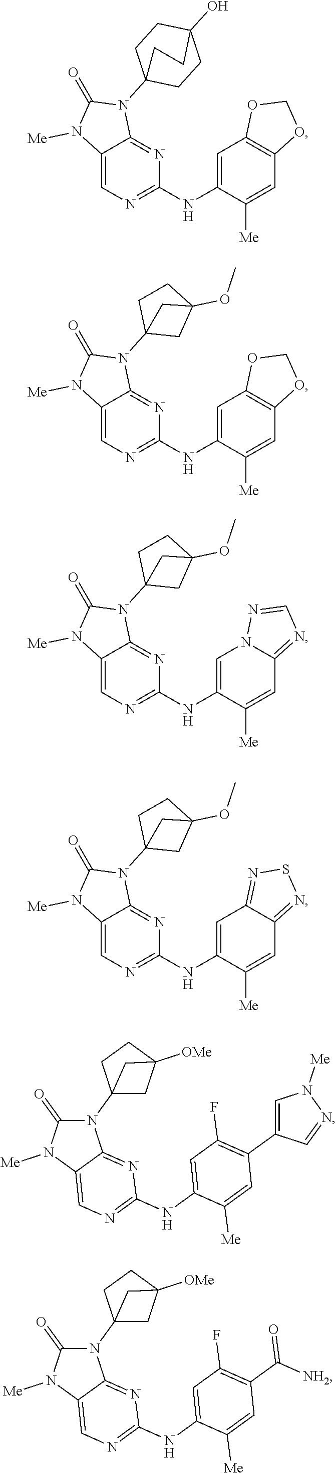

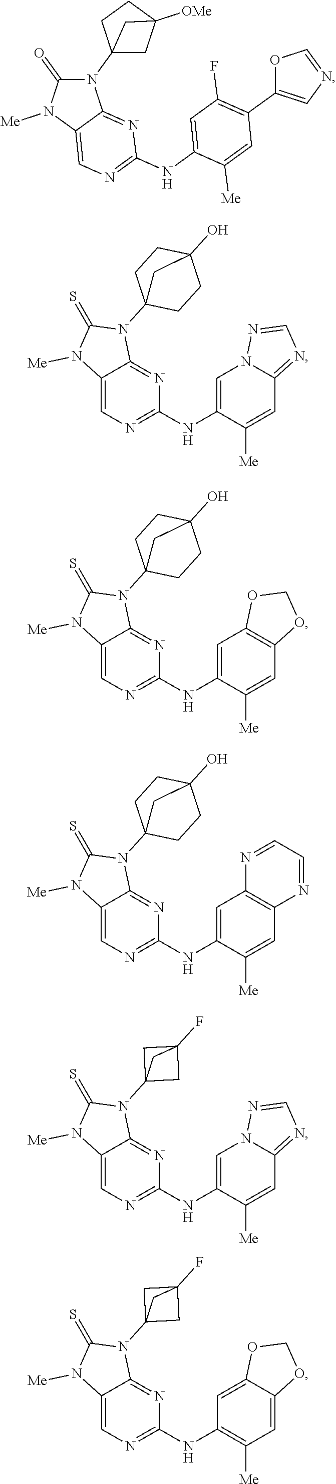

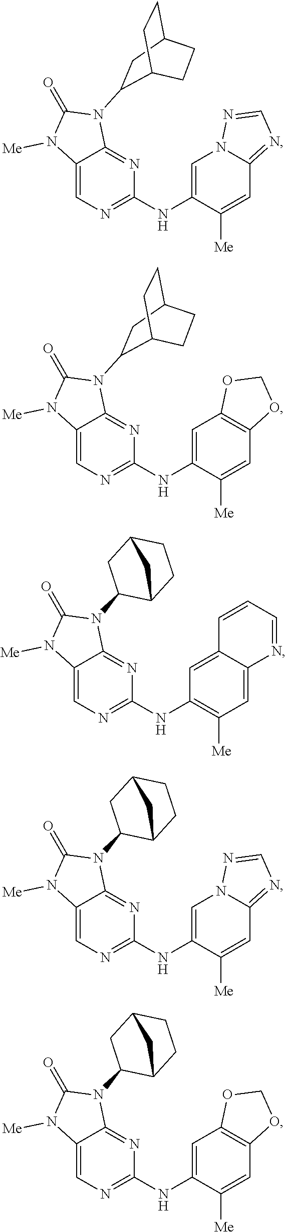 [SMILES: Cc1cc2ncnn2cc1Nc1ncc2c(n1)n(C1CC3CCC1CC3)c(=O)n2C ; Cc1cc2c(cc1Nc1ncc3c(n1)n(C1CC4CCC1CC4)c(=O)n3C)OCO2 ; Cc1cc2ncccc2cc1Nc1ncc2c(n1)n([C@H]1C[C@@H]3CC[C@H]1C3)c(=O)n2C ; Cc1cc2ncnn2cc1Nc1ncc2c(n1)n([C@H]1C[C@@H]3CC[C@H]1C3)c(=O)n2C ; Cc1cc2c(cc1Nc1ncc3c(n1)n([C@H]1C[C@@H]4CC[C@H]1C4)c(=O)n3C)OCO2]

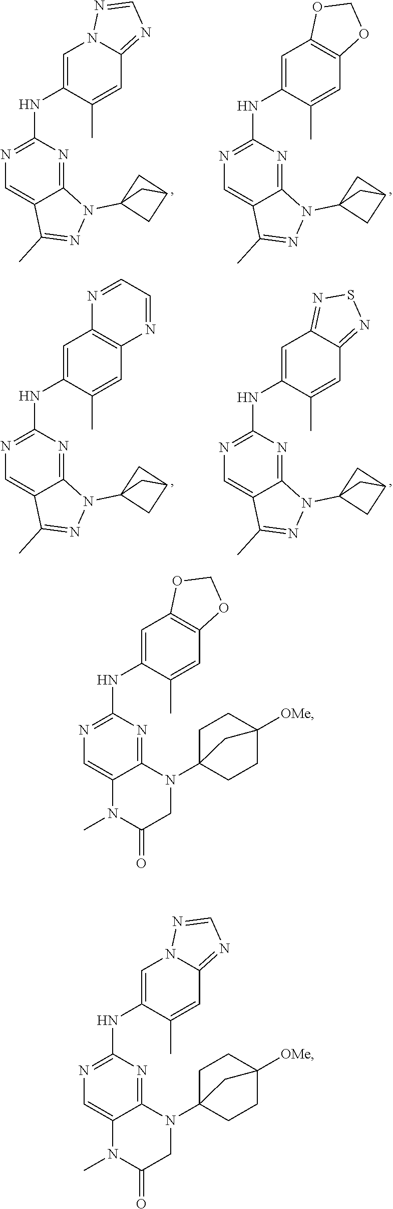

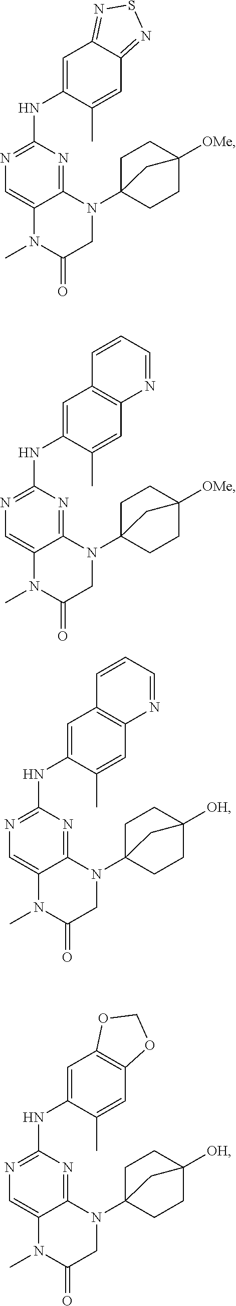 [SMILES: COC12CCC(N3CC(=O)N(C)c4cnc(Nc5cc6nsnc6cc5C)nc43)(CC1)C2 ; COC12CCC(N3CC(=O)N(C)c4cnc(Nc5cc6cccnc6cc5C)nc43)(CC1)C2 ; Cc1cc2ncccc2cc1Nc1ncc2c(n1)N(C13CCC(O)(CC1)C3)CC(=O)N2C ; Cc1cc2c(cc1Nc1ncc3c(n1)N(C14CCC(O)(CC1)C4)CC(=O)N3C)OCO2]

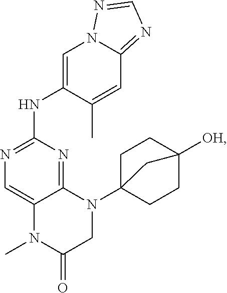 [SMILES: Cc1cc2ncnn2cc1Nc1ncc2c(n1)N(C13CCC(O)(CC1)C3)CC(=O)N2C]

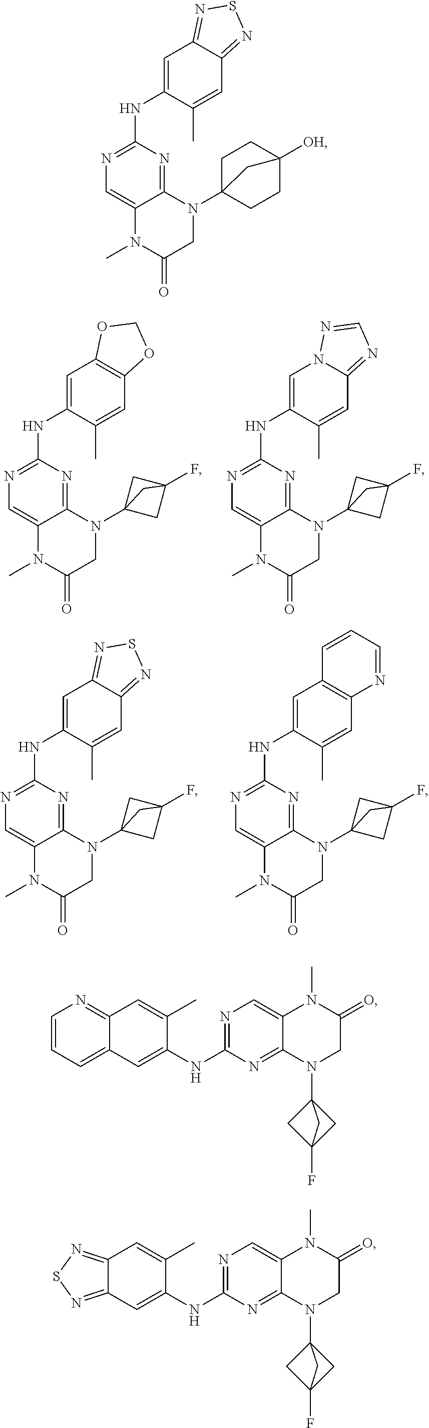

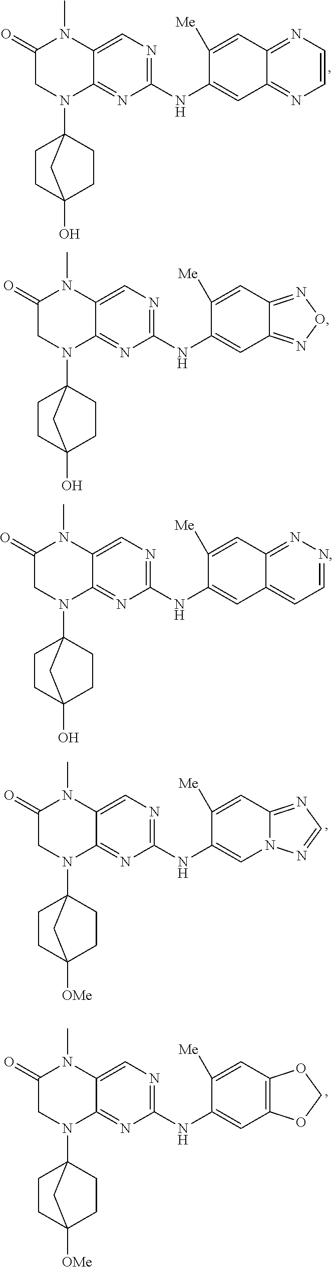

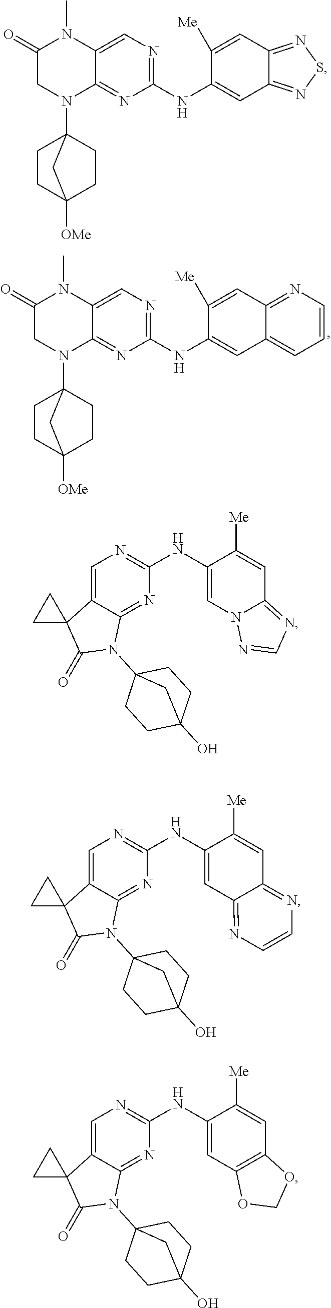

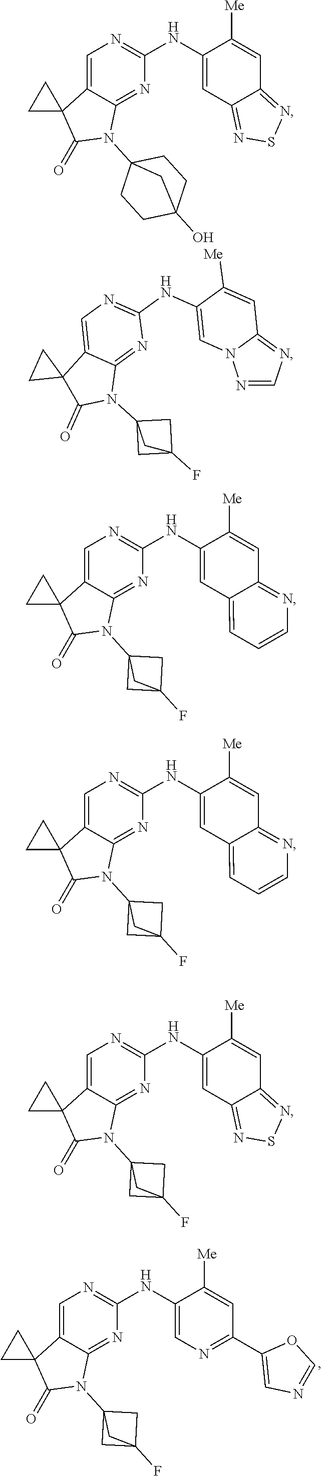

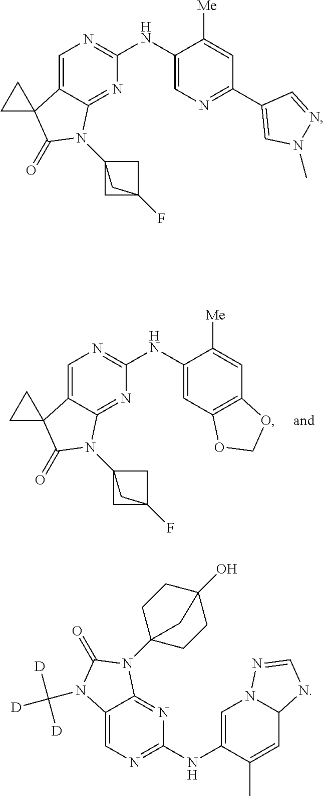

Enumerated Embodiment 38. The compound of enumerated embodiment 1 or 3, wherein the compound is selected from the group consisting of:

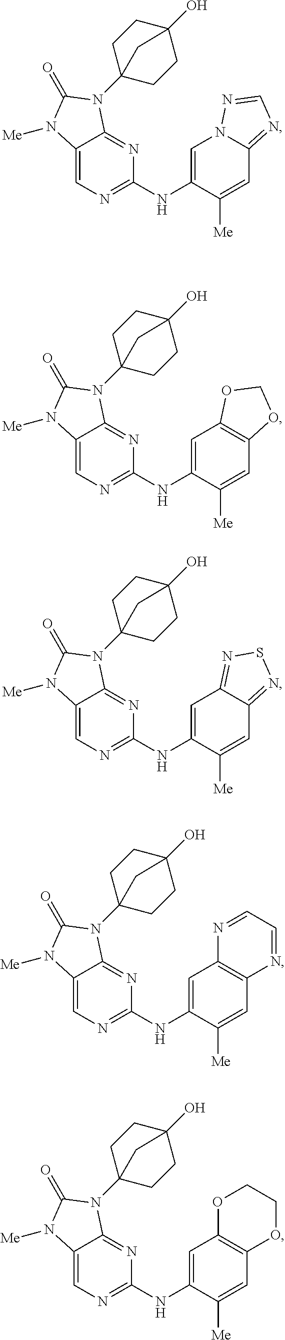 [SMILES: Cc1cc2ncnn2cc1Nc1ncc2c(n1)n(C13CCC(O)(CC1)C3)c(=O)n2C ; Cc1cc2c(cc1Nc1ncc3c(n1)n(C14CCC(O)(CC1)C4)c(=O)n3C)OCO2 ; Cc1cc2nsnc2cc1Nc1ncc2c(n1)n(C13CCC(O)(CC1)C3)c(=O)n2C ; Cc1cc2nccnc2cc1Nc1ncc2c(n1)n(C13CCC(O)(CC1)C3)c(=O)n2C ; Cc1cc2c(cc1Nc1ncc3c(n1)n(C14CCC(O)(CC1)C4)c(=O)n3C)OCCO2]

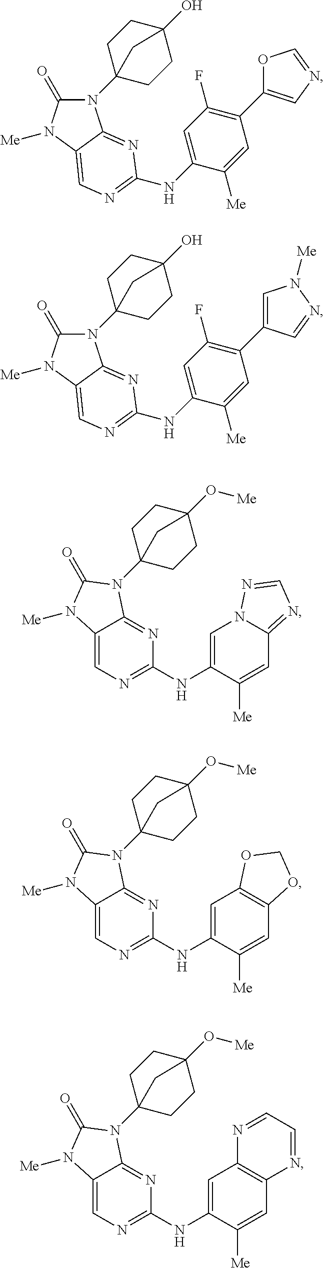 [SMILES: Cc1cc(-c2cnco2)c(F)cc1Nc1ncc2c(n1)n(C13CCC(O)(CC1)C3)c(=O)n2C ; Cc1cc(-c2cnn(C)c2)c(F)cc1Nc1ncc2c(n1)n(C13CCC(O)(CC1)C3)c(=O)n2C ; COC12CCC(n3c(=O)n(C)c4cnc(Nc5cn6ncnc6cc5C)nc43)(CC1)C2 ; COC12CCC(n3c(=O)n(C)c4cnc(Nc5cc6c(cc5C)OCO6)nc43)(CC1)C2 ; COC12CCC(n3c(=O)n(C)c4cnc(Nc5cc6nccnc6cc5C)nc43)(CC1)C2]

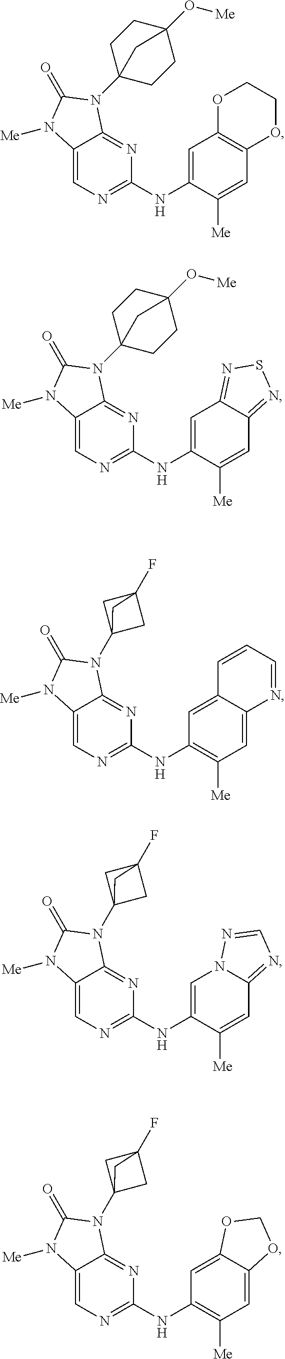 [SMILES: COC12CCC(n3c(=O)n(C)c4cnc(Nc5cc6c(cc5C)OCCO6)nc43)(CC1)C2 ; COC12CCC(n3c(=O)n(C)c4cnc(Nc5cc6nsnc6cc5C)nc43)(CC1)C2 ; Cc1cc2ncccc2cc1Nc1ncc2c(n1)n(C13CC(F)(C1)C3)c(=O)n2C ; Cc1cc2ncnn2cc1Nc1ncc2c(n1)n(C13CC(F)(C1)C3)c(=O)n2C ; Cc1cc2c(cc1Nc1ncc3c(n1)n(C14CC(F)(C1)C4)c(=O)n3C)OCO2]

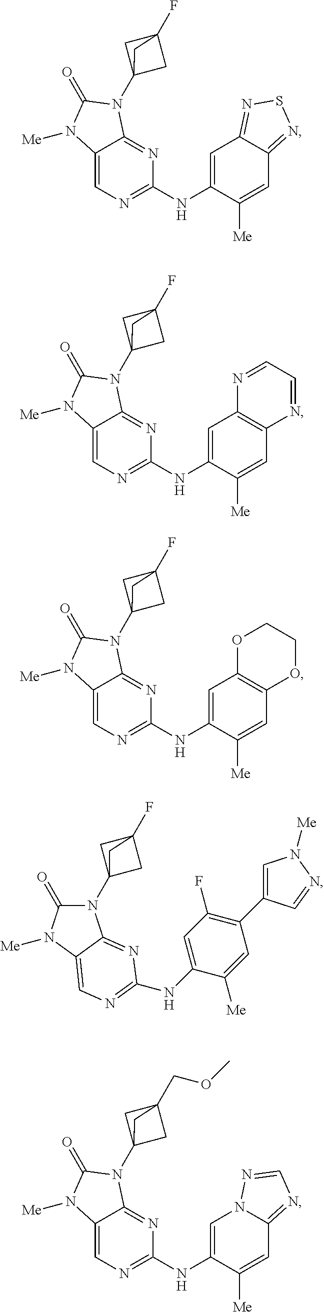 [SMILES: Cc1cc2nsnc2cc1Nc1ncc2c(n1)n(C13CC(F)(C1)C3)c(=O)n2C ; Cc1cc2nccnc2cc1Nc1ncc2c(n1)n(C13CC(F)(C1)C3)c(=O)n2C ; Cc1cc2c(cc1Nc1ncc3c(n1)n(C14CC(F)(C1)C4)c(=O)n3C)OCCO2 ; Cc1cc(-c2cnn(C)c2)c(F)cc1Nc1ncc2c(n1)n(C13CC(F)(C1)C3)c(=O)n2C ; COCC12CC(n3c(=O)n(C)c4cnc(Nc5cn6ncnc6cc5C)nc43)(C1)C2]

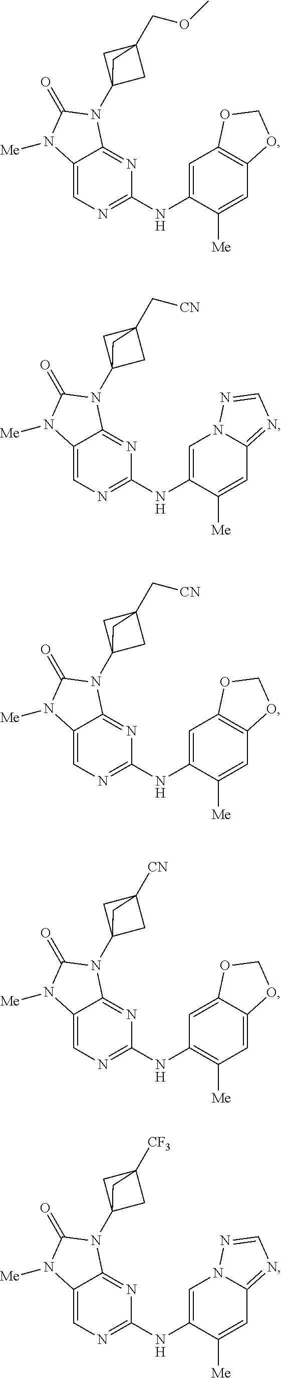 [SMILES: COCC12CC(n3c(=O)n(C)c4cnc(Nc5cc6c(cc5C)OCO6)nc43)(C1)C2 ; Cc1cc2ncnn2cc1Nc1ncc2c(n1)n(C13CC(CC#N)(C1)C3)c(=O)n2C ; Cc1cc2c(cc1Nc1ncc3c(n1)n(C14CC(CC#N)(C1)C4)c(=O)n3C)OCO2 ; Cc1cc2c(cc1Nc1ncc3c(n1)n(C14CC(C#N)(C1)C4)c(=O)n3C)OCO2 ; Cc1cc2ncnn2cc1Nc1ncc2c(n1)n(C13CC(C(F)(F)F)(C1)C3)c(=O)n2C]

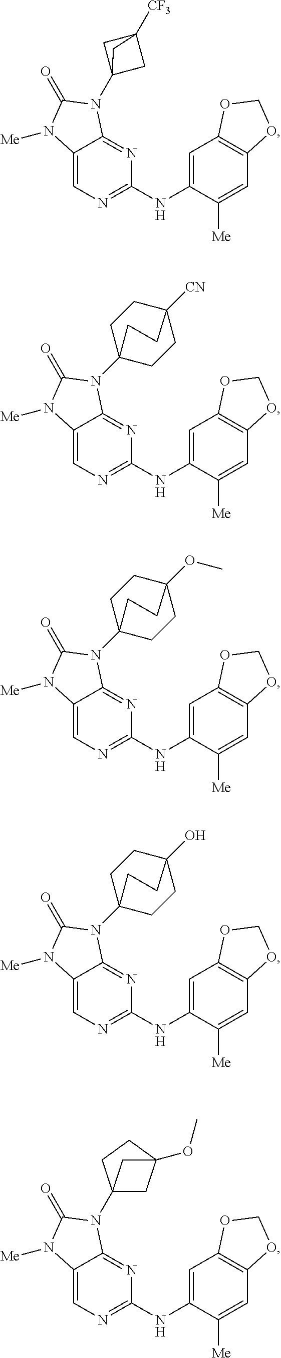 [SMILES: Cc1cc2c(cc1Nc1ncc3c(n1)n(C14CC(C(F)(F)F)(C1)C4)c(=O)n3C)OCO2 ; Cc1cc2c(cc1Nc1ncc3c(n1)n(C14CCC(C#N)(CC1)CC4)c(=O)n3C)OCO2 ; COC12CCC(n3c(=O)n(C)c4cnc(Nc5cc6c(cc5C)OCO6)nc43)(CC1)CC2 ; Cc1cc2c(cc1Nc1ncc3c(n1)n(C14CCC(O)(CC1)CC4)c(=O)n3C)OCO2 ; COC12CCC(n3c(=O)n(C)c4cnc(Nc5cc6c(cc5C)OCO6)nc43)(C1)C2]

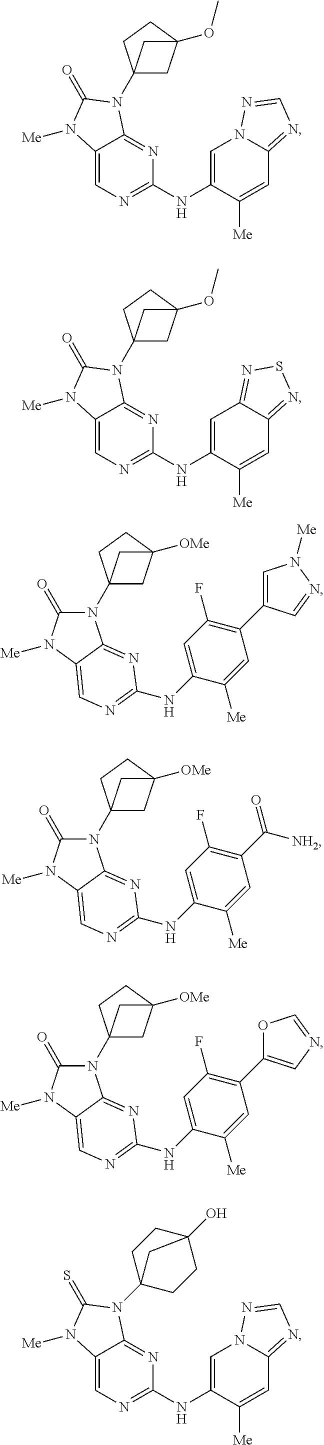

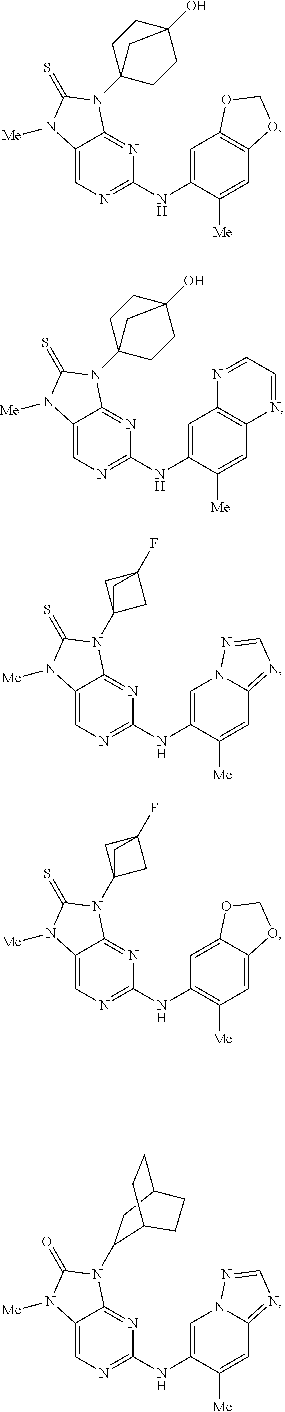 [SMILES: Cc1cc2c(cc1Nc1ncc3c(n1)n(C14CCC(O)(CC1)C4)c(=S)n3C)OCO2 ; Cc1cc2nccnc2cc1Nc1ncc2c(n1)n(C13CCC(O)(CC1)C3)c(=S)n2C ; Cc1cc2ncnn2cc1Nc1ncc2c(n1)n(C13CC(F)(C1)C3)c(=S)n2C ; Cc1cc2c(cc1Nc1ncc3c(n1)n(C14CC(F)(C1)C4)c(=S)n3C)OCO2 ; Cc1cc2ncnn2cc1Nc1ncc2c(n1)n(C1CC3CCC1CC3)c(=O)n2C]

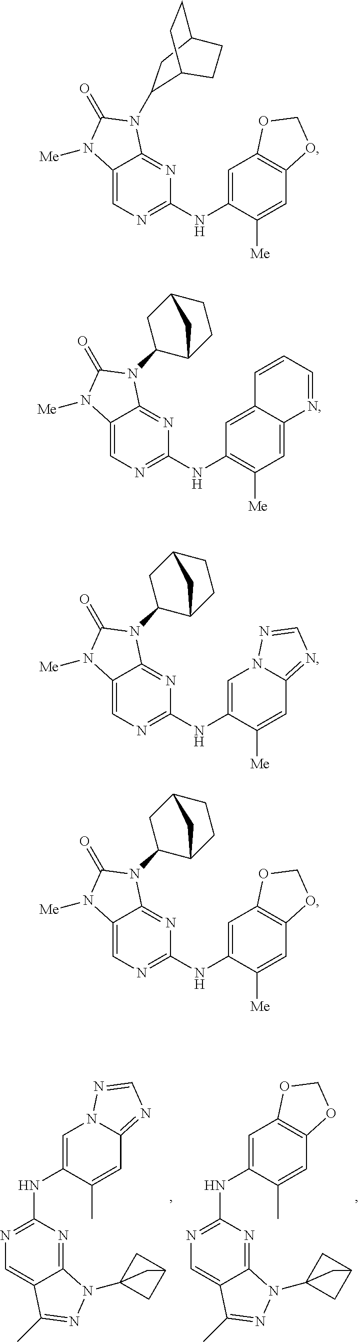

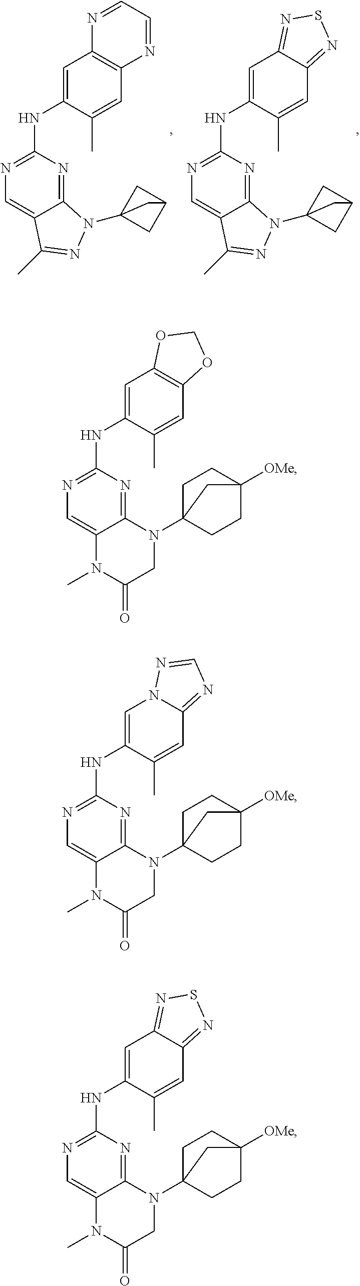

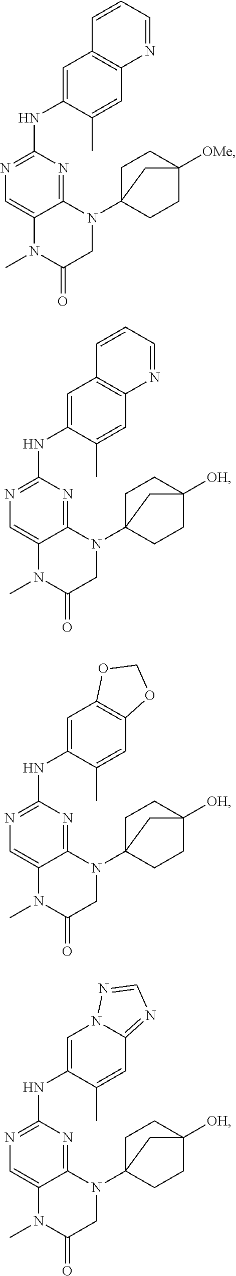 [SMILES: COC12CCC(N3CC(=O)N(C)c4cnc(Nc5cc6cccnc6cc5C)nc43)(CC1)C2 ; Cc1cc2ncccc2cc1Nc1ncc2c(n1)N(C13CCC(O)(CC1)C3)CC(=O)N2C ; Cc1cc2c(cc1Nc1ncc3c(n1)N(C14CCC(O)(CC1)C4)CC(=O)N3C)OCO2 ; Cc1cc2ncnn2cc1Nc1ncc2c(n1)N(C13CCC(O)(CC1)C3)CC(=O)N2C]

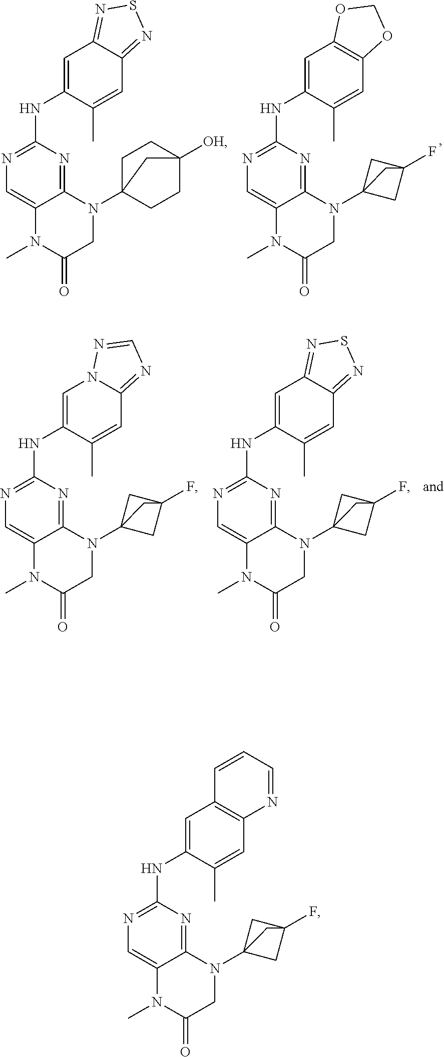

or a pharmaceutically acceptable salt, isomer, solvate, prodrug, or tautomer thereof.

Enumerated Embodiment 39. The compound of enumerated embodiment 1 or 3, wherein the compound is selected from:

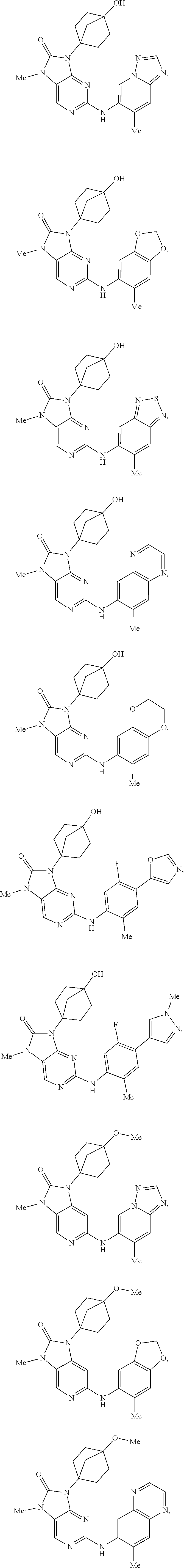

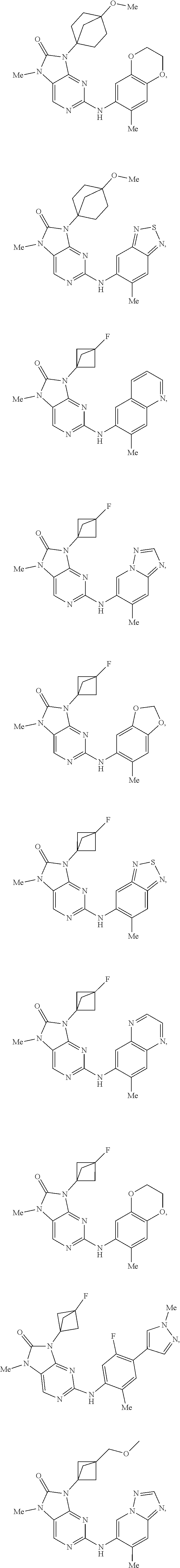

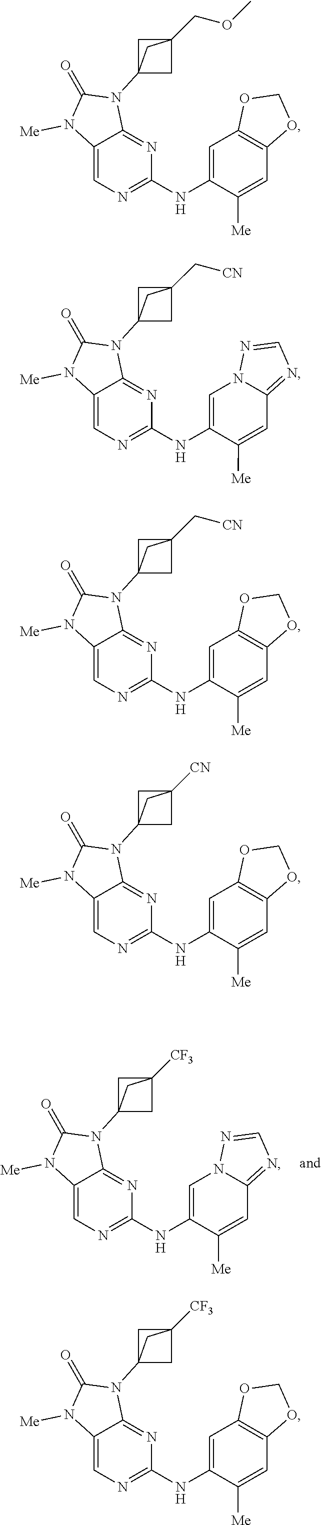

or a pharmaceutically acceptable salt, isomer, solvate, prodrug, or tautomer thereof.

Enumerated Embodiment 40. The compound of claim 1 or 3, wherein the compound is selected from

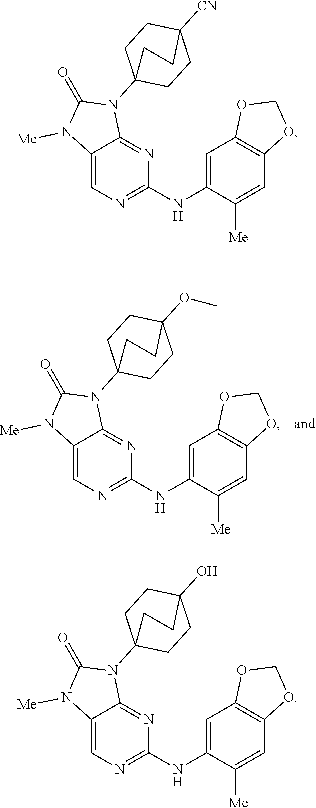

or a pharmaceutically acceptable salt, isomer, solvate, prodrug, or tautomer thereof.

Enumerated Embodiment 41. The compound of enumerated embodiment 1 or 3, wherein the compound is selected from

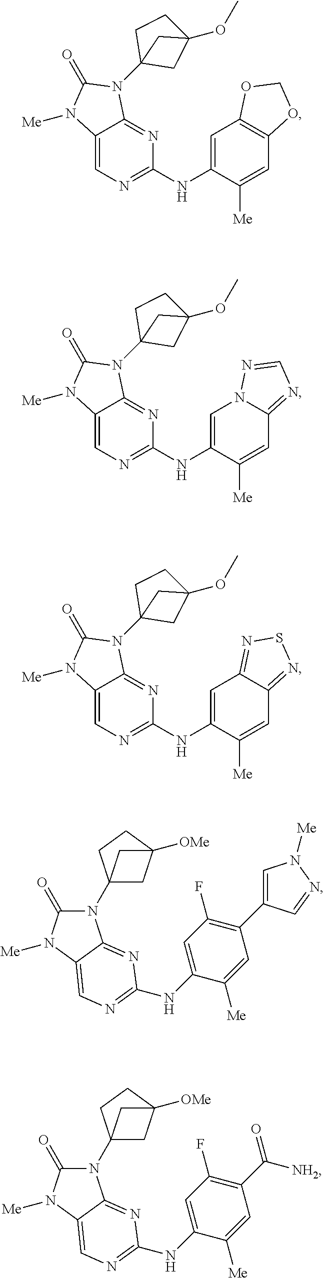 [SMILES: COC12CCC(n3c(=O)n(C)c4cnc(Nc5cc6c(cc5C)OCO6)nc43)(C1)C2 ; COC12CCC(n3c(=O)n(C)c4cnc(Nc5cn6ncnc6cc5C)nc43)(C1)C2 ; COC12CCC(n3c(=O)n(C)c4cnc(Nc5cc6nsnc6cc5C)nc43)(C1)C2 ; COC12CCC(n3c(=O)n(C)c4cnc(Nc5cc(F)c(-c6cnn(C)c6)cc5C)nc43)(C1)C2 ; COC12CCC(n3c(=O)n(C)c4cnc(Nc5cc(F)c(C(N)=O)cc5C)nc43)(C1)C2]

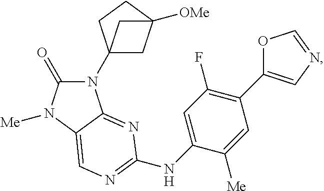 [SMILES: COC12CCC(n3c(=O)n(C)c4cnc(Nc5cc(F)c(-c6cnco6)cc5C)nc43)(C1)C2]

or a pharmaceutically acceptable salt, isomer, solvate, prodrug, or tautomer thereof.

Enumerated Embodiment 42. The compound of claim 1 or 3, wherein the compound is selected from

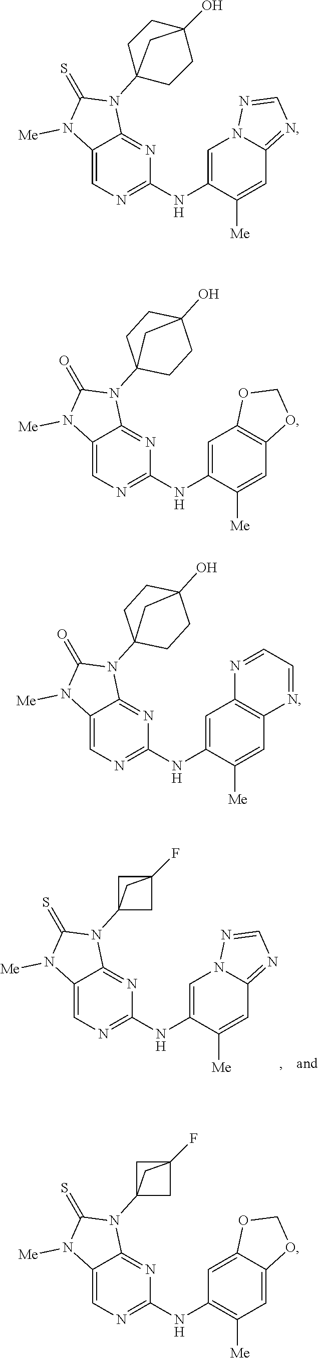 [SMILES: Cc1cc2ncnn2cc1Nc1ncc2c(n1)n(C13CCC(O)(CC1)C3)c(=S)n2C ; Cc1cc2c(cc1Nc1ncc3c(n1)n(C14CCC(O)(CC1)C4)c(=O)n3C)OCO2 ; Cc1cc2nccnc2cc1Nc1ncc2c(n1)n(C13CCC(O)(CC1)C3)c(=O)n2C ; Cc1cc2ncnn2cc1Nc1ncc2c(n1)n(C13CC(F)(C1)C3)c(=S)n2C ; Cc1cc2c(cc1Nc1ncc3c(n1)n(C14CC(F)(C1)C4)c(=S)n3C)OCO2]

or a pharmaceutically acceptable salt, isomer, solvate, prodrug, or tautomer thereof.

Enumerated Embodiment 43. The compound of claim 1 or 3, wherein the compound is selected from

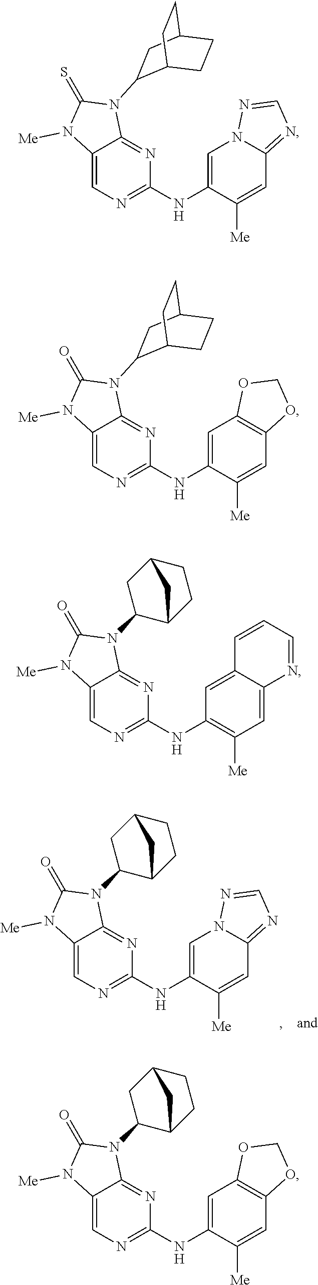 [SMILES: Cc1cc2ncnn2cc1Nc1ncc2c(n1)n(C1CC3CCC1CC3)c(=S)n2C ; Cc1cc2c(cc1Nc1ncc3c(n1)n(C1CC4CCC1CC4)c(=O)n3C)OCO2 ; Cc1cc2ncccc2cc1Nc1ncc2c(n1)n([C@H]1C[C@@H]3CC[C@H]1C3)c(=O)n2C ; Cc1cc2ncnn2cc1Nc1ncc2c(n1)n([C@H]1C[C@@H]3CC[C@H]1C3)c(=O)n2C ; Cc1cc2c(cc1Nc1ncc3c(n1)n([C@H]1C[C@@H]4CC[C@H]1C4)c(=O)n3C)OCO2]

or a pharmaceutically acceptable salt, isomer, solvate, prodrug, or tautomer thereof.

Enumerated Embodiment 44. The compound of enumerated embodiment 1 or 3, wherein the compound is selected from:

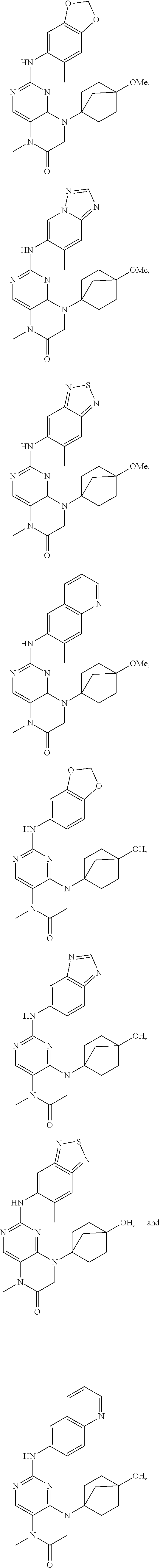

or a pharmaceutically acceptable salt, isomer, solvate, prodrug, or tautomer thereof.

Enumerated Embodiment 45. The compound of enumerated embodiment 1 or 3, wherein the compound is selected from

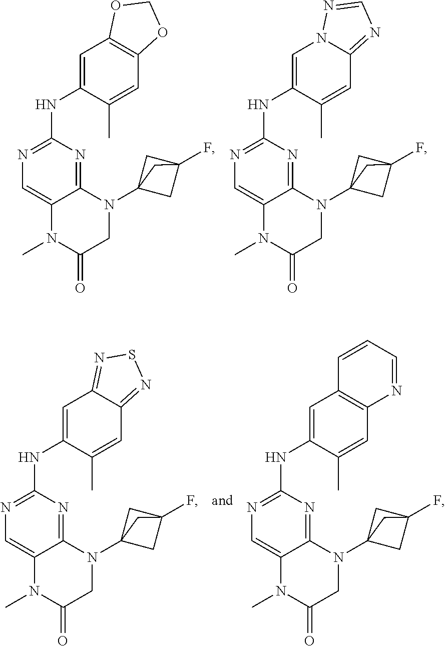

or a pharmaceutically acceptable salt, isomer, solvate, prodrug, or tautomer thereof.

Enumerated Embodiment 46. The compound of enumerated embodiment 1 or 3, wherein the compound is selected from:

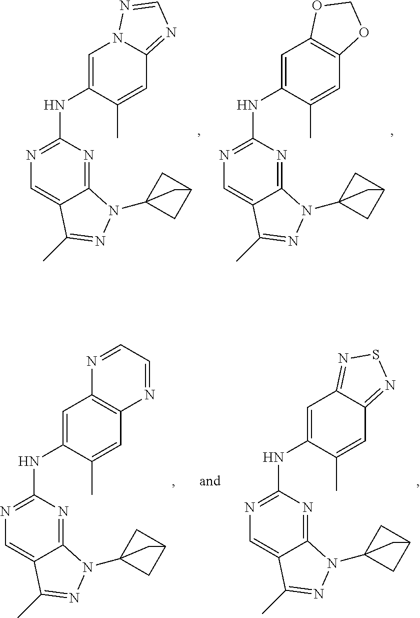

or a pharmaceutically acceptable salt, isomer, solvate, prodrug, or tautomer thereof.

Enumerated Embodiment 47. A pharmaceutically acceptable composition comprising the compound according to any one of enumerated embodiments 1-46, and a pharmaceutically acceptable carrier.

Enumerated Embodiment 48. A composition comprising:
a) a DNA protein kinase inhibitor (DNA-PKI); and
b) a DNA cutting agent;
wherein the DNA-PKI is a compound according to any one of enumerated embodiments 1-46.

Enumerated Embodiment 49. The composition of enumerated embodiment 48, further comprising a cell.

Enumerated Embodiment 50. The composition of enumerated embodiment 48 or 49, further comprising a donor DNA.

Enumerated Embodiment 51. The composition of any one of enumerated embodiments 48-50, wherein the concentration of the DNA-PKI in the composition is about 1 μM or less.

Enumerated Embodiment 52. The composition of any one of enumerated embodiments 48-50, wherein the concentration of the DNA-PKI in the composition is from about 0.1-10 PM.

Enumerated Embodiment 53. The composition of enumerated embodiment 52, wherein the concentration of the DNA-PKI in the composition is from about 0.25-5 μM.

Enumerated Embodiment 54. The composition of any one of enumerated embodiments 49-53, wherein the cell is a eukaryotic cell.

Enumerated Embodiment 55. The composition of any one of enumerated embodiments 49-53, wherein the cell is useful in adoptive cell therapy (ACT).

Enumerated Embodiment 56. The composition of enumerated embodiments 55, wherein the cell is a stem cell.

Enumerated Embodiment 57. The composition of enumerated embodiments 56, wherein the stem cell is a hematopoietic stem cell (HSC) or an induced pluripotent stem cell (iPSC).

Enumerated Embodiment 58. The composition of any one of enumerated embodiments 55-57, wherein the cell is an immune cell.

Enumerated Embodiment 59. The composition of enumerated embodiment 58, wherein the immune cell is a leukocyte or a lymphocyte.

Enumerated Embodiment 60. The composition of enumerated embodiment 59, wherein the immune cell is a lymphocyte.

Enumerated Embodiment 61. The composition of enumerated embodiment 60, wherein the lymphocyte is a T cell, a B cell, or an NK cell.

Enumerated Embodiment 62. The composition of enumerated embodiment 60, wherein the lymphocyte is a T cell.

Enumerated Embodiment 63. The composition of enumerated embodiment 62, wherein T cell is a primary T cell.

Enumerated Embodiment 64. The composition of enumerated embodiment 62, wherein T cell is a regulatory T cell.

Enumerated Embodiment 65. The composition of any one of enumerated embodiments 62-64, wherein the lymphocyte is an activated T cell.

Enumerated Embodiment 66. The composition of any one of enumerated embodiments 62-64, wherein the lymphocyte is a non-activated T cell.

Enumerated Embodiment 67. The composition of any one of enumerated embodiments 49-66, wherein the cell is a human cell.

Enumerated Embodiment 68. The composition of any one of enumerated embodiments 48-67, wherein the DNA cutting agent comprises a CRISPR/Cas nuclease component and optionally a guide RNA component.

Enumerated Embodiment 69. The composition of any one of enumerated embodiments 48-68, wherein the DNA cutting agent comprises a CRISPR/Cas nuclease that generates a double strand DNA break or single strand DNA break.

Enumerated Embodiment 70. The composition of any one of enumerated embodiments 48-67, wherein the DNA cutting agent is selected from a zinc finger nuclease, a TALE effector domain nuclease (TALEN), a CRISPR/Cas nuclease component, and combinations thereof.

Enumerated Embodiment 71. The composition of enumerated embodiment 68, wherein the DNA cutting agent is a CRISPR/Cas nuclease component and a guide RNA component.

Enumerated Embodiment 72. The composition of enumerated embodiment 71, wherein the CRISPR/Cas nuclease component comprises a Cas nuclease or an mRNA encoding the Cas nuclease.

Enumerated Embodiment 73. The composition of enumerated embodiment 71, wherein the CRISPR/Cas nuclease component comprises the Cas nuclease.

Enumerated Embodiment 74. The composition of enumerated embodiment 72 or 73, wherein the Cas nuclease is a Class 2, Type II Cas nuclease.

Enumerated Embodiment 75. The composition of enumerated embodiment 74, wherein the Cas nuclease is a Cas9 nuclease.

Enumerated Embodiment 76. The composition of enumerated embodiment 75, wherein the Cas nuclease is a S. pyogenes Cas9 nuclease.

Enumerated Embodiment 77. The composition of enumerated embodiment 72 or 73, wherein the Cas nuclease is a Class 2, Type V Cas nuclease.

Enumerated Embodiment 78. The composition of enumerated embodiment 72 or 73, wherein the Cas nuclease is a Cas12a nuclease.

Enumerated Embodiment 79. The composition of enumerated embodiment 78, wherein the Cas nuclease is a Acidaminococcus sp. Cas12a nuclease.

Enumerated Embodiment 80. The composition of any one of enumerated embodiment 72-79, wherein the Cas nuclease generates a single strand DNA break.

Enumerated Embodiment 81. The composition of any one of enumerated embodiments 48-80, comprising a modified RNA.

Enumerated Embodiment 82. The composition of any one of enumerated embodiments 68-81, wherein the guide RNA component is a guide RNA nucleic acid.

Enumerated Embodiment 83. The composition of enumerated embodiment 82 wherein the guide RNA nucleic acid is a guide RNA (gRNA).

Enumerated Embodiment 84. The composition of enumerated embodiment 82 or 83, wherein the guide RNA nucleic acid is or encodes a dual-guide RNA (dgRNA) composed of a crRNA and tracrRNA.

Enumerated Embodiment 85. The composition of enumerated embodiment 82 or 83, wherein the guide RNA nucleic acid is or encodes a single-guide (sgRNA).

Enumerated Embodiment 86. The composition of any one of enumerated embodiments 83-85, wherein the gRNA is a modified gRNA.

Enumerated Embodiment 87. The composition of enumerated embodiment 86, wherein the cutting agent is Cas9 and the modified gRNA comprises a modification at one or more of the first five nucleotides at the 5′ end.

Enumerated Embodiment 88. The composition of enumerated embodiment 86, wherein the cutting agent is Cas12a and the modified gRNA comprises a DNA/RNA hybrid molecule.

Enumerated Embodiment 89. The composition of enumerated embodiments 86-88, wherein the modified gRNA comprises a modification at one or more of the last five nucleotides at the 3′ end.

Enumerated Embodiment 90. The composition of any one of enumerated embodiments 48-89, wherein the composition comprises a guide RNA nucleic acid and a Class 2, Type II or Class 2, Type V Cas nuclease; and the molar ratio of the guide RNA to Cas nuclease is from about 4:1 to 1:4.

Enumerated Embodiment 91. The composition of any one of enumerated embodiments 50-90, wherein the donor DNA comprises a template comprising a sequence encoding a protein, a regulatory sequence, or a sequence encoding structural RNA.

Enumerated Embodiment 92. The composition of any one of enumerated embodiments 48-91, further comprising a vector.

Enumerated Embodiment 93. The composition of enumerated embodiment 92, wherein the vector encodes the donor DNA.

Enumerated Embodiment 94. The composition of enumerated embodiment 92 or 93, wherein the vector is a viral vector.

Enumerated Embodiment 95. The composition of enumerated embodiment 92 or 93, wherein the vector is a non-viral vector.

Enumerated Embodiment 96. The composition of enumerated embodiment 94, wherein the vector is an AAV.

Enumerated Embodiment 97. The composition of enumerated embodiment 49, wherein the cell is not a cancer cell.

Enumerated Embodiment 98. The composition of any one of enumerated embodiment 48-97, further comprising an inhibitor of the microhomology mediated end joining (MMEJ) pathway.

Enumerated Embodiment 99. A method for targeted genome editing in a cell, comprising contacting the cell with a DNA cutting agent and a DNA-PKI, wherein the DNA-PKI is a compound according to any one of enumerated embodiments 1-46.

Enumerated Embodiment 100. A method of repairing a double stranded DNA break in the genome of a cell, comprising contacting the cell with a DNA cutting agent and a DNA-PKI, wherein the DNA-PKI is a compound according to any one of enumerated embodiments 1-46.

Enumerated Embodiment 101. A method of inhibiting or suppressing repair of a DNA break in a cell via a nonhomologous end joining (NHEJ) pathway, comprising contacting the cell with a DNA cutting agent and a DNA-PKI, wherein the DNA-PKI is a compound according to any one of enumerated embodiments 1-46.

Enumerated Embodiment 102. The method of enumerated embodiment 101 further comprising contacting the cell with an inhibitor of the microhomology mediated end joining (MMEJ) pathway.

Enumerated Embodiment 103. A method of targeted insertion of a donor DNA into the genome of a cell, comprising contacting the cell with a DNA cutting agent, the donor DNA, and a DNA-PKI, wherein the DNA-PKI is a compound according to any one of enumerated embodiments 1-46.

Enumerated Embodiment 104. The method of any one of enumerated embodiments 99-103, comprising growing the cell in a cell medium free of the DNA-PKI and adding the DNA-PKI to the cell medium.

Enumerated Embodiment 105. The method of any one of enumerated embodiments 99-104, comprising contacting the cell with the DNA cutting agent before contacting the cell with the DNA-PKI.

Enumerated Embodiment 106. The method of enumerated embodiment 105, comprising contacting the cell with the DNA-PKI within about six hours of contacting the cell with the DNA cutting agent.

Enumerated Embodiment 107. The method of enumerated embodiment 106, comprising contacting the cell with the DNA-PKI within about three hours of contacting the cell with the DNA cutting agent.

Enumerated Embodiment 108. The method of any one of enumerated embodiments 99-104, comprising contacting the cell with the DNA cutting agent simultaneously with the DNA-PKI.

Enumerated Embodiment 109. The method of any one of enumerated embodiments 99-104, comprising contacting the cell with the DNA cutting agent after contacting the cell with the DNA-PKI.

Enumerated Embodiment 110. The method of any one of enumerated embodiments 99-109, wherein contacting the cell with the DNA cutting agent comprises electroporation.

Enumerated Embodiment 111. The method of enumerated embodiment 109 or 110, comprising contacting the cell with the DNA cutting agent within about three hours of contacting the cell with the DNA-PKI.

Enumerated Embodiment 112. The method of any one of enumerated embodiments 109-111, comprising growing the cell in a cell medium comprising the DNA-PKI.

Enumerated Embodiment 113. The method of any one of enumerated embodiments 109-112, wherein the cell is contacted with the DNA cutting agent and the DNA-PKI for at least about one day.

Enumerated Embodiment 114. The method of enumerated embodiment 113, wherein the cell is contacted with the DNA cutting agent and the DNA-PKI for about one day to about two weeks.

Enumerated Embodiment 115. The method of enumerated embodiment 113, wherein the cell is contacted with the DNA cutting agent and the DNA-PKI for about two weeks.

Enumerated Embodiment 116. The method of any one of enumerated embodiments 99-115, wherein the cell is contacted with the DNA-PKI in a cell medium, wherein the concentration of the DNA-PKI in the cell medium is about 10 μM or less.

Enumerated Embodiment 117. The method of any one of enumerated embodiments 99-116, wherein the cell is contacted with the DNA-PKI in a cell medium, wherein the concentration of the DNA-PKI in the cell medium is from about 0.1-10 μM.

Enumerated Embodiment 118. The method of enumerated embodiment 117, wherein the concentration of the DNA-PKI in the cell medium is from about 0.1-5 μM.

Enumerated Embodiment 119. The method of any one of enumerated embodiments 99-118, wherein the cell is a eukaryotic cell.

Enumerated Embodiment 120. The method of any one of enumerated embodiments 99-119, wherein the cell is for use in adoptive cell therapy (ACT).

Enumerated Embodiment 121. The method of enumerated embodiment 120, wherein the cell is for use in autologous cell therapy.

Enumerated Embodiment 122. The method of enumerated embodiment 120, wherein the cell is for use in allogeneic cell therapy.

Enumerated Embodiment 123. The method of any one of enumerated embodiments 99-119, wherein the cell is a stem cell.

Enumerated Embodiment 124. The method of enumerated embodiment 123, wherein the stem cell is a hematopoietic stem cell (HSC).

Enumerated Embodiment 125. The method of enumerated embodiment 123, wherein the cell is an induced pluripotent stem cell (iPSC).

Enumerated Embodiment 126. The method of enumerated embodiment 120 or 121, wherein the cell is an immune cell.

Enumerated Embodiment 127. The method of enumerated embodiment 126, wherein the immune cell is a leukocyte or a lymphocyte.

Enumerated Embodiment 128. The method of enumerated embodiment 127, wherein the immune cell is a lymphocyte.

Enumerated Embodiment 129. The method of enumerated embodiment 128, wherein the lymphocyte is a T cell, a B cell, or an NK cell.

Enumerated Embodiment 130. The method of enumerated embodiment 129, wherein the lymphocyte is a T cell.

Enumerated Embodiment 131. The method of enumerated embodiment 130, wherein T cell is a primary T cell.

Enumerated Embodiment 132. The method of enumerated embodiment 130, wherein T cell is a regulatory T cell.

Enumerated Embodiment 133. The method of any one of enumerated embodiments 129-132, wherein the lymphocyte is an activated T cell.

Enumerated Embodiment 134. The method of any one of enumerated embodiments 129-132, wherein the lymphocyte is a non-activated T cell.

Enumerated Embodiment 135. The method of any one of enumerated embodiments 99-134, wherein the cell is a human cell.

Enumerated Embodiment 136. The method of any one of enumerated embodiments 99-135, wherein the DNA cutting agent is selected from a zinc finger nuclease, a TALE effector domain nuclease (TALEN), a CRISPR/Cas nuclease component, and combinations thereof.

Enumerated Embodiment 137. The method of enumerated embodiment 136, wherein the DNA cutting agent is a CRISPR/Cas nuclease component.

Enumerated Embodiment 138. The method of enumerated embodiment 137, wherein the CRISPR/Cas nuclease component comprises a Cas nuclease or an mRNA encoding the Cas nuclease.

Enumerated Embodiment 139. The method of enumerated embodiment 138, wherein the CRISPR/Cas nuclease component comprises an mRNA encoding the Cas nuclease.

Enumerated Embodiment 140. The method of enumerated embodiment 138 or 139, wherein the Cas nuclease is a Class 2, Type II Cas nuclease.

Enumerated Embodiment 141. The method of enumerated embodiment 138-139, wherein the Cas nuclease is a Class 2, Type V Cas nuclease.

Enumerated Embodiment 142. The method of enumerated embodiment 140, wherein the Cas nuclease is a Cas9 nuclease.

Enumerated Embodiment 143. The method of enumerated embodiment 142, wherein the Cas nuclease is a S. pyogenes Cas9 nuclease.

Enumerated Embodiment 144. The method of enumerated embodiment 141, wherein the Cas nuclease is a Cas12a nuclease.

Enumerated Embodiment 145. The method of any one of enumerated embodiments 99-144, further comprising contacting the cell with a modified RNA.

Enumerated Embodiment 146. The method of any one of enumerated embodiments 99-145, further comprising contacting the cell with a guide RNA nucleic acid.

Enumerated Embodiment 147. The method of enumerated embodiment 146, wherein the guide RNA nucleic acid is a gRNA.

Enumerated Embodiment 148. The method of enumerated embodiment 146 or 147, wherein the guide RNA nucleic acid is or encodes a dual-guide RNA (dgRNA).

Enumerated Embodiment 149. The method of enumerated embodiment 146 or 147, wherein the guide RNA nucleic acid is or encodes a single-guide (sgRNA).

Enumerated Embodiment 150. The method of any one of enumerated embodiments 147-149, wherein the gRNA is a modified gRNA.

Enumerated Embodiment 151. The method of enumerated embodiment 150, wherein the modified gRNA comprises a modification at one or more of the first five nucleotides at the 5′ end.

Enumerated Embodiment 152. The method of enumerated embodiment 150 or 151, wherein the modified gRNA comprises a modification at one or more of the last five nucleotides at the 3′ end.

Enumerated Embodiment 153. The method of any one of enumerated embodiments 146-152, wherein the DNA cutting agent is a Class 2, Type II Cas nuclease or Class 2, Type V Cas nuclease mRNA; and the ratio of the guide RNA nucleic acid to Cas nuclease is from about 4:1 to 1:4 by molar ratio.

Enumerated Embodiment 154. The method of any one of enumerated embodiments 99-153, further comprising contacting the cell with a donor DNA.

Enumerated Embodiment 155. The method of enumerated 154, comprising contacting the cell with a vector comprising the donor DNA.

Enumerated Embodiment 156. The method enumerated embodiment 154 or 155, wherein the donor DNA comprises a template comprising a sequence encoding a protein, a regulatory sequence, or a sequence encoding structural RNA.

Enumerated Embodiment 157. The method of enumerated embodiment 156, wherein the template sequence is integrated into the genome of the cell via homology directed repair (HDR).

Enumerated Embodiment 158. The method of any one of enumerated embodiments 99-157, further comprising contacting the cell with a vector.

Enumerated Embodiment 159. The method of enumerated embodiment 158, wherein the vector encodes the DNA cutting agent.

Enumerated Embodiment 160. The method of enumerated embodiment 158 or 159, wherein the vector encodes a donor DNA.

Enumerated Embodiment 161. The method of any one of enumerated embodiments 158-160, wherein the vector is a viral vector.

Enumerated Embodiment 162. The method of any one of enumerated 158-160, wherein the vector is a non-viral vector.

Enumerated Embodiment 163. The method of enumerated embodiment 161, wherein the vector is an AAV.

Enumerated Embodiment 164. The method of any one of enumerated embodiments 99-163, wherein the DNA cutting agent interacts with a target sequence within the genome of the cell, resulting in a double stranded DNA break (DSB).

Enumerated Embodiment 165. The method of any one of enumerated embodiments 99-164, wherein the method results in a gene knockout.

Enumerated Embodiment 166. The method of any one of enumerated embodiments 99-165, wherein the method results in a gene correction.

Enumerated Embodiment 167. The method of any one of enumerated embodiments 99-166, wherein the method results in a gene insertion.

Enumerated Embodiment 168. The method of any one of enumerated embodiments 156-167, wherein the donor DNA comprises a template comprising an exogenous nucleic acid encoding a protein.

Enumerated Embodiment 169. The method of enumerated embodiment 168, wherein the protein is selected from the group consisting of a cytokine, an immunosuppressor, an antibody, a receptor, and an enzyme.

Enumerated Embodiment 170. The method of enumerated embodiment 169, wherein the protein is a receptor.

Enumerated Embodiment 171. The method of enumerated embodiment 169 or 170, wherein the receptor is selected from the group consisting of an immunological receptor, a T-cell receptor (TCR), and a chimeric antigen receptor.

Enumerated Embodiment 172. The method of enumerated embodiment 171, wherein the receptor is an immunological receptor.

Enumerated Embodiment 173. The method of enumerated embodiment 171, wherein the receptor is a TCR.

Enumerated Embodiment 174. The method of enumerated embodiment 168, wherein the exogenous nucleic acid encodes a TCR alpha chain and/or a TCR beta chain.

Enumerated Embodiment 175. The method of enumerated embodiment 171, wherein the receptor a chimeric antigen receptor.

Enumerated Embodiment 176. The method of any one of enumerated embodiments 156-175, wherein the DNA cutting agent interacts with a target sequence within the TRAC gene of the T-cell.

Enumerated Embodiment 177. The method of enumerated embodiment 176, comprising contacting the cell with at least two different DNA cutting agents that target different loci.

Enumerated Embodiment 178. The method of any one of enumerated embodiments 168-177, wherein the template comprises a first homology arm and a second homology arm that are complementary to sequences located upstream and downstream of the cleavage site, respectively.

1.-8. (canceled)

9. A compound selected from the group consisting of: 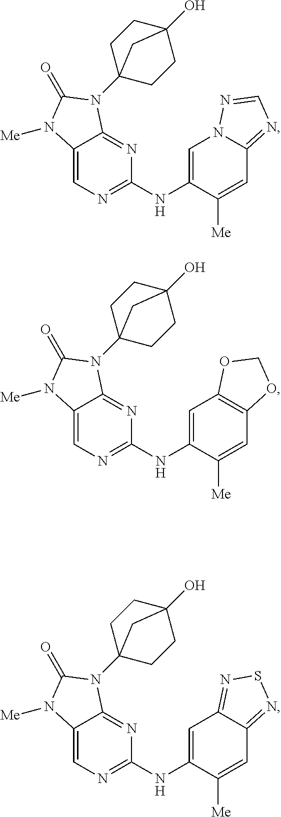 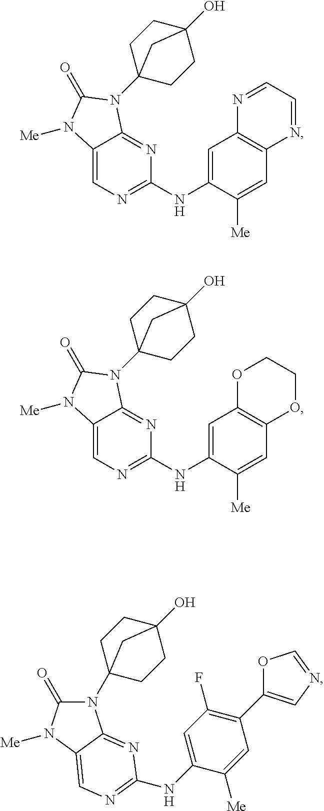 [SMILES: Cc1cc2nccnc2cc1Nc1ncc2c(n1)n(C13CCC(O)(CC1)C3)c(=O)n2C ; Cc1cc2c(cc1Nc1ncc3c(n1)n(C14CCC(O)(CC1)C4)c(=O)n3C)OCCO2 ; Cc1cc(-c2cnco2)c(F)cc1Nc1ncc2c(n1)n(C13CCC(O)(CC1)C3)c(=O)n2C] 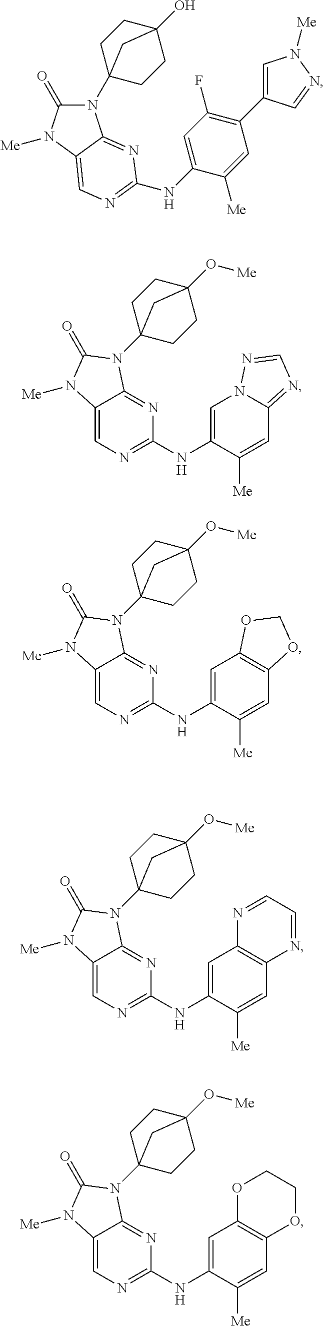 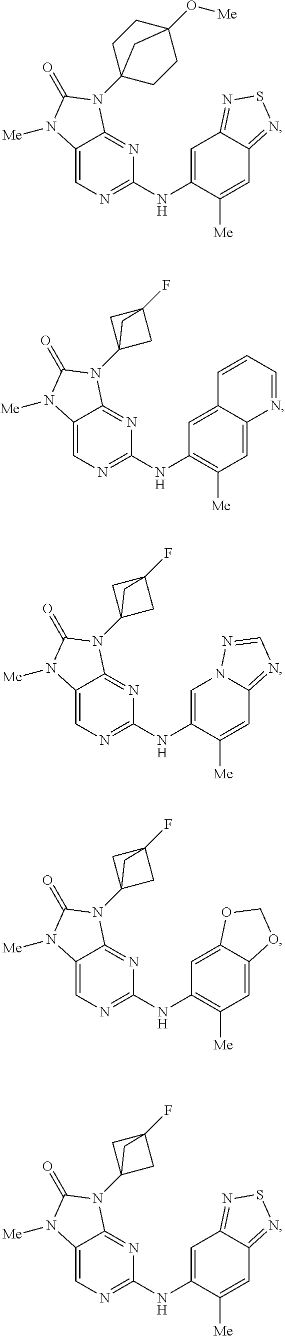 [SMILES: COC12CCC(n3c(=O)n(C)c4cnc(Nc5cc6nsnc6cc5C)nc43)(CC1)C2 ; Cc1cc2ncccc2cc1Nc1ncc2c(n1)n(C13CC(F)(C1)C3)c(=O)n2C ; Cc1cc2ncnn2cc1Nc1ncc2c(n1)n(C13CC(F)(C1)C3)c(=O)n2C ; Cc1cc2c(cc1Nc1ncc3c(n1)n(C14CC(F)(C1)C4)c(=O)n3C)OCO2 ; Cc1cc2nsnc2cc1Nc1ncc2c(n1)n(C13CC(F)(C1)C3)c(=O)n2C] 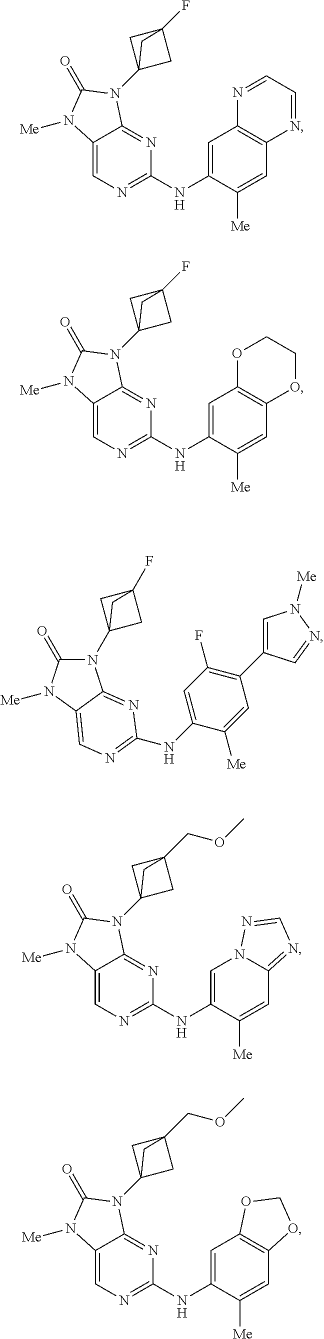 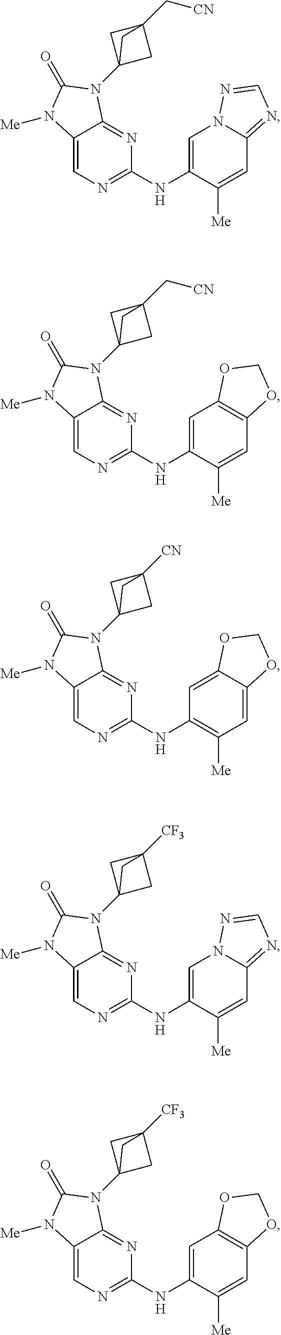 [SMILES: Cc1cc2ncnn2cc1Nc1ncc2c(n1)n(C13CC(CC#N)(C1)C3)c(=O)n2C ; Cc1cc2c(cc1Nc1ncc3c(n1)n(C14CC(CC#N)(C1)C4)c(=O)n3C)OCO2 ; Cc1cc2c(cc1Nc1ncc3c(n1)n(C14CC(C#N)(C1)C4)c(=O)n3C)OCO2 ; Cc1cc2ncnn2cc1Nc1ncc2c(n1)n(C13CC(C(F)(F)F)(C1)C3)c(=O)n2C ; Cc1cc2c(cc1Nc1ncc3c(n1)n(C14CC(C(F)(F)F)(C1)C4)c(=O)n3C)OCO2] 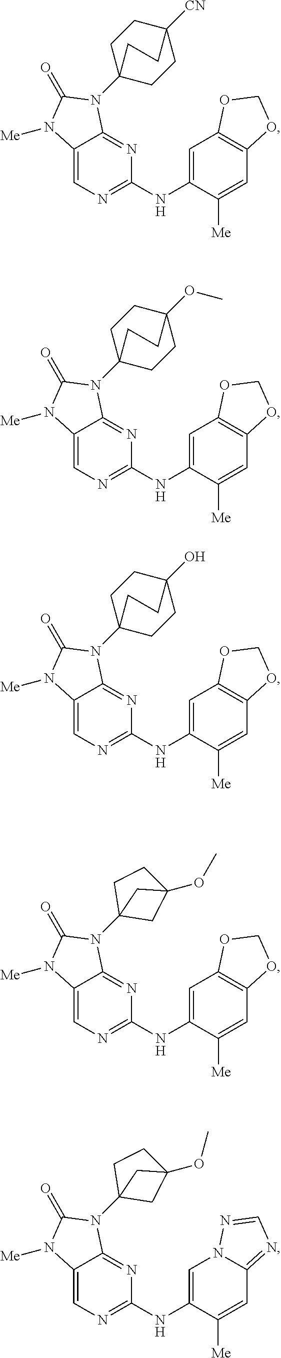 [SMILES: Cc1cc2c(cc1Nc1ncc3c(n1)n(C14CCC(C#N)(CC1)CC4)c(=O)n3C)OCO2 ; COC12CCC(n3c(=O)n(C)c4cnc(Nc5cc6c(cc5C)OCO6)nc43)(CC1)CC2 ; Cc1cc2c(cc1Nc1ncc3c(n1)n(C14CCC(O)(CC1)CC4)c(=O)n3C)OCO2 ; COC12CCC(n3c(=O)n(C)c4cnc(Nc5cc6c(cc5C)OCO6)nc43)(C1)C2 ; COC12CCC(n3c(=O)n(C)c4cnc(Nc5cn6ncnc6cc5C)nc43)(C1)C2] 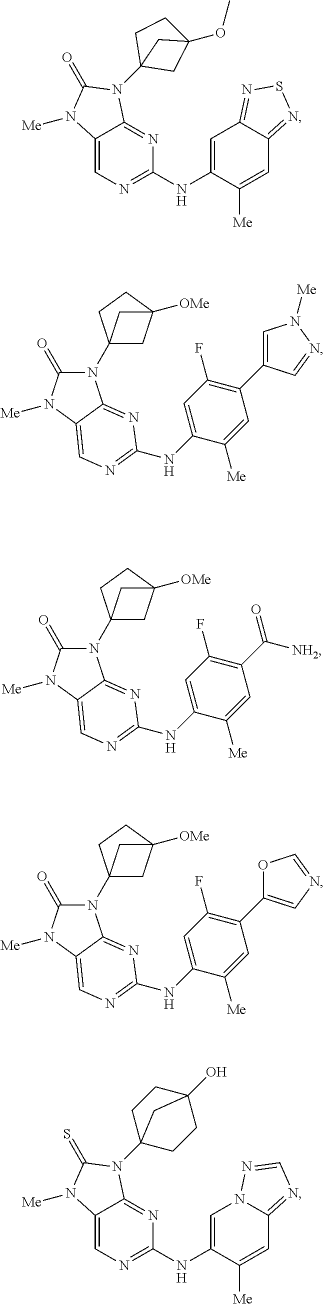 [SMILES: COC12CCC(n3c(=O)n(C)c4cnc(Nc5cc6nsnc6cc5C)nc43)(C1)C2 ; COC12CCC(n3c(=O)n(C)c4cnc(Nc5cc(F)c(-c6cnn(C)c6)cc5C)nc43)(C1)C2 ; COC12CCC(n3c(=O)n(C)c4cnc(Nc5cc(F)c(C(N)=O)cc5C)nc43)(C1)C2 ; COC12CCC(n3c(=O)n(C)c4cnc(Nc5cc(F)c(-c6cnco6)cc5C)nc43)(C1)C2 ; Cc1cc2ncnn2cc1Nc1ncc2c(n1)n(C13CCC(O)(CC1)C3)c(=S)n2C] 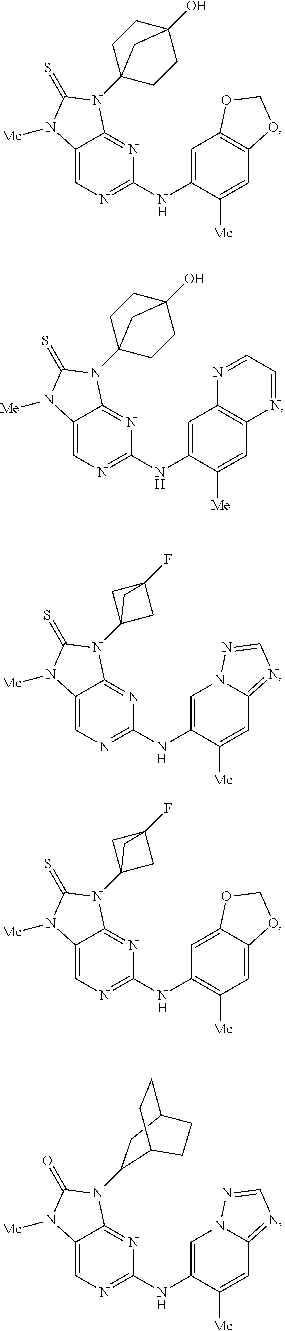 [SMILES: Cc1cc2c(cc1Nc1ncc3c(n1)n(C14CCC(O)(CC1)C4)c(=S)n3C)OCO2 ; Cc1cc2nccnc2cc1Nc1ncc2c(n1)n(C13CCC(O)(CC1)C3)c(=S)n2C ; Cc1cc2ncnn2cc1Nc1ncc2c(n1)n(C13CC(F)(C1)C3)c(=S)n2C ; Cc1cc2c(cc1Nc1ncc3c(n1)n(C14CC(F)(C1)C4)c(=S)n3C)OCO2 ; Cc1cc2ncnn2cc1Nc1ncc2c(n1)n(C1CC3CCC1CC3)c(=O)n2C] 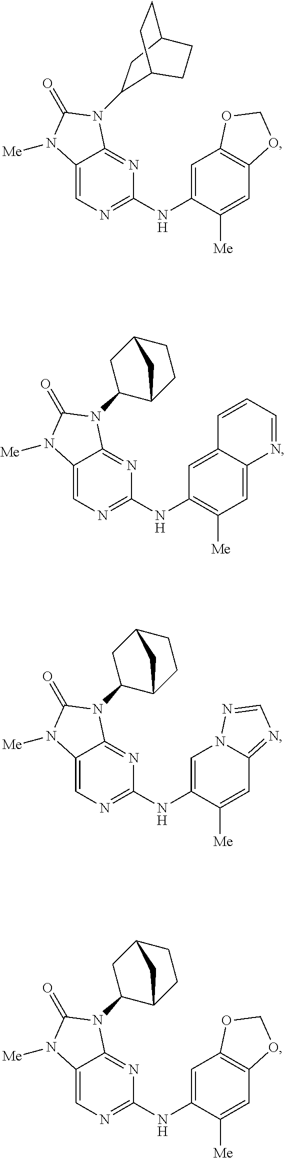 [SMILES: Cc1cc2c(cc1Nc1ncc3c(n1)n(C1CC4CCC1CC4)c(=O)n3C)OCO2 ; Cc1cc2ncccc2cc1Nc1ncc2c(n1)n([C@H]1C[C@@H]3CC[C@H]1C3)c(=O)n2C ; Cc1cc2ncnn2cc1Nc1ncc2c(n1)n([C@H]1C[C@@H]3CC[C@H]1C3)c(=O)n2C ; Cc1cc2c(cc1Nc1ncc3c(n1)n([C@H]1C[C@@H]4CC[C@H]1C4)c(=O)n3C)OCO2] 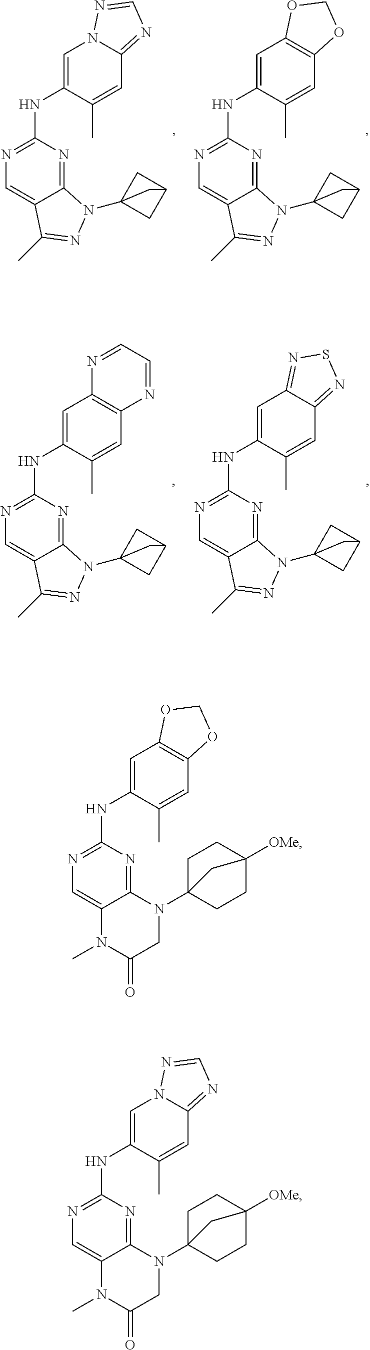 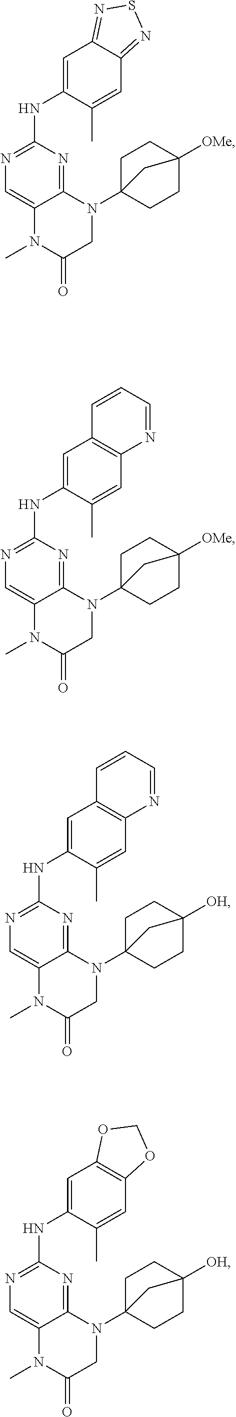 [SMILES: COC12CCC(N3CC(=O)N(C)c4cnc(Nc5cc6nsnc6cc5C)nc43)(CC1)C2 ; COC12CCC(N3CC(=O)N(C)c4cnc(Nc5cc6cccnc6cc5C)nc43)(CC1)C2 ; Cc1cc2ncccc2cc1Nc1ncc2c(n1)N(C13CCC(O)(CC1)C3)CC(=O)N2C ; Cc1cc2c(cc1Nc1ncc3c(n1)N(C14CCC(O)(CC1)C4)CC(=O)N3C)OCO2] 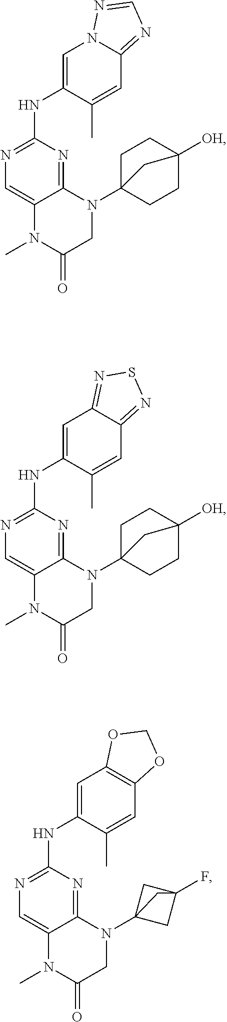 [SMILES: Cc1cc2ncnn2cc1Nc1ncc2c(n1)N(C13CCC(O)(CC1)C3)CC(=O)N2C ; Cc1cc2nsnc2cc1Nc1ncc2c(n1)N(C13CCC(O)(CC1)C3)CC(=O)N2C ; Cc1cc2c(cc1Nc1ncc3c(n1)N(C14CC(F)(C1)C4)CC(=O)N3C)OCO2] 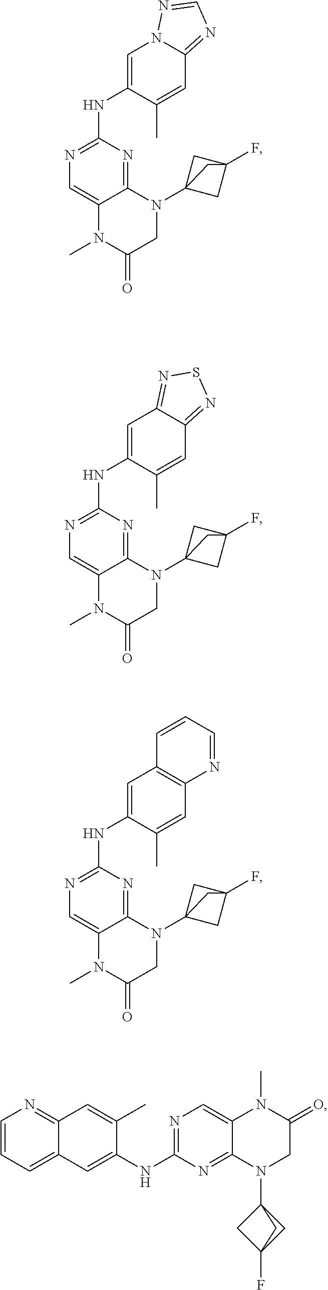 [SMILES: Cc1cc2ncnn2cc1Nc1ncc2c(n1)N(C13CC(F)(C1)C3)CC(=O)N2C ; Cc1cc2nsnc2cc1Nc1ncc2c(n1)N(C13CC(F)(C1)C3)CC(=O)N2C ; Cc1cc2ncccc2cc1Nc1ncc2c(n1)N(C13CC(F)(C1)C3)CC(=O)N2C ; Cc1cc2ncccc2cc1Nc1ncc2c(n1)N(C13CC(F)(C1)C3)CC(=O)N2C] 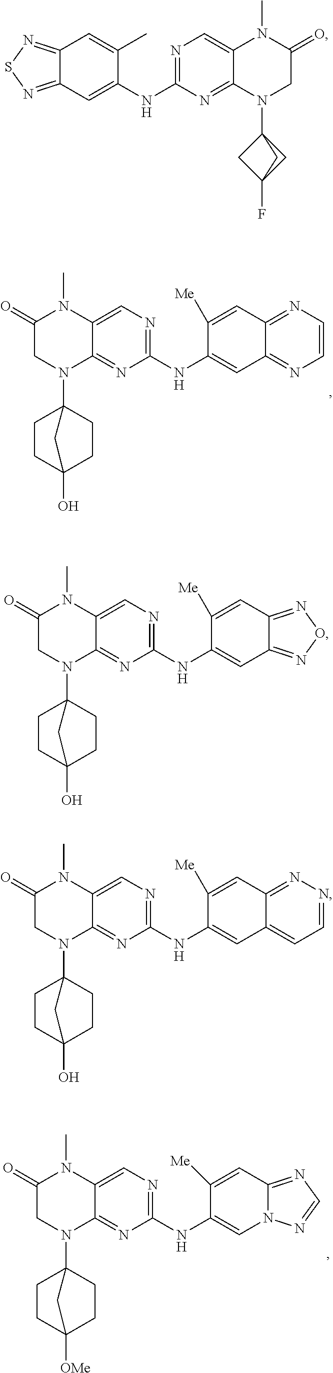 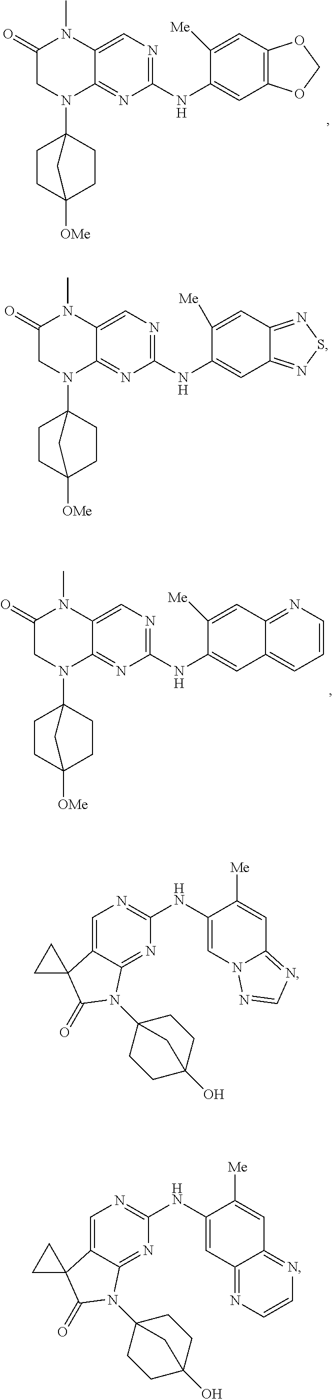 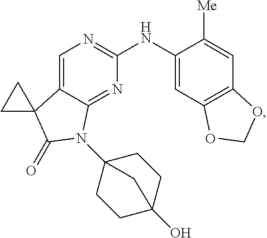 [SMILES: Cc1cc2c(cc1Nc1ncc3c(n1)N(C14CCC(O)(CC1)C4)C(=O)C31CC1)OCO2] 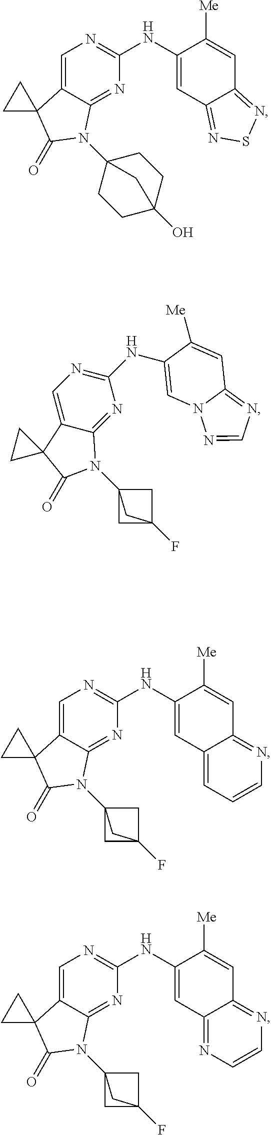 [SMILES: Cc1cc2nsnc2cc1Nc1ncc2c(n1)N(C13CCC(O)(CC1)C3)C(=O)C21CC1 ; Cc1cc2ncnn2cc1Nc1ncc2c(n1)N(C13CC(F)(C1)C3)C(=O)C21CC1 ; Cc1cc2ncccc2cc1Nc1ncc2c(n1)N(C13CC(F)(C1)C3)C(=O)C21CC1 ; Cc1cc2nccnc2cc1Nc1ncc2c(n1)N(C13CC(F)(C1)C3)C(=O)C21CC1] 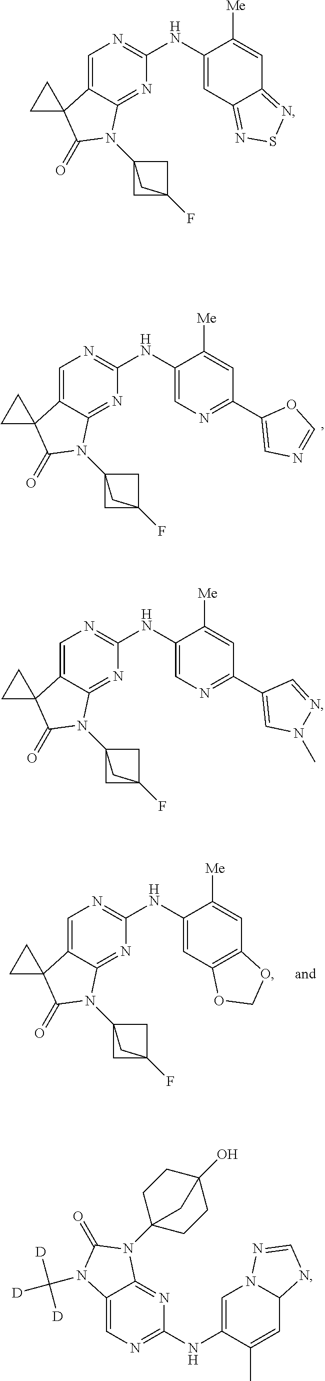
or a pharmaceutically acceptable salt, isomer, solvate, prodrug, or tautomer thereof.

10. The compound of claim 9, wherein the compound is: 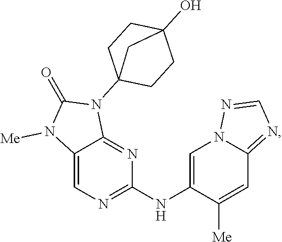 [SMILES: Cc1cc2ncnn2cc1Nc1ncc2c(n1)n(C13CCC(O)(CC1)C3)c(=O)n2C]
or a pharmaceutically acceptable salt, isomer, solvate, prodrug, or tautomer thereof.

11. A composition comprising:
a) a DNA protein kinase inhibitor (DNA-PKI); and
b) a DNA cutting agent;
wherein the DNA-PKI is a compound according to claim 9.

12. The composition of claim 11, further comprising a cell.

13. The composition of claim 12, further comprising a donor DNA.

14. The composition of claim 13, wherein the DNA cutting agent comprises a CRISPR/Cas nuclease component and optionally a guide RNA component.

15. The composition of claim 13, further comprising a vector.

16. The composition of claim 15, further comprising an inhibitor of the microhomology mediated end joining (MMEJ) pathway.

17. A method for targeted genome editing in a cell, comprising contacting the cell with a DNA cutting agent and a DNA-PKI, wherein the DNA-PKI is a compound according to claim 9.

18. A method of repairing a double stranded DNA break in the genome of a cell, comprising contacting the cell with a DNA cutting agent and a DNA-PKI, wherein the DNA-PKI is a compound according to claim 9.

19. A method of inhibiting or suppressing repair of a DNA break in a cell via a nonhomologous end joining (NHEJ) pathway, comprising contacting the cell with a DNA cutting agent and a DNA-PKI, wherein the DNA-PKI is a compound according to claim 9.

20. A method of targeted insertion of a donor DNA into the genome of a cell, comprising contacting the cell with a DNA cutting agent, the donor DNA, and a DNA-PKI, wherein the DNA-PKI is a compound according to claim 9.


SEQUENCE LISTING (199 sequences)

SEQ ID NO: 1-5 - skipped (5 entries)

SEQ ID NO: 6 - protein, 127 aa, synthetic construct
MEAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 7-15 - skipped (9 entries)

SEQ ID NO: 16 - protein, 30 aa, synthetic construct
SAWSHPQFEKGGGSGGGSGGSAWSHPQFEK

SEQ ID NO: 17-27 - skipped (11 entries)

SEQ ID NO: 28 - RNA, 20 nt, synthetic construct
  source 1..20: mol_type = other RNA
gagaatcaaaatcggtgaat

SEQ ID NO: 29 - protein, 228 aa, Homo sapiens
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - skipped

SEQ ID NO: 31 - protein, 119 aa, synthetic construct [sequence omitted]

SEQ ID NO: 32 - protein, 106 aa, synthetic construct [sequence omitted]

SEQ ID NO: 33 - protein, 116 aa, synthetic construct
LQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTV

SEQ ID NO: 34 - protein, 108 aa, synthetic construct
DIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKR

SEQ ID NO: 35 - skipped

SEQ ID NO: 36 - protein, 118 aa, synthetic construct
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 37 - protein, 105 aa, synthetic construct
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 38-55 - skipped (18 entries)

SEQ ID NO: 56 - protein, 5 aa, synthetic construct
DYYVY

SEQ ID NO: 57 - protein, 17 aa, synthetic construct
WINPNSGGTNYAQKFQG

SEQ ID NO: 58 - protein, 9 aa, synthetic construct
SQRDGYMDY

SEQ ID NO: 59 - protein, 8 aa, synthetic construct
TGTSSDVG

SEQ ID NO: 60 - protein, 7 aa, synthetic construct
EDSKRPS

SEQ ID NO: 61 - protein, 10 aa, synthetic construct
SSNTRSSTLV

SEQ ID NO: 62-178 - skipped (117 entries)

SEQ ID NO: 179 - DNA, 684 bp, synthetic construct
  source 1..684: mol_type = other DNA
gaatctaagtacggaccgccctgccctccctgccctgctcctcctgtggctggaccaagcgtgttcctgtttccacctaagcctaaagataccctgatgatttcccgcacacctgaagtgacttgcgtggtcgtggacgtgagccaggaggatccagaagtgcagttcaactggtacgtggacggcgtggaagtccacaatgctaagactaaaccccgagaggaacagtttcagtcaacttaccgggtcgtgagcgtgctgaccgtcctgcatcaggattggctgaacgggaaggagtataagtgcaaagtgtctaataagggactgcctagctccatcgagaaaacaattagtaaggcaaaagggcagcctcgagaaccacaggtgtataccctgccccctagccaggaggaaatgaccaagaaccaggtgtccctgacatgtctggtcaaaggcttctatccaagtgacatcgccgtggagtgggaatcaaatgggcagcccgagaacaattacaagaccacaccacccgtgctggactctgatggaagtttctttctgtattccaggctgaccgtggataaatctcgctggcaggagggcaacgtgttctcttgcagtgtcatgcacgaagccctgcacaatcattatacacagaagtcactgagcctgtccctgggcaaa

SEQ ID NO: 180 - protein, 244 aa, synthetic construct
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 181-182 - skipped (2 entries)

SEQ ID NO: 183 - DNA, 684 bp, synthetic construct
  source 1..684: mol_type = other DNA
gagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaag

SEQ ID NO: 184 - protein, 228 aa, synthetic construct; identical to SEQ ID NO: 29

SEQ ID NO: 185 - DNA, 84 bp, synthetic construct
  source 1..84: mol_type = other DNA
atgttctgggtgctcgtggtcgttggcggagtgctggcctgttacagcctgctggttaccgtggccttcatcatcttttgggtc

SEQ ID NO: 186 - protein, 28 aa, synthetic construct
MFWVLVVVGGVLACYSLLVTVAFIIFWV

SEQ ID NO: 187 - DNA, 126 bp, synthetic construct
  source 1..126: mol_type = other DNA
aagcggggcagaaagaagctgctctacatcttcaagcagcccttcatgcggcccgtgcagaccacacaagaggaagatggctgctcctgcagattccccgaggaagaagaaggcggctgcgagctg

SEQ ID NO: 188 - protein, 42 aa, synthetic construct
KRGRKKLLYIFKQPFMRPVQTTQEEDGCSCRFPEEEEGGCEL

SEQ ID NO: 189 - DNA, 336 bp, synthetic construct
  source 1..336: mol_type = other DNA
agagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 190 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 191 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 192 - DNA, 60 bp, Homo sapiens
  source 1..60: mol_type = unassigned DNA
atggtgctgcagacccaggtgttcatcagcctgctgctgtggatctctggcgcctatgga

SEQ ID NO: 193 - protein, 15 aa, synthetic construct
GGGGSGGGGSGGGGS

SEQ ID NO: 194 - DNA, 133 bp, synthetic construct
  source 1..133: mol_type = other DNA
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 195 - DNA, 611 bp, synthetic construct
  source 1..611: mol_type = other DNA
ctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 196 - DNA, 628 bp, synthetic construct
  source 1..628: mol_type = other DNA
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtg

SEQ ID NO: 197 - DNA, 2025 bp, synthetic construct
  source 1..2025: mol_type = other DNA
atggtgctgcagacccaggtgttcatcagcctgctgctgtggatctctggcgcctatggacagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctagataa

SEQ ID NO: 198 - protein, 674 aa, synthetic construct [sequence omitted]

SEQ ID NO: 199 - DNA, 3978 bp, synthetic construct
  source 1..3978: mol_type = other DNA
ctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgatggatccggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccaccatggtgctgcagacccaggtgttcatcagcctgctgctgtggatctctggcgcctatggacagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctagataaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaagggccctttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtg